(19) United States
(12) Patent Application Publication
(10) Pub. No.: US 2016/0186203 A1
(43) Pub. Date: Jun. 30, 2016

(54) GHO/SEC24B2 AND SEC24B1 NUCLEIC ACID MOLECULES TO CONTROL COLEOPTERAN AND HEMIPTERAN PESTS

(71) Applicants: DOW AGROSCIENCES LLC, Indianapolis, IN (US); THE BOARD OF REGENTS OF THE UNIVERSITY OF NEBRASKA, Lincoln, NE (US)

(72) Inventors: Kenneth E. Narva, Zionsville, IN (US); Kanika Arora, West New York, NJ (US); Sarah Worden, Indianapolis, IN (US); Murugesan Rangasamy, Zionsville, IN (US); Huarong Li, Zionsville, IN (US); Meghan Frey, Indianapolis, IN (US); Blair Siegfried, Lincoln, NE (US); Chitvan Khajuria, Ballwin, MO (US); Elane Fishilevich, Indianapolis, IN (US)

(21) Appl. No.: 14/878,852

(22) Filed: Oct. 8, 2015

Related U.S. Application Data
(60) Provisional application No. 62/061,608, filed on Oct. 8, 2014.

(51) Int. Cl. C12N 15/82; C07K 14/325; C12N 15/113

(52) U.S. Cl. CPC C12N 15/8286; C12N 15/113; C07K 14/325

(57) ABSTRACT

This disclosure concerns nucleic acid molecules and methods of use thereof for control of insect pests through RNA interference-mediated inhibition of target coding and transcribed non-coding sequences in insect pests, including coleopteran and/or hemipteran pests. The disclosure also concerns methods for making transgenic plants that express nucleic acid molecules useful for the control of insect pests, and the plant cells and plants obtained thereby.

### CROSS-REFERENCE TO RELATED APPLICATION

This application claim priority to U.S. Patent Application Ser. No. 62/061,608 filed Oct. 8, 2014, the disclosure of which is hereby incorporated herein in its entirety by this reference.

### FIELD OF THE DISCLOSURE

The present invention relates generally to genetic control of plant damage caused by insect pests (e.g., coleopteran pests and hemipteran pests). In particular embodiments, the present invention relates to identification of target coding and non-coding polynucleotides, and the use of recombinant DNA technologies for post-transcriptionally repressing or inhibiting expression of target coding and non-coding polynucleotides in the cells of an insect pest to provide a plant protective effect.

### BACKGROUND

The western corn rootworm (WCR), Diabrotica virgifera virgifera LeConte, is one of the most devastating corn rootworm species in North America and is a particular concern in corn-growing areas of the Midwestern United States. The northern corn rootworm (NCR), Diabrotica barberi Smith and Lawrence, is a closely-related species that co-inhabits much of the same range as WCR. There are several other related subspecies of Diabrotica that are significant pests in North America: the Mexican corn rootworm (MCR), D. virgifera zeae Krysan and Smith; the southern corn rootworm (SCR), D. undecimpunctata howardi Barber; D. balteata LeConte; D. undecimpunctata tenella; and D. u. undecimpunctata Mannerheim. The United States Department of Agriculture estimates that corn rootworms cause $1 billion in lost revenue each year, including $800 million in yield loss and $200 million in treatment costs.

Both WCR and NCR are deposited in the soil as eggs during the summer. The insects remain in the egg stage throughout the winter. The eggs are oblong, white, and less than 0.004 inches in length. The larvae hatch in late May or early June, with the precise timing of egg hatching varying from year to year due to temperature differences and location. The newly hatched larvae are white worms that are less than 0.125 inches in length. Once hatched, the larvae begin to feed on corn roots. Corn rootworms go through three larval instars. After feeding for several weeks, the larvae molt into the pupal stage. They pupate in the soil, and then they emerge from the soil as adults in July and August. Adult rootworms are about 0.25 inches in length.

Corn rootworm larvae complete development on corn and several other species of grasses. Larvae reared on yellow foxtail emerge later and have a smaller head capsule size as adults than larvae reared on corn. Ellsbury et al. (2005) Environ. Entomol. 34:627-634. WCR adults feed on corn silk, pollen, and kernels on exposed ear tips. If WCR adults emerge before corn reproductive tissues are present, they may feed on leaf tissue, thereby slowing plant growth and occasionally killing the host plant. However, the adults will quickly shift to preferred silks and pollen when they become available. NCR adults also feed on reproductive tissues of the corn plant, but in contrast rarely feed on corn leaves.

Most of the rootworm damage in corn is caused by larval feeding. Newly hatched rootworms initially feed on fine corn root hairs and burrow into root tips. As the larvae grow larger, they feed on and burrow into primary roots. When corn rootworms are abundant, larval feeding often results in the pruning of roots all the way to the base of the corn stalk. Severe root injury interferes with the roots' ability to transport water and nutrients into the plant, reduces plant growth, and results in reduced grain production, thereby often drastically reducing overall yield. Severe root injury also often results in lodging of corn plants, which makes harvest more difficult and further decreases yield. Furthermore, feeding by adults on the corn reproductive tissues can result in pruning of silks at the ear tip. If this “silk clipping” is severe enough during pollen shed, pollination may be disrupted.

Control of corn rootworms may be attempted by crop rotation, chemical insecticides, biopesticides (e.g., the spore-forming gram-positive bacterium, Bacillus thuringiensis), or a combination thereof. Crop rotation suffers from the significant disadvantage of placing unwanted restrictions upon the use of farmland. Moreover, oviposition of some rootworm species may occur in crop fields other than corn or extended diapauses results in egg hatching over multiple years, thereby mitigating the effectiveness of crop rotation practiced with corn and soybean.

Chemical insecticides are the most heavily relied upon strategy for achieving corn rootworm control. Chemical insecticide use, though, is an imperfect corn rootworm control strategy; over $1 billion may be lost in the United States each year due to corn rootworm when the costs of the chemical insecticides are added to the costs of the rootworm damage that may occur despite the use of the insecticides. High populations of larvae, heavy rains, and improper application of the insecticide(s) may all result in inadequate corn rootworm control. Furthermore, the continual use of insecticides may select for insecticide-resistant rootworm strains, as well as raise significant environmental concerns due to the toxicity of many of them to non-target species.

Stink bugs and other hemipteran insects (heteroptera) comprise another important agricultural pest complex. Worldwide over 50 closely related species of stink bugs are known to cause crop damage. McPherson & McPherson (2000) Stink bugs of economic importance in America north of Mexico, CRC Press. These insects are present in a large number of important crops including maize, soybean, fruit, vegetables, and cereals.

Stink bugs go through multiple nymph stages before reaching the adult stage. The time to develop from eggs to adults is about 30-40 days. Both nymphs and adults feed on sap from soft tissues into which they also inject digestive enzymes causing extra-oral tissue digestion and necrosis. Digested plant material and nutrients are then ingested. Depletion of water and nutrients from the plant vascular system results in plant tissue damage. Damage to developing grain and seeds is the most significant as yield and germination are significantly reduced. Multiple generations occur in warm climates resulting in significant insect pressure. Current management of stink bugs relies on insecticide treatment on an individual field basis. Therefore, alternative management strategies are urgently needed to minimize ongoing crop losses.

RNA interference (RNAi) is a process utilizing endogenous cellular pathways, whereby an interfering RNA (iRNA) molecule (e.g., a double-stranded RNA (dsRNA) molecule) that is specific for all, or any portion of adequate size, of a target gene sequence results in the degradation of the mRNA encoded thereby. In recent years, RNAi has been used to perform gene “knockdown” in a number of species and experimental systems; for example, Caenorhabditis elegans, plants, insect embryos, and cells in tissue culture. See, e.g., Fire et al. (1998) Nature 391:806-811; Martinez et al. (2002) Cell 110:563-574; McManus and Sharp (2002) Nature Rev. Genetics 3:737-747.

RNAi accomplishes degradation of mRNA through an endogenous pathway including the DICER protein complex. DICER cleaves long dsRNA molecules into short fragments of approximately 20 nucleotides, termed small interfering RNA (siRNA). The siRNA is unwound into two single-stranded RNAs: the passenger strand and the guide strand. The passenger strand is degraded, and the guide strand is incorporated into the RNA-induced silencing complex (RISC). Post-transcriptional gene silencing occurs when the guide strand binds specifically to a complementary mRNA molecule and induces cleavage by Argonaute, the catalytic component of the RISC complex. This process is known to spread systemically throughout some eukaryotic organisms despite initially limited concentrations of siRNA and/or miRNA such as plants, nematodes, and some insects.

U.S. Pat. No. 7,612,194 and U.S. Patent Publication Nos. 2007/0050860, 2010/0192265, and 2011/0154545 disclose a library of 9112 expressed sequence tag (EST) sequences isolated from D. v. virgifera LeConte pupae. It is suggested in U.S. Pat. No. 7,612,194 and U.S. Patent Publication No. 2007/0050860 to operably link to a promoter a nucleic acid molecule that is complementary to one of several particular partial sequences of D. v. virgifera vacuolar-type H⁺-ATPase (V-ATPase) disclosed therein for the expression of anti-sense RNA in plant cells. U.S. Patent Publication No. 2010/0192265 suggests operably linking a promoter to a nucleic acid molecule that is complementary to a particular partial sequence of a D. v. virgifera gene of unknown and undisclosed function (the partial sequence is stated to be 58% identical to C56C10.3 gene product in C. elegans) for the expression of anti-sense RNA in plant cells. U.S. Patent Publication No. 2011/0154545 suggests operably linking a promoter to a nucleic acid molecule that is complementary to two particular partial sequences of D. v. virgifera coatomer beta subunit genes for the expression of anti-sense RNA in plant cells. Further, U.S. Pat. No. 7,943,819 discloses a library of 906 expressed sequence tag (EST) sequences isolated from D. v. virgifera LeConte larvae, pupae, and dissected midguts, and suggests operably linking a promoter to a nucleic acid molecule that is complementary to a particular partial sequence of a D. v. virgifera charged multivesicular body protein 4b gene for the expression of double-stranded RNA in plant cells.

No further suggestion is provided in U.S. Pat. No. 7,612,194, and U.S. Patent Publication Nos. 2007/0050860, 2010/0192265, and 2011/0154545 to use any particular sequence of the more than nine thousand sequences listed therein for RNA interference, other than the several particular partial sequences of V-ATPase and the particular partial sequences of genes of unknown function. Furthermore, none of U.S. Pat. No. 7,612,194, and U.S. Patent Publication Nos. 2007/0050860 and 2010/0192265, and 2011/0154545 provides any guidance as to which other of the over nine thousand sequences provided would be lethal, or even otherwise useful, in species of corn rootworm when used as dsRNA or siRNA. U.S. Pat. No. 7,943,819 provides no suggestion to use any particular sequence of the more than nine hundred sequences listed therein for RNA interference, other than the particular partial sequence of a charged multivesicular body protein 4b gene. Furthermore, U.S. Pat. No. 7,943,819 provides no guidance as to which other of the over nine hundred sequences provided would be lethal, or even otherwise useful, in species of corn rootworm when used as dsRNA or siRNA. U.S. Patent Application Publication No. U.S. 2013/040173 and PCT Application Publication No. WO 2013/169923 describe the use of a sequence derived from a Diabrotica virgifera Snf7 gene for RNA interference in maize. (Also disclosed in Bolognesi et al. (2012) PLOS ONE 7(10): e47534. doi:10.1371/journal.pone.0047534).

The overwhelming majority of sequences complementary to corn rootworm DNAs (such as the foregoing) do not provide a plant protective effect from species of corn rootworm when used as dsRNA or siRNA. For example, Baum et al. (2007) Nature Biotechnology 25:1322-1326, describes the effects of inhibiting several WCR gene targets by RNAi. These authors reported that 8 of the 26 target genes they tested were not able to provide experimentally significant coleopteran pest mortality at a very high iRNA (e.g., dsRNA) concentration of more than 520 ng/cm².

The authors of U.S. Pat. No. 7,612,194 and U.S. Patent Publication No. 2007/0050860 made the first report of in planta RNAi in corn plants targeting the western corn rootworm. Baum et al. (2007) Nat. Biotechnol. 25(11):1322-6. These authors describe a high-throughput in vivo dietary RNAi system to screen potential target genes for developing transgenic RNAi maize. Of an initial gene pool of 290 targets, only 14 exhibited larval control potential. One of the most effective double-stranded RNAs (dsRNA) targeted a gene encoding vacuolar ATPase subunit A (V-ATPase), resulting in a rapid suppression of corresponding endogenous mRNA and triggering a specific RNAi response with low concentrations of dsRNA. Thus, these authors documented for the first time the potential for in planta RNAi as a possible pest management tool, while simultaneously demonstrating that effective targets could not be accurately identified a priori, even from a relatively small set of candidate genes.

### SUMMARY OF THE DISCLOSURE

Disclosed herein are nucleic acid molecules (e.g., target genes, DNAs, dsRNAs, siRNAs, miRNAs, shRNAs, and hpRNAs), and methods of use thereof, for the control of insect pests, including, for example, coleopteran pests, such as D. v. virgifera LeConte (western corn rootworm, “WCR”); D. barberi Smith and Lawrence (northern corn rootworm, “NCR”); D. u. howardi Barber (southern corn rootworm, “SCR”); D. v. zeae Krysan and Smith (Mexican corn rootworm, “MCR”); D. balteata LeConte; D. u. tenella; D. u. undecimpunctata Mannerheim, and hemipteran pests, such as Euschistus heros (Fabr.) (Neotropical Brown Stink Bug, “BSB”); E. servus (Say) (Brown Stink Bug); Nezara viridula (L.) (Southern Green Stink Bug); Piezodorus guildinii (Westwood) (Red-banded Stink Bug); Halyomorpha halys (Stål) (Brown Marmorated Stink Bug); Chinavia hilare (Say) (Green Stink Bug); C. marginatum (Palisot de Beauvois); Dichelops melacanthus (Dallas); D. furcatus (F.); Edessa meditabunda (F.); Thyanta perditor (F.) (Neotropical Red Shouldered Stink Bug); Horcias nobilellus (Berg) (Cotton Bug); Taedia stigmosa (Berg); Dysdercus peruvianus (Guérin-Méneville); Neomegalotomus parvus (Westwood); Leptoglossus zonatus (Dallas); Niesthrea sidae (F.); Lygus hesperus (Knight) (Western Tarnished Plant Bug); and L. lineolaris (Palisot de Beauvois). In particular examples, exemplary nucleic acid molecules are disclosed that may be homologous to at least a portion of one or more native nucleic acids in an insect pest.

In these and further examples, the native nucleic acid may be a target gene, the product of which may be, for example and without limitation: involved in a metabolic process or involved in larval/nymphal development. In some examples, post-translational inhibition of the expression of a target gene by a nucleic acid molecule comprising a polynucleotide homologous thereto may be lethal in coleopteran and/or hemipteran pests, or result in reduced growth and/or development thereof. In specific examples, a Gho/Sec24B2 gene or Sec24B1 gene may be selected as a target gene for post-transcriptional silencing. In particular examples, a target gene useful for post-transcriptional inhibition is a novel Diabrotica gene referred to herein as Sec24B2 (e.g., SEQ ID NO:1 and SEQ ID NO:107), a novel Euschistus heros gene referred to herein as BSB_Gho (e.g., SEQ ID NO:84 and SEQ ID NO:85), or a novel Diabrotica gene referred to herein as Sec24B1 (e.g., SEQ ID NO:102). An isolated nucleic acid molecule comprising the polynucleotide of SEQ ID NO:1, SEQ ID NO:84, SEQ ID NO:85, SEQ ID NO:102, or SEQ ID NO:107; the complement of SEQ ID NO:1, SEQ ID NO:84, SEQ ID NO:85, SEQ ID NO:102, or SEQ ID NO:107; and fragments of any of the foregoing (e.g., SEQ ID NOs:3-6, SEQ ID NOs:86-88, SEQ ID NO:104, and SEQ ID NO:109) are therefore disclosed herein.

Also disclosed are nucleic acid molecules comprising a polynucleotide that encodes a polypeptide that is at least about 85% identical to an amino acid sequence within a target gene product (for example, the product of a Gho/Sec24B2 gene or Sec24B1 gene). For example, a nucleic acid molecule may comprise a polynucleotide encoding a polypeptide that is at least 85% identical to GHO/SEC24B2 (e.g., SEQ ID NO:2 (SEC24B2), SEQ ID NO:98 (BSB_GHO), SEQ ID NO:99 (BSB-GHO), and SEQ ID NO:108 (SEC24B2)); an amino acid sequence within a product of Gho/Sec24B2; SEC24B1 (e.g., SEQ ID NO:103); and/or an amino acid sequence within a product of Sec24B1. Further disclosed are nucleic acid molecules comprising a polynucleotide that is the reverse complement of a polynucleotide that encodes a polypeptide at least 85% identical to an amino acid sequence within a target gene product.

Also disclosed are cDNA polynucleotides that may be used for the production of iRNA (e.g., dsRNA, siRNA, miRNA, shRNA, and hpRNA) molecules that are complementary to all or part of a coleopteran and/or hemipteran pest target gene, for example, a Gho/Sec24B2 gene or Sec24B1 gene. In particular embodiments, dsRNAs, siRNAs, miRNAs, shRNAs, and/or hpRNAs may be produced in vitro, or in vivo by a genetically-modified organism, such as a plant or bacterium. In particular examples, cDNA molecules are disclosed that may be used to produce iRNA molecules that are complementary to all or part of the novel Diabrotica gene referred to herein as Sec24B2 (e.g., SEQ ID NO:1 and SEQ ID NO:107), the novel Euschistus heros gene referred to herein as BSB_Gho (e.g., SEQ ID NO:84 and SEQ ID NO:85), or the novel Diabrotica gene referred to herein as Sec24B1 (e.g., SEQ ID NO:102).

Further disclosed are means for inhibiting expression of an essential gene in a coleopteran pest, and means for protecting a plant from coleopteran pests. A means for inhibiting expression of an essential gene in a coleopteran pest is a double-stranded RNA molecule encoded by a polynucleotide selected from the group consisting of SEQ ID NO:18 and SEQ ID NO:19; and the complements thereof. Functional equivalents of means for inhibiting expression of an essential gene in a coleopteran pest include single- or double-stranded RNA molecules that are substantially homologous to all or part of a transcript of a WCR gene comprising SEQ ID NO:1, SEQ ID NO:102, and/or SEQ ID NO:107. A means for protecting a plant from coleopteran pests is a DNA molecule comprising a polynucleotide encoding a means for inhibiting expression of an essential gene in a coleopteran pest operably linked to a promoter, wherein the DNA molecule is capable of being integrated into the genome of plants, such as, for example, maize.

Further disclosed are means for inhibiting expression of an essential gene in a hemipteran pest, and means for protecting a plant from hemipteran pests. A means for inhibiting expression of an essential gene in a hemipteran pest is a single-stranded RNA molecule encoded by a polynucleotide selected from the group consisting of any of SEQ ID NOs:86-88. Functional equivalents of means for inhibiting expression of an essential gene in a hemipteran pest include single-stranded RNA molecules that are substantially homologous to all or part of a transcript of a BSB gene comprising SEQ ID NO:84 or SEQ ID NO:85. A means for protecting a plant from hemipteran pests is a DNA molecule comprising a polynucleotide encoding a means for inhibiting expression of an essential gene in a hemipteran pest operably linked to a promoter, wherein the DNA molecule is capable of being integrated into the genome of plants, such as, for example, maize.

Disclosed are methods for controlling a population of an insect pest (e.g., a coleopteran or hemipteran pest), comprising providing to an insect pest (e.g., a coleopteran or hemipteran pest) an iRNA (e.g., dsRNA, siRNA, shRNA, miRNA, and hpRNA) molecule that functions upon being taken up by the pest to inhibit a biological function within the pest, wherein the iRNA molecule comprises all or part of (e.g., at least 15 contiguous nucleotides of) a polynucleotide selected from the group consisting of: any of SEQ ID NO:112; the complement of SEQ ID NO:112; SEQ ID NO:113; the complement of SEQ ID NO:113; SEQ ID NO:114; the complement of SEQ ID NO:114; SEQ ID NO:115; the complement of SEQ ID NO:115; SEQ ID NO:116; the complement of SEQ ID NO:116; SEQ ID NO:119; the complement of SEQ ID NO:119; SEQ ID NO:120; the complement of SEQ ID NO:120; SEQ ID NO:121; the complement of SEQ ID NO:121; SEQ ID NO:122; the complement of SEQ ID NO:122; SEQ ID NO:123; the complement of SEQ ID NO:123; SEQ ID NO:124; the complement of SEQ ID NO:124; SEQ ID NO:125; the complement of SEQ ID NO:125; SEQ ID NO:126; the complement of SEQ ID NO:126; SEQ ID NO:127; the complement of SEQ ID NO:127; a polynucleotide that hybridizes to a native coding polynucleotide of a Diabrotica organism (e.g., WCR) comprising all or part of any of SEQ ID NOs:1, 102, and/or 107; the complement of a polynucleotide that hybridizes to a native coding polynucleotide of a Diabrotica organism comprising all or part of any of SEQ ID NOs:1, 102, and/or 107; a polynucleotide that hybridizes to a native coding polynucleotide of a Euschistus heros organism comprising all or part of SEQ ID NO:84 and/or SEQ ID NO:85; and the complement of a polynucleotide that hybridizes to a native coding polynucleotide of a Euschistus heros organism comprising all or part of SEQ ID NO:84 and/or SEQ ID NO:85.

In particular embodiments, an iRNA that functions upon being taken up by an insect pest to inhibit a biological function within the pest is transcribed from a DNA comprising all or part of (e.g., at least 15 contiguous nucleotides of) a polynucleotide selected from the group consisting of: SEQ ID NO:1; the complement of SEQ ID NO:1; SEQ ID NO:3; the complement of SEQ ID NO:3; SEQ ID NO:4; the complement of SEQ ID NO:4; SEQ ID NO:5; the complement of SEQ ID NO:5; SEQ ID NO:6; the complement of SEQ ID NO:6; SEQ ID NO:84; the complement of SEQ ID NO:84; SEQ ID NO:84; the complement of SEQ ID NO:84; SEQ ID NO:85; the complement of SEQ ID NO:85; SEQ ID NO:86; the complement of SEQ ID NO:86; SEQ ID NO:87; the complement of SEQ ID NO:87; SEQ ID NO:88; the complement of SEQ ID NO:88; SEQ ID NO:102; the complement of SEQ ID NO:102; SEQ ID NO:104; the complement of SEQ ID NO:104; SEQ ID NO:107; the complement of SEQ ID NO:107; SEQ ID NO:109; the complement of SEQ ID NO:109; a native coding polynucleotide of a Diabrotica organism (e.g., WCR) comprising all or part of any of SEQ ID NOs:1, 3-6, 102, 104, 107, and 109; the complement of a native coding polynucleotide of a Diabrotica organism comprising all or part of any of SEQ ID NOs:1, 3-6, 102, 104, 107, and 109; a native coding polynucleotide of a Euschistus heros organism comprising all or part of any of SEQ ID NOs:84-88; and the complement of a native coding polynucleotide of a Euschistus heros organism comprising all or part of any of SEQ ID NOs:84-88.

Also disclosed herein are methods wherein dsRNAs, siRNAs, shRNAs, miRNAs, and/or hpRNAs may be provided to an insect pest in a diet-based assay, or in genetically-modified plant cells expressing the dsRNAs, siRNAs, shRNAs, miRNAs, and/or hpRNAs. In these and further examples, the dsRNAs, siRNAs, shRNAs, miRNAs, and/or hpRNAs may be ingested by the pest. Ingestion of dsRNAs, siRNA, shRNAs, miRNAs, and/or hpRNAs of the invention may then result in RNAi in the pest, which in turn may result in silencing of a gene essential for development of the pest and leading ultimately to mortality. In particular examples, the coleopteran and/or hemipteran pest controlled by use of nucleic acid molecules of the invention may be WCR, NCR, Euschistus heros, E. servus, Piezodorus guildinii, Halyomorpha halys, Nezara viridula, Chinavia hilare, C. marginatum, Dichelops melacanthus, D. furcatus, Edessa meditabunda, Thyanta perditor, Horcias nobilellus, Taedia stigmosa, Dysdercus peruvianus, Neomegalotomus parvus, Leptoglossus zonatus, Niesthrea sidae, and/or Lygus lineolaris.

The foregoing and other features will become more apparent from the following Detailed Description of several embodiments, which proceeds with reference to the accompanying Figures.

### BRIEF DESCRIPTION OF THE FIGURES

FIG. 1 includes a depiction of the strategy used to generate dsRNA from a single transcription template with a single pair of primers.

FIG. 2 includes a depiction of the strategy used to generate dsRNA from two transcription templates.

FIG. 3 includes a summary of data showing effects of particular dsRNAs on WCR mortality. Depicted are the percent mortality of adult Diabrotica v. virgifera after feeding WCR adults exposed to 500 ng Gho/Sec24B2 or Sec24B1 dsRNA/diet plug, or the same amount of GFP dsRNA, or water.

### SEQUENCE LISTING

The nucleic acid sequences listed in the accompanying sequence listing are shown using standard letter abbreviations for nucleotide bases, as defined in 37 C.F.R. §1.822. The nucleic acid and amino acid sequences listed define molecules (i.e., polynucleotides and polypeptides, respectively) having the nucleotide and amino acid monomers arranged in the manner described. The nucleic acid and amino acid sequences listed also each define a genus of polynucleotides or polypeptides that comprise the nucleotide and amino acid monomers arranged in the manner described. In view of the redundancy of the genetic code, it will be understood that a nucleotide sequence including a coding sequence also describes the genus of polynucleotides encoding the same polypeptide as a polynucleotide consisting of the reference sequence. It will further be understood that an amino acid sequence describes the genus of polynucleotide ORFs encoding that polypeptide.

Only one strand of each nucleic acid sequence is shown, but the complementary strand is understood as included by any reference to the displayed strand. As the complement and reverse complement of a primary nucleic acid sequence are necessarily disclosed by the primary sequence, the complementary sequence and reverse complementary sequence of a nucleic acid sequence are included by any reference to the nucleic acid sequence, unless it is explicitly stated to be otherwise (or it is clear to be otherwise from the context in which the sequence appears). Furthermore, as it is understood in the art that the nucleotide sequence of an RNA strand is determined by the sequence of the DNA from which it was transcribed (but for the substitution of uracil (U) nucleobases for thymine (T)), an RNA sequence is included by any reference to the DNA sequence encoding it. In the accompanying sequence listing:

SEQ ID NO:1 shows a DNA comprising an exemplary Diabrotica Sec24B2 polynucleotide:

GATGTCAACTGGACCTCCAACATTATCAAATGTGCCTCCAACGTTA
TCTAGTGGGCCTCCAACAAGTGGTTCTCCTCAAACAGGCCATTTAG
GTGCTCCACCAAATCAATCTCCCTTGTCTGGAGGAGTTCCACCTCA
AATGGGACCTAATCAACAATTAGGACAGCCACCATCGGCAGCTGGT
CCACCAAGCCACCTTGGACAGACTTCTTTGACTAACCCCCCACCCC
ATCCAGGTCAACCGAATCTCCCCTGGCGCCCACCTCAATCTGTAGG
TCAACCTGGTGGCCCTCCTGGATATCCTCCATTGCCAGGACATCAA
GGACAACCCACATCACAGTTCGACACACAAGGTCCAATGTCACAAA
ATGGACCTCCAAACATGTATGGAAATCCACCAAATCAATTTAATAA
TCAGATGGGTCCTCCAAAAGTGGGACAATTTCCTCAACAACAAAGG
CCAATGCAACCTCCCCTACCTGGACAGCCGCCTATGCCGGGACAAG
GTCCTTTAATCAGTGCTCCAGGTCCATACGGACCTTCTTCAGGACC
AGCACACCAAATGCCACCTCATCAAGGACAACCACCTCATCAAGGA
CAATCACCATATGGACCTGGCCAAATAACTAGTCAGTTGCAGCAAA
TGAATTTATCTGGTCCAAAGCCGGCTTATCCAGTACCACCAGGCGG
TCCCATGAGACCGATGAACGGAGACAGCGGTCCGCATATGCCTCCA
GCAATGAACCAACCGGGATATATGAATAATCAACAGGGCAGAGTTC
CTCCTGGACCTGGTTATCCACCGATGCCGGGGCAAGCACCGATGCA
AGGACAAGGACACATGCCTGGTCAAGGGCAATACCCAGGACCTGGT
GGGGGGTATCCGCAAGGCAACTACCAACAAGCTGCGCCGGCGCAAC
ACAAGATTGATCCTGATCATGTGCCGAATCCAATTCAAGTTATCCG
AGATGATCAGCAAGACAGGGACAGCGTTTTTGTTACTAATCAAAAA
GGACTTGTACCGCCTATGGTAACTACCAATTTTATTGTTCAAGATC
AAGGAAATTGCAGTCCACGATTCATGAGATCTACCATATATAATGT
TCCAATTTCACAGGATTTGTTAAAACAATCTGCACTTCCATTCAGT
CTTTTAATAAGTCCAATGGCCAGGCAAGTAGAGCAAGAATACCCTC
CACCAATCGTTAATTTCGGAAGCCTCGGTCCTGTCAGATGCATCCG
TTGCAAGGCCTACATGTGTCCGTTCATGCAGTTCGTCGATTCTGGA
AGGAGGTTCCAGTGTCTGTTTTGTAACGCAACTACTGATGTTCCAA
CAGAATATTTCCAGCATCTAGATCAGACCGGCCTAAGAATGGACCG
CTTTGAACGACCAGAATTGATCCTTGGTACCTACGAATTCGTCGCT
ACCCCCGATTACTGCCGAAACAACGTTCTGCCCAAACCGCCAGCCG
TCATTTTCGTTATCGACGTTTCATATAACAACATTAAATCCGGAAT
GGTTTCCTTGTTGTGCAATCAGATGAAAGAGATCATTCAAAATCTT
CCGGTGGACCAAGGCCACGAAAAGAGCAACATGAAAGTTGGATTTA
TTACGTATAATAGTTCGGTGCATTTTTATAATATCAAGGGAAGTTT
GACAGCTCCACAAATGTTGGTGGTAGGAGATGTCCAAGAAATGTTC
ATGCCTTTGTTGGATGGTTTCTTATGTACTCCAGAAGAATCGGGAC
CCGTAATAGATCTACTCATGCAACAGATTCCCGCAATGTTTGCAGA
TACTAAGGAAACCGAAGTCGTTTTGCTTCCCGCAATTCAAGCTGGA
TTAGAAGCCCTAAAGGCTTCCGAAAGTACAGGCAAACTTCTAGTAT
TCCACTCCACTTTACCAATAGCAGAGGCTCCAGGTAAATTGAAGAA
CCGCGACGATAGAAAAGTCTTAGGAACCGATAAAGAAAAAACTGTC
TTGACACCACAAACACAAGCATACAACCAATTGGGCCAGGAATGCG
TCAGCAACGGTTGCTCCGTTGATATGTATATCTTCAATAACGCTTA
CATCGATATAGCGACTATTGGTCAAGTGTCTAGATTGACGGGAGGA
GAAGTGTTTAAGTATACTTATTTCCAGGCTGATATTGATGGAGAAC
GTTTCATAACAGACGTTATCTTAAATATTAGTCGACCAATAGCGTT
TGATGCTGTAATGAGGGTTAGAACGTCAACAGGAGTGAGGCCCACT
GACTTTTATGGTCATTTCTACATGTCAAATACTACGGATATCGAAC
TAGCGGCAGTAGATTGCGATAAAGCCATAGCAGTCGAAATAAAACA
CGACGACAAACTGAATGAAGACACGGGGGTATTCATTCAAACGGCG
CTGTTATACACATCGTGCTCAGGACAGCGACGGTTGCGAATTATGA
ATCTTTCACTGAAGACTTGCTCACAAATGGCCGATCTCTTTAGAAG
TTGTGATTTAGATACTTTAATCAATTACATGAGTAAACAGGCTACG
TATAAATTATTGGACGGCAGCCCCAGCGTTGTAAAGGAGGGACTTG
TCCATAGAGCCGCTCAGATCTTAGCAATATACAGGAAGCACTGCGC
AAGTCCAAGTAGCGCGGGTCAACTAATTCTTCCCGAATGCATGAAG
CTGCTACCGATCTACACCAATTGTCTTCTCAAGAACGACGCTATCT
CAGGAGGTTCGGATATGACCATCGACGACAAATCGTTCGTCATGCA
GGTGGTCTTGAGCATGGACCTTAACTTCTCGGTGTACTATTTCTAT
CCTAGGTTAATTCCACTACACGATATCGATCCCAACCAGGATCCTA
TCACAGTTCCGAATCCTATGAGGTGTAGTTATGATAAAATGAATGA
ACAGGGAGTGTATATATTAGAAAACGGAATCCATATGTTCTTATGG
TTTGGTCTCGGCGTGAATCCCAACTTTATTCAGCAACTCTTTGGTG
CGCCTTCAGCAATACAAGTTGATATCGATAGGAGTAGTTTGCCGGA
ATTAGATAACCCATTGTCGGTAGCAGTTAGGACAATAATAGACGAA
ATCAGGATACAGAAACATAGGTGTATGAGGTTAACCCTGGTTAGAC
AAAGAGAAAAACTGGAACCAGTCTTCAAGCATTTCTTAGTAGAGGA
CCGCGGCACAGACGGTTCAGCCAGCTATGTCGACTTCCTATGTCAT
ATGCACAGAGAAATCAGAAACATCCTCAGCTAGCACAGAAGGTGAT
CCAAAGGCAGACGGAAGATAAGATGATAGAAAATCTTGAAATTTGT
ACTCTGATCCTCGATAACATATTTCCTCTTGTATAAAGTATTATTA
AGATCTATTTTTGTATAGCGCATGCGTTTGTAAAGGGTGCCAGACG
GTGTTCTTTTGGATTTCTAGATATTCTATTATATTATGCATTATTT
TGGGGTCTAGCTTGTCGGTGCTTTTACATATTAAAGAAAATCAGTT
TGTTTCCGTATGCTCAGGAAACAAACAACGCTTTTTTTTCTATTTT
ATTGGTTATTACACGTCGACAGAACTATCTGAAAGGTCAGATCGAA
AACTTTCGTTACGCGACGTTGTCAGATTAATCGAAGTTTAAAGGTT
TTCCGGTTTTTATTTGTTACCTGTTTCACA

SEQ ID NO:2 shows the amino acid sequence of a Diabrotica SEC24B2 polypeptide encoded by an exemplary Diabrotica Sec24B2 DNA:

MSTGPPTLSNVPPTLSSGPPTSGSPQTGHLGAPPNQSPLSGGVPPQ
MGPNQQLGQPPSAAGPPSHLGQTSLTNPPPHPGQPNLPWRPPQSVG
QPGGPPGYPPLPGHQGQPTSQFDTQGPMSQNGPPNMYGNPPNQFNN
QMGPPKVGQFPQQQRPMQPPLPGQPPMPGQGPLISAPGPYGPSSGP
AHQMPPHQGQPPHQGQSPYGPGQITSQLQQMNLSGPKPAYPVPPGG
PMRPMNGDSGPHMPPAMNQPGYMNNQQGRVPPGPGYPPMPGQAPMQ
GQGHMPGQGQYPGPGGGYPQGNYQQAAPAQHKIDPDHVPNPIQVIR
DDQQDRDSVFVTNQKGLVPPMVTTNFIVQDQGNCSPRFMRSTIYNV
PISQDLLKQSALPFSLLISPMARQVEQEYPPPIVNFGSLGPVRCIR
CKAYMCPFMQFVDSGRRFQCLFCNATTDVPTEYFQHLDQTGLRMDR
FERPELILGTYEFVATPDYCRNNVLPKPPAVIFVIDVSYNNIKSGM
VSLLCNQMKEIIQNLPVDQGHEKSNMKVGFITYNSSVHFYNIKGSL
TAPQMLVVGDVQEMFMPLLDGFLCTPEESGPVIDLLMQQIPAMFAD
TKETEVVLLPAIQAGLEALKASESTGKLLVFHSTLPIAEAPGKLKN
RDDRKVLGTDKEKTVLTPQTQAYNQLGQECVSNGCSVDMYIFNNAY
IDIATIGQVSRLTGGEVFKYTYFQADIDGERFITDVILNISRPIAF
DAVMRVRTSTGVRPTDFYGHFYMSNTTDIELAAVDCDKAIAVEIKH
DDKLNEDTGVFIQTALLYTSCSGQRRLRIMNLSLKTCSQMADLFRS
CDLDTLINYMSKQATYKLLDGSPSVVKEGLVHRAAQILAIYRKHCA
SPSSAGQLILPECMKLLPIYTNCLLKNDAISGGSDMTIDDKSFVMQ
VVLSMDLNFSVYYFYPRLIPLHDIDPNQDPITVPNPMRCSYDKMNE
QGVYILENGIHMFLWFGLGVNPNFIQQLFGAPSAIQVDIDRSSLPE
LDNPLSVAVRTIIDEIRIQKHRCMRLTLVRQREKLEPVFKHFLVED
RGTDGSASYVDFLCHMHREIRNILS

SEQ ID NO:3 shows an exemplary Diabrotica Sec24B2 DNA, referred to herein in some places as Sec24B2 reg1, which is used in some examples for the production of a dsRNA:

TATATCTTCAATAACGCTTACATCGATATAGCGACTATTGGTCAAG
TGTCTAGATTGACGGGAGGAGAAGTGTTTAAGTATACTTATTTCCA
GGCTGATATTGATGGAGAACGTTTCATAACAGACGTTATCTTAAAT
ATTAGTCGACCAATAGCGTTTGATGCTGTAATGAGGGTTAGAACGT
CAACAGGAGTGAGGCCCACTGACTTTTATGGTCATTTCTACATGTC
AAATACTACGGATATCGAACTAGCGGCAGTAGATTGCGATAAAGCC
ATAGCAGTCGAAATAAAACACGACGACAAACTGAATGAAGACAC

SEQ ID NO:4 shows an exemplary Diabrotica Sec24B2 DNA, referred to herein in some places as Sec24B2 reg2, which is used in some examples for the production of a dsRNA:

CTAAGGAAACCGAAGTCGTTTTGCTTCCCGCAATTCAAGCTGGATT
AGAAGCCCTAAAGGCTTCCGAAAGTACAGGCAAACTTCTAGTATTC
CACTCCACTTTACCAATAGCAGAGGCTCCAGGTAAATTGAAGAACC
GCGACGATAGAAAAGTCTTAGGAACCGATAAAGAAAAAACTGTCTT
GACACCACAAACACAAGCATACAACCAATTGGGCCAGGAATGCGTC
AGCAACGGTTGCTCCGTTGATATGTATATCTTCAATAACGCTTACA
TCGATATAGCGACTATTGGTCAAGTGTCTAGATTGACGGGAGGAGA
AGTGTTTAAGTATACTTATTTCCAGGCTGATATTGATGGAGAACGT
TTCATAACAGACGTTATCTTAAATATTAGTCGACCAATAGCGTTTG
ATGC

SEQ ID NO:5 shows an exemplary Diabrotica Sec24B2 DNA, referred to herein in some places as Sec24B2 ver1, which is used in some examples for the production of a dsRNA:

TCGTTTTGCTTCCCGCAATTCAAGCTGGATTAGAAGCCCTAAAGGC
TTCCGAAAGTACAGGCAAACTTCTAGTATTCCACTCCACTTTACCA
ATAGCAGAGGCTCCAGGTAAATTGAAGAACCGCGACGATAGAAAAG
TCTTAGGAACCGATAAAGAAAAAACTGTCTTGACACCACAAACACA
AGCATACAACCAATTGGGCCAGGAATGCGTCAGCAACGGTTGCTCC
GTTGATATGTATATCTTCAATAACGCTTACATCGATATAGCGACTA
TTGGTCAAGTG

SEQ ID NO:6 shows an exemplary Diabrotica Sec24B2 DNA, referred to herein in some places as Sec24B2 ver2, which is used in some examples for the production of a dsRNA:

GTCGTTTTGCTTCCCGCAATTCAAGCTGGATTAGAAGCCCTAAAGG
CTTCCGAAAGTACAGGCAAACTTCTAGTATTCCACTCCACTTTACC
AATAGCAGAGGCTCCAGGTAAATTGAAGAACCGCGA

SEQ ID NO:7 shows the nucleotide sequence of a T7 phage promoter.

SEQ ID NO:8 shows the DNA template for the sense strand of a YFP dsRNA.

SEQ ID NO:9 shows the DNA template for the sense strand of a GFP dsRNA.

SEQ ID NOs:10-17 show primers used to amplify gene regions (i.e., Sec24B2 reg1, Sec24B2 ver1, and Sec24B2 ver2) of exemplary Diabrotica Sec24B2 genes.

SEQ ID NO:18 shows an exemplary DNA encoding a Diabrotica Sec24B2 v1 hairpin-forming RNA; containing sense polynucleotides, a loop sequence comprising an intron (underlined), and antisense polynucleotide (bold font):

TCGTTTTGCTTCCCGCAATTCAAGCTGGATTAGAAGCCCTAAAGGC
TTCCGAAAGTACAGGCAAACTTCTAGTATTCCACTCCACTTTACCA
ATAGCAGAGGCTCCAGGTAAATTGAAGAACCGCGACGATAGAAAAG
TCTTAGGAACCGATAAAGAAAAAACTGTCTTGACACCACAAACACA
AGCATACAACCAATTGGGCCAGGAATGCGTCAGCAACGGTTGCTCC
GTTGATATGTATATCTTCAATAACGCTTACATCGATATAGCGACTA
TTGGTCAAGTGGAATCCTTGCGTCATTTGGTGACTAGTACCGGTTG
GGAAAGGTATGTTTCTGCTTCTACCTTTGATATATATATAATAATT
ATCACTAATTAGTAGTAATATAGTATTTCAAGTATTTTTTTCAAAA
TAAAAGAATGTAGTATATAGCTATTGCTTTTCTGTAGTTTATAAGT
GTGTATATTTTAATTTATAACTTTTCTAATATATGACCAAAACATG
GTGATGTGCAGGTTGATCCGCGGTTAAGTTGTGCGTGAGTCCATTG
CACTTGACCAATAGTCGCTATATCGATGTAAGCGTTATTGAAGATA
TACATATCAACGGAGCAACCGTTGCTGACGCATTCCTGGCCCAATT
GGTTGTATGCTTGTGTTTGTGGTGTCAAGACAGTTTTTTCTTTATC
GGTTCCTAAGACTTTTCTATCGTCGCGGTTCTTCAATTTACCTGGA
GCCTCTGCTATTGGTAAAGTGGAGTGGAATACTAGAAGTTTGCCTG
TACTTTCGGAAGCCTTTAGGGCTTCTAATCCAGCTTGAATTGCGGG
AAGCAAAACGA

SEQ ID NO:19 shows an exemplary DNA encoding a Diabrotica Sec24B2 v2 hairpin-forming RNA; containing sense polynucleotides, a loop sequence comprising an intron (underlined), and antisense polynucleotide (bold font):

GTCGTTTTGCTTCCCGCAATTCAAGCTGGATTAGAAGCCCTAAAGG
CTTCCGAAAGTACAGGCAAACTTCTAGTATTCCACTCCACTTTACC
AATAGCAGAGGCTCCAGGTAAATTGAAGAACCGCGAGAATCCTTGC
GTCATTTGGTGACTAGTACCGGTTGGGAAAGGTATGTTTCTGCTTC
TACCTTTGATATATATATAATAATTATCACTAATTAGTAGTAATAT
AGTATTTCAAGTATTTTTTTCAAAATAAAAGAATGTAGTATATAGC
TATTGCTTTTCTGTAGTTTATAAGTGTGTATATTTTAATTTATAAC
TTTTCTAATATATGACCAAAACATGGTGATGTGCAGGTTGATCCGC
GGTTAAGTTGTGCGTGAGTCCATTGTCGCGGTTCTTCAATTTACCT
GGAGCCTCTGCTATTGGTAAAGTGGAGTGGAATACTAGAAGTTTGC
CTGTACTTTCGGAAGCCTTTAGGGCTTCTAATCCAGCTTGAATTGC
GGGAAGCAAAACGAC

SEQ ID NO:20 shows an exemplary DNA encoding a YFP v2 hairpin-forming RNA; containing sense polynucleotides, a loop sequence comprising an intron (underlined), and antisense polynucleotide (bold font):

ATGTCATCTGGAGCACTTCTCTTTCATGGGAAGATTCCTTACGTTG
TGGAGATGGAAGGGAATGTTGATGGCCACACCTTTAGCATACGTGG
GAAAGGCTACGGAGATGCCTCAGTGGGAAAGGACTAGTACCGGTTG
GGAAAGGTATGTTTCTGCTTCTACCTTTGATATATATATAATAATT
ATCACTAATTAGTAGTAATATAGTATTTCAAGTATTTTTTTCAAAA
TAAAAGAATGTAGTATATAGCTATTGCTTTTCTGTAGTTTATAAGT
GTGTATATTTTAATTTATAACTTTTCTAATATATGACCAAAACATG
GTGATGTGCAGGTTGATCCGCGGTTACTTTCCCACTGAGGCATCTC
CGTAGCCTTTCCCACGTATGCTAAAGGTGTGGCCATCAACATTCCC
TTCCATCTCCACAACGTAAGGAATCTTCCCATGAAAGAGAAGTGCT
CCAGATGACAT

SEQ ID NO:21 shows an exemplary DNA comprising an ST-LS1 intron.

SEQ ID NO:22 shows a YFP protein coding sequence.

SEQ ID NO:23 shows a DNA sequence of annexin region 1.

SEQ ID NO:24 shows a DNA sequence of annexin region 2.

SEQ ID NO:25 shows a DNA sequence of beta spectrin 2 region 1.

SEQ ID NO:26 shows a DNA sequence of beta spectrin 2 region 2.

SEQ ID NO:27 shows a DNA sequence of mtRP-L4 region 1.

SEQ ID NO:28 shows a DNA sequence of mtRP-L4 region 2.

SEQ ID NOs:29-58 show primers used to amplify gene regions of gfp, yfp, annexin, beta spectrin 2, and mtRP-L4 for dsRNA synthesis.

SEQ ID NO:59 shows a maize TIP41-like protein coding sequence.

SEQ ID NO:60 shows the nucleotide sequence of a T20VN primer oligonucleotide.

SEQ ID NOs:61-65 show primers and probes used for dsRNA transcript expression analyses.

SEQ ID NO:66 shows a nucleotide sequence of a portion of a SpecR coding region used for binary vector backbone detection.

SEQ ID NO:67 shows a nucleotide sequence of an AAD1 coding region used for genomic copy number analysis.

SEQ ID NO:68 shows a maize invertase gene.

SEQ ID NOs:69-77 show primers and probes used for gene copy number analyses.

SEQ ID NOs:78-80 show primers and probes used for maize expression analysis.

SEQ ID NO:81 shows a DNA comprising an actin gene.

SEQ ID NOs:82 and 83 show primers used to amplify gene regions of actin for dsRNA synthesis.

SEQ ID NO:84 shows a DNA comprising an exemplary Euschistus heros BSB_Gho polynucleotide:

ACGTAACCTCACTTTCTTGACAGCTTCCGCCAGACTGTTTTTCATT
TAGGCTAGTTTGCCTTCGCAGTCTTGTTATATTGATAAAAACTTTC
GTTAAGCTTAGTTAAAATTAAAGATACAACAATCTCGTAAGTATTT
ACAACTCGGGCGAAGTAAAAATGTTACTGTTTCGCTGTTTGGTTTC
ATGTGTGCTATAACCAAAGATTTATCTTAAGGGGAAAAACGGTGCT
ATTTCATGCGTCTCGAAGCTTAAACTAATTTAAACAAGTAGTTTTA
ATTTAAGGAACAGTTGAGTTTTATATATTATCTTTTAAATGGTACC
GTTAATGCTTACACGGAGCGCATCGTAGTAACTTGGGAAAGGGGAG
TGACATATAAGTGTAACCGTCCATATATCAGACTTCTATTTGTAAT
TTAATTAATCATTTGAAAGTTTTTAAGCTGATTCATGTTTTCAAAT
TAACTAAGGAGCCCTCAACTACCTTTTGTAATTTTGAATAATGAAC
GGCCAATCTTGCACTTATTCTGACTCTGGAAATGGTACACCAACAC
CTTCATCCACAAGCTATCCAGCTAGTTTATCATCACAATCTTCCCG
TGATACATCCCCCTCCCGCCTTCATCCTAACCTTAATCATATAAAT
TCTGAAAAATCAATTAATTCATCTGGTAACTATATGAATTATAAAA
TACACGATACGTATACAAATGCCAATTCTGTTTATGGGCAAATATA
TTCAGACTCAACTACACCTACTAACAGGGCAACAGTTCCCCCGTAC
ATCAGTGACACTAATAACGACATTAATCAATCTCAAAGACTGGGGC
AACCGCAGCTCCGACCTTCAACAACATCATCACAAATAATAACTAG
TTTAGGGTCTTCGGTTTCTAAACCTGTCTATAGTTCATCACATTTA
AATCAAATATCGAATGATCAGAAACAGTATGTTAATCAATATAGCA
CACAAAAGTTAGATAGCGTTATGCAGCCTAAAACATCAGAGAGTAA
CATCATTAAAAATCATGAAACTATGCCTACATCTAATTTAGCAATA
TCTGATTATTATCAGGGATATACTCAAACGATGAATAATCCCTACA
GGCAAGAAAATGTATTGCCTAACCAGACAATGAAGCCCGAACAACA
GTACCATGCTCAAACCCAAGGGTATCAAGTTCAAAAACCCTTGATG
TCTCCAACATCAAATCCATACATGAATTCAGTGCCTCAAGATAACC
AAAACTACCCCCAATCACCAGGTGATGTCCCCAGGTCTACTTTCCA
GCAGGGTTATTATCAGCATCAACCTCAACCTCAACCTCAACCACAA
CCACCTTCAGTAATGAGTGGAAGACCGCAGATGAATTTGCCTTTGA
CTCAGTCTAGATCACTTGATGAACCTATTTCTTCAGGGCCTCCAAG
AACAAACGTCTTGGGAATCATTCCTTATGCCACTGAACCTGCTACT
TCGCAAGTTTCGAGGCCTAAATTACCCGATGGTGGAGGGTATTATC
AGCCCATGCAACCACAACAGCAACCACCGCAGATGCAGCAGCCACA
GATGCAGCAACCGCAGATGCAGCAGCAACAGCCACCACGAGTGGCA
CCAAGACCCCCAGCGCCTAAACCTAAAGGCTACCCTCCACCACCAT
ATCAACAATATCCATCTTATTCCCATCCTCAAAACAATGCTGGTTT
ACCTCCTTACAGTCAAACAATGGGTGGTTATTACCCGAGCGGAGAT
GAACTTGCTAATCAGATGTCACAGCTTAGCGTTTCTCAACTTGGTT
TTAATAAATTATGGGGAAGGGATACAGTGGACTTGATGAAGAGTCG
TGATGTTTTGCCCCCTACTCGGGTCGAAGCTCCTCCAGTTCGTCTT
TCTCAGGAGTACTATGATTCGACTAAAGTTAGCCCTGAGATATTTA
GATGTACGCTAACTAAAATACCCGAGACCAAATCTCTTCTTGATAA
ATCTAGGCTTCCCCTTGGCGTCTTGATCCACCCATTCAAGGACCTA
AATCAATTGTCGGTGATCCAGTGCACAGTAATAGTACGATGTAGAG
CGTGTAGGACTTATATAAATCCTTTTGTATTCTTTGTCGACTCGAA
GCATTGGAAATGCAATCTCTGCTTTAGGGTGAATGATTTGCCAGAA
GAATTTCAATATGACCCATTAACAAAGACTTATGGAGACCCTACTA
GACGACCAGAAATAAAATCTGCTACTATAGAATTCATAGCTCCATC
GGAATATATGGTGAGGCCGCCGCAACCGGCTGCTTACGTGTTTGTA
TTAGACGTGTCAAGACTAGCGGTCGAGAGTGGTTACTTGCGTATCT
TCTGTGACTGCCTCCTTTCCCAGCTGGAGGCGTTGCCAGGCGATTC
GAGGACAGCTGTGGCTTTTATCACCTACGACTCTGCTGTCCACTAT
TATAGCCTTGCTGATACCCAGGCTCAGCCACATCAGATGGTCGTAG
TGGACATTGATGATATGTTCGTACCATGCCCTGAAAACCTGCTGGT
GAACCTGAGTGAGTGCCTGGGGCTAGTACGGGACCTTCTGCGGGAA
CTGCCTAATAAGTATAGAGATTCCTATGACACAGGCACTGCCGTCG
GTCCTGCTTTACAAGCAGCTTACAAATTATTGGCCGCAACTGGTGG
AAGAGTGACTTTGGTAACGAGCTGCTTGGCGAACAGCGGACCAGGA
AAACTGCCATCTCGAGAGGACCCGAACCAGAGGAGCGGGGAAGGGT
TGAACCAGTCACATCTCAACCCAGTCACTGACTTCTACAAGAAATT
GGCCCTCGATTGCTCAGGCCAACAGATTGCTGTCGATCTTTTCGTA
CTTAACAGTCAATTTGTTGACCTTGCTTCTCTGAGTGGTGTTTCGA
GGTTTTCCGGTGGGTGTATCCATCATTTCCCTCTGTTCTCTGTGAA
GAACCCTCATCATGTTGAATCATTCCAGCGTAGTCTACAGAGGTAT
CTGTGTCGTAAGATTGGTTTTGAATCTGTCATGAGGTTGCGCTGCA
CCAGGGGGTTATCTATTCATACATTCCATGGAAACTTCTTTGTTCG
TTCAACGGACCTCCTCTCTCTACCCAATGTAAACCCAGATGCTGGT
TTCGGAATGCAGGTGTCTATTGACGAGAACCTGACTGATATACAGA
CCGTATGTTTCCAAGCAGCACTTCTGTATACTTCGAGTAAAGGAGA
AAGAAGAATCCGTGTTCACACTTTGTGCCTTCCAATAGCTTCTAAC
CTTTCAGACGTTCTGCATGGAGCAGACCAGCAATGTATCGTAGGTC
TTCTGGCTAAGATGGCTGTTGATAGGTGTCATCAGTCGTCGCTGAG
TGATGCAAGGGAGGCTTTTGTGAACGTAGTTGCTGATATGTTATCA
GCGTTCCGGATCACCCAGTCTGGCGTATCACCTACCTCACTAGTCG
CTCCCATTAGTCTCTCCCTTCTTCCACTCTATGTACTCGCTTTGCT
CAAATATATTGCTTTCCGTGTCGGCCAGAGCACAAGGCTGGACGAT
CGAGTCTTCGCTATGTGCCAAATGAAGTCTCTACCTCTCTCTCAGT
TAATACAGGCCATTTACCCTGATCTCTATCCAATAGCCAATATCAA
CGAATTGCCACTTGTTACTATTGGAGAAGACCAAGTAGTCCAACCA
CCATTACTTCACCTCTCAGCTGAAAGAATAGACTCGACGGGGGTCT
ACTTGATGGATGATGGAACAACAATAATTATCTACGTCGGCCACAA
CATTAATCCATCAATTGCTGTTTCCTTCTTCGGGGTACCTTCATTT
TCAGCTATAAATTCTAATATGTTTGAACTACCTGAACTGAATACGC
CGGAGTCTAAAAAACTGAGAGGTTTCATTAGCTATTTACAGAATGA
GAAGCCCGTAGCTCCGACTGTACTCATCATTAGGGATGACAGCCAG
CAGAGACATTTATTTGTCGAGAAGCTCATAGAAGACAAAACTGAAT
CCGGTCATTCTTACTACGAATTTTTGCAGAGAGTGAAGGTACTCGT
TAAGTAACAAACAGCTGAGATATTCTCACTCTATACCAATCTACCA
AAGACTATGTCGTGTGTTGATGGGGCATGGCAACACATCTTATGTC
CATTATAGATTTCTAACTTTTTTATATTTTCTGCTTCTTATTCGTC
GTAATGAGAAGTTTTAATTGATGTTTCATCAACTACAAAACTTTTA
TCCTGTATAACACATCATTTTATATAGTATTATATATATAA

SEQ ID NO:85 shows a DNA comprising a further exemplary Euschistus heros BSB_Gho polynucleotide:

ATGGAATAAAATTTTTATTTACAGAAAATAATCATCAACATTATCT
ACAAATTTATTTTCTATAATTTATATATAATAACACATTACCAAAC
AAAAATAACATATCGTAGTTATAACAATTGTTTATATATAAATACA
TACACATGTCACACCATACACCGCATAACCTTCGAACTCGGCTACA
CAAGATCTTAAGGAGCGCACAACATAAATACAACATAAAGCAAAGT
ATCAATGTAAATAAGGGAAACTTAGGTACAAGTGTCTGTTCATGGG
GAACATATATATCTATATATGATATAACAATTATTAGTGTTAAAAA
TAATATTTAATTAAAATAATATTTACTGGCAACATATAATAAAAAT
ATTTGATTACATAAATTACCTAGATAAAGCAACAGCTTGATATAAT
CCTCGTTAAACATATACTGCACGCAGTTGGTTCTTTTATAATGTAC
TGTAGGAAATTTTGATACATAAAAAAAAAAAAAAAATAATGGAAAG
AAGAAGAAAAGTGCACTGGTGGCAAGTTTAATTTGACAAGTTGGAA
GTATACGTATCATACGCCATTTTTTATCTTTAGATAGTAAGTACTC
AGATGCACTATCAATAACTTTTGCTAATATTTTTAAAATTTTTATT
TTTTAAGTCCAATTCACGTAGATATATTTATGTACAGTTTAATAAA
TTTCCTCCCTCTGTAAAAAATAAAATAAAACAAAATATAACCAATG
ATATAAACAAATTTTGATAATTAAATTTAAAACAATAATATTAATC
ACATCCCACATTTTAAAGGAAGTAGAAAGAAAACAATACATTATTT
ATGATACAATCCCGTTATAATATACATCATCAAACAAACAGTTGTA
AGCTTACCCGTTAAATGAGAAACTGTTACTTAATAATAATGAATTA
TAACAATTTCATCAGCTATAAAAATATCAAATCGAAATTTCATACA
ATTGAAGGATAATGATAAATTTTACAGGTTCGATAGGAAATGTCAA
GCCAACAATTGGCAGTCGTAATCTGCATAATAGTCTGCTGTGGAGG
TCGCTAACTAAGCATATTACGAATTTCTTTGTGAAGATGACATAGA
AAATCTACATAAGATGAAGATCCATCCAAACCTCTGTCCTCGACCA
AGAAGTGCTTCATCACCATTTCCATTTTGTCCCGTTGTCTCACTAT
TGTCAGCCTCATTGTCCTATGATTGCTGTCAGCAATTGATGAGATT
GCATTCCTGACTCTTTCTGAAATCGGGTTTTCAAGGGGTGGTAATC
TATGTCTATCGGTATCGACCTGAGCTGCACTTGGAACTCCAAATAC
TGACATCACCCAATCTGAAGGAGTAGCTAGACCCAGCCAGATGAAC
ATGTAAATACCGTTTACTAGTAAATATACTCCACTATCCACCATTT
TTTCAGATGAACATCTTATGCACGGTGGTGGTACAGAATCCTCTAG
CTCTAATAGAGAATATAACCGTGGGTAGAAGTATACAAGAGAAGAA
GGAACATCCATCGTCAGAACTGCAGCCATCACAAACCATTTGTCGT
CAACTGTCATGTCTTTGCCTCCAGAGATAGCATCACTTTTCAAGAG
GCAGTTGACATACAGAGGTAACAACTTCATGCACTCAGGAAGGATC
AGCTGTCCAGCAGAAGTAGGAGAAGCACAATTCTTACGATAGCACG
CCAGAATCTGAGCTGACCTGTTTATTAATGATTCTTTAACAGCTTT
TGCCGATGCATCTAAAAGCTTGAACACACTCTGTTTGGAAAAGAAG
TTGATGATAGTGTCGAGTTCACAGGTTCTATAGAGGTCGGACATCT
GTGAGCAAGCCTTCAATACCAGGTTGAGAACTCTGATCCTCCGCTG
TCCTGACAGCGAAGTATACAACAATGCGACTTGGATATATACACCT
TCTTCTTCAGAAAGTTTGTCATCATGCTTAATCTCGACAGCTATTC
CCTTGTCTGGATCTATAGAGGCAAGTTCAACATCTGTGGTATTCGA
CATGTAGAAATGTCCATAGAAATCAGTCGGTCGAATACCCGTTGAT
GTCCTAACTCTCATAATAGCATCAAAAGCGCAAAGCCTCCTGATAT
TTTTCTCAACATCAGCTACAAGCCTCTCTCCATCTAGTTCAGCCTG
GAAGTATGTATACTTGTAAATTTCTCCACCAGTGAGCCTTGAAACT
TGACCGATAGTTGCCAGGTCAATATAGGAATTGTTAGTAATAAATA
AATCAACGCTCACTCCAGCACCAACACAGTCCTGTCCCAAGGTGTT
GTAAACAGTGTTCTGTGGCAATAAAATTGTCTTTTCTTTATCAGTC
CCCAATAACGACCTGTCATCCCTATTTTTCAACTTTCCAGGAGCTT
CTGCGATAGGAAGAGACGAGTGGAACACGAGCAGTTTACCAGCGCA
CCCAGACGCTTTAAGAGCTTCAAGGCCGGCCTGTATAGCAGGAGCC
AGTATTGTTTCTGTCTCACGGGTGTCAGCAAACATCATCGGTATAT
TCGTCATTAGTGCGTCTATTAAACCTTCAGACTCTTCAGGATCGAC
CAGGAAACCGTCCAATAGAGGCATGAACATTTCTTGAGTATCACCG
ACTACTAACATCTGGGGTTGTCCTAGGTTAGGTCTAATATTGTAGA
AATGGACAGCACTGTTATAAGTTATAAATCCAACTTTCATAGTAGA
CTTCTCCATTCCCCTTTCTTTAGGAAGATTGCGAAGAATATTTTTC
ATTTGATGACATAACAGTGAAACGAGTCCAGATTTAACATTATTGT
AAGACACATCAATAACGAATATAAGTGCAGGTGGATTAGGGAATTG
ATTGTCTTTACAATATTCTCTTGTTGCTATAATATCATAGGTCCCT
AACACAAGTTCAGCTCTTTCAAAACGATCAACTCGTTGACCAGTAT
GGTCTAAATGCTGGAAGTATTCAGCTGGTACATCAGTAGTTGCTTT
GCATAGAAGACAGTGGAAGCGCCTACCACCATCAATGAACTGCATG
TTCGGGCACATATAAGCCTTGCAACGAATACATCTTACTGGACCGA
GCTCGCCAAAAGAAACCAACGGAGGAGGATGTTCTTTATCTGCGAC
TTCCGCCATAGGACTCAACACCAAACCAAAAGGTACAGACGCCTGT
TTCATCAAATCAGAAGTTATAGGAACGTTGTACATCGTTGACCTCA
TAAACCTTGGACTGGCATTGCCCTGATCTTGAACGACGAATTCCGT
AGTAACAAGTGGAGGGACTTGGCCTTTCTGGTGTGTATAAAACACG
CCTGATCTTGTCTTCTGGTCATCTTCCATTACCTGCATTGGACTAG
GCATCTGGTCTGGGTCAAGCCTGCGAGGTTGCTGTTGAGGATACTG
CGGCTGCCCAACTCCACCAGGATAACCAGGTTGAGGCTGCGGTGGG
AAACCAGGTTGAGGGGGATAGCCCTGCTGCGGCGATGGAAGGTATG
CAGAAGTTTGTCCTCCTGATTCAGGCATTGGAGGATATCTGGATTG
AGGAGGCCTGCCAGGACCACTTGTATCAGGAAGGCCATTCATCGCC
TGACTGGGTGGGCCACCATTCACGGCTGGGTATCGAGATGGTTGTC
CAGGAGGAGCATACCCCATAGATGGCGGACCTTGCAAACCACCTCC
AGGATAGTCCCCTTGGTGTTGCTGATTCATTGGTGGCATCGGCTGC
CCATTTATGTTCATGCTGGACATCTGCCCTGCCAGCTGGTTCACCT
GAGGCATTGGTGGCCTGCCGATCTGCTGAGAACCTGGAGGATACAT
GGATGGGTGTGGTGGACCACCAGGACCGGGAGAGCTGACAGGACCA
GGCATCGAAGGGGCTCCTACAGCAGGTGGTGCTCCTGGGTGCGAAG
GTGCTGAGTTATATCCTAGAGGCACATTACTAGATGGTGGTTGAAA
GCTGTTAGGCATAGGAGCACCGCGCTGTTGAGGAGGCATTGGACCA
CCATGGTGTTGGTGTGGCAAAGAACCAGGATGCTGTTGAGGTGGCA
TTTGACCACTCTGTTGAGGTGGCACAGAACCAACTGGTTTTTGAGG
TTGCATTGGACTAAGCTGTTGTTGAGGTGGCATGGGACCACCAGGC
TGTTGAGGAGGCATAGAACTATTCTGCTGCTGAAGGGGCTTTGGAC
CACCATAGTGTTGAGACATCATTGGACCACCCTGCTGTTGAGGAGG
GGCTGGACCGCCATGCTGTGGAGGAACCATTGGACCACTGTGTTGC
TGAGGGGGCACCGGACCGCCCTGTAGTGGAGGAGGTGGCATCATGT
TGGCAGGGGAAGTCCCAGCTGGTCGGTAAGGTTGAGAAAATGCTGA
TGGTGATGCCATATTTGTTTTAGAAGGAATACCTGGATAACTTTGC
TGTGGTGGAAAAGCATTAGGTTGAAGAGGGCTTGCAGCTGGTGGCG
GAGGCGAATTTGGAACACCATAACCAGTATGAGGTCCATAACCACC
TGGTTGTGATACATACTGAGGATTCATCTTGTAAGTCTTGCCTTCA
CTTATATGGAATCTAAAACTTAATAATCTTCATAATTTTAACAAAA
CAAAAAAAAACACGAAACTAAATAATATAAGCTACTAATATCAGCT
GCAGTAGCACCACTCCACTACCCCTGCCACGTAAGGCAGAACTGCA
CAGGCGCAGTAAGATTACACGTCAAGAAATCTTCAGCGCTACCCCT
TGTGGTGGTCTACAATACAACTAGGTTATCCTAATCAAAATCAGTG
CTACTCTAGTGAAAACTAATTTCAG

SEQ ID NO:86 shows an exemplary E. heros BSB_Gho DNA, referred to herein in some places as BSB_Gho-1, which is used in some examples for the production of a dsRNA:

GATTCGACTAAAGTTAGCCCTGAGATATTTAGATGTACGCTAACTA
AAATACCCGAGACCAAATCTCTTCTTGATAAATCTAGGCTTCCCCT
TGGCGTCTTGATCCACCCATTCAAGGACCTAAATCAATTGTCGGTG
ATCCAGTGCACAGTAATAGTACGATGTAGAGCGTGTAGGACTTATA
TAAATCCTTTTGTATTCTTTGTCGACTCGAAGCATTGGAAATGCAA
TCTCTGCTTTAGGGTGAATGATTTGCCAGAAGAATTTCAATATGAC
CCATTAACAAAGACTTATGGAGACCCTACTAGACGACCAGAAATAA
AATCTGCTACTATAGAATTCATAGCTCCATCGGAATATATGGTGAG
GCCGCCGCAACCGGCTGCTTACGTGTTTG

SEQ ID NO:87 shows an exemplary E. heros BSB_Gho DNA, referred to herein in some places as BSB_Gho-2, which is used in some examples for the production of a dsRNA:

CTTTTCAAGAGGCAGTTGACATACAGAGGTAACAACTTCATGCACT
CAGGAAGGATCAGCTGTCCAGCAGAAGTAGGAGAAGCACAATTCTT
ACGATAGCACGCCAGAATCTGAGCTGACCTGTTTATTAATGATTCT
TTAACAGCTTTTGCCGATGCATCTAAAAGCTTGAACACACTCTGTT
TGGAAAAGAAGTTGATGATAGTGTCGAGTTCACAGGTTCTATAGAG
GTCGGACATCTGTGAGCAAGCCTTCAATACCAGGTTGAGAACTCTG
ATCCTCCGCTGTCCTGACAGCGAAGTATACAACAATGCGACTTGGA
TATATACACCTTCTTCTTCAGAAAGTTTGTCATCATGCTTAATCTC
GACAGCTATTCCCTTGTCTGGATCTATAGAGGCAAGTTCAACATCT
GTGGTATTCGACATGTAGAAATGTCCATAGAAATCAGTCGGTCGAA
TACCCGTTGATGTCCTAACTCTCATAATAGCATC

SEQ ID NO:88 shows an exemplary E. heros BSB_Gho DNA, referred to herein in some places as BSB_Gho-3, which is used in some examples for the production of a dsRNA:

GGACTGGCATTGCCCTGATCTTGAACGACGAATTCCGTAGTAACAA
GTGGAGGGACTTGGCCTTTCTGGTGTGTATAAAACACGCCTGATCT
TGTCTTCTGGTCATCTTCCATTACCTGCATTGGACTAGGCATCTGG
TCTGGGTCAAGCCTGCGAGGTTGCTGTTGAGGATACTGCGGCTGCC
CAACTCCACCAGGATAACCAGGTTGAGGCTGCGGTGGGAAACCAGG
TTGAGGGGGATAGCCCTGCTGCGGCGATGGAAGGTATGCAGAAGTT
TGTCCTCCTGATTCAGGCATTGGAGGATATCTGGATTGAGGAGGCC
TGCCAGGACCACTTGTATCAGGAAGGCCATTCATCGCCTGACTGGG
TGGGCCACCATTCACGGCTGGGTATCGAGATGGTTGTCCAGGAGGA
GCATACCCCATAGATGGCGGACCTTGCAAACCACCTCCAGGATAGT
CCCCTTGGTGTTGCTGATTCATTGG

SEQ ID NOs:89-94 show primers used to amplify gene regions (i.e., BSB_Gho-1, BSB_Gho-2, and BSB_Gho-3) of exemplary BSB_Gho genes.

SEQ ID NO:95 shows an exemplary YFP DNA, of which the complementary strand is transcribed to become the sense strand a YFP dsRNA (YFP v2).

SEQ ID NOs:96 and 97 show primers used to amplify portions of YFP v2.

SEQ ID NO:98 shows the amino acid sequence of a E. heros BSB_GHO polypeptide encoded by an exemplary BSB_Gho DNA:

MNGQSCTYSDSGNGTPTPSSTSYPASLSSQSSRDTSPSRLHPNLN
HINSEKSINSSGNYMNYKIHDTYTNANSVYGQIYSDSTTPTNRAT
VPPYISDTNNDINQSQRLGQPQLRPSTTSSQIITSLGSSVSKPVY
SSSHLNQISNDQKQYVNQYSTQKLDSVMQPKTSESNIIKNHETMP
TSNLAISDYYQGYTQTMNNPYRQENVLPNQTMKPEQQYHAQTQGY
QVQKPLMSPTSNPYMNSVPQDNQNYPQSPGDVPRSTFQQGYYQHQ
PQPQPQPQPPSVMSGRPQMNLPLTQSRSLDEPISSGPPRTNVLGI
IPYATEPATSQVSRPKLPDGGGYYQPMQPQQQPPQMQQPQMQQPQ
MQQQQPPRVAPRPPAPKPKGYPPPPYQQYPSYSHPQNNAGLPPYS
QTMGGYYPSGDELANQMSQLSVSQLGFNKLWGRDTVDLMKSRDVL
PPTRVEAPPVRLSQEYYDSTKVSPEIFRCTLTKIPETKSLLDKSR
LPLGVLIHPFKDLNQLSVIQCTVIVRCRACRTYINPFVFFVDSKH
WKCNLCFRVNDLPEEFQYDPLTKTYGDPTRRPEIKSATIEFIAPS
EYMVRPPQPAAYVFVLDVSRLAVESGYLRIFCDCLLSQLEALPGD
SRTAVAFITYDSAVHYYSLADTQAQPHQMVVVDIDDMFVPCPENL
LVNLSECLGLVRDLLRELPNKYRDSYDTGTAVGPALQAAYKLLAA
TGGRVTLVTSCLANSGPGKLPSREDPNQRSGEGLNQSHLNPVTDF
YKKLALDCSGQQIAVDLFVLNSQFVDLASLSGVSRFSGGCIHHFP
LFSVKNPHHVESFQRSLQRYLCRKIGFESVMRLRCTRGLSIHTFH
GNFFVRSTDLLSLPNVNPDAGFGMQVSIDENLTDIQTVCFQAALL
YTSSKGERRIRVHTLCLPIASNLSDVLHGADQQCIVGLLAKMAVD
RCHQSSLSDAREAFVNVVADMLSAFRITQSGVSPTSLVAPISLSL
LPLYVLALLKYIAFRVGQSTRLDDRVFAMCQMKSLPLSQLIQAIY
PDLYPIANINELPLVTIGEDQVVQPPLLHLSAERIDSTGVYLMDD
GTTIIIYVGHNINPSIAVSFFGVPSFSAINSNMFELPELNTPESK
KLRGFISYLQNEKPVAPTVLIIRDDSQQRHLFVEKLIEDKTESGH
SYYEFLQRVKVLVK

SEQ ID NO:99 shows the amino acid sequence of a E. heros BSB_GHO polypeptide encoded by a further exemplary BSB_Gho DNA:

MNPQYVSQPGGYGPHTGYGVPNSPPPPAASPLQPNAFPPQQSYPG
IPSKTNMASPSAFSQPYRPAGTSPANMMPPPPLQGGPVPPQQHSG
PMVPPQHGGPAPPQQQGGPMMSQHYGGPKPLQQQNSSMPPQQPGG
PMPPQQQLSPMQPQKPVGSVPPQQSGQMPPQQHPGSLPHQHHGGP
MPPQQRGAPMPNSFQPPSSNVPLGYNSAPSHPGAPPAVGAPSMPG
PVSSPGPGGPPHPSMYPPGSQQIGRPPMPQVNQLAGQMSSMNING
QPMPPMNQQHQGDYPGGGLQGPPSMGYAPPGQPSRYPAVNGGPPS
QAMNGLPDTSGPGRPPQSRYPPMPESGGQTSAYLPSPQQGYPPQP
GFPPQPQPGYPGGVGQPQYPQQQPRRLDPDQMPSPMQVMEDDQKT
RSGVFYTHQKGQVPPLVTTEFVVQDQGNASPRFMRSTMYNVPITS
DLMKQASVPFGLVLSPMAEVADKEHPPPLVSFGELGPVRCIRCKA
YMCPNMQFIDGGRRFHCLLCKATTDVPAEYFQHLDHTGQRVDRFE
RAELVLGTYDIIATREYCKDNQFPNPPALIFVIDVSYNNVKSGLV
SLLCHQMKNILRNLPKERGMEKSTMKVGFITYNSAVHFYNIRPNL
GQPQMLVVGDTQEMFMPLLDGFLVDPEESEGLIDALMTNIPMMFA
DTRETETILAPAIQAGLEALKASGCAGKLLVFHSSLPIAEAPGKL
KNRDDRSLLGTDKEKTILLPQNTVYNTLGQDCVGAGVSVDLFITN
NSYIDLATIGQVSRLTGGEIYKYTYFQAELDGERLVADVEKNIRR
LCAFDAIMRVRTSTGIRPTDFYGHFYMSNTTDVELASIDPDKGIA
VEIKHDDKLSEEEGVYIQVALLYTSLSGQRRIRVLNLVLKACSQM
SDLYRTCELDTIINFFSKQSVFKLLDASAKAVKESLINRSAQILA
CYRKNCASPTSAGQLILPECMKLLPLYVNCLLKSDAISGGKDMTV
DDKWFVMAAVLTMDVPSSLVYFYPRLYSLLELEDSVPPPCIRCSS
EKMVDSGVYLLVNGIYMFIWLGLATPSDWVMSVFGVPSAAQVDTD
RHRLPPLENPISERVRNAISSIADSNHRTMRLTIVRQRDKMEMVM
KHFLVEDRGLDGSSSYVDFLCHLHKEIRNMLS

SEQ ID NO:100 shows an exemplary DNA encoding a YFP v2-1 hairpin-forming RNA; containing a sense polynucleotide, an RTM1 intron loop (underlined), and an antisense polynucleotide (bold font):

ATGTCATCTGGAGCACTTCTCTTTCATGGGAAGATTCCTTACGTT
GTGGAGATGGAAGGGAATGTTGATGGCCACACCTTTAGCATACGT
GGGAAAGGCTACGGAGATGCCTCAGTGGGAAAGTCCGGCAACATG
TTTGACGTTTGTTTGACGTTGTAAGTCTGATTTTTGACTCTTCTT
TTTTCTCCGTCACAATTTCTACTTCCAACTAAAATGCTAAGAACA
TGGTTATAACTTTTTTTTTATAACTTAATATGTGATTTGGACCCA
GCAGATAGAGCTCATTACTTTCCCACTGAGGCATCTCCGTAGCCT
TTCCCACGTATGCTAAAGGTGTGGCCATCAACATTCCCTTCCATC
TCCACAACGTAAGGAATCTTCCCATGAAAGAGAAGTGCTCCAGAT
GACAT

SEQ ID NO:101 shows a probe used to measure maize transcript levels.

SEQ ID NO:102 shows a DNA comprising an exemplary Diabrotica Sec24B1 polynucleotide:

TCTACTCCCTGAAATTCAAGAATACGGGCCCTGGAATAATAGATA
TAACGTTAATATCATCTGTGACATATCCACATACTTGTGGAATAG
AAGTATTTCTGCAATAAAAGCAGAAGCAGAACTCCGAAGAGTTGG
CAACATTGTGCCAGCCACGTAAGATTGACAATGACGTTTGTGAAA
ATGATTATTTCTGTCCAAAAAGATTATTCAGAAAAAATGTACAGT
GCACTAATTTTTAACTGATATTTTTAATAGGAAATTATTTATTTA
ATACATAATTTCAATGTCATCATGGCTGACAGAAACGTTAATGGA
ATTTCACCGAACCCTGAAACCCTAAAACACAATGCTATATACGAG
GAAAAACTACATCAACAATTTAATGGGGTCCATTCATCACAATCA
TCAAGGAGTTCATCACCTGGTACACGCCTCGGATATGTACCCCCT
TCTCAGCTGCCTCCAAGTAGGCCTATCCCTCAATCTCAACTTCCT
CCTTCCCGATCTGCGCCGGGAAATATAACTCAACAATTCGGGGCA
TTAAACCTTAACCAAAATGCTCCCAGACATAGTCCACAATTCGGA
GCTCCTGCAACTCAACCCACTAGTTCCAGCCCCTACACAATTCCT
CCTTTTAGTCAAGTCAGTAAGGAAAGTATAAATAGTCAATCATCT
GCTATCTTACCGCCAACTTCAAATACTTCGAGTACAGTAACTTCG
TCGCAAATGTCTACACCTCTTCAACAAGGACCATTCAGTGCTCAA
CCTACAAGTGGTTTTCAGAAACCTGATCCATTTCAAGCAATTAAA
CCAGCACAAACCAATAATACTCAGCCGACTTCTAATGTAAATAAT
CAACCATCGCAAAATCCAATGCAATTTAATCAGAACTCTCCTAAT
GTCAGGCTTCAACCTAACCAAGTACCAGTGCAAAATAATATGGGC
GTTCCAACTAATTCAAACATGCCTAGGATAAGCCCGGTTCCACCT
CAACAGAACTTTCAACCTAGTCCTAATAGATCAGCTTTTGGTCCA
ATACCACCGCCTGGAATACAGAATCCGATAGTTAGTCAAATTAGT
CCAAACAGGACAGGTTTAGTTCAGGGACCACCGTTACAAACACAA
TACAGAGCTCCTAATCAAATTCCTGGGCCACCGCCACAAGCTGGT
GTACTTCAAGCAAACCAGCAAAGGTCATACCAAGCATCCCCAATT
CAACAAAATAATAACCAAAGATTTAACAATGCTATTGCTACCCAA
AATATCAATAATGGTCCAACTATGAACGCAAATTTTCCTCCACAA
GCTGCACCTTCTAACTACCCACAAATGAATAGTGCACCACCGCCC
CAAACAAACGTGGCACCGAAAACGAATGTACATTCAAACAGGTAT
CCTACGATGCAGTCAAACAGCTACCAACAACCCGCCCCATCTCAA
TATCAGCAACAGCCACCTTCTGGCCAGTATCAGTATCAACAACCA
ATGCAACAACCAGTACAACAACCAATGAATTCGTATCCAAGTCAA
AATAATCAGCAGTCTCCTTACCAAGGAGTAGTAAATACTGGCTTT
AATAAATTATGGGGTATGGAACAGTTTGACCTTCTTCAAACTCCA
AATATATTGCAACCATCGAAAGTCGAAGCTCCTCAAATTCGTTTG
GGCCAAGACTTGTTGGATCAAGCCAATTGCAGCCCAGACGTGTTT
CGTTGCACTATGACGAAAATTCCAGAAAATAATTCTCTTTTACAG
AAGTCGAGATTGCCTTTAGGGGTGTTAATTCATCCGTTTAGGGAT
CTTTCTCATTTACCTGTAATTCAGTGCAGTGTAATAGTTAGGTGT
AGAGCGTGTCGCACCTATATAAATCCCTTTGTCCTTTTTGTTGAT
AATAAACGCTGGAAGTGCAATTTGTGCTATAGAATCAACGAGTTA
CCCGAAGAATTTCAGTACGATCCGATGACGAAAACGTACGGAGAC
CCTTCTAGAAGACCAGAGATTAAATCCAGCACTTTGGAATACATT
GCACCTGCTGAATATATGTTGAGGCCACCCCAGCCTGCAGTATAC
CTTTATTTACTGGACGTATCTCGATTGGCAATGGAAAGTGGTTAT
TTGAATATTGTATGTAGTATTTTATTGGAAGAATTGAAGAATTTG
CCTGGAGATGCAAGAACGCAAATTGGATTTATTGCTTATAACTCT
GCTCTACATTTTTATTCTTTGCCAGAGGGTATCACCCAACCACAC
GAGATGACAATTCTCGACATAGACGATATATTCCTCCCTACACCC
GATAATTTATTAGTCAATTTAAAGGATAGAATGGACTTAATAGCA
GACCTTTTGAGGCTCTTACCGAACAGATTTGCCAACACATTTGAC
ACCAACTCTGCTCTTGGTGCTGCATTGCAAGTTGCATTCAAGATG
ATGGGTGCAACAGGTGGTAGAGTTACTGTATTCCAAGCATCACTG
CCAAACATCGGACCTGGAGCGCTTATCTCAAGAGAAGATCCATCC
AATAGAGCATCAGCCGAAGTTGCGCATCTAAACCCTGCTAACGAT
TTCTATAAACGCTTGGCGTTGGAGTGCAGCGGTCAGCAGATTGCA
GTCGATCTGTTCGTAGTAAACTCTCAGTATGTAGATATAGCTACT
ATTTCAGGAATTAGCAGATTCAGCGGGGGTTGTATGCATCACTTC
CCTTTACTCAAACCTACAAAGCCAGTAGTCTGTGATCGTTTTGCT
AGATCTTTTAGGAGGTATATCACCAGGAAAATTGGTTTTGAGGCC
GTGATGAGATTGAGGTGTACAAGAGGACTTTCTATTCATACCTTC
CACGGTAATTTCTTCGTTCGATCGACAGATTTACTATCTTTGCCT
AACATTAATCCCGATGCAGGGTTTGGCATGCAAGTTGCTATCGAA
GAGAGTTTATCCGATGTTCAGACTGTATGTTTCCAGGCAGCATTA
CTATACACGTCGAGCAAAGGCGAAAGAAGAATAAGAGTTCATACG
ATGTGCTTGCCGGTGGCTACGACTATACAAGACGTCATCCACTCT
GCCGACCAGCAATGCATCATAGGCTTATTGTCAAAAATGGCTGTT
GATAGATCGATGCAATCTAGTCTTTCAGATGCCCGCGAGGCGTTT
ATCAACGTAGCAATAGATATTCTATCGAGTTTTAAAATGAGTCTG
AACATGGGTAGTCCCGTAACGGGTCTGTTAGTGCCGAATTGTATG
CGAATATTGCCTTTGTATATATCAGCTCTTCTTAAACATTTAGCG
TTTAGAACAGGTAGTTCTACTAGGTTAGATGACAGAGTAATGAAA
ATGATAGAGATGAAAACGAAACCATTGTACATGCTCATACAGGAT
ATATACCCCGATCTGTTCCCCATCCATAATTTAGAACACCAAGAA
GTGATCATGAATTCTGAAGAGGAACCAGTTTCTATGCCACCTAGG
TTACAACTCACCGCCAGATGTCTGGAGAATAAAGGTGCGTTTTTG
CTGGATACGGGCGAGCATATGATCATCCTAGTTTGTCCAAATGTG
CCACAAGAATTTTTAACCGAAGCTCTGGGAGTTTCCCAATATAGC
GCCATTCCGGATGATATGTATGAAATACCCGTGTTAGATAATCTT
AGAAATCAAAGACTTCATCAATTTATTACATATTTAAATGAGGAA
AAGCCGTATCCGGCCACGTTACAAGTGATTAGAGACAATAGTACG
AATAGAGTTGTATTTTTCGAGAGATTAATAGAGGACCGAGTCGAA
GATGCACTTTCTTATCACGAATTTTTGCAACATTTAAAAACTCAA
GTGAAGTAAGGTTAAGTGTACATTTATTATTTTTATCTTTTTATT
TAAATTGTGCAGATTTATTGCTTGTGCAAAGACCACTCCGAAATT
ATTTCCGTATAAAATAACTAGGTATTTTACAGATCCAGGAACGTC
CAATTATATGTTTGTAACTTCAGAGTATGGTCAAACCACAGCCAT
ATAATACCCAAGACTGCGCGCTGTAATATAAAACCGTGCAGTCCT
TACATCACTTTTTAATGAGCGGGGTTTATCGACCACGTGACAATC
CCACTAGGGATTGTTTAGTAGTTAGAAAGAGATGCAAGGACTGCT
CGCAATCTGCTTTCTCTGTCGCATTGGGGAAATGGTTTTAAATTA
CAGCGTGTAGTCTAAGTATTATATGTCTATGGGTGAAACAATGTA
TCCAGTGACATGTTCCATTTCAACTTAAACTTAACGACTATATTA
AATTTACAGTCAAGATGCAGTG

SEQ ID NO:103 shows the amino acid sequence of a Diabrotica SEC24B1 polypeptide encoded by an exemplary Diabrotica Sec24B1 DNA:

MADRNVNGISPNPETLKHNAIYEEKLHQQFNGVHSSQSSRSSSPG
TRLGYVPPSQLPPSRPIPQSQLPPSRSAPGNITQQFGALNLNQNA
PRHSPQFGAPATQPTSSSPYTIPPFSQVSKESINSQSSAILPPTS
NTSSTVTSSQMSTPLQQGPFSAQPTSGFQKPDPFQAIKPAQTNNT
QPTSNVNNQPSQNPMQFNQNSPNVRLQPNQVPVQNNMGVPTNSNM
PRISPVPPQQNFQPSPNRSAFGPIPPPGIQNPIVSQISPNRTGLV
QGPPLQTQYRAPNQIPGPPPQAGVLQANQQRSYQASPIQQNNNQR
FNNAIATQNINNGPTMNANFPPQAAPSNYPQMNSAPPPQTNVAPK
TNVHSNRYPTMQSNSYQQPAPSQYQQQPPSGQYQYQQPMQQPVQQ
PMNSYPSQNNQQSPYQGVVNTGFNKLWGMEQFDLLQTPNILQPSK
VEAPQIRLGQDLLDQANCSPDVFRCTMTKIPENNSLLQKSRLPLG
VLIHPFRDLSHLPVIQCSVIVRCRACRTYINPFVLFVDNKRWKCN
LCYRINELPEEFQYDPMTKTYGDPSRRPEIKSSTLEYIAPAEYML
RPPQPAVYLYLLDVSRLAMESGYLNIVCSILLEELKNLPGDARTQ
IGFIAYNSALHFYSLPEGITQPHEMTILDIDDIFLPTPDNLLVNL
KDRMDLIADLLRLLPNRFANTFDTNSALGAALQVAFKMMGATGGR
VTVFQASLPNIGPGALISREDPSNRASAEVAHLNPANDFYKRLAL
ECSGQQIAVDLFVVNSQYVDIATISGISRFSGGCMHHFPLLKPTK
PVVCDRFARSFRRYITRKIGFEAVMRLRCTRGLSIHTFHGNFFVR
STDLLSLPNINPDAGFGMQVAIEESLSDVQTVCFQAALLYTSSKG
ERRIRVHTMCLPVATTIQDVIHSADQQCIIGLLSKMAVDRSMQSS
LSDAREAFINVAIDILSSFKMSLNMGSPVTGLLVPNCMRILPLYI
SALLKHLAFRTGSSTRLDDRVMKMIEMKTKPLYMLIQDIYPDLFP
IHNLEHQEVIMNSEEEPVSMPPRLQLTARCLENKGAFLLDTGEHM
IILVCPNVPQEFLTEALGVSQYSAIPDDMYEIPVLDNLRNQRLHQ
FITYLNEEKPYPATLQVIRDNSTNRVVFFERLIEDRVEDALSYHE
FLQHLKTQVK

SEQ ID NO:104 shows an exemplary Diabrotica Sec24B1 DNA, referred to herein in some places as Sec24B1 reg1, which is used in some examples for the production of a dsRNA:

CTCAGTATGTAGATATAGCTACTATTTCAGGAATTAGCAGATTCA
GCGGGGGTTGTATGCATCACTTCCCTTTACTCAAACCTACAAAGC
CAGTAGTCTGTGATCGTTTTGCTAGATCTTTTAGGAGGTATATCA
CCAGGAAAATTGGTTTTGAGGCCGTGATGAGATTGAGGTGTACAA
GAGGACTTTCTATTCATACCTTCCA

SEQ ID NOs:105-106 show primers used to amplify a gene region (Sec24B1 reg1) of a Diabrotica Sec24B1 gene.

SEQ ID NO:107 shows a DNA comprising a further exemplary Diabrotica Sec24B2 polynucleotide:

GACACTTGTCTAAGTTCCGAACTTGGTATAATTTTCAGGTTATGG
TCATTCAATGCCAAAAAAAATATGATCACGTGTCACTTATCTGTC
AACAGTACGAATATTTATTTAACAATCATTTATGATGAAGAAATA
AAAAATAAATAATTATTTTTGATAAACTTGCTTCTAGAAGATGAT
TAAAATGCTGGAATAATAGATATAACGTTAATATCATCTGTGACA
TATCCACATACTTGTGGAATAGAAGTATTTCTGCAATAAAAGCAG
AAGCAGAACTCCGAAGAGTTGGCAACATTGTGCCAGCCACGTAAG
ATTGACAATGACGTTTGTGAAAATGATTATTTCTGTCCAAAAAGA
TTATTCAGAAAAAATGTACAGTGCACTAATTTTTAACTGATATTT
TTAATAGGAAATTATTTATTTAATACATAATTTCAATGTCATCAT
GGCTGACAGAAACGTTAATGGAATTTCACCGAACCCTGAAACCCT
AAAACACAATGCTATATACGAGGAAAAACTACATCAACAATTTAA
TGGGGTCCATTCATCACAATCATCAAGGAGTTCATCACCTGGTAC
ACGCCTCGGATATGTACCCCCTTCTCAGCTGCCTCCAAGTAGGCC
TATCCCTCAATCTCAACTTCCTCCTTCCCGATCTGCGCCGGGAAA
TATAACTCAACAATTCGGGGCATTAAACCTTAACCAAAATGCTCC
CAGACATAGTCCACAATTCGGAGCTCCTGCAACTCAACCCACTAG
TTCCAGCCCCTACACAATTCCTCCTTTTAGTCAAGTCAGTAAGGA
AAGTATAAATAGTCAATCATCTGCTATCTTACCGCCAACTTCAAA
TACTTCGAGTACAGTAACTTCGTCGCAAATGTCTACACCTCTTCA
ACAAGGACCATTCAGTGCTCAACCTACAAGTGGTTTTCAGAAACC
TGATCCATTTCAAGCAATTAAACCAGCACAAACCAATAATACTCA
GCCGACTTCTAATGTAAATAATCAACCATCGCAAAATCCAATGCA
ATTTAATCAGAACTCTCCTAATGTCAGGCTTCAACCTAACCAAGT
ACCAGTGCAAAATAATATGGGCGTTCCAACTAATTCAAACATGCC
TAGGATAAGCCCGGTTCCACCTCAACAGAACTTTCAACCTAGTCC
TAATAGATCAGCTTTTGGTCCAATACCACCGCCTGGAATACAGAA
TCCGATAGTTAGTCAAATTAGTCCAAACAGGACAGGTTTAGTTCA
GGGACCACCGTTACAAACACAATACAGAGCTCCTAATCAAATTCC
TGGGCCACCGCCACAAGCTGGTGTACTTCAAGCAAACCAGCAAAG
GTCATACCAAGCATCCCCAATTCAACAAAATAATAACCAAAGATT
TAACAATGCTATTGCTACCCAAAATATCAATAATGGTCCAACTAT
GAACGCAAATTTTCCTCCACAAGCTGCACCTTCTAACTACCCACA
AATGAATAGTGCACCACCGCCCCAAACAAACGTGGCACCGAAAAC
GAATGTACATTCAAACAGGTATCCTACGATGCAGTCAAACAGCTA
CCAACAACCCGCCCCATCTCAATATCAGCAACAGCCACCTTCTGG
CCAGTATCAGTATCAACAACCAATGCAACAACCAGTACAACAACC
AATGAATTCGTATCCAAGTCAAAATAATCAGCAGTCTCCTTACCA
AGGAGTAGTAAATACTGGCTTTAATAAATTATGGGGTATGGAACA
GTTTGACCTTCTTCAAACTCCAAATATATTGCAACCATCGAAAGT
CGAAGCTCCTCAAATTCGTTTGGGCCAAGACTTGTTGGATCAAGC
CAATTGCAGCCCAGACGTGTTTCGTTGCACTATGACGAAAATTCC
AGAAAATAATTCTCTTTTACAGAAGTCGAGATTGCCTTTAGGGGT
GTTAATTCATCCGTTTAGGGATCTTTCTCATTTACCTGTAATTCA
GTGCAGTGTAATAGTTAGGTGTAGAGCGTGTCGCACCTATATAAA
TCCCTTTGTCCTTTTTGTTGATAATAAACGCTGGAAGTGCAATTT
GTGCTATAGAATCAACGAGTTACCCGAAGAATTTCAGTACGATCC
GATGACGAAAACGTACGGAGACCCTTCTAGAAGACCAGAGATTAA
ATCCAGCACTTTGGAATACATTGCACCTGCTGAATATATGTTGAG
GCCACCCCAGCCTGCAGTATACCTTTATTTACTGGACGTATCTCG
ATTGGCAATGGAAAGTGGTTATTTGAATATTGTATGTAGTATTTT
ATTGGAAGAATTGAAGAATTTGCCTGGAGATGCAAGAACGCAAAT
TGGATTTATTGCTTATAACTCTGCTCTACATTTTTATTCTTTGCC
AGAGGGTATCACCCAACCACACGAGATGACAATTCTCGACATAGA
CGATATATTCCTCCCTACACCCGATAATTTATTAGTCAATTTAAA
GGATAGAATGGACTTAATAGCAGACCTTTTGAGGCTCTTACCGAA
CAGATTTGCCAACACATTTGACACCAACTCTGCTCTTGGTGCTGC
ATTGCAAGTTGCATTCAAGATGATGGGTGCAACAGGTGGTAGAGT
TACTGTATTCCAAGCATCACTGCCAAACATCGGACCTGGAGCGCT
TATCTCAAGAGAAGATCCATCCAATAGAGCATCAGCCGAAGTTGC
GCATCTAAACCCTGCTAACGATTTCTATAAACGCTTGGCGTTGGA
GTGCAGCGGTCAGCAGATTGCAGTCGATCTGTTCGTAGTAAACTC
TCAGTATGTAGATATAGCTACTATTTCAGGAATTAGCAGATTCAG
CGGGGGTTGTATGCATCACTTCCCTTTACTCAAACCTACAAAGCC
AGTAGTCTGTGATCGTTTTGCTAGATCTTTTAGGAGGTATATCAC
CAGGAAAATTGGTTTTGAGGCCGTGATGAGATTGAGGTGTACAAG
AGGACTTTCTATTCATACCTTCCACGGTAATTTCTTCGTTCGATC
GACAGATTTACTATCTTTGCCTAACATTAATCCCGATGCAGGGTT
TGGCATGCAAGTTGCTATCGAAGAGAGTTTATCCGATGTTCAGAC
TGTATGTTTCCAGGCAGCATTACTATACACGTCGAGCAAAGGCGA
AAGAAGAATAAGAGTTCATACGATGTGCTTGCCGGTGGCTACGAC
TATACAAGACGTCATCCACTCTGCCGACCAGCAATGCATCATAGG
CTTATTGTCAAAAATGGCTGTTGATAGATCGATGCAATCTAGTCT
TTCAGATGCCCGCGAGGCGTTTATCAACGTAGCAATAGATATTCT
ATCGAGTTTTAAAATGAGTCTGAACATGGGTAGTCCCGTAACGGG
TCTGTTAGTGCCGAATTGTATGCGAATATTGCCTTTGTATATATC
AGCTCTTCTTAAACATTTAGCGTTTAGAACAGGTAGTTCTACTAG
GTTAGATGACAGAGTAATGAAAATGATAGAGATGAAAACGAAACC
ATTGTACATGCTCATACAGGATATATACCCCGATCTGTTCCCCAT
CCATAATTTAGAACACCAAGAAGTGATCATGAATTCTGAAGAGGA
ACCAGTTTCTATGCCACCTAGGTTACAACTCACCGCCAGATGTCT
GGAGAATAAAGGTGCGTTTTTGCTGGATACGGGCGAGCATATGAT
CATCCTAGTTTGTCCAAATGTGCCACAAGAATTTTTAACCGAAGC
TCTGGGAGTTTCCCAATATAGCGCCATTCCGGATGATATGTATGA
AATACCCGTGTTAGATAATCTTAGAAATCAAAGACTTCATCAATT
TATTACATATTTAAATGAGGAAAAGCCGTATCCGGCCACGTTACA
AGTGATTAGAGACAATAGTACGAATAGAGTTGTATTTTTCGAGAG
ATTAATAGAGGACCGAGTCGAAGATGCACTTTCTTATCACGAATT
TTTGCAACATTTAAAAACTCAAGTGAAGTAAGGTTAAGTGTACAT
TTATTATTTTTATCTTTTTATTTAAATTGTGCAGATTTATTGCTT
GTGCAAAGACCACTCCGAAATTATTTCCGTATAAAATAACTAGGT
ATTTTACAGATCCAGGAACGTCCAATTATATGTTTGTAACTTCAG
AGTATGGTCAAACCACAGCCATATAATACCCAAGACTGCGCGCTG
TAATATAAAACCGTGCAGTCCTTACATCACTTTTTAATGAGCGGG
GTTTATCGACCACGTGACAATCCCACTAGGGATTGTTTAGTAGTT
AGAAAGAGATGCAAGGACTGCTCGCAATCTGCTTTCTCTGTCGCA
TTGGGGAAATGGTTTTAAATTACAGCGTGTAGTCTAAGTATTATA
TGTCTATGGGTGAAACAATGTATCCAGTGACATGTTCCATTTCAA
CTTAAACTTAACGACTATATTAAATTTACAGTCAAGATGCAGTGG
AGGTGGACAGACCAAGACACGTTAAATGCTACT

SEQ ID NO:108 shows the amino acid sequence of a Diabrotica SEC24B2 polypeptide encoded by an exemplary Diabrotica Sec24B2 DNA:

MADRNVNGISPNPETLKHNAIYEEKLHQQFNGVHSSQSSRSSSPG
TRLGYVPPSQLPPSRPIPQSQLPPSRSAPGNITQQFGALNLNQNA
PRHSPQFGAPATQPTSSSPYTIPPFSQVSKESINSQSSAILPPTS
NTSSTVTSSQMSTPLQQGPFSAQPTSGFQKPDPFQAIKPAQTNNT
QPTSNVNNQPSQNPMQFNQNSPNVRLQPNQVPVQNNMGVPTNSNM
PRISPVPPQQNFQPSPNRSAFGPIPPPGIQNPIVSQISPNRTGLV
QGPPLQTQYRAPNQIPGPPPQAGVLQANQQRSYQASPIQQNNNQR
FNNAIATQNINNGPTMNANFPPQAAPSNYPQMNSAPPPQTNVAPK
TNVHSNRYPTMQSNSYQQPAPSQYQQQPPSGQYQYQQPMQQPVQQ
PMNSYPSQNNQQSPYQGVVNTGFNKLWGMEQFDLLQTPNILQPSK
VEAPQIRLGQDLLDQANCSPDVFRCTMTKIPENNSLLQKSRLPLG
VLIHPFRDLSHLPVIQCSVIVRCRACRTYINPFVLFVDNKRWKCN
LCYRINELPEEFQYDPMTKTYGDPSRRPEIKSSTLEYIAPAEYML
RPPQPAVYLYLLDVSRLAMESGYLNIVCSILLEELKNLPGDARTQ
IGFIAYNSALHFYSLPEGITQPHEMTILDIDDIFLPTPDNLLVNL
KDRMDLIADLLRLLPNRFANTFDTNSALGAALQVAFKMMGATGGR
VTVFQASLPNIGPGALISREDPSNRASAEVAHLNPANDFYKRLAL
ECSGQQIAVDLFVVNSQYVDIATISGISRFSGGCMHHFPLLKPTK
PVVCDRFARSFRRYITRKIGFEAVMRLRCTRGLSIHTFHGNFFVR
STDLLSLPNINPDAGFGMQVAIEESLSDVQTVCFQAALLYTSSKG
ERRIRVHTMCLPVATTIQDVIHSADQQCIIGLLSKMAVDRSMQSS
LSDAREAFINVAIDILSSFKMSLNMGSPVTGLLVPNCMRILPLYI
SALLKHLAFRTGSSTRLDDRVMKMIEMKTKPLYMLIQDIYPDLFP
IHNLEHQEVIMNSEEEPVSMPPRLQLTARCLENKGAFLLDTGEHM
IILVCPNVPQEFLTEALGVSQYSAIPDDMYEIPVLDNLRNQRLHQ
FITYLNEEKPYPATLQVIRDNSTNRVVFFERLIEDRVEDALSYHE
FLQHLKTQVK

SEQ ID NO:109 shows an exemplary Diabrotica Sec24B2 DNA, referred to herein in some places as Sec24B2 reg3, which is used in some examples for the production of a dsRNA:

GCTTATAACTCTGCTCTACATTTTTATTCTTTGCCAGAGGGTATC
ACCCAACCACACGAGATGACAATTCTCGACATAGACGATATATTC
CTCCCTACACCCGATAATTTATTAGTCAATTTAAAGGATAGAATG
GACTTAATAGCAGACCTTTTGAGGCTCTTACCGAACAGATTTGCC
AACACATTTGACACCAACTCTGCTCTTGGTGCTGCATTGCAAGTT
GCATTCAAGATGATGGGTGCAACAGGTGGTAGAGTTACTGTATTC
CAAGCATCACTGCCAAACATCGGACCTGGAGCGCTTATCTCAAGA
GAAGATCCATCCAATAGAGCATCAGCCGAAGTTGCGCATCTAAAC
CCTGCTAACGATTTCTATAAACGCTTGGCGTTGGAGTGCAGCGGT
CAGCAGATTGCAGTCGATCTGTTCGTAGTAAACTCTCAG

SEQ ID NOs:110 and 111 show primers used to amplify a gene region (Sec24B2 reg3) of a Diabrotica Sec24B2 gene.

SEQ ID NOs:112-127 show exemplary iRNAs that are used in particular examples to reduce the expression of a target gene in a coleopteran and/or hemipteran pest.

### DETAILED DESCRIPTION

### I. Overview of Several Embodiments

We developed RNA interference (RNAi) as a tool for insect pest management, using one of the most likely target pest species for transgenic plants that express dsRNA; the western corn rootworm. Thus far, most genes proposed as targets for RNAi in rootworm larvae do not actually achieve their purpose. Herein, we describe RNAi-mediated knockdown of Gho/Sec24B2 and/or Sec24B1 in the exemplary insect pests, western corn rootworm and neotropical brown stink bug, which is shown to have a lethal phenotype when, for example, iRNA molecules are delivered via ingestion or injection of Gho/Sec24B2 or Sec24B1 dsRNA. In embodiments herein, the ability to deliver Gho/Sec24B2 or Sec24B1 dsRNA by feeding to insects confers an RNAi effect that is very useful for insect (e.g., coleopteran and hemipteran) pest management. By combining Gho/Sec24B2 and/or Sec24B1-mediated RNAi with other useful RNAi targets, the potential to affect multiple target sequences, for example, with multiple modes of action, may increase opportunities to develop sustainable approaches to insect pest management involving RNAi technologies.

Disclosed herein are methods and compositions for genetic control of insect (e.g., coleopteran and/or hemipteran) pest infestations. Methods for identifying one or more gene(s) essential to the lifecycle of an insect pest for use as a target gene for RNAi-mediated control of an insect pest population are also provided. DNA plasmid vectors encoding an RNA molecule may be designed to suppress one or more target gene(s) essential for growth, survival, and/or development. In some embodiments, the RNA molecule may be capable of forming dsRNA molecules. In some embodiments, methods are provided for post-transcriptional repression of expression or inhibition of a target gene via nucleic acid molecules that are complementary to a coding or non-coding sequence of the target gene in an insect pest. In these and further embodiments, a pest may ingest one or more dsRNA, siRNA, shRNA, miRNA, and/or hpRNA molecules transcribed from all or a portion of a nucleic acid molecule that is complementary to a coding or non-coding sequence of a target gene, thereby providing a plant-protective effect.

Thus, some embodiments involve sequence-specific inhibition of expression of target gene products, using iRNA (e.g., dsRNA, siRNA, shRNA, miRNA and/or hpRNA) that is complementary to coding and/or non-coding sequences of the target gene(s) to achieve at least partial control of an insect (e.g., coleopteran and/or hemipteran) pest. Disclosed is a set of isolated and purified nucleic acid molecules comprising a polynucleotide, for example, as set forth in any of SEQ ID NOs:1, 84, 85, 102, and 107, and fragments thereof. In some embodiments, a stabilized dsRNA molecule may be expressed from these polynucleotides, fragments thereof, or a gene comprising one or more of these polynucleotides, for the post-transcriptional silencing or inhibition of a target gene. In certain embodiments, isolated and purified nucleic acid molecules comprise all or part of any of SEQ ID NOs:1, 3-6, 84-88, 102, 104, 107, and 109. In some embodiments, an iRNA used to achieve at least partial control of a coleopteran and/or hemipteran pest comprises all or part of the complement of a RNA molecule transcribed from any of SEQ ID NOs:1, 84, 85, 102, and 107. In certain embodiments, an iRNA comprises all or part of any of SEQ ID NOs:112-127.

Some embodiments involve a recombinant host cell (e.g., a plant cell) having in its genome at least one recombinant DNA encoding at least one iRNA (e.g., dsRNA) molecule(s). In particular embodiments, the dsRNA molecule(s) may be produced when ingested by a coleopteran and/or hemipteran pest to post-transcriptionally silence or inhibit the expression of a target gene in the pest. The recombinant DNA may comprise, for example, any of SEQ ID NOs:1, 3-6, 84-88, 102, 104, 107, and 109; fragments of any of SEQ ID NOs:1, 3-6, 84-88, 102, 104, 107, and 109; a polynucleotide consisting of a partial sequence of a gene comprising one of SEQ ID NOs:1, 3-6, 84-88, 102, 104, 107, and 109; and/or complements thereof.

Some embodiments involve a recombinant host cell having in its genome a recombinant DNA encoding at least one iRNA (e.g., dsRNA) molecule(s) comprising all or part of an RNA encoded by SEQ ID NO:1, SEQ ID NO:84, SEQ ID NO:85, SEQ ID NO:102, and/or SEQ ID NO:107; and/or the complement thereof (e.g., at least one polynucleotide selected from a group comprising SEQ ID NOs:112-127). When ingested by an insect (e.g., coleopteran and/or hemipteran) pest, the iRNA molecule(s) may silence or inhibit the expression of a target Gho/Sec24B2 and/or Sec24B1 DNA (e.g., a DNA comprising all or part of a polynucleotide selected from the group consisting of SEQ ID NOs:1, 84, 85, 102, and/or 107) in the pest, and thereby result in cessation of feeding, growth, and/or developmentin the pest.

In some embodiments, a recombinant host cell having in its genome at least one recombinant DNA encoding at least one RNA molecule capable of forming a dsRNA molecule may be a transformed plant cell. Some embodiments involve transgenic plants comprising such a transformed plant cell. In addition to such transgenic plants, progeny plants of any transgenic plant generation, transgenic seeds, and transgenic plant products, are all provided, each of which comprises recombinant DNA(s). In particular embodiments, an RNA molecule capable of forming a dsRNA molecule may be expressed in a transgenic plant cell. Therefore, in these and other embodiments, a dsRNA molecule may be isolated from a transgenic plant cell. In particular embodiments, the transgenic plant is a plant selected from the group comprising corn (Zea mays), soybean (Glycine max), and plants of the family Poaceae.

Some embodiments involve a method for modulating the expression of a target gene in an insect (e.g., coleopteran and/or hemipteran) pest cell. In these and other embodiments, a nucleic acid molecule may be provided, wherein the nucleic acid molecule comprises a polynucleotide encoding an RNA molecule capable of forming a dsRNA molecule. In particular embodiments, a polynucleotide encoding an RNA molecule capable of forming a dsRNA molecule may be operatively linked to a promoter, and may also be operatively linked to a transcription termination sequence. In particular embodiments, a method for modulating the expression of a target gene in an insect pest cell may comprise: (a) transforming a plant cell with a vector comprising a polynucleotide encoding an RNA molecule capable of forming a dsRNA molecule; (b) culturing the transformed plant cell under conditions sufficient to allow for development of a plant cell culture comprising a plurality of transformed plant cells; (c) selecting for a transformed plant cell that has integrated the vector into its genome; and (d) determining that the selected transformed plant cell comprises the RNA molecule capable of forming a dsRNA molecule encoded by the polynucleotide of the vector. A plant may be regenerated from a plant cell that has the vector integrated in its genome and comprises the dsRNA molecule encoded by the polynucleotide of the vector.

Thus, also disclosed is a transgenic plant comprising a vector having a polynucleotide encoding an RNA molecule capable of forming a dsRNA molecule integrated in its genome, wherein the transgenic plant comprises the dsRNA molecule encoded by the polynucleotide of the vector. In particular embodiments, expression of an RNA molecule capable of forming a dsRNA molecule in the plant is sufficient to modulate the expression of a target gene in a cell of an insect (e.g., coleopteran or hemipteran) pest that contacts the transformed plant or plant cell (for example, by feeding on the transformed plant, a part of the plant (e.g., root) or plant cell), such that growth and/or survival of the pest is inhibited. Transgenic plants disclosed herein may display resistance and/or enhanced tolerance to insect pest infestations. Particular transgenic plants may display resistance and/or enhanced protection from one or more coleopteran and/or hemipteran pest(s) selected from the group consisting of: WCR; NCR; SCR; MCR; D. balteata LeConte; D. u. tenella; D. u. undecimpunctata Mannerheim; Euschistus heros; Piezodorus guildinii; Halyomorpha halys; Nezara viridula; Chinavia hilare; Euschistus servus; Dichelops melacanthus; Dichelops furcatus; Edessa meditabunda; Thyanta perditor; Chinavia marginatum; Horcias nobilellus; Taedia stigmosa; Dysdercus peruvianus; Neomegalotomus parvus; Leptoglossus zonatus; Niesthrea sidae; Lygus hesperus; and Lygus lineolaris.

Also disclosed herein are methods for delivery of control agents, such as an iRNA molecule, to an insect (e.g., coleopteran and/or hemipteran) pest. Such control agents may cause, directly or indirectly, an impairment in the ability of an insect pest population to feed, grow or otherwise cause damage in a host. In some embodiments, a method is provided comprising delivery of a stabilized dsRNA molecule to an insect pest to suppress at least one target gene in the pest, thereby causing RNAi and reducing or eliminating plant damage in a pest host. In some embodiments, a method of inhibiting expression of a target gene in the insect pest may result in cessation of growth, survival, and/or development, in the pest.

In some embodiments, compositions (e.g., a topical composition) are provided that comprise an iRNA (e.g., dsRNA) molecule for use with plants, animals, and/or the environment of a plant or animal to achieve the elimination or reduction of an insect (e.g., coleopteran and/or hemipteran) pest infestation. In particular embodiments, the composition may be a nutritional composition or food source to be fed to the insect pest. Some embodiments comprise making the nutritional composition or food source available to the pest. Ingestion of a composition comprising iRNA molecules may result in the uptake of the molecules by one or more cells of the pest, which may in turn result in the inhibition of expression of at least one target gene in cell(s) of the pest. Ingestion of or damage to a plant or plant cell by an insect pest infestation may be limited or eliminated in or on any host tissue or environment in which the pest is present by providing one or more compositions comprising an iRNA molecule in the host of the pest.

The compositions and methods disclosed herein may be used together in combinations with other methods and compositions for controlling damage by insect (e.g., coleopteran and/or hemipteran) pests. For example, an iRNA molecule as described herein for protecting plants from insect pests may be used in a method comprising the additional use of one or more chemical agents effective against an insect pest, biopesticides effective against such a pest, crop rotation, recombinant genetic techniques that exhibit features different from the features of RNAi-mediated methods and RNAi compositions (e.g., recombinant production of proteins in plants that are harmful to an insect pest (e.g., Bt toxins)).

### II. Abbreviations

| | |
|---|---|
| BSB | Neotropical brown stink bug (Euschistus heros) |
| dsRNA | double-stranded ribonucleic acid |
| EST | expressed sequence tag |
| GI | growth inhibition |
| NCBI | National Center for Biotechnology Information |
| gDNA | genomic DNA |
| iRNA | inhibitory ribonucleic acid |
| ORF | open reading frame |
| RNAi | ribonucleic acid interference |
| miRNA | micro ribonucleic acid |
| siRNA | small inhibitory ribonucleic acid |
| shRNA | short hairpin ribonucleic acid |
| hpRNA | hairpin ribonucleic acid |
| UTR | untranslated region |
| WCR | western corn rootworm (Diabrotica virgifera |
| | virgifera LeConte) |
| NCR | northern corn rootworm (Diabrotica barberi |
| | Smith and Lawrence) |
| MCR | Mexican corn rootworm (Diabrotica virgifera zeae |
| | Krysan and Smith) |
| PCR | Polymerase chain reaction |
| qPCR | quantative polymerase chain reaction |
| RISC | RNA-induced Silencing Complex |
| SCR | southern corn rootworm (Diabrotica |
| | undecimpunctata howardi Barber) |
| SEM | standard error of the mean |
| YFP | yellow fluorescent protein |

### III. Terms

In the description and tables which follow, a number of terms are used. In order to provide a clear and consistent understanding of the specification and claims, including the scope to be given such terms, the following definitions are provided:

Coleopteran pest: As used herein, the term “coleopteran pest” refers to insects of the order Coleoptera, including pest insects in the genus Diabrotica, which feed upon agricultural crops and crop products, including corn and other true grasses. In particular examples, a coleopteran pest is selected from a list comprising D. v. virgifera LeConte (WCR); D. barberi Smith and Lawrence (NCR); D. u. howardi (SCR); D. v. zeae (MCR); D. balteata LeConte; D. u. tenella; and D. u. undecimpunctata Mannerheim.

Contact (with an organism): As used herein, the term “contact with” or “uptake by” an organism (e.g., a coleopteran or hemipteran pest), with regard to a nucleic acid molecule, includes internalization of the nucleic acid molecule into the organism, for example and without limitation: ingestion of the molecule by the organism (e.g., by feeding); contacting the organism with a composition comprising the nucleic acid molecule; and soaking of organisms with a solution comprising the nucleic acid molecule.

Contig: As used herein the term “contig” refers to a DNA sequence that is reconstructed from a set of overlapping DNA segments derived from a single genetic source.

Corn plant: As used herein, the term “corn plant” refers to a plant of the species, Zea mays (maize).

Expression: As used herein, “expression” of a coding polynucleotide (for example, a gene or a transgene) refers to the process by which the coded information of a nucleic acid transcriptional unit (including, e.g., gDNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, northern blot, RT-PCR, western blot, or in vitro, in situ, or in vivo protein activity assay(s).

Genetic material: As used herein, the term “genetic material” includes all genes, and nucleic acid molecules, such as DNA and RNA.

Hemipteran pest: As used herein, the term “hemipteran pest” refers to insects of the order Hemiptera, including, for example and without limitation, insects in the families Pentatomidae, Miridae, Pyrrhocoridae, Coreidae, Alydidae, and Rhopalidae, which feed on a wide range of host plants and have piercing and sucking mouth parts. In particular examples, a hemipteran pest is selected from the list comprising, Euschistus heros (Fabr.) (Neotropical Brown Stink Bug), Nezara viridula (L.) (Southern Green Stink Bug), Piezodorus guildinii (Westwood) (Red-banded Stink Bug), Halyomorpha halys (Stål) (Brown Marmorated Stink Bug), Chinavia hilare (Say) (Green Stink Bug), Euschistus servus (Say) (Brown Stink Bug), Dichelops melacanthus (Dallas), Dichelops furcatus (F.), Edessa meditabunda (F.), Thyanta perditor (F.) (Neotropical Red Shouldered Stink Bug), Chinavia marginatum (Palisot de Beauvois), Horcias nobilellus (Berg) (Cotton Bug), Taedia stigmosa (Berg), Dysdercus peruvianus (Guérin-Méneville), Neomegalotomus parvus (Westwood), Leptoglossus zonatus (Dallas), Niesthrea sidae (F.), Lygus hesperus (Knight) (Western Tarnished Plant Bug), and Lygus lineolaris (Palisot de Beauvois).

Inhibition: As used herein, the term “inhibition,” when used to describe an effect on a coding polynucleotide (for example, a gene), refers to a measurable decrease in the cellular level of mRNA transcribed from the coding polynucleotide and/or peptide, polypeptide, or protein product of the coding polynucleotide. In some examples, expression of a coding polynucleotide may be inhibited such that expression is approximately eliminated. “Specific inhibition” refers to the inhibition of a target coding polynucleotide without consequently affecting expression of other coding polynucleotides (e.g., genes) in the cell wherein the specific inhibition is being accomplished.

Insect: As used herein with regard to pests, the term “insect pest” specifically includes coleopteran insect pests and hemipteran insect pests. In some embodiments, the term also includes some other insect pests.

Isolated: An “isolated” biological component (such as a nucleic acid or protein) has been substantially separated, produced apart from, or purified away from other biological components in the cell of the organism in which the component naturally occurs (i.e., other chromosomal and extra-chromosomal DNA and RNA, and proteins), while effecting a chemical or functional change in the component (e.g., a nucleic acid may be isolated from a chromosome by breaking chemical bonds connecting the nucleic acid to the remaining DNA in the chromosome). Nucleic acid molecules and proteins that have been “isolated” include nucleic acid molecules and proteins purified by standard purification methods. The term also embraces nucleic acids and proteins prepared by recombinant expression in a host cell, as well as chemically-synthesized nucleic acid molecules, proteins, and peptides.

Nucleic acid molecule: As used herein, the term “nucleic acid molecule” may refer to a polymeric form of nucleotides, which may include both sense and anti-sense strands of RNA, cDNA, gDNA, and synthetic forms and mixed polymers of the above. A nucleotide or nucleobase may refer to a ribonucleotide, deoxyribonucleotide, or a modified form of either type of nucleotide. A “nucleic acid molecule” as used herein is synonymous with “nucleic acid” and “polynucleotide.” A nucleic acid molecule is usually at least 10 bases in length, unless otherwise specified. By convention, the nucleotide sequence of a nucleic acid molecule is read from the 5′ to the 3′ end of the molecule. The “complement” of a nucleic acid molecule refers to a polynucleotide having nucleobases that may form base pairs with the nucleobases of the nucleic acid molecule (i.e., A-T/U, and G-C).

Some embodiments include nucleic acids comprising a template DNA that is transcribed into an RNA molecule that is the complement of an mRNA molecule. In these embodiments, the complement of the nucleic acid transcribed into the mRNA molecule is present in the 5′ to 3′ orientation, such that RNA polymerase (which transcribes DNA in the 5′ to 3′ direction) will transcribe a nucleic acid from the complement that can hybridize to the mRNA molecule. Unless explicitly stated otherwise, or it is clear to be otherwise from the context, the term “complement” therefore refers to a polynucleotide having nucleobases, from 5′ to 3′, that may form base pairs with the nucleobases of a reference nucleic acid. Similarly, unless it is explicitly stated to be otherwise (or it is clear to be otherwise from the context), the “reverse complement” of a nucleic acid refers to the complement in reverse orientation. The foregoing is demonstrated in the following illustration:

polynucleotide
5′ ATGATGATG 3′
“complement” of the polynucleotide
5′ TACTACTAC 3′
“reverse complement” of the polynucleotide
5′ CATCATCAT 3′

Some embodiments of the invention include hairpin RNA-forming iRNA molecules. In these iRNAs, both the complement of a nucleic acid to be targeted by RNA interference and the reverse complement may be found in the same molecule, such that the single-stranded RNA molecule may “fold over” and hybridize to itself over region comprising the complementary and reverse complementary polynucleotides, as demonstrated in the following illustration:

5′ AUGAUGAUG-linker polynucleotide-CAUCAUCAU 3′,

which hybridizes to form:

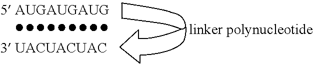

“Nucleic acid molecules” include all polynucleotides, for example: single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), mRNA (messenger RNA), miRNA (micro-RNA), shRNA (small hairpin RNA), hpRNA (hairpin RNA), tRNA (transfer RNAs, whether charged or discharged with a corresponding acylated amino acid), and cRNA (complementary RNA). The term “deoxyribonucleic acid” (DNA) is inclusive of cDNA, gDNA, and DNA-RNA hybrids. The terms “polynucleotide,” “nucleic acid,” “segments” thereof, and “fragments” thereof will be understood by those in the art to include, for example, gDNAs; ribosomal RNAs; transfer RNAs; RNAs; messenger RNAs; operons; smaller engineered polynucleotides that encode or may be adapted to encode peptides, polypeptides, or proteins; and structural and/or functional elements within a nucleic acid molecule that are delineated by their corresponding nucleotide sequence.

Oligonucleotide: An oligonucleotide is a short nucleic acid polymer. Oligonucleotides may be formed by cleavage of longer nucleic acid segments, or by polymerizing individual nucleotide precursors. Automated synthesizers allow the synthesis of oligonucleotides up to several hundred bases in length. Because oligonucleotides may bind to a complementary nucleic acid, they may be used as probes for detecting DNA or RNA. Oligonucleotides composed of DNA (oligodeoxyribonucleotides) may be used in PCR, a technique for the amplification of DNA and RNA (reverse transcribed into a cDNA) sequences. In PCR, the oligonucleotide is typically referred to as a “primer,” which allows a DNA polymerase to extend the oligonucleotide and replicate the complementary strand.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

As used herein with respect to DNA, the term “coding polynucleotide,” “structural polynucleotide,” or “structural nucleic acid molecule” refers to a polynucleotide that is ultimately translated into a polypeptide, via transcription and mRNA, when placed under the control of appropriate regulatory elements. With respect to RNA, the term “coding polynucleotide” refers to a polynucleotide that is translated into a peptide, polypeptide, or protein. The boundaries of a coding polynucleotide are determined by a translation start codon at the 5′-terminus and a translation stop codon at the 3′-terminus. Coding polynucleotides include, but are not limited to: gDNA; cDNA; ESTs; and recombinant polynucleotides.

As used herein, “transcribed non-coding polynucleotide” refers to segments of mRNA molecules such as 5′UTR, 3′UTR and intron segments that are not translated into a peptide, polypeptide, or protein. Further, “transcribed non-coding polynucleotide” refers to a nucleic acid that is transcribed into an RNA that functions in the cell, for example, structural RNAs (e.g., ribosomal RNA (rRNA) as exemplified by 5S rRNA, 5.8S rRNA, 16S rRNA, 18S rRNA, 23S rRNA, and 28S rRNA, and the like); transfer RNA (tRNA); and snRNAs such as U4, U5, U6, and the like. Transcribed non-coding polynucleotides also include, for example and without limitation, small RNAs (sRNA), which term is often used to describe small bacterial non-coding RNAs; small nucleolar RNAs (snoRNA); microRNAs; small interfering RNAs (siRNA); Piwi-interacting RNAs (piRNA); and long non-coding RNAs. Further still, “transcribed non-coding polynucleotide” refers to a polynucleotide that may natively exist as an intragenic “spacer” in a nucleic acid and which is transcribed into an RNA molecule.

Lethal RNA interference: As used herein, the term “lethal RNA interference” refers to RNA interference that results in death or a reduction in viability of the subject individual to which, for example, a dsRNA, miRNA, siRNA, and/or hpRNA is delivered.

Genome: As used herein, the term “genome” refers to chromosomal DNA found within the nucleus of a cell, and also refers to organelle DNA found within subcellular components of the cell. In some embodiments of the invention, a DNA molecule may be introduced into a plant cell, such that the DNA molecule is integrated into the genome of the plant cell. In these and further embodiments, the DNA molecule may be either integrated into the nuclear DNA of the plant cell, or integrated into the DNA of the chloroplast or mitochondrion of the plant cell. The term “genome,” as it applies to bacteria, refers to both the chromosome and plasmids within the bacterial cell. In some embodiments of the invention, a DNA molecule may be introduced into a bacterium such that the DNA molecule is integrated into the genome of the bacterium. In these and further embodiments, the DNA molecule may be either chromosomally-integrated or located as or in a stable plasmid.

Sequence identity: The term “sequence identity” or “identity,” as used herein in the context of two polynucleotides or polypeptides, refers to the residues in the sequences of the two molecules that are the same when aligned for maximum correspondence over a specified comparison window.

As used herein, the term “percentage of sequence identity” may refer to the value determined by comparing two optimally aligned sequences (e.g., nucleic acid sequences or polypeptide sequences) of a molecule over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa.

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default BLOSUM62 matrix set to default parameters. Nucleic acids with even greater sequence similarity to the sequences of the reference polynucleotides will show increasing percentage identity when assessed by this method.

Specifically hybridizable/Specifically complementary: As used herein, the terms “Specifically hybridizable” and “Specifically complementary” are terms that indicate a sufficient degree of complementarity such that stable and specific binding occurs between the nucleic acid molecule and a target nucleic acid molecule. Hybridization between two nucleic acid molecules involves the formation of an anti-parallel alignment between the nucleobases of the two nucleic acid molecules. The two molecules are then able to form hydrogen bonds with corresponding bases on the opposite strand to form a duplex molecule that, if it is sufficiently stable, is detectable using methods well known in the art. A polynucleotide need not be 100% complementary to its target nucleic acid to be specifically hybridizable. However, the amount of complementarity that must exist for hybridization to be specific is a function of the hybridization conditions used.

Hybridization conditions resulting in particular degrees of stringency will vary depending upon the nature of the hybridization method of choice and the composition and length of the hybridizing nucleic acids. Generally, the temperature of hybridization and the ionic strength (especially the Na⁺ and/or Mg⁺⁺ concentration) of the hybridization buffer will determine the stringency of hybridization, though wash times also influence stringency. Calculations regarding hybridization conditions required for attaining particular degrees of stringency are known to those of ordinary skill in the art, and are discussed, for example, in Sambrook et al. (ed.) Molecular Cloning: A Laboratory Manual, 2^{nd} ed., vol. 1-3, Cold Spring Harbor Laboratory Press, Cold Spring Harbor, N. Y., 1989, chapters 9 and 11; and Hames and Higgins (eds.) Nucleic Acid Hybridization, IRL Press, Oxford, 1985. Further detailed instruction and guidance with regard to the hybridization of nucleic acids may be found, for example, in Tijssen, “Overview of principles of hybridization and the strategy of nucleic acid probe assays,” in Laboratory Techniques in Biochemistry and Molecular Biology-Hybridization with Nucleic Acid Probes, Part I, Chapter 2, Elsevier, N.Y., 1993; and Ausubel et al., Eds., Current Protocols in Molecular Biology, Chapter 2, Greene Publishing and Wiley-Interscience, NY, 1995, and updates.

As used herein, “stringent conditions” encompass conditions under which hybridization will only occur if there is less than 20% mismatch between the sequence of the hybridization molecule and a homologous polynucleotide within the target nucleic acid molecule. “Stringent conditions” include further particular levels of stringency. Thus, as used herein, “moderate stringency” conditions are those under which polynucleotides with more than 20% sequence mismatch will not hybridize; conditions of “high stringency” are those under which polynucleotides with more than 10% mismatch will not hybridize; and conditions of “very high stringency” are those under which polynucleotides with more than 5% mismatch will not hybridize.

The following are representative, non-limiting hybridization conditions.

High Stringency condition (detects polynucleotides that share at least 90% sequence identity): Hybridization in 5×SSC buffer at 65° C. for 16 hours; wash twice in 2×SSC buffer at room temperature for 15 minutes each; and wash twice in 0.5×SSC buffer at 65° C. for 20 minutes each.

Moderate Stringency condition (detects polynucleotides that share at least 80% sequence identity): Hybridization in 5×-6×SSC buffer at 65-70° C. for 16-20 hours; wash twice in 2×SSC buffer at room temperature for 5-20 minutes each; and wash twice in 1×SSC buffer at 55-70° C. for 30 minutes each.

Non-stringent control condition (polynucleotides that share at least 50% sequence identity will hybridize): Hybridization in 6×SSC buffer at room temperature to 55° C. for 16-20 hours; wash at least twice in 2×-3×SSC buffer at room temperature to 55° C. for 20-30 minutes each.

As used herein, the term “substantially homologous” or “substantial homology,” with regard to a nucleic acid, refers to a polynucleotide having contiguous nucleobases that hybridize under stringent conditions to the reference nucleic acid. For example, nucleic acids that are substantially homologous to a reference nucleic acid of any of SEQ ID NOs:1, 3-6, 84-88, 102, 104, 107, and 109 are those nucleic acids that hybridize under stringent conditions (e.g., the Moderate Stringency conditions set forth, supra) to the reference nucleic acid of any of SEQ ID NOs:1, 3-6, 84-88, 102, 104, 107, and 109. Substantially homologous polynucleotides may have at least 80% sequence identity. For example, substantially homologous polynucleotides may have from about 80% to 100% sequence identity, such as 79%; 80%; about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; and about 100%. The property of substantial homology is closely related to specific hybridization. For example, a nucleic acid molecule is specifically hybridizable when there is a sufficient degree of complementarity to avoid non-specific binding of the nucleic acid to non-target polynucleotides under conditions where specific binding is desired, for example, under stringent hybridization conditions.

As used herein, the term “ortholog” refers to a gene in two or more species that has evolved from a common ancestral nucleic acid, and may retain the same function in the two or more species.

As used herein, two nucleic acid molecules are said to exhibit “complete complementarity” when every nucleotide of a polynucleotide read in the 5′ to 3′ direction is complementary to every nucleotide of the other polynucleotide when read in the 3′ to 5′ direction. A polynucleotide that is complementary to a reference polynucleotide will exhibit a sequence identical to the reverse complement of the reference polynucleotide. These terms and descriptions are well defined in the art and are easily understood by those of ordinary skill in the art.

Operably linked: A first polynucleotide is operably linked with a second polynucleotide when the first polynucleotide is in a functional relationship with the second polynucleotide. When recombinantly produced, operably linked polynucleotides are generally contiguous, and, where necessary to join two protein-coding regions, in the same reading frame (e.g., in a translationally fused ORF). However, nucleic acids need not be contiguous to be operably linked.

The term, “operably linked,” when used in reference to a regulatory genetic element and a coding polynucleotide, means that the regulatory element affects the expression of the linked coding polynucleotide. “Regulatory elements,” or “control elements,” refer to polynucleotides that influence the timing and level/amount of transcription, RNA processing or stability, or translation of the associated coding polynucleotide. Regulatory elements may include promoters; translation leaders; introns; enhancers; stem-loop structures; repressor binding polynucleotides; polynucleotides with a termination sequence; polynucleotides with a polyadenylation recognition sequence; etc. Particular regulatory elements may be located upstream and/or downstream of a coding polynucleotide operably linked thereto. Also, particular regulatory elements operably linked to a coding polynucleotide may be located on the associated complementary strand of a double-stranded nucleic acid molecule.

Promoter: As used herein, the term “promoter” refers to a region of DNA that may be upstream from the start of transcription, and that may be involved in recognition and binding of RNA polymerase and other proteins to initiate transcription. A promoter may be operably linked to a coding polynucleotide for expression in a cell, or a promoter may be operably linked to a polynucleotide encoding a signal peptide which may be operably linked to a coding polynucleotide for expression in a cell. A “plant promoter” may be a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred”. Promoters which initiate transcription only in certain tissues are referred to as “tissue-specific”. A “cell type-specific” promoter primarily drives expression in certain cell types in one or more organs, for example, vascular cells in roots or leaves. An “inducible” promoter may be a promoter which may be under environmental control. Examples of environmental conditions that may initiate transcription by inducible promoters include anaerobic conditions and the presence of light. Tissue-specific, tissue-preferred, cell type specific, and inducible promoters constitute the class of “non-constitutive” promoters. A “constitutive” promoter is a promoter which may be active under most environmental conditions or in most tissue or cell types.

Any inducible promoter can be used in some embodiments of the invention. See Ward et al. (1993) Plant Mol. Biol. 22:361-366. With an inducible promoter, the rate of transcription increases in response to an inducing agent. Exemplary inducible promoters include, but are not limited to: Promoters from the ACEI system that respond to copper; In2 gene from maize that responds to benzenesulfonamide herbicide safeners; Tet repressor from Tn10; and the inducible promoter from a steroid hormone gene, the transcriptional activity of which may be induced by a glucocorticosteroid hormone (Schena et al. (1991) Proc. Natl. Acad. Sci. USA 88:0421).

Exemplary constitutive promoters include, but are not limited to: Promoters from plant viruses, such as the 35S promoter from Cauliflower Mosaic Virus (CaMV); promoters from rice actin genes; ubiquitin promoters; pEMU; MAS; maize H3 histone promoter; and the ALS promoter, Xba1/NcoI fragment 5′ to the Brassica napus ALS3 structural gene (or a polynucleotide similar to said Xba1/NcoI fragment) (International PCT Publication No. WO96/30530).

Additionally, any tissue-specific or tissue-preferred promoter may be utilized in some embodiments of the invention. Plants transformed with a nucleic acid molecule comprising a coding polynucleotide operably linked to a tissue-specific promoter may produce the product of the coding polynucleotide exclusively, or preferentially, in a specific tissue. Exemplary tissue-specific or tissue-preferred promoters include, but are not limited to: A seed-preferred promoter, such as that from the phaseolin gene; a leaf-specific and light-induced promoter such as that from cab or rubisco; an anther-specific promoter such as that from LAT52; a pollen-specific promoter such as that from Zm13; and a microspore-preferred promoter such as that from apg.

Soybean plant: As used herein, the term “soybean plant” refers to a plant of the species Glycine sp.; for example, G. max.

Transformation: As used herein, the term “transformation” or “transduction” refers to the transfer of one or more nucleic acid molecule(s) into a cell. A cell is “transformed” by a nucleic acid molecule transduced into the cell when the nucleic acid molecule becomes stably replicated by the cell, either by incorporation of the nucleic acid molecule into the cellular genome, or by episomal replication. As used herein, the term “transformation” encompasses all techniques by which a nucleic acid molecule can be introduced into such a cell. Examples include, but are not limited to: transfection with viral vectors; transformation with plasmid vectors; electroporation (Fromm et al. (1986) Nature 319:791-3); lipofection (Feigner et al. (1987) Proc. Natl. Acad. Sci. USA 84:7413-7); microinjection (Mueller et al. (1978) Cell 15:579-85); Agrobacterium-mediated transfer (Fraley et al. (1983) Proc. Natl. Acad. Sci. USA 80:4803-7); direct DNA uptake; and microprojectile bombardment (Klein et al. (1987) Nature 327:70).

Transgene: An exogenous nucleic acid. In some examples, a transgene may be a DNA that encodes one or both strand(s) of an RNA capable of forming a dsRNA molecule that comprises a polynucleotide that is complementary to a nucleic acid molecule found in a coleopteran and/or hemipteran pest. In further examples, a transgene may be a gene (e.g., a herbicide-tolerance gene, a gene encoding an industrially or pharmaceutically useful compound, or a gene encoding a desirable agricultural trait). In these and other examples, a transgene may contain regulatory elements operably linked to a coding polynucleotide of the transgene (e.g., a promoter).

Vector: A nucleic acid molecule as introduced into a cell, for example, to produce a transformed cell. A vector may include genetic elements that permit it to replicate in the host cell, such as an origin of replication. Examples of vectors include, but are not limited to: a plasmid; cosmid; bacteriophage; or virus that carries exogenous DNA into a cell. A vector may also include one or more genes, including ones that produce antisense molecules, and/or selectable marker genes and other genetic elements known in the art. A vector may transduce, transform, or infect a cell, thereby causing the cell to express the nucleic acid molecules and/or proteins encoded by the vector. A vector optionally includes materials to aid in achieving entry of the nucleic acid molecule into the cell (e.g., a liposome, protein coating, etc.).

Yield: A stabilized yield of about 100% or greater relative to the yield of check varieties in the same growing location growing at the same time and under the same conditions. In particular embodiments, “improved yield” or “improving yield” means a cultivar having a stabilized yield of 105% or greater relative to the yield of check varieties in the same growing location containing significant densities of the coleopteran and/or hemipteran pests that are injurious to that crop growing at the same time and under the same conditions, which pests are targeted by the compositions and methods herein.

Unless specifically indicated or implied, the terms “a,” “an,” and “the” signify “at least one,” as used herein.

Unless otherwise specifically explained, all technical and scientific terms used herein have the same meaning as commonly understood by those of ordinary skill in the art to which this disclosure belongs. Definitions of common terms in molecular biology can be found in, for example, Lewin's Genes X, Jones & Bartlett Publishers, 2009 (ISBN 10 0763766321); Krebs et al. (eds.), The Encyclopedia of Molecular Biology, Blackwell Science Ltd., 1994 (ISBN 0-632-02182-9); and Meyers R. A. (ed.), Molecular Biology and Biotechnology: A Comprehensive Desk Reference, VCH Publishers, Inc., 1995 (ISBN 1-56081-569-8). All percentages are by weight and all solvent mixture proportions are by volume unless otherwise noted. All temperatures are in degrees Celsius.

### IV. Nucleic Acid Molecules Comprising an Insect Pest Polynucleotide

### A. Overview

Described herein are nucleic acid molecules useful for the control of insect pests. In some examples, the insect pest is a coleopteran or hemipteran insect pest. Described nucleic acid molecules include target polynucleotides (e.g., native genes, and non-coding polynucleotides), dsRNAs, siRNAs, shRNAs, hpRNAs, and miRNAs. For example, dsRNA, siRNA, miRNA, shRNA, and/or hpRNA molecules are described in some embodiments that may be specifically complementary to all or part of one or more native nucleic acids in a coleopteran and/or hemipteran pest. In these and further embodiments, the native nucleic acid(s) may be one or more target gene(s), the product of which may be, for example and without limitation: involved in in larval/nymphal development. Nucleic acid molecules described herein, when introduced into a cell comprising at least one native nucleic acid(s) to which the nucleic acid molecules are specifically complementary, may initiate RNAi in the cell, and consequently reduce or eliminate expression of the native nucleic acid(s). In some examples, reduction or elimination of the expression of a target gene by a nucleic acid molecule specifically complementary thereto may result in reduction or cessation of growth, development, and/or feeding of the pest.

In some embodiments, at least one target gene in an insect pest may be selected, wherein the target gene comprises a Gho/Sec24B2 or Sec24B1 polynucleotide. In particular examples, a target gene in a coleopteran or hemipteran pest is selected, wherein the target gene comprises a polynucleotide selected from among SEQ ID NOs:1, 84, 85, 102, and 107.

In some embodiments, a target gene may be a nucleic acid molecule comprising a polynucleotide that can be translated in silico to a polypeptide comprising a contiguous amino acid sequence that is at least about 85% identical (e.g., at least 84%, 85%, about 90%, about 95%, about 96%, about 97%, about 98%, about 99%, about 100%, or 100% identical) to the amino acid sequence of a protein product of a Gho/Sec24B2 or Sec24B1 polynucleotide. A target gene may be any Gho/Sec24B2 or Sec24B1 nucleic acid in an insect pest, the post-transcriptional inhibition of which has a deleterious effect on the growth and/or survival of the pest, for example, to provide a protective benefit against the pest to a plant. In particular examples, a target gene is a nucleic acid molecule comprising a polynucleotide that can be reverse translated in silico to a polypeptide comprising a contiguous amino acid sequence that is at least about 85% identical, about 90% identical, about 95% identical, about 96% identical, about 97% identical, about 98% identical, about 99% identical, about 100% identical, or 100% identical to the amino acid sequence of SEQ ID NO:2, SEQ ID NO:98, SEQ ID NO:99, SEQ ID NO:103, or SEQ ID NO:108.

Provided in some embodiments are DNAs, the expression of which results in an RNA molecule comprising a polynucleotide that is specifically complementary to all or part of a native RNA molecule that is encoded by a coding polynucleotide in an insect (e.g., coleopteran and/or hemipteran) pest. In some embodiments, after ingestion of the expressed RNA molecule by an insect pest, down-regulation of the coding polynucleotide in cells of the pest may be obtained. In particular embodiments, down-regulation of the coding sequence in cells of the insect pest may result in a deleterious effect on the growth, development, and/or survival of the pest.

In some embodiments, target polynucleotides include transcribed non-coding RNAs, such as 5′UTRs; 3′UTRs; spliced leaders; introns; outrons (e.g., 5′UTR RNA subsequently modified in trans splicing); donatrons (e.g., non-coding RNA required to provide donor sequences for trans splicing); and other non-coding transcribed RNA of target insect pest genes. Such polynucleotides may be derived from both mono-cistronic and poly-cistronic genes.

Thus, also described herein in connection with some embodiments are iRNA molecules (e.g., dsRNAs, siRNAs, miRNAs, shRNAs, and hpRNAs) that comprise at least one polynucleotide that is specifically complementary to all or part of a target nucleic acid in an insect (e.g., coleopteran and/or hemipteran) pest. In some embodiments an iRNA molecule may comprise polynucleotide(s) that are complementary to all or part of a plurality of target nucleic acids; for example, 2, 3, 4, 5, 6, 7, 8, 9, 10, or more target nucleic acids. In particular embodiments, an iRNA molecule may be produced in vitro, or in vivo by a genetically-modified organism, such as a plant or bacterium. Also disclosed are cDNAs that may be used for the production of dsRNA molecules, siRNA molecules, miRNA molecules, shRNA molecules, and/or hpRNA molecules that are specifically complementary to all or part of a target nucleic acid in an insect pest. Further described are recombinant DNA constructs for use in achieving stable transformation of particular host targets. Transformed host targets may express effective levels of dsRNA, siRNA, miRNA, shRNA, and/or hpRNA molecules from the recombinant DNA constructs. Therefore, also described is a plant transformation vector comprising at least one polynucleotide operably linked to a heterologous promoter functional in a plant cell, wherein expression of the polynucleotide(s) results in an RNA molecule comprising a string of contiguous nucleobases that is specifically complementary to all or part of a target nucleic acid in an insect pest.

In particular examples, nucleic acid molecules useful for the control of insect (e.g., coleopteran and/or hemipteran) pests may include: all or part of a native nucleic acid isolated from Diabrotica comprising a Gho/Sec24B2 or Sec24B1 polynucleotide (e.g., any of SEQ ID NOs:1, 102, and 107); DNAs that when expressed result in an RNA molecule comprising a polynucleotide that is specifically complementary to all or part of a native RNA molecule that is encoded by Diabrotica Gho/Sec24B2 or Sec24B1; iRNA molecules (e.g., dsRNAs, siRNAs, miRNAs, shRNAs, and hpRNAs) that comprise at least one polynucleotide that is specifically complementary to all or part of Diabrotica Gho/Sec24B2 or Sec24B1; cDNAs that may be used for the production of dsRNA molecules, siRNA molecules, miRNA molecules, shRNA molecules, and/or hpRNA molecules that are specifically complementary to all or part of Diabrotica Gho/Sec24B2 or Sec24B1; all or part of a native nucleic acid isolated from Euschistus heros comprising a Gho/Sec24B2 or Sec24B1 polynucleotide (e.g., SEQ ID NO:84 and SEQ ID NO:85); DNAs that when expressed result in an RNA molecule comprising a polynucleotide that is specifically complementary to all or part of a native RNA molecule that is encoded by E. heros Gho/Sec24B2 or Sec24B1; iRNA molecules (e.g., dsRNAs, siRNAs, miRNAs, shRNAs, and hpRNAs) that comprise at least one polynucleotide that is specifically complementary to all or part of E. heros Gho/Sec24B2 or Sec24B1; cDNAs that may be used for the production of dsRNA molecules, siRNA molecules, miRNA molecules, shRNA molecules, and/or hpRNA molecules that are specifically complementary to all or part of E. heros Gho/Sec24B2 or Sec24B1; and recombinant DNA constructs for use in achieving stable transformation of particular host targets, wherein a transformed host target comprises one or more of the foregoing nucleic acid molecules.

### B. Nucleic Acid Molecules

The present invention provides, inter alia, iRNA (e.g., dsRNA, siRNA, miRNA, shRNA, and hpRNA) molecules that inhibit target gene expression in a cell, tissue, or organ of an insect (e.g., coleopteran and/or hemipteran) pest; and DNA molecules capable of being expressed as an iRNA molecule in a cell or microorganism to inhibit target gene expression in a cell, tissue, or organ of an insect pest.

Some embodiments of the invention provide an isolated nucleic acid molecule comprising at least one (e.g., one, two, three, or more) polynucleotide(s) selected from the group consisting of: any of SEQ ID NOs:1, 84, 85, 102, and 107; the complement of any of SEQ ID NOs:1, 84, 85, 102, and 107; a fragment of at least 15 contiguous nucleotides of any of SEQ ID NOs:1, 84, 85, 102, and 107 (e.g., any of SEQ ID NOs: 3-6, 86-88, 104, and 109); the complement of a fragment of at least 15 contiguous nucleotides of any of SEQ ID NOs:1, 84, 85, 102, and 107; a native coding polynucleotide of a Diabrotica organism (e.g., WCR) comprising SEQ ID NO:1, 102, or 107; the complement of a native coding polynucleotide of a Diabrotica organism comprising SEQ ID NO:1, 102, or 107; a fragment of at least 15 contiguous nucleotides of a native coding polynucleotide of a Diabrotica organism comprising SEQ ID NO:1, 102, or 107; the complement of a fragment of at least 15 contiguous nucleotides of a native coding polynucleotide of a Diabrotica organism comprising SEQ ID NO:1, 102, or 107; a native coding polynucleotide of a Euschistus heros organism comprising SEQ ID NO:84 or SEQ ID NO:85; the complement of a native coding polynucleotide of a E. heros organism comprising SEQ ID NO:84 or SEQ ID NO:85; a fragment of at least 15 contiguous nucleotides of a native coding polynucleotide of a E. heros organism comprising SEQ ID NO:84 or SEQ ID NO:85; and the complement of a fragment of at least 15 contiguous nucleotides of a native coding polynucleotide of a E. heros organism comprising SEQ ID NO:84 or SEQ ID NO:85. In particular embodiments, contact with or uptake by an insect (e.g., coleopteran and/or hemipteran) pest of an iRNA transcribed from the isolated polynucleotide inhibits the growth, development and/or feeding of the pest.

In some embodiments, an isolated nucleic acid molecule of the invention may comprise at least one (e.g., one, two, three, or more) polynucleotide(s) selected from the group consisting of: SEQ ID NO:112; the complement of SEQ ID NO:112; SEQ ID NO:113; the complement of SEQ ID NO:113; SEQ ID NO:114; the complement of SEQ ID NO:114; SEQ ID NO:115; the complement of SEQ ID NO:115; SEQ ID NO:116; the complement of SEQ ID NO:116; SEQ ID NO:119; the complement of SEQ ID NO:119; SEQ ID NO:120; the complement of SEQ ID NO:120; SEQ ID NO:121; the complement of SEQ ID NO:121; SEQ ID NO:122; the complement of SEQ ID NO:122; SEQ ID NO:123; the complement of SEQ ID NO:123; SEQ ID NO:124; the complement of SEQ ID NO:124; SEQ ID NO:125; the complement of SEQ ID NO:125; SEQ ID NO:126; the complement of SEQ ID NO:126; SEQ ID NO:127; the complement of SEQ ID NO:127; a fragment of at least 15 contiguous nucleotides of any of SEQ ID NOs:112-116 and 119-127; the complement of a fragment of at least 15 contiguous nucleotides of any of SEQ ID NOs:112-116 and 119-127; a native polyribonucleotide transcribed in a Diabrotica organism from a gene comprising SEQ ID NO:1, SEQ ID NO:3, or SEQ ID NO:5; the complement of a native polyribonucleotide transcribed in a Diabrotica organism from a gene comprising SEQ ID NO:1, SEQ ID NO:3, SEQ ID NO:4, SEQ ID NO:5, or SEQ ID NO: 6; a fragment of at least 15 contiguous nucleotides of a native polyribonucleotide transcribed in a Diabrotica organism from a gene comprising SEQ ID NO:1, SEQ ID NO:102, SEQ ID NO:104 SEQ ID NO:107, or SEQ ID NO:109; the complement of a fragment of at least 15 contiguous nucleotides of a native polyribonucleotide transcribed in a Diabrotica organism from a gene comprising SEQ ID NO:1, SEQ ID NO:102, or SEQ ID NO:107; a native polyribonucleotide transcribed in a Euschistus heros organism from a gene comprising SEQ ID NO:84 or SEQ ID NO:85; the complement of a native polyribonucleotide transcribed in a E. heros organism from a gene comprising SEQ ID NO:84 or SEQ ID NOs:85-88; a fragment of at least 15 contiguous nucleotides of a native polyribonucleotide transcribed in a E. heros organism from a gene comprising SEQ ID NO:84 or SEQ ID NO:85; and the complement of a fragment of at least 15 contiguous nucleotides of a native polyribonucleotide transcribed in a E. heros organism from a gene comprising SEQ ID NO:84 or SEQ ID NO:85. In particular embodiments, contact with or uptake by a coleopteran and/or hemipteran pest of the isolated polynucleotide inhibits the growth, development and/or feeding of the pest.

In some embodiments, a nucleic acid molecule of the invention may comprise at least one (e.g., one, two, three, or more) DNA(s) capable of being expressed as an iRNA molecule in a cell or microorganism to inhibit target gene expression in a cell, tissue, or organ of a coleopteran and/or hemipteran pest. Such DNA(s) may be operably linked to a promoter that functions in a cell comprising the DNA molecule to initiate or enhance the transcription of the encoded RNA capable of forming a dsRNA molecule(s). In one embodiment, the at least one (e.g., one, two, three, or more) DNA(s) may be derived from a polynucleotide selected from a group comprising SEQ ID NOs:1 and 72. Derivatives of SEQ ID NOs:1 and 72 include fragments of the SEQ ID NO:1 and/or SEQ ID NO:72. In some embodiments, such a fragment may comprise, for example, at least about 15 contiguous nucleotides of SEQ ID NO:1, SEQ ID NO:72, or a complement thereof. Thus, such a fragment may comprise, for example, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200 or more contiguous nucleotides of SEQ ID NO:1 and/or SEQ ID NO:72, or a complement thereof. In some examples, such a fragment may comprise, for example, at least 19 contiguous nucleotides (e.g., 19, 20, 21, 22, 23, 24, 25, 26, 27, 28, 29, or 30 contiguous nucleotides) of SEQ ID NO:1 and/or SEQ ID NO:72, or a complement thereof.

Some embodiments comprise introducing partially- or fully-stabilized dsRNA molecules into a coleopteran and/or hemipteran pest to inhibit expression of a target gene in a cell, tissue, or organ of the coleopteran and/or hemipteran pest. When expressed as an iRNA molecule (e.g., dsRNA, siRNA, miRNA, shRNA, and hpRNA) and taken up by a coleopteran and/or hemipteran pest, polynucleotides comprising one or more fragments of any of SEQ ID NOs:1, 3, 67, 72, 73, and the complements thereof, may cause one or more of death, developmental arrest, growth inhibition, change in sex ratio, reduction in brood size, cessation of infection, and/or cessation of feeding by a coleopteran and/or hemipteran pest. In particular examples, polynucleotides comprising one or more fragments (e.g., polynucleotides including about 15 to about 300 nucleotides) of any of SEQ ID NOs:1, 3, 67, 72, 73, and the complements thereof cause a reduction in the capacity of an existing generation of the pest to produce a subsequent generation of the pest.

In certain embodiments, dsRNA molecules provided by the invention comprise polynucleotides complementary to a transcript from a target gene comprising any of SEQ ID NOs:1, 84, 85, 102, and 107, and fragments thereof, the inhibition of which target gene in an insect pest results in the reduction or removal of a polypeptide or polynucleotide agent that is essential for the pest's growth, development, or other biological function. A selected polynucleotide may exhibit from about 80% to about 100% sequence identity to any of SEQ ID NOs:1, 84, 85, 102, and 107; a contiguous fragment of any of SEQ ID NOs:1, 84, 85, 102, and 107; and/or the complement of any of the foregoing. For example, a selected polynucleotide may exhibit 79%; 80%; about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; or about 100% sequence identity to any of SEQ ID NOs:1, 3-6, 102, 84-88, 107, and 109; a contiguous fragment of any of SEQ ID NOs:1, 3-6, 84-88, 102, 104, 107, and 109; and the complement of any of the foregoing.

In some embodiments, a DNA molecule capable of being expressed as an iRNA molecule in a cell or microorganism to inhibit target gene expression may comprise a single polynucleotide that is specifically complementary to all or part of a native polynucleotide found in one or more target insect pest species (e.g., a coleopteran or hemipteran pest species), or the DNA molecule can be constructed as a chimera from a plurality of such specifically complementary polynucleotides.

In some embodiments, a nucleic acid molecule may comprise a first and a second polynucleotide separated by a “spacer.” A spacer may be a region comprising any sequence of nucleotides that facilitates secondary structure formation between the first and second polynucleotides, where this is desired. In one embodiment, the spacer is part of a sense or antisense coding polynucleotide for mRNA. The spacer may alternatively comprise any combination of nucleotides or homologues thereof that are capable of being linked covalently to a nucleic acid molecule. In some examples, the spacer may be an intron (e.g., an ST-LS1 intron or a RTM1 intron).

For example, in some embodiments, the DNA molecule may comprise a polynucleotide coding for one or more different iRNA molecules, wherein each of the different iRNA molecules comprises a first polynucleotide and a second polynucleotide, wherein the first and second polynucleotides are complementary to each other. The first and second polynucleotides may be connected within an RNA molecule by a spacer. The spacer may constitute part of the first polynucleotide or the second polynucleotide. Expression of an RNA molecule comprising the first and second nucleotide polynucleotides may lead to the formation of a dsRNA molecule, by specific intramolecular base-pairing of the first and second nucleotide polynucleotides. The first polynucleotide or the second polynucleotide may be substantially identical to a polynucleotide (e.g., a target gene, or transcribed non-coding polynucleotide) native to an insect pest (e.g., a coleopteran or hemipteran pest), a derivative thereof, or a complementary polynucleotide thereto.

dsRNA nucleic acid molecules comprise double strands of polymerized ribonucleotides, and may include modifications to either the phosphate-sugar backbone or the nucleoside. Modifications in RNA structure may be tailored to allow specific inhibition. In one embodiment, dsRNA molecules may be modified through a ubiquitous enzymatic process so that siRNA molecules may be generated. This enzymatic process may utilize an RNase III enzyme, such as DICER in eukaryotes, either in vitro or in vivo. See Elbashir et al. (2001) Nature 411:494-8; and Hamilton and Baulcombe (1999) Science 286(5441):950-2. DICER or functionally-equivalent RNAse III enzymes cleave larger dsRNA strands and/or hpRNA molecules into smaller oligonucleotides (e.g., siRNAs), each of which is about 19-25 nucleotides in length. The siRNA molecules produced by these enzymes have 2 to 3 nucleotide 3′ overhangs, and 5′ phosphate and 3′ hydroxyl termini. The siRNA molecules generated by RNAse III enzymes are unwound and separated into single-stranded RNA in the cell. The siRNA molecules then specifically hybridize with RNAs transcribed from a target gene, and both RNA molecules are subsequently degraded by an inherent cellular RNA-degrading mechanism. This process may result in the effective degradation or removal of the RNA encoded by the target gene in the target organism. The outcome is the post-transcriptional silencing of the targeted gene. In some embodiments, siRNA molecules produced by endogenous RNAse III enzymes from heterologous nucleic acid molecules may efficiently mediate the down-regulation of target genes in coleopteran and/or hemipteran pests.

In some embodiments, a nucleic acid molecule may include at least one non-naturally occurring polynucleotide that can be transcribed into a single-stranded RNA molecule capable of forming a dsRNA molecule in vivo through intermolecular hybridization. Such dsRNAs typically self-assemble, and can be provided in the nutrition source of an insect (e.g., coleopteran or hemipteran) pest to achieve the post-transcriptional inhibition of a target gene. In these and further embodiments, a nucleic acid molecule may comprise two different non-naturally occurring polynucleotides, each of which is specifically complementary to a different target gene in an insect pest. When such a nucleic acid molecule is provided as a dsRNA molecule to, for example, a coleopteran and/or hemipteran pest, the dsRNA molecule inhibits the expression of at least two different target genes in the pest.

### C. Obtaining Nucleic Acid Molecules

A variety of polynucleotides in insect (e.g., coleopteran and hemipteran) pests may be used as targets for the design of nucleic acid molecules, such as iRNAs and DNA molecules encoding iRNAs. Selection of native polynucleotides is not, however, a straight-forward process. For example, only a small number of native polynucleotides in a coleopteran or hemipteran pest will be effective targets. It cannot be predicted with certainty whether a particular native polynucleotide can be effectively down-regulated by nucleic acid molecules of the invention, or whether down-regulation of a particular native polynucleotide will have a detrimental effect on the growth, development and/or survival of an insect pest. The vast majority of native coleopteran and hemipteran pest polynucleotides, such as ESTs isolated therefrom (for example, the coleopteran pest polynucleotides listed in U.S. Pat. No. 7,612,194), do not have a detrimental effect on the growth, development, and/or survival of the pest. Neither is it predictable which of the native polynucleotides that may have a detrimental effect on an insect pest are able to be used in recombinant techniques for expressing nucleic acid molecules complementary to such native polynucleotides in a host plant and providing the detrimental effect on the pest upon feeding without causing harm to the host plant.

In some embodiments, nucleic acid molecules (e.g., dsRNA molecules to be provided in the host plant of an insect (e.g., coleopteran or hemipteran) pest) are selected to target cDNAs that encode proteins or parts of proteins essential for pest development, such as polypeptides involved in metabolic or catabolic biochemical pathways, cell division, energy metabolism, digestion, host plant recognition, and the like. As described herein, ingestion of compositions by a target pest organism containing one or more dsRNAs, at least one segment of which is specifically complementary to at least a substantially identical segment of RNA produced in the cells of the target pest organism, can result in the death or other inhibition of the target. A polynucleotide, either DNA or RNA, derived from an insect pest can be used to construct plant cells resistant to infestation by the pests. The host plant of the coleopteran and/or hemipteran pest (e.g., Z. mays or G. max), for example, can be transformed to contain one or more polynucleotides derived from the coleopteran and/or hemipteran pest as provided herein. The polynucleotide transformed into the host may encode one or more RNAs that form into a dsRNA structure in the cells or biological fluids within the transformed host, thus making the dsRNA available if/when the pest forms a nutritional relationship with the transgenic host. This may result in the suppression of expression of one or more genes in the cells of the pest, and ultimately death or inhibition of its growth or development.

Thus, in some embodiments, a gene is targeted that is essentially involved in the growth and development of an insect (e.g., coleopteran or hemipteran) pest. Other target genes for use in the present invention may include, for example, those that play important roles in pest movement, migration, growth, development, infectivity, and establishment of feeding sites. A target gene may therefore be a housekeeping gene or a transcription factor. Additionally, a native insect pest polynucleotide for use in the present invention may also be derived from a homolog (e.g., an ortholog), of a plant, viral, bacterial or insect gene, the function of which is known to those of skill in the art, and the polynucleotide of which is specifically hybridizable with a target gene in the genome of the target pest. Methods of identifying a homolog of a gene with a known nucleotide sequence by hybridization are known to those of skill in the art.

In some embodiments, the invention provides methods for obtaining a nucleic acid molecule comprising a polynucleotide for producing an iRNA (e.g., dsRNA, siRNA, miRNA, shRNA, and hpRNA) molecule. One such embodiment comprises: (a) analyzing one or more target gene(s) for their expression, function, and phenotype upon dsRNA-mediated gene suppression in an insect (e.g., coleopteran or hemipteran) pest; (b) probing a cDNA or gDNA library with a probe comprising all or a portion of a polynucleotide or a homolog thereof from a targeted pest that displays an altered (e.g., reduced) growth or development phenotype in a dsRNA-mediated suppression analysis; (c) identifying a DNA clone that specifically hybridizes with the probe; (d) isolating the DNA clone identified in step (b); (e) sequencing the cDNA or gDNA fragment that comprises the clone isolated in step (d), wherein the sequenced nucleic acid molecule comprises all or a substantial portion of the RNA or a homolog thereof; and (f) chemically synthesizing all or a substantial portion of a gene, or an siRNA, miRNA, hpRNA, mRNA, shRNA, or dsRNA.

In further embodiments, a method for obtaining a nucleic acid fragment comprising a polynucleotide for producing a substantial portion of an iRNA (e.g., dsRNA, siRNA, miRNA, shRNA, and hpRNA) molecule includes: (a) synthesizing first and second oligonucleotide primers specifically complementary to a portion of a native polynucleotide from a targeted insect (e.g., coleopteran or hemipteran) pest; and (b) amplifying a cDNA or gDNA insert present in a cloning vector using the first and second oligonucleotide primers of step (a), wherein the amplified nucleic acid molecule comprises a substantial portion of a siRNA, miRNA, hpRNA, mRNA, shRNA, or dsRNA molecule.

Nucleic acids can be isolated, amplified, or produced by a number of approaches. For example, an iRNA (e.g., dsRNA, siRNA, miRNA, shRNA, and hpRNA) molecule may be obtained by PCR amplification of a target polynucleotide (e.g., a target gene or a target transcribed non-coding polynucleotide) derived from a gDNA or cDNA library, or portions thereof. DNA or RNA may be extracted from a target organism, and nucleic acid libraries may be prepared therefrom using methods known to those ordinarily skilled in the art. gDNA or cDNA libraries generated from a target organism may be used for PCR amplification and sequencing of target genes. A confirmed PCR product may be used as a template for in vitro transcription to generate sense and antisense RNA with minimal promoters. Alternatively, nucleic acid molecules may be synthesized by any of a number of techniques (See, e.g., Ozaki et al. (1992) Nucleic Acids Research, 20: 5205-5214; and Agrawal et al. (1990) Nucleic Acids Research, 18: 5419-5423), including use of an automated DNA synthesizer (for example, a P.E. Biosystems, Inc. (Foster City, Calif.) model 392 or 394 DNA/RNA Synthesizer), using standard chemistries, such as phosphoramidite chemistry. See, e.g., Beaucage et al. (1992) Tetrahedron, 48: 2223-2311; U.S. Pat. Nos. 4,980,460, 4,725,677, 4,415,732, 4,458,066, and 4,973,679. Alternative chemistries resulting in non-natural backbone groups, such as phosphorothioate, phosphoramidate, and the like, can also be employed.

An RNA, dsRNA, siRNA, miRNA, shRNA, or hpRNA molecule of the present invention may be produced chemically or enzymatically by one skilled in the art through manual or automated reactions, or in vivo in a cell comprising a nucleic acid molecule comprising a polynucleotide encoding the RNA, dsRNA, siRNA, miRNA, shRNA, or hpRNA molecule. RNA may also be produced by partial or total organic synthesis—any modified ribonucleotide can be introduced by in vitro enzymatic or organic synthesis. An RNA molecule may be synthesized by a cellular RNA polymerase or a bacteriophage RNA polymerase (e.g., T3 RNA polymerase, T7 RNA polymerase, and SP6 RNA polymerase). Expression constructs useful for the cloning and expression of polynucleotides are known in the art. See, e.g., International PCT Publication No. WO97/32016; and U.S. Pat. Nos. 5,593,874, 5,698,425, 5,712,135, 5,789,214, and 5,804,693. RNA molecules that are synthesized chemically or by in vitro enzymatic synthesis may be purified prior to introduction into a cell. For example, RNA molecules can be purified from a mixture by extraction with a solvent or resin, precipitation, electrophoresis, chromatography, or a combination thereof. Alternatively, RNA molecules that are synthesized chemically or by in vitro enzymatic synthesis may be used with no or a minimum of purification, for example, to avoid losses due to sample processing. The RNA molecules may be dried for storage or dissolved in an aqueous solution. The solution may contain buffers or salts to promote annealing, and/or stabilization of dsRNA molecule duplex strands.

In embodiments, a dsRNA molecule may be formed by a single self-complementary RNA strand or from two complementary RNA strands. dsRNA molecules may be synthesized either in vivo or in vitro. An endogenous RNA polymerase of the cell may mediate transcription of the one or two RNA strands in vivo, or cloned RNA polymerase may be used to mediate transcription in vivo or in vitro. Post-transcriptional inhibition of a target gene in an insect pest may be host-targeted by specific transcription in an organ, tissue, or cell type of the host (e.g., by using a tissue-specific promoter); stimulation of an environmental condition in the host (e.g., by using an inducible promoter that is responsive to infection, stress, temperature, and/or chemical inducers); and/or engineering transcription at a developmental stage or age of the host (e.g., by using a developmental stage-specific promoter). RNA strands that form a dsRNA molecule, whether transcribed in vitro or in vivo, may or may not be polyadenylated, and may or may not be capable of being translated into a polypeptide by a cell's translational apparatus.

### D. Recombinant Vectors and Host Cell Transformation

In some embodiments, the invention also provides a DNA molecule for introduction into a cell (e.g., a bacterial cell, a yeast cell, or a plant cell), wherein the DNA molecule comprises a polynucleotide that, upon expression to RNA and ingestion by an insect (e.g., coleopteran and/or hemipteran) pest, achieves suppression of a target gene in a cell, tissue, or organ of the pest. Thus, some embodiments provide a recombinant nucleic acid molecule comprising a polynucleotide capable of being expressed as an iRNA (e.g., dsRNA, siRNA, miRNA, shRNA, and hpRNA) molecule in a plant cell to inhibit target gene expression in an insect pest. In order to initiate or enhance expression, such recombinant nucleic acid molecules may comprise one or more regulatory elements, which regulatory elements may be operably linked to the polynucleotide capable of being expressed as an iRNA. Methods to express a gene suppression molecule in plants are known, and may be used to express a polynucleotide of the present invention. See, e.g., International PCT Publication No. WO06/073727; and U.S. Patent Publication No. 2006/0200878 A1)

In specific embodiments, a recombinant DNA molecule of the invention may comprise a polynucleotide encoding an RNA that may form a dsRNA molecule. Such recombinant DNA molecules may encode RNAs that may form dsRNA molecules capable of inhibiting the expression of endogenous target gene(s) in an insect (e.g., coleopteran and/or hemipteran) pest cell upon ingestion. In many embodiments, a transcribed RNA may form a dsRNA molecule that may be provided in a stabilized form; e.g., as a hairpin and stem and loop structure.

In some embodiments, one strand of a dsRNA molecule may be formed by transcription from a polynucleotide which is substantially homologous to a polynucleotide selected from the group consisting of any of SEQ ID NOs:1, 84, 85, 102, and 107; the complements of any of SEQ ID NOs:1, 84, 85, 102, and 107; a fragment of at least 15 contiguous nucleotides of any of SEQ ID NOs:1, 84, 85, 102, and 107 (e.g., SEQ ID NOs:3-6, 86-88, 104, and 109); the complement of a fragment of at least 15 contiguous nucleotides of any of SEQ ID NOs:1, 84, 85, 102, and 107; a native coding polynucleotide of a Diabrotica organism (e.g., WCR) comprising any of any of SEQ ID NOs:1, 3-6, 102, 104, 107, and 109; the complement of a native coding polynucleotide of a Diabrotica organism comprising any of SEQ ID NOs:1, 3-6, 102, 104, 107, and 109; a fragment of at least 15 contiguous nucleotides of a native coding polynucleotide of a Diabrotica organism comprising any of SEQ ID NOs:1, 3-6, 102, 104, 107, and 109; the complement of a fragment of at least 15 contiguous nucleotides of a native coding polynucleotide of a Diabrotica organism comprising any of SEQ ID NOs:1, 3-6, 102, 104, 107, and 109; a native coding polynucleotide of a Euschistus heros organism (i.e., BSB) comprising any of SEQ ID NOs:84-88; the complement of a native coding polynucleotide of a E. heros organism comprising any of SEQ ID NOs:84-88; a fragment of at least 15 contiguous nucleotides of a native coding polynucleotide of a E. heros organism comprising any of SEQ ID NOs:84-88; and the complement of a fragment of at least 15 contiguous nucleotides of a native coding polynucleotide of a E. heros organism comprising any of SEQ ID NOs:84-88.

In some embodiments, one strand of a dsRNA molecule may be formed by transcription from a polynucleotide that is substantially homologous to a polynucleotide selected from the group consisting of any of SEQ ID NOs:3-6, 86-88, 104, and 109; the complement of any of SEQ ID NOs:3-6, 86-88, 104, and 109; fragments of at least 15 contiguous nucleotides of any of SEQ ID NOs:3-6, 86-88, 104, and 109; and the complements of fragments of at least 15 contiguous nucleotides of any of SEQ ID NOs:3-6, 86-88, 104, and 109. In particular examples, a dsRNA is formed by transcription from a polynucleotide that comprises SEQ ID NO:18 or SEQ ID NO:19.

In particular embodiments, a recombinant DNA molecule encoding an RNA that may form a dsRNA molecule may comprise a coding region wherein at least two polynucleotides are arranged such that one polynucleotide is in a sense orientation, and the other polynucleotide is in an antisense orientation, relative to at least one promoter, wherein the sense polynucleotide and the antisense polynucleotide are linked or connected by a spacer of, for example, from about five (˜5) to about one thousand (˜1000) nucleotides. The spacer may form a loop between the sense and antisense polynucleotides. The sense polynucleotide or the antisense polynucleotide may be substantially homologous to a target gene (e.g., a Gho/Sec24B2 gene or Sec24B1 gene comprising SEQ ID NO:1, SEQ ID NO:84, SEQ ID NO:85, SEQ ID NO:102, or SEQ ID NO:107) or fragment thereof. In some embodiments, however, a recombinant DNA molecule may encode an RNA that may form a dsRNA molecule without a spacer. In embodiments, a sense coding polynucleotide and an antisense coding polynucleotide may be different lengths.

Polynucleotides identified as having a deleterious effect on an insect pest or a plant-protective effect with regard to the pest may be readily incorporated into expressed dsRNA molecules through the creation of appropriate expression cassettes in a recombinant nucleic acid molecule of the invention. For example, such polynucleotides may be expressed as a hairpin with stem and loop structure by taking a first segment corresponding to a target gene polynucleotide (e.g., a Gho/Sec24B2 gene or Sec24B1 gene comprising SEQ ID NO:1, SEQ ID NO:84, SEQ ID NO:85, SEQ ID NO:102, or SEQ ID NO:107, and fragments of any of the foregoing); linking this polynucleotide to a second segment spacer region that is not homologous or complementary to the first segment; and linking this to a third segment, wherein at least a portion of the third segment is substantially complementary to the first segment. Such a construct forms a stem and loop structure by intramolecular base-pairing of the first segment with the third segment, wherein the loop structure forms comprising the second segment. See, e.g., U.S. Patent Publication Nos. 2002/0048814 and 2003/0018993; and International PCT Publication Nos. WO94/01550 and WO98/05770. A dsRNA molecule may be generated, for example, in the form of a double-stranded structure such as a stem-loop structure (e.g., hairpin), whereby production of siRNA targeted for a native insect (e.g., coleopteran and/or hemipteran) pest polynucleotide is enhanced by co-expression of a fragment of the targeted gene, for instance on an additional plant expressible cassette, that leads to enhanced siRNA production, or reduces methylation to prevent transcriptional gene silencing of the dsRNA hairpin promoter.

Embodiments of the invention include introduction of a recombinant nucleic acid molecule of the present invention into a plant (i.e., transformation) to achieve insect (e.g., coleopteran and/or hemipteran) pest-inhibitory levels of expression of one or more iRNA molecules. A recombinant DNA molecule may, for example, be a vector, such as a linear or a closed circular plasmid. The vector system may be a single vector or plasmid, or two or more vectors or plasmids that together contain the total DNA to be introduced into the genome of a host. In addition, a vector may be an expression vector. Nucleic acids of the invention can, for example, be suitably inserted into a vector under the control of a suitable promoter that functions in one or more hosts to drive expression of a linked coding polynucleotide or other DNA element. Many vectors are available for this purpose, and selection of the appropriate vector will depend mainly on the size of the nucleic acid to be inserted into the vector and the particular host cell to be transformed with the vector. Each vector contains various components depending on its function (e.g., amplification of DNA or expression of DNA) and the particular host cell with which it is compatible.

To impart protection from insect (e.g., coleopteran and/or hemipteran) pests to a transgenic plant, a recombinant DNA may, for example, be transcribed into an iRNA molecule (e.g., a RNA molecule that forms a dsRNA molecule) within the tissues or fluids of the recombinant plant. An iRNA molecule may comprise a polynucleotide that is substantially homologous and specifically hybridizable to a corresponding transcribed polynucleotide within an insect pest that may cause damage to the host plant species. The pest may contact the iRNA molecule that is transcribed in cells of the transgenic host plant, for example, by ingesting cells or fluids of the transgenic host plant that comprise the iRNA molecule. Thus, in particular examples, expression of a target gene is suppressed by the iRNA molecule within coleopteran and/or hemipteran pests that infest the transgenic host plant. In some embodiments, suppression of expression of the target gene in a target coleopteran and/or hemipteran pest may result in the plant being protected from attack by the pest.

In order to enable delivery of iRNA molecules to an insect pest in a nutritional relationship with a plant cell that has been transformed with a recombinant nucleic acid molecule of the invention, expression (i.e., transcription) of iRNA molecules in the plant cell is required. Thus, a recombinant nucleic acid molecule may comprise a polynucleotide of the invention operably linked to one or more regulatory elements, such as a heterologous promoter element that functions in a host cell, such as a bacterial cell wherein the nucleic acid molecule is to be amplified, and a plant cell wherein the nucleic acid molecule is to be expressed.

Promoters suitable for use in nucleic acid molecules of the invention include those that are inducible, viral, synthetic, or constitutive, all of which are well known in the art. Non-limiting examples describing such promoters include U.S. Pat. No. 6,437,217 (maize RS81 promoter); U.S. Pat. No. 5,641,876 (rice actin promoter); U.S. Pat. No. 6,426,446 (maize RS324 promoter); U.S. Pat. No. 6,429,362 (maize PR-1 promoter); U.S. Pat. No. 6,232,526 (maize A3 promoter); U.S. Pat. No. 6,177,611 (constitutive maize promoters); U.S. Pat. Nos. 5,322,938, 5,352,605, 5,359,142, and 5,530,196 (CaMV 35S promoter); U.S. Pat. No. 6,433,252 (maize L3 oleosin promoter); U.S. Pat. No. 6,429,357 (rice actin 2 promoter, and rice actin 2 intron); U.S. Pat. No. 6,294,714 (light-inducible promoters); U.S. Pat. No. 6,140,078 (salt-inducible promoters); U.S. Pat. No. 6,252,138 (pathogen-inducible promoters); U.S. Pat. No. 6,175,060 (phosphorous deficiency-inducible promoters); U.S. Pat. No. 6,388,170 (bidirectional promoters); U.S. Pat. No. 6,635,806 (gamma-coixin promoter); and U.S. Patent Publication No. 2009/757,089 (maize chloroplast aldolase promoter). Additional promoters include the nopaline synthase (NOS) promoter (Ebert et al. (1987) Proc. Natl. Acad. Sci. USA 84(16):5745-9) and the octopine synthase (OCS) promoters (which are carried on tumor-inducing plasmids of Agrobacterium tumefaciens); the caulimovirus promoters such as the cauliflower mosaic virus (CaMV) 19S promoter (Lawton et al. (1987) Plant Mol. Biol. 9:315-24); the CaMV 35S promoter (Odell et al. (1985) Nature 313:810-2; the figwort mosaic virus 35S-promoter (Walker et al. (1987) Proc. Natl. Acad. Sci. USA 84(19):6624-8); the sucrose synthase promoter (Yang and Russell (1990) Proc. Natl. Acad. Sci. USA 87:4144-8); the R gene complex promoter (Chandler et al. (1989) Plant Cell 1:1175-83); the chlorophyll a/b binding protein gene promoter; CaMV 35S (U.S. Pat. Nos. 5,322,938, 5,352,605, 5,359,142, and 5,530,196); FMV 35S (U.S. Pat. Nos. 6,051,753, and 5,378,619); a PC1SV promoter (U.S. Pat. No. 5,850,019); the SCP1 promoter (U.S. Pat. No. 6,677,503); and AGRtu.nos promoters (GenBank™ Accession No. V00087; Depicker et al. (1982) J. Mol. Appl. Genet. 1:561-73; Bevan et al. (1983) Nature 304:184-7).

In particular embodiments, nucleic acid molecules of the invention comprise a tissue-specific promoter, such as a root-specific promoter. Root-specific promoters drive expression of operably-linked coding polynucleotides exclusively or preferentially in root tissue. Examples of root-specific promoters are known in the art. See, e.g., U.S. Pat. Nos. 5,110,732; 5,459,252 and 5,837,848; and Opperman et al. (1994) Science 263:221-3; and Hirel et al. (1992) Plant Mol. Biol. 20:207-18. In some embodiments, a polynucleotide or fragment for coleopteran and/or hemipteran pest control according to the invention may be cloned between two root-specific promoters oriented in opposite transcriptional directions relative to the polynucleotide or fragment, and which are operable in a transgenic plant cell and expressed therein to produce RNA molecules in the transgenic plant cell that subsequently may form dsRNA molecules, as described, supra. The iRNA molecules expressed in plant tissues may be ingested by an insect pest so that suppression of target gene expression is achieved.

Additional regulatory elements that may optionally be operably linked to a nucleic acid include 5′UTRs located between a promoter element and a coding polynucleotide that function as a translation leader element. The translation leader element is present in fully-processed mRNA, and it may affect processing of the primary transcript, and/or RNA stability. Examples of translation leader elements include maize and petunia heat shock protein leaders (U.S. Pat. No. 5,362,865), plant virus coat protein leaders, plant rubisco leaders, and others. See, e.g., Turner and Foster (1995) Molecular Biotech. 3(3):225-36. Non-limiting examples of 5′UTRs include GmHsp (U.S. Pat. No. 5,659,122); PhDnaK (U.S. Pat. No. 5,362,865); AtAnt1; TEV (Carrington and Freed (1990) J. Virol. 64:1590-7); and AGRtunos (GenBank™ Accession No. V00087; and Bevan et al. (1983) Nature 304:184-7).

Additional regulatory elements that may optionally be operably linked to a nucleic acid also include 3′ non-translated elements, 3′ transcription termination regions, or polyadenylation regions. These are genetic elements located downstream of a polynucleotide, and include polynucleotides that provide polyadenylation signal, and/or other regulatory signals capable of affecting transcription or mRNA processing. The polyadenylation signal functions in plants to cause the addition of polyadenylate nucleotides to the 3′ end of the mRNA precursor. The polyadenylation element can be derived from a variety of plant genes, or from T-DNA genes. A non-limiting example of a 3′ transcription termination region is the nopaline synthase 3′ region (nos 3; Fraley et al. (1983) Proc. Natl. Acad. Sci. USA 80:4803-7). An example of the use of different 3′ non-translated regions is provided in Ingelbrecht et al., (1989) Plant Cell 1:671-80. Non-limiting examples of polyadenylation signals include one from a Pisum sativum RbcS2 gene (Ps.RbcS2-E9; Coruzzi et al. (1984) EMBO J. 3:1671-9) and AGRtu.nos (GenBank™ Accession No. E01312).

Some embodiments may include a plant transformation vector that comprises an isolated and purified DNA molecule comprising at least one of the above-described regulatory elements operatively linked to one or more polynucleotides of the present invention. When expressed, the one or more polynucleotides result in one or more iRNA molecule(s) comprising a polynucleotide that is specifically complementary to all or part of a native RNA molecule in an insect (e.g., coleopteran and/or hemipteran) pest. Thus, the polynucleotide(s) may comprise a segment encoding all or part of a polyribonucleotide present within a targeted coleopteran and/or hemipteran pest RNA transcript, and may comprise inverted repeats of all or a part of a targeted pest transcript. A plant transformation vector may contain polynucleotides specifically complementary to more than one target polynucleotide, thus allowing production of more than one dsRNA for inhibiting expression of two or more genes in cells of one or more populations or species of target insect pests. Segments of polynucleotides specifically complementary to polynucleotides present in different genes can be combined into a single composite nucleic acid molecule for expression in a transgenic plant. Such segments may be contiguous or separated by a spacer.

In some embodiments, a plasmid of the present invention already containing at least one polynucleotide(s) of the invention can be modified by the sequential insertion of additional polynucleotide(s) in the same plasmid, wherein the additional polynucleotide(s) are operably linked to the same regulatory elements as the original at least one polynucleotide(s). In some embodiments, a nucleic acid molecule may be designed for the inhibition of multiple target genes. In some embodiments, the multiple genes to be inhibited can be obtained from the same insect (e.g., coleopteran or hemipteran) pest species, which may enhance the effectiveness of the nucleic acid molecule. In other embodiments, the genes can be derived from different insect pests, which may broaden the range of pests against which the agent(s) is/are effective. When multiple genes are targeted for suppression or a combination of expression and suppression, a polycistronic DNA element can be engineered.

A recombinant nucleic acid molecule or vector of the present invention may comprise a selectable marker that confers a selectable phenotype on a transformed cell, such as a plant cell. Selectable markers may also be used to select for plants or plant cells that comprise a recombinant nucleic acid molecule of the invention. The marker may encode biocide resistance, antibiotic resistance (e.g., kanamycin, Geneticin (G418), bleomycin, hygromycin, etc.), or herbicide tolerance (e.g., glyphosate, etc.). Examples of selectable markers include, but are not limited to: a neo gene which codes for kanamycin resistance and can be selected for using kanamycin, G418, etc.; a bar gene which codes for bialaphos resistance; a mutant EPSP synthase gene which encodes glyphosate tolerance; a nitrilase gene which confers resistance to bromoxynil; a mutant acetolactate synthase (ALS) gene which confers imidazolinone or sulfonylurea tolerance; and a methotrexate resistant DHFR gene. Multiple selectable markers are available that confer resistance to ampicillin, bleomycin, chloramphenicol, gentamycin, hygromycin, kanamycin, lincomycin, methotrexate, phosphinothricin, puromycin, spectinomycin, rifampicin, streptomycin and tetracycline, and the like. Examples of such selectable markers are illustrated in, e.g., U.S. Pat. Nos. 5,550,318; 5,633,435; 5,780,708 and 6,118,047.

A recombinant nucleic acid molecule or vector of the present invention may also include a screenable marker. Screenable markers may be used to monitor expression. Exemplary screenable markers include a β-glucuronidase or uidA gene (GUS) which encodes an enzyme for which various chromogenic substrates are known (Jefferson et al. (1987) Plant Mol. Biol. Rep. 5:387-405); an R-locus gene, which encodes a product that regulates the production of anthocyanin pigments (red color) in plant tissues (Dellaporta et al. (1988) “Molecular cloning of the maize R-nj allele by transposon tagging with Ac.” In 18^{th} Stadler Genetics Symposium, P. Gustafson and R. Appels, eds. (New York: Plenum), pp. 263-82); a β-lactamase gene (Sutcliffe et al. (1978) Proc. Natl. Acad. Sci. USA 75:3737-41); a gene which encodes an enzyme for which various chromogenic substrates are known (e.g., PADAC, a chromogenic cephalosporin); a luciferase gene (Ow et al. (1986) Science 234:856-9); an xylE gene that encodes a catechol dioxygenase that can convert chromogenic catechols (Zukowski et al. (1983) Gene 46(2-3):247-55); an amylase gene (Ikatu et al. (1990) Bio/Technol. 8:241-2); a tyrosinase gene which encodes an enzyme capable of oxidizing tyrosine to DOPA and dopaquinone which in turn condenses to melanin (Katz et al. (1983) J. Gen. Microbiol. 129:2703-14); and an α-galactosidase.

In some embodiments, recombinant nucleic acid molecules, as described, supra, may be used in methods for the creation of transgenic plants and expression of heterologous nucleic acids in plants to prepare transgenic plants that exhibit reduced susceptibility to insect (e.g., coleopteran and/or hemipteran) pests. Plant transformation vectors can be prepared, for example, by inserting nucleic acid molecules encoding iRNA molecules into plant transformation vectors and introducing these into plants.

Suitable methods for transformation of host cells include any method by which DNA can be introduced into a cell, such as by transformation of protoplasts (See, e.g., U.S. Pat. No. 5,508,184), by desiccation/inhibition-mediated DNA uptake (See, e.g., Potrykus et al. (1985) Mol. Gen. Genet. 199:183-8), by electroporation (See, e.g., U.S. Pat. No. 5,384,253), by agitation with silicon carbide fibers (See, e.g., U.S. Pat. Nos. 5,302,523 and 5,464,765), by Agrobacterium-mediated transformation (See, e.g., U.S. Pat. Nos. 5,563,055; 5,591,616; 5,693,512; 5,824,877; 5,981,840; and 6,384,301) and by acceleration of DNA-coated particles (See, e.g., U.S. Pat. Nos. 5,015,580; 5,550,318; 5,538,880; 6,160,208; 6,399,861; and 6,403,865), etc. Techniques that are particularly useful for transforming corn are described, for example, in U.S. Pat. Nos. 7,060,876 and 5,591,616; and International PCT Publication WO95/06722. Through the application of techniques such as these, the cells of virtually any species may be stably transformed. In some embodiments, transforming DNA is integrated into the genome of the host cell. In the case of multicellular species, transgenic cells may be regenerated into a transgenic organism. Any of these techniques may be used to produce a transgenic plant, for example, comprising one or more nucleic acids encoding one or more iRNA molecules in the genome of the transgenic plant.

The most widely utilized method for introducing an expression vector into plants is based on the natural transformation system of Agrobacterium. A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. The Ti (tumor-inducing)-plasmids contain a large segment, known as T-DNA, which is transferred to transformed plants. Another segment of the Ti plasmid, the Vir region, is responsible for T-DNA transfer. The T-DNA region is bordered by terminal repeats. In modified binary vectors, the tumor-inducing genes have been deleted, and the functions of the Vir region are utilized to transfer foreign DNA bordered by the T-DNA border elements. The T-region may also contain a selectable marker for efficient recovery of transgenic cells and plants, and a multiple cloning site for inserting polynucleotides for transfer such as a dsRNA encoding nucleic acid.

Thus, in some embodiments, a plant transformation vector is derived from a Ti plasmid of A. tumefaciens (See, e.g., U.S. Pat. Nos. 4,536,475, 4,693,977, 4,886,937, and 5,501,967; and European Patent No. EP 0 122 791) or a Ri plasmid of A. rhizogenes. Additional plant transformation vectors include, for example and without limitation, those described by Herrera-Estrella et al. (1983) Nature 303:209-13; Bevan et al. (1983) Nature 304:184-7; Klee et al. (1985) Bio/Technol. 3:637-42; and in European Patent No. EP 0 120 516, and those derived from any of the foregoing. Other bacteria such as Sinorhizobium, Rhizobium, and Mesorhizobium that interact with plants naturally can be modified to mediate gene transfer to a number of diverse plants. These plant-associated symbiotic bacteria can be made competent for gene transfer by acquisition of both a disarmed Ti plasmid and a suitable binary vector.

After providing exogenous DNA to recipient cells, transformed cells are generally identified for further culturing and plant regeneration. In order to improve the ability to identify transformed cells, one may desire to employ a selectable or screenable marker gene, as previously set forth, with the transformation vector used to generate the transformant. In the case where a selectable marker is used, transformed cells are identified within the potentially transformed cell population by exposing the cells to a selective agent or agents. In the case where a screenable marker is used, cells may be screened for the desired marker gene trait.

Cells that survive the exposure to the selective agent, or cells that have been scored positive in a screening assay, may be cultured in media that supports regeneration of plants. In some embodiments, any suitable plant tissue culture media (e.g., MS and N6 media) may be modified by including further substances, such as growth regulators. Tissue may be maintained on a basic medium with growth regulators until sufficient tissue is available to begin plant regeneration efforts, or following repeated rounds of manual selection, until the morphology of the tissue is suitable for regeneration (e.g., at least 2 weeks), then transferred to media conducive to shoot formation. Cultures are transferred periodically until sufficient shoot formation has occurred. Once shoots are formed, they are transferred to media conducive to root formation. Once sufficient roots are formed, plants can be transferred to soil for further growth and maturation.

To confirm the presence of a nucleic acid molecule of interest (for example, a DNA encoding one or more iRNA molecules that inhibit target gene expression in a coleopteran and/or hemipteran pest) in the regenerating plants, a variety of assays may be performed. Such assays include, for example: molecular biological assays, such as Southern and northern blotting, PCR, and nucleic acid sequencing; biochemical assays, such as detecting the presence of a protein product, e.g., by immunological means (ELISA and/or western blots) or by enzymatic function; plant part assays, such as leaf or root assays; and analysis of the phenotype of the whole regenerated plant.

Integration events may be analyzed, for example, by PCR amplification using, e.g., oligonucleotide primers specific for a nucleic acid molecule of interest. PCR genotyping is understood to include, but not be limited to, polymerase-chain reaction (PCR) amplification of gDNA derived from isolated host plant callus tissue predicted to contain a nucleic acid molecule of interest integrated into the genome, followed by standard cloning and sequence analysis of PCR amplification products. Methods of PCR genotyping have been well described (for example, Rios, G. et al. (2002) Plant J. 32:243-53) and may be applied to gDNA derived from any plant species (e.g., Z. mays or G. max) or tissue type, including cell cultures.

A transgenic plant formed using Agrobacterium-dependent transformation methods typically contains a single recombinant DNA inserted into one chromosome. The polynucleotide of the single recombinant DNA is referred to as a “transgenic event” or “integration event”. Such transgenic plants are heterozygous for the inserted exogenous polynucleotide. In some embodiments, a transgenic plant homozygous with respect to a transgene may be obtained by sexually mating (selfing) an independent segregant transgenic plant that contains a single exogenous gene to itself, for example a T₀ plant, to produce T₁ seed. One fourth of the T₁ seed produced will be homozygous with respect to the transgene. Germinating T₁ seed results in plants that can be tested for heterozygosity, typically using an SNP assay or a thermal amplification assay that allows for the distinction between heterozygotes and homozygotes (i.e., a zygosity assay).

In particular embodiments, at least 2, 3, 4, 5, 6, 7, 8, 9 or 10 or more different iRNA molecules are produced in a plant cell that have an insect (e.g., coleopteran and/or hemipteran) pest-inhibitory effect. The iRNA molecules (e.g., dsRNA molecules) may be expressed from multiple nucleic acids introduced in different transformation events, or from a single nucleic acid introduced in a single transformation event. In some embodiments, a plurality of iRNA molecules are expressed under the control of a single promoter. In other embodiments, a plurality of iRNA molecules are expressed under the control of multiple promoters. Single iRNA molecules may be expressed that comprise multiple polynucleotides that are each homologous to different loci within one or more insect pests (for example, the loci defined by SEQ ID NOs:1, 84, 85, 102, and 107), both in different populations of the same species of insect pest, or in different species of insect pests.

In addition to direct transformation of a plant with a recombinant nucleic acid molecule, transgenic plants can be prepared by crossing a first plant having at least one transgenic event with a second plant lacking such an event. For example, a recombinant nucleic acid molecule comprising a polynucleotide that encodes an iRNA molecule may be introduced into a first plant line that is amenable to transformation to produce a transgenic plant, which transgenic plant may be crossed with a second plant line to introgress the polynucleotide that encodes the iRNA molecule into the second plant line.

In some aspects, seeds and commodity products produced by transgenic plants derived from transformed plant cells are included, wherein the seeds or commodity products comprise a detectable amount of a nucleic acid of the invention. In some embodiments, such commodity products may be produced, for example, by obtaining transgenic plants and preparing food or feed from them. Commodity products comprising one or more of the polynucleotides of the invention includes, for example and without limitation: meals, oils, crushed or whole grains or seeds of a plant, and any food product comprising any meal, oil, or crushed or whole grain of a recombinant plant or seed comprising one or more of the nucleic acids of the invention. The detection of one or more of the polynucleotides of the invention in one or more commodity or commodity products is de facto evidence that the commodity or commodity product is produced from a transgenic plant designed to express one or more of the iRNA molecules of the invention for the purpose of controlling insect (e.g., coleopteran and/or hemipteran) pests.

In some embodiments, a transgenic plant or seed comprising a nucleic acid molecule of the invention also may comprise at least one other transgenic event in its genome, including without limitation: a transgenic event from which is transcribed an iRNA molecule targeting a locus in an insect pest other than the one defined by SEQ ID NOs:1, 84, 85, 102, and 107, such as, for example, one or more loci selected from the group consisting of Caf1-180 (U.S. Patent Application Publication No. 2012/0174258), VatpaseC (U.S. Patent Application Publication No. 2012/0174259), Rho1 (U.S. Patent Application Publication No. 2012/0174260), VatpaseH (U.S. Patent Application Publication No. 2012/0198586), PPI-87B (U.S. Patent Application Publication No. 2013/0091600), RPA70 (U.S. Patent Application Publication No. 2013/0091601), and RPS6 (U.S. Patent Application Publication No. 2013/0097730); a transgenic event from which is transcribed an iRNA molecule targeting a gene in an organism other than a coleopteran and/or hemipteran pest (e.g., a plant-parasitic nematode); a gene encoding an insecticidal protein (e.g., a Bacillus thuringiensis insecticidal protein); an herbicide tolerance gene (e.g., a gene providing tolerance to glyphosate); and a gene contributing to a desirable phenotype in the transgenic plant, such as increased yield, altered fatty acid metabolism, or restoration of cytoplasmic male sterility). In particular embodiments, polynucleotides encoding iRNA molecules of the invention may be combined with other insect control and disease traits in a plant to achieve desired traits for enhanced control of plant disease and insect damage. Combining insect control traits that employ distinct modes-of-action may provide protected transgenic plants with superior durability over plants harboring a single control trait, for example, because of the reduced probability that resistance to the trait(s) will develop in the field.

### V. Target Gene Suppression in a Coleopteran and/or Hemipteran Pest

### A. Overview

In some embodiments of the invention, at least one nucleic acid molecule useful for the control of coleopteran and/or hemipteran pests may be provided to a coleopteran and/or hemipteran pest, wherein the nucleic acid molecule leads to RNAi-mediated gene silencing in the pest(s). In particular embodiments, an iRNA molecule (e.g., dsRNA, siRNA, miRNA, shRNA, and hpRNA) may be provided to the coleopteran and/or hemipteran host. In some embodiments, a nucleic acid molecule useful for the control of coleopteran and/or hemipteran pests may be provided to a pest by contacting the nucleic acid molecule with the pest. In these and further embodiments, a nucleic acid molecule useful for the control of coleopteran and/or hemipteran pests may be provided in a feeding substrate of the pest, for example, a nutritional composition. In these and further embodiments, a nucleic acid molecule useful for the control of a coleopteran and/or hemipteran pest may be provided through ingestion of plant material comprising the nucleic acid molecule that is ingested by the pest. In certain embodiments, the nucleic acid molecule is present in plant material through expression of a recombinant nucleic acid introduced into the plant material, for example, by transformation of a plant cell with a vector comprising the recombinant nucleic acid and regeneration of a plant material or whole plant from the transformed plant cell.

### B. RNAi-Mediated Target Gene Suppression

In embodiments, the invention provides iRNA molecules (e.g., dsRNA, siRNA, miRNA, shRNA, and hpRNA) that may be designed to target essential native polynucleotides (e.g., essential genes) in the transcriptome of an insect pest (for example, a coleopteran (e.g., WCR or NCR) or hemipteran (e.g., BSB) pest), for example by designing an iRNA molecule that comprises at least one strand comprising a polynucleotide that is specifically complementary to the target polynucleotide. The sequence of an iRNA molecule so designed may be identical to that of the target polynucleotide, or may incorporate mismatches that do not prevent specific hybridization between the iRNA molecule and its target polynucleotide.

iRNA molecules of the invention may be used in methods for gene suppression in an insect (e.g., coleopteran and/or hemipteran) pest, thereby reducing the level or incidence of damage caused by the pest on a plant (for example, a protected transformed plant comprising an iRNA molecule). As used herein the term “gene suppression” refers to any of the well-known methods for reducing the levels of protein produced as a result of gene transcription to mRNA and subsequent translation of the mRNA, including the reduction of protein expression from a gene or a coding polynucleotide including post-transcriptional inhibition of expression and transcriptional suppression. Post-transcriptional inhibition is mediated by specific homology between all or a part of an mRNA transcribed from a gene targeted for suppression and the corresponding iRNA molecule used for suppression. Additionally, post-transcriptional inhibition refers to the substantial and measurable reduction of the amount of mRNA available in the cell for binding by ribosomes.

In embodiments wherein an iRNA molecule is a dsRNA molecule, the dsRNA molecule may be cleaved by the enzyme, DICER, into short siRNA molecules (approximately 20 nucleotides in length). The double-stranded siRNA molecule generated by DICER activity upon the dsRNA molecule may be separated into two single-stranded siRNAs; the “passenger strand” and the “guide strand”. The passenger strand may be degraded, and the guide strand may be incorporated into RISC. Post-transcriptional inhibition occurs by specific hybridization of the guide strand with a specifically complementary polynucleotide of an mRNA molecule, and subsequent cleavage by the enzyme, Argonaute (catalytic component of the RISC complex).

In embodiments of the invention, any form of iRNA molecule may be used. Those of skill in the art will understand that dsRNA molecules typically are more stable during preparation and during the step of providing the iRNA molecule to a cell than are single-stranded RNA molecules, and are typically also more stable in a cell. Thus, while siRNA and miRNA molecules, for example, may be equally effective in some embodiments, a dsRNA molecule may be chosen due to its stability.

In particular embodiments, a nucleic acid molecule is provided that comprises a polynucleotide, which polynucleotide may be expressed in vitro to produce an iRNA molecule that is substantially homologous to a nucleic acid molecule encoded by a polynucleotide within the genome of an insect (e.g., coleopteran and/or hemipteran) pest. In certain embodiments, the in vitro transcribed iRNA molecule may be a stabilized dsRNA molecule that comprises a stem-loop structure. After an insect pest contacts the in vitro transcribed iRNA molecule, post-transcriptional inhibition of a target gene in the pest (for example, an essential gene) may occur.

In some embodiments of the invention, expression of a nucleic acid molecule comprising at least 15 contiguous nucleotides (e.g., at least 19 contiguous nucleotides) of a polynucleotide are used in a method for post-transcriptional inhibition of a target gene in an insect (e.g., coleopteran and/or hemipteran) pest, wherein the polynucleotide is selected from the group consisting of: SEQ ID NO:112; the complement of SEQ ID NO:112; SEQ ID NO:113; the complement of SEQ ID NO:113; SEQ ID NO:114; the complement of SEQ ID NO:114; SEQ ID NO:115; the complement of SEQ ID NO:115; SEQ ID NO:116; the complement of SEQ ID NO:116; SEQ ID NO:119; the complement of SEQ ID NO:119; SEQ ID NO:120; the complement of SEQ ID NO:120; SEQ ID NO:121; the complement of SEQ ID NO:121; SEQ ID NO:122; the complement of SEQ ID NO:122; SEQ ID NO:123; the complement of SEQ ID NO:123; SEQ ID NO:124; the complement of SEQ ID NO:124; SEQ ID NO:125; the complement of SEQ ID NO:125; SEQ ID NO:126; the complement of SEQ ID NO:126; SEQ ID NO:127; the complement of SEQ ID NO:127; an RNA expressed from a native coding polynucleotide of a Diabrotica organism comprising SEQ ID NO:1; the complement of an RNA expressed from a native coding polynucleotide of a Diabrotica organism comprising SEQ ID NO:1; an RNA expressed from a native coding polynucleotide of a Diabrotica organism comprising SEQ ID NO:102; the complement of an RNA expressed from a native coding polynucleotide of a Diabrotica organism comprising SEQ ID NO:102; an RNA expressed from a native coding polynucleotide of a Diabrotica organism comprising SEQ ID NO:107; the complement of an RNA expressed from a native coding polynucleotide of a Diabrotica organism comprising SEQ ID NO:107; an RNA expressed from a native coding polynucleotide of a Euschistus heros organism comprising SEQ ID NO:84; the complement of an RNA expressed from a native coding polynucleotide of a E. heros organism comprising SEQ ID NO:84; an RNA expressed from a native coding polynucleotide of a Euschistus heros organism comprising SEQ ID NO:85; and the complement of an RNA expressed from a native coding polynucleotide of a E. heros organism comprising SEQ ID NO:85. Nucleic acid molecules comprising at least 15 contiguous nucleotides of the foregoing polynucleotides include, for example and without limitation, fragments comprising at least 15 contiguous nucleotides of a polynucleotide selected from the group consisting of SEQ ID NOs:112-116 and 119-127. In certain embodiments, expression of a nucleic acid molecule that is at least about 80% identical (e.g., 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, and 100%) with any of the foregoing may be used. In these and further embodiments, a nucleic acid molecule may be expressed that specifically hybridizes to an RNA molecule present in at least one cell of an insect (e.g., coleopteran and/or hemipteran) pest.

It is an important feature of some embodiments herein that the RNAi post-transcriptional inhibition system is able to tolerate sequence variations among target genes that might be expected due to genetic mutation, strain polymorphism, or evolutionary divergence. The introduced nucleic acid molecule may not need to be absolutely homologous to either a primary transcription product or a fully-processed mRNA of a target gene, so long as the introduced nucleic acid molecule is specifically hybridizable to either a primary transcription product or a fully-processed mRNA of the target gene. Moreover, the introduced nucleic acid molecule may not need to be full-length, relative to either a primary transcription product or a fully processed mRNA of the target gene.

Inhibition of a target gene using the iRNA technology of the present invention is sequence-specific; i.e., polynucleotides substantially homologous to the iRNA molecule(s) are targeted for genetic inhibition. In some embodiments, an RNA molecule comprising a polynucleotide with a nucleotide sequence that is identical to that of a portion of a target gene may be used for inhibition. In these and further embodiments, an RNA molecule comprising a polynucleotide with one or more insertion, deletion, and/or point mutations relative to a target polynucleotide may be used. In particular embodiments, an iRNA molecule and a portion of a target gene may share, for example, at least from about 80%, at least from about 81%, at least from about 82%, at least from about 83%, at least from about 84%, at least from about 85%, at least from about 86%, at least from about 87%, at least from about 88%, at least from about 89%, at least from about 90%, at least from about 91%, at least from about 92%, at least from about 93%, at least from about 94%, at least from about 95%, at least from about 96%, at least from about 97%, at least from about 98%, at least from about 99%, at least from about 100%, and 100% sequence identity. Alternatively, the duplex region of a dsRNA molecule may be specifically hybridizable with a portion of a target gene transcript. In specifically hybridizable molecules, a less than full length polynucleotide exhibiting a greater homology compensates for a longer, less homologous polynucleotide. The length of the polynucleotide of a duplex region of a dsRNA molecule that is identical to a portion of a target gene transcript may be at least about 25, 50, 100, 200, 300, 400, 500, or at least about 1000 bases. In some embodiments, a polynucleotide of greater than 20-100 nucleotides may be used. In particular embodiments, a polynucleotide of greater than about 200-300 nucleotides may be used. In particular embodiments, a polynucleotide of greater than about 500-1000 nucleotides may be used, depending on the size of the target gene.

In certain embodiments, expression of a target gene in a pest (e.g., coleopteran or hemipteran) pest may be inhibited by at least 10%; at least 33%; at least 50%; or at least 80% within a cell of the pest, such that a significant inhibition takes place. Significant inhibition refers to inhibition over a threshold that results in a detectable phenotype (e.g., cessation of growth, cessation of feeding, cessation of development, induced mortality, etc.), or a detectable decrease in RNA and/or gene product corresponding to the target gene being inhibited. Although, in certain embodiments of the invention, inhibition occurs in substantially all cells of the pest, in other embodiments inhibition occurs only in a subset of cells expressing the target gene.

In some embodiments, transcriptional suppression is mediated by the presence in a cell of a dsRNA molecule exhibiting substantial sequence identity to a promoter DNA or the complement thereof to effect what is referred to as “promoter trans suppression.” Gene suppression may be effective against target genes in an insect pest that may ingest or contact such dsRNA molecules, for example, by ingesting or contacting plant material containing the dsRNA molecules. dsRNA molecules for use in promoter trans suppression may be specifically designed to inhibit or suppress the expression of one or more homologous or complementary polynucleotides in the cells of the insect pest. Post-transcriptional gene suppression by antisense or sense oriented RNA to regulate gene expression in plant cells is disclosed in U.S. Pat. Nos. 5,107,065; 5,759,829; 5,283,184; and 5,231,020.

### C. Expression of iRNA Molecules Provided to an Insect Pest

Expression of iRNA molecules for RNAi-mediated gene inhibition in an insect (e.g., coleopteran and/or hemipteran) pest may be carried out in any one of many in vitro or in vivo formats. The iRNA molecules may then be provided to an insect pest, for example, by contacting the iRNA molecules with the pest, or by causing the pest to ingest or otherwise internalize the iRNA molecules. Some embodiments include transformed host plants of a coleopteran and/or hemipteran pest, transformed plant cells, and progeny of transformed plants. The transformed plant cells and transformed plants may be engineered to express one or more of the iRNA molecules, for example, under the control of a heterologous promoter, to provide a pest-protective effect. Thus, when a transgenic plant or plant cell is consumed by an insect pest during feeding, the pest may ingest iRNA molecules expressed in the transgenic plants or cells. The polynucleotides of the present invention may also be introduced into a wide variety of prokaryotic and eukaryotic microorganism hosts to produce iRNA molecules. The term “microorganism” includes prokaryotic and eukaryotic species, such as bacteria and fungi.

Modulation of gene expression may include partial or complete suppression of such expression. In another embodiment, a method for suppression of gene expression in an insect (e.g., coleopteran and/or hemipteran) pest comprises providing in the tissue of the host of the pest a gene-suppressive amount of at least one dsRNA molecule formed following transcription of a polynucleotide as described herein, at least one segment of which is complementary to an mRNA within the cells of the insect pest. A dsRNA molecule, including its modified form such as an siRNA, miRNA, shRNA, or hpRNA molecule, ingested by an insect pest may be at least from about 80%, 81%, 82%, 83%, 84%, 85%, 86%, 87%, 88%, 89%, 90%, 91%, 92%, 93%, 94%, 95%, 96%, 97%, 98%, 99%, or about 100% identical to an RNA molecule transcribed from a Gho/Sec24B2 or Sec24B1 DNA molecule, for example, comprising a polynucleotide selected from the group consisting of SEQ ID NOs:112-116 and 119-127. Isolated and substantially purified nucleic acid molecules including, but not limited to, non-naturally occurring polynucleotides and recombinant DNA constructs for providing dsRNA molecules are therefore provided, which suppress or inhibit the expression of an endogenous coding polynucleotide or a target coding polynucleotide in an insect pest when introduced thereto.

Particular embodiments provide a delivery system for the delivery of iRNA molecules for the post-transcriptional inhibition of one or more target gene(s) in an insect (e.g., coleopteran and/or hemipteran) plant pest and control of a population of the plant pest. In some embodiments, the delivery system comprises ingestion of a host transgenic plant cell or contents of the host cell comprising RNA molecules transcribed in the host cell. In these and further embodiments, a transgenic plant cell or a transgenic plant is created that contains a recombinant DNA construct providing a stabilized dsRNA molecule of the invention. Transgenic plant cells and transgenic plants comprising nucleic acids encoding a particular iRNA molecule may be produced by employing recombinant DNA technologies (which basic technologies are well-known in the art) to construct a plant transformation vector comprising a polynucleotide encoding an iRNA molecule of the invention (e.g., a stabilized dsRNA molecule); to transform a plant cell or plant; and to generate the transgenic plant cell or the transgenic plant that contains the transcribed iRNA molecule.

To impart protection from insect (e.g., coleopteran and/or hemipteran) pests to a transgenic plant, a recombinant DNA molecule may, for example, be transcribed into an iRNA molecule, such as a dsRNA molecule, an siRNA molecule, an miRNA molecule, an shRNA molecule, or an hpRNA molecule. In some embodiments, an RNA molecule transcribed from a recombinant DNA molecule may form a dsRNA molecule within the tissues or fluids of the recombinant plant. Such a dsRNA molecule may be comprised in part of a polynucleotide that is identical to a corresponding polynucleotide transcribed from a DNA within an insect pest of a type that may infest the host plant. Expression of a target gene within the pest is suppressed by the dsRNA molecule, and the suppression of expression of the target gene in the pest results in the transgenic plant being resistant to the pest. The modulatory effects of dsRNA molecules have been shown to be applicable to a variety of genes expressed in pests, including, for example, endogenous genes responsible for cellular metabolism or cellular transformation, including house-keeping genes; transcription factors; molting-related genes; and other genes which encode polypeptides involved in cellular metabolism or normal growth and development.

For transcription from a transgene in vivo or an expression construct, a regulatory region (e.g., promoter, enhancer, silencer, and polyadenylation signal) may be used in some embodiments to transcribe the RNA strand (or strands). Therefore, in some embodiments, as set forth, supra, a polynucleotide for use in producing iRNA molecules may be operably linked to one or more promoter elements functional in a plant host cell. The promoter may be an endogenous promoter, normally resident in the host genome. The polynucleotide of the present invention, under the control of an operably linked promoter element, may further be flanked by additional elements that advantageously affect its transcription and/or the stability of a resulting transcript. Such elements may be located upstream of the operably linked promoter, downstream of the 3′ end of the expression construct, and may occur both upstream of the promoter and downstream of the 3′ end of the expression construct.

Some embodiments provide methods for reducing the damage to a host plant (e.g., a corn plant) caused by an insect (e.g., coleopteran and/or hemipteran) pest that feeds on the plant, wherein the method comprises providing in the host plant a transformed plant cell expressing at least one nucleic acid molecule of the invention, wherein the nucleic acid molecule(s) functions upon being taken up by the pest(s) to inhibit the expression of a target polynucleotide within the pest(s), which inhibition of expression results in mortality and/or reduced growth of the pest(s), thereby reducing the damage to the host plant caused by the pest(s). In some embodiments, the nucleic acid molecule(s) comprise dsRNA molecules. In these and further embodiments, the nucleic acid molecule(s) comprise dsRNA molecules that each comprise more than one polynucleotide that is specifically hybridizable to a nucleic acid molecule expressed in a coleopteran and/or hemipteran pest cell. In some embodiments, the nucleic acid molecule(s) consist of one polynucleotide that is specifically hybridizable to a nucleic acid molecule expressed in an insect pest cell.

In some embodiments, a method for increasing the yield of a corn crop is provided, wherein the method comprises introducing into a corn plant at least one nucleic acid molecule of the invention; cultivating the corn plant to allow the expression of an iRNA molecule comprising the nucleic acid, wherein expression of an iRNA molecule comprising the nucleic acid inhibits insect (e.g., coleopteran and/or hemipteran) pest damage and/or growth, thereby reducing or eliminating a loss of yield due to pest infestation. In some embodiments, the iRNA molecule is a dsRNA molecule. In these and further embodiments, the nucleic acid molecule(s) comprise dsRNA molecules that each comprise more than one polynucleotide that is specifically hybridizable to a nucleic acid molecule expressed in an insect pest cell. In some examples, the nucleic acid molecule(s) comprises a polynucleotide that is specifically hybridizable to a nucleic acid molecule expressed in a coleopteran and/or hemipteran pest cell.

In some embodiments, a method for modulating the expression of a target gene in an insect (e.g., coleopteran and/or hemipteran) pest is provided, the method comprising: transforming a plant cell with a vector comprising a polynucleotide encoding at least one iRNA molecule of the invention, wherein the polynucleotide is operatively-linked to a promoter and a transcription termination element; culturing the transformed plant cell under conditions sufficient to allow for development of a plant cell culture including a plurality of transformed plant cells; selecting for transformed plant cells that have integrated the polynucleotide into their genomes; screening the transformed plant cells for expression of an iRNA molecule encoded by the integrated polynucleotide; selecting a transgenic plant cell that expresses the iRNA molecule; and feeding the selected transgenic plant cell to the insect pest. Plants may also be regenerated from transformed plant cells that express an iRNA molecule encoded by the integrated nucleic acid molecule. In some embodiments, the iRNA molecule is a dsRNA molecule. In these and further embodiments, the nucleic acid molecule(s) comprise dsRNA molecules that each comprise more than one polynucleotide that is specifically hybridizable to a nucleic acid molecule expressed in an insect pest cell. In some examples, the nucleic acid molecule(s) comprises a polynucleotide that is specifically hybridizable to a nucleic acid molecule expressed in a coleopteran and/or hemipteran pest cell.

iRNA molecules of the invention can be incorporated within the seeds of a plant species (e.g., corn), either as a product of expression from a recombinant gene incorporated into a genome of the plant cells, or as incorporated into a coating or seed treatment that is applied to the seed before planting. A plant cell comprising a recombinant gene is considered to be a transgenic event. Also included in embodiments of the invention are delivery systems for the delivery of iRNA molecules to insect (e.g., coleopteran and/or hemipteran) pests. For example, the iRNA molecules of the invention may be directly introduced into the cells of a pest(s). Methods for introduction may include direct mixing of iRNA with plant tissue from a host for the insect pest(s), as well as application of compositions comprising iRNA molecules of the invention to host plant tissue. For example, iRNA molecules may be sprayed onto a plant surface. Alternatively, an iRNA molecule may be expressed by a microorganism, and the microorganism may be applied onto the plant surface, or introduced into a root or stem by a physical means such as an injection. As discussed, supra, a transgenic plant may also be genetically engineered to express at least one iRNA molecule in an amount sufficient to kill the insect pests known to infest the plant. iRNA molecules produced by chemical or enzymatic synthesis may also be formulated in a manner consistent with common agricultural practices, and used as spray-on products for controlling plant damage by an insect pest. The formulations may include the appropriate adjuvants (e.g., stickers and wetters) required for efficient foliar coverage, as well as UV protectants to protect iRNA molecules (e.g., dsRNA molecules) from UV damage. Such additives are commonly used in the bioinsecticide industry, and are well known to those skilled in the art. Such applications may be combined with other spray-on insecticide applications (biologically based or otherwise) to enhance plant protection from the pests.

All references, including publications, patents, and patent applications, cited herein are hereby incorporated by reference to the extent they are not inconsistent with the explicit details of this disclosure, and are so incorporated to the same extent as if each reference were individually and specifically indicated to be incorporated by reference and were set forth in its entirety herein. The references discussed herein are provided solely for their disclosure prior to the filing date of the present application. Nothing herein is to be construed as an admission that the inventors are not entitled to antedate such disclosure by virtue of prior invention.

The following EXAMPLES are provided to illustrate certain particular features and/or aspects. These EXAMPLES should not be construed to limit the disclosure to the particular features or aspects described.

### EXAMPLES

### Example 1

### Materials and Methods

Sample Preparation and Bioassays.

A number of dsRNA molecules (including those corresponding to Gho/Sec24B2 reg1 (SEQ ID NO:3), Gho/Sec24B2 reg2 (SEQ ID NO:4), Gho/Sec24B2 ver1 (SEQ ID NO:5), Gho/Sec24B2 ver2 (SEQ ID NO:6), Sec24B1 reg1 (SEQ ID NO:104), and Gho/Sec24B2 reg3 (SEQ ID NO:109) were synthesized and purified using a MEGASCRIPT® T7 RNAi kit (LIFE TECHNOLOGIES, Carlsbad, Calif.) or T7 Quick High Yield RNA Synthesis Kit (NEW ENGLAND BIOLABS, Whitby, Ontario). The purified dsRNA molecules were prepared in TE buffer, and all bioassays contained a control treatment consisting of this buffer, which served as a background check for mortality or growth inhibition of WCR (Diabrotica virgifera virgifera LeConte). The concentrations of dsRNA molecules in the bioassay buffer were measured using a NANODROP™ 8000 spectrophotometer (THERMO SCIENTIFIC, Wilmington, Del.).

Samples were tested for insect activity in bioassays conducted with adult insect larvae on artificial insect diet. WCR eggs were obtained from CROP CHARACTERISTICS, INC. (Farmington, Minn.).

The bioassays were conducted in 128-well plastic trays specifically designed for insect bioassays (C-D INTERNATIONAL, Pitman, N.J.). Each well contained approximately 1.0 mL of an artificial diet designed for growth of coleopteran insects. A 60 μL aliquot of dsRNA sample was delivered by pipette onto the surface of the diet of each well (40 μL/cm²). dsRNA sample concentrations were calculated as the amount of dsRNA per square centimeter (ng/cm²) of surface area (1.5 cm²) in the well. The treated trays were held in a fume hood until the liquid on the diet surface evaporated or was absorbed into the diet.

Within a few hours of eclosion, individual larvae were picked up with a moistened camel hair brush and deposited on the treated diet (one or two larvae per well). The infested wells of the 128-well plastic trays were then sealed with adhesive sheets of clear plastic, and vented to allow gas exchange. Bioassay trays were held under controlled environmental conditions (28° C., ˜40% Relative Humidity, 16:8 (Light:Dark)) for 9 days, after which time the total number of insects exposed to each sample, the number of dead insects, and the weight of surviving insects were recorded. Average percent mortality and average growth inhibition were calculated for each treatment. Growth inhibition (GI) was calculated as follows:

GI=[1−(TWIT/TNIT)/(TWIBC/TNIBC)],

where TWIT is the Total Weight of live Insects in the Treatment;

TNIT is the Total Number of Insects in the Treatment;

TWIBC is the Total Weight of live Insects in the Background Check (Buffer control); and

TNIBC is the Total Number of Insects in the Background Check (Buffer control).

The LC₅₀ (Lethal Concentration) is defined as the dosage at which 50% of the test insects are killed. The GI₅₀ (Growth Inhibition) is defined as the dosage at which the mean growth (e.g., live weight) of the test insects is 50% of the mean value seen in Background Check samples. The statistical analysis was done using JMP™ software (SAS, Cary, N.C.).

Replicated bioassays demonstrated that ingestion of particular samples resulted in a surprising and unexpected mortality and growth inhibition of corn rootworm larvae.

### Example 2

### Identification of Candidate Target Genes from Diabrotica

Insects from multiple stages of WCR (Diabrotica virgifera virgifera LeConte) development were selected for pooled transcriptome analysis to provide candidate target gene sequences for control by RNAi transgenic plant insect protection technology.

In one exemplification, total RNA was isolated from about 0.9 gm whole first-instar WCR larvae; (4 to 5 days post-hatch; held at 16° C.), and purified using the following phenol/TRI REAGENT®-based method (MOLECULAR RESEARCH CENTER, Cincinnati, Ohio).

Larvae were homogenized at room temperature in a 15 mL homogenizer with 10 mL of TRI REAGENT® until a homogenous suspension was obtained. Following 5 min. incubation at room temperature, the homogenate was dispensed into 1.5 mL microfuge tubes (1 mL per tube), 200 μL of chloroform was added, and the mixture was vigorously shaken for 15 seconds. After allowing the extraction to sit at room temperature for 10 min, the phases were separated by centrifugation at 12,000×g at 4° C. The upper phase (comprising about 0.6 mL) was carefully transferred into another sterile 1.5 mL tube, and an equal volume of room temperature isopropanol was added. After incubation at room temperature for 5 to 10 min, the mixture was centrifuged 8 min at 12,000×g (4° C. or 25° C.).

The supernatant was carefully removed and discarded, and the RNA pellet was washed twice by vortexing with 75% ethanol, with recovery by centrifugation for 5 min at 7,500×g (4° C. or 25° C.) after each wash. The ethanol was carefully removed, the pellet was allowed to air-dry for 3 to 5 min, and then was dissolved in nuclease-free sterile water. RNA concentration was determined by measuring the absorbance (A) at 260 nm and 280 nm. A typical extraction from about 0.9 gm of larvae yielded over 1 mg of total RNA, with an A₂₆₀/A₂₈₀ ratio of 1.9. The RNA thus extracted was stored at −80° C. until further processed.

RNA quality was determined by running an aliquot through a 1% agarose gel. The agarose gel solution was made using autoclaved 10×TAE buffer (Tris-acetate EDTA; 1× concentration is 0.04 M Tris-acetate, 1 mM EDTA (ethylenediamine tetra-acetic acid sodium salt), pH 8.0) diluted with DEPC (diethyl pyrocarbonate)-treated water in an autoclaved container. 1×TAE was used as the running buffer. Before use, the electrophoresis tank and the well-forming comb were cleaned with RNaseAway™ (INVITROGEN INC., Carlsbad, Calif.). Two μL of RNA sample were mixed with 8 μL of TE buffer (10 mM Tris HCl pH 7.0; 1 mM EDTA) and 10 μL of RNA sample buffer (NOVAGEN® Catalog No 70606; EMD4 Bioscience, Gibbstown, N.J.). The sample was heated at 70° C. for 3 min, cooled to room temperature, and 5 μL (containing 1 μg to 2 μg RNA) were loaded per well. Commercially available RNA molecular weight markers were simultaneously run in separate wells for molecular size comparison. The gel was run at 60 volts for 2 hrs.

A normalized cDNA library was prepared from the larval total RNA by a commercial service provider (EUROFINS MWG Operon, Huntsville, Ala.), using random priming. The normalized larval cDNA library was sequenced at ½ plate scale by GS FLX 454 Titanium™ series chemistry at EUROFINS MWG Operon, which resulted in over 600,000 reads with an average read length of 348 bp. 350,000 reads were assembled into over 50,000 contigs. Both the unassembled reads and the contigs were converted into BLASTable databases using the publicly available program, FORMATDB (available from NCBI).

Total RNA and normalized cDNA libraries were similarly prepared from materials harvested at other WCR developmental stages. A pooled transcriptome library for target gene screening was constructed by combining cDNA library members representing the various developmental stages.

Candidate genes for RNAi targeting were hypothesized to be essential for survival and growth in pest insects. Selected target gene homologs were identified in the transcriptome sequence database, as described below. Full-length or partial sequences of the target genes were amplified by PCR to prepare templates for double-stranded RNA (dsRNA) production.

TBLASTN searches using candidate protein coding sequences were run against BLASTable databases containing the unassembled Diabrotica sequence reads or the assembled contigs. Significant hits to a Diabrotica sequence (defined as better than e⁻²⁰ for contigs homologies and better than e⁻¹⁰ for unassembled sequence reads homologies) were confirmed using BLASTX against the NCBI non-redundant database. The results of this BLASTX search confirmed that the Diabrotica homolog candidate gene sequences identified in the TBLASTN search indeed comprised Diabrotica genes, or were the best hit to the non-Diabrotica candidate gene sequence present in the Diabrotica sequences. In most cases, Tribolium candidate genes which were annotated as encoding a protein gave an unambiguous sequence homology to a sequence or sequences in the Diabrotica transcriptome sequences. In a few cases, it was clear that some of the Diabrotica contigs or unassembled sequence reads selected by homology to a non-Diabrotica candidate gene overlapped, and that the assembly of the contigs had failed to join these overlaps. In those cases, Sequencher™ v4.9 (GENE CODES CORPORATION, Ann Arbor, Mich.) was used to assemble the sequences into longer contigs.

Candidate target genes encoding Diabrotica Gho/Sec24B2 (SEQ ID NO:1 and SEQ ID NO:107) and Sec24B1 (SEQ ID NO:102) were identified as genes that may lead to coleopteran pest mortality, inhibition of growth, or inhibition of developmentin WCR. Gho/Sec24B2 and Sec24B1 are components of the coat protein complex II (COPII) which promotes the formation of transport vesicles from the endoplasmic reticulum (ER) to the Golgi complex (See, on the world-wide-web, uniprot.org/uniprot/P40482). The coat has two main functions, the physical deformation of the ER membrane into vesicles and the selection of cargo molecules. Sec23 and Sec24 are structurally related and form a heterodimer. Sec24 is largely responsible for the cargo recruitment to COPII vesicles, and the Sec23/Sec24 inner shellcomplex forms a platform for the COPII outer coat (See, on the world-wide-web, cshperspectives.cshlp.org/content/5/2/a013367.long).

Our results herein indicated that genes encoding proteins of the Sec23/Sec24 complex (e.g., Diabrotica virgifera proteins) are candidate target genes that may lead to insect pest mortality, inhibition of growth, or inhibition of development, for example, in coleopteran pests.

The sequences of SEQ ID NO:1 and SEQ ID NO:102 are novel. The sequences are not provided in public databases and are not disclosed in WO/2011/025860; U.S. Patent Application No. 20070124836; U.S. Patent Application No. 20090306189; U.S. Patent Application No. US20070050860; U.S. Patent Application No. 20100192265; or U.S. Pat. No. 7,612,194. There was no significant homologous nucleotide sequence found with a search in GENBANK. The closest homolog of the Diabrotica GHO/SEC24B2 amino acid sequence (SEQ ID NO:2) is a Tribolium casetanum protein having GENBANK Accession No. XP 971886.1 (77% similar; 67% identical over the homology region). The closest homolog of the Diabrotica SEC24B1 amino acid sequence (SEQ ID NO:103) is a Tribolium casetanum protein having GENBANK Accession No. XP_974325.2 (84% similar; 74% identical over the homology region). Thus, even these encoded polypeptides have no significant homology with any known protein of more than 85%.

Gho/Sec24B2 and Sec24B1 dsRNA transgenes can be combined with other dsRNA molecules to provide redundant RNAi targeting and synergistic RNAi effects. Transgenic corn events expressing dsRNA that targets Gho/Sec24B2 and/or Sec24B1 are useful for preventing root feeding damage by corn rootworm. Gho/Sec24B2 and Sec24B1 dsRNA transgenes represent new modes of action for combining with Bacillus thuringiensis insecticidal protein technology in Insect Resistance Management gene pyramids to mitigate the development of rootworm populations resistant to either of these rootworm control technologies.

### Example 3

### Amplification of Target Genes from Diabrotica

Full-length or partial clones of sequences of Gho/Sec24B2 and Sec24B1 candidate genes were used to generate PCR amplicons for dsRNA synthesis. Primers were designed to amplify portions of coding regions of each target gene by PCR. See Table 1. Where appropriate, a T7 phage promoter sequence (TTAATACGACTCACTATAGGGAGA; SEQ ID NO:13) was incorporated into the 5′ ends of the amplified sense or antisense strands. See Table 1. Total RNA was extracted from WCR using TRIzol® (Life Technologies, Grand Island, N.Y.), and was then used to make first-strand cDNA with SuperScriptIII® First-Strand Synthesis System and manufacturers Oligo dT primed instructions (Life Technologies, Grand Island, N.Y.). First-strand cDNA was used as template for PCR reactions using opposing primers positioned to amplify all or part of the native target gene sequence. dsRNA was also amplified from a DNA clone comprising the coding region for a yellow fluorescent protein (YFP) (SEQ ID NO:8; Shagin et al. (2004) Mol. Biol. Evol. 21(5):841-50).

TABLE 1
Primers and Primer Pairs used to amplify portions
of coding regions of exemplary Gho/Sec24B2 or
Sec24B1 target genes and YFP negative control gene.

| | Gene ID | Primer ID | Sequence |
|---|---|---|---|
| Pair 1 | Gho/Sec24B2 reg1 | sec24BT7_F | TTAATACGACTCACTATAGGGAGATATATCTTCAA |
| | | | TAACGCTTAC (SEQ ID NO: 10) |
| | | sec24BT7_R | TTAATACGACTCACTATAGGGAGAGTGTCTTCATT |
| | | | CAGTTTG (SEQ ID NO: 11) |
| Pair 2 | Gho/Sec24B2 reg2 | gho-2F | TTAATACGACTCACTATAGGGAGACTAAGGAAACC |
| | | | GAAGTCGTTTTGC (SEQ ID NO: 12) |
| | | gho-2R | TTAATACGACTCACTATAGGGAGAGCATCAAACGC |
| | | | TATTGGTCGAC (SEQ ID NO: 13) |
| Pair 3 | Gho/Sec24B2 ver1 | Gho_v1F | TTAATACGACTCACTATAGGGAGATCGTTTTGCTT |
| | | | CCCGCAATTC (SEQ ID NO: 14) |
| | | Gho_v1R | TTAATACGACTCACTATAGGGAGACACTTGACCAA |
| | | | TAGTCGCTATATCG (SEQ ID NO: 15) |
| Pair 4 | Gho/Sec24B2_ver2 | Gho_v2F | TTAATACGACTCACTATAGGGAGAGTCGTTTTGCT |
| | | | TCCCGCAATTC (SEQ ID NO: 16) |
| | | Gho_v2R | TTAATACGACTCACTATAGGGAGATCGCGGTTCTT |
| | | | CAATTTACCTG (SEQ ID NO: 17) |
| Pair 5 | Sec24B1 | Sec24B1_F | TTAATACGACTCACTATAGGGAGACTCAGTATGTA |
| | | | GATATAGC (SEQ ID NO: 105) |
| | | Sec24B1_R | TTAATACGACTCACTATAGGGAGATGGAAGGTATG |
| | | | AATAGAA (SEQ ID NO: 106) |
| Pair 6 | Sec24B2 | Sec24B2_Reg3_F | TTAATACGACTCACTATAGGGAGAGCTTATAACTC |
| | | | TGCTCTACATTTTTATTC (SEQ ID NO: 110) |
| | | Sec24B2_Reg3_R | TTAATACGACTCACTATAGGGAGACTGAGAGTTTA |
| | | | CTACGAACAGATCG (SEQ ID NO: 111) |
| Pair 7 | YFP | YFP-F_T7 | TTAATACGACTCACTATAGGGAGACACCATGGGCT |
| | | | CCAGCGGCGCCC (SEQ ID NO: 31) |
| | | YFP-R_T7 | TTAATACGACTCACTATAGGGAGAAGATCTTGAAG |
| | | | GCGCTCTTCAGG (SEQ ID NO: 34) |

### Example 4

### RNAi Constructs

Template Preparation by PCR and dsRNA Synthesis.

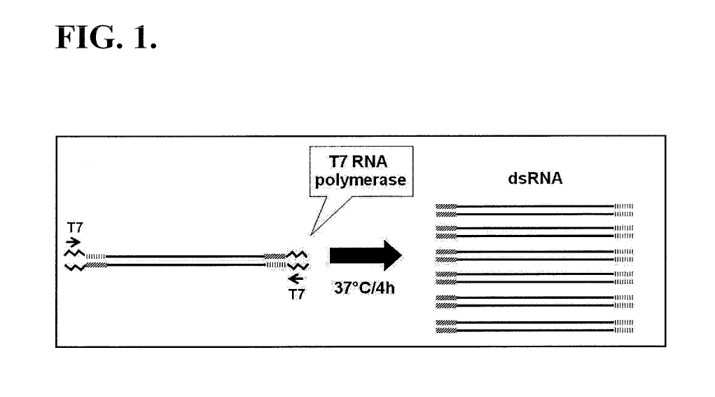

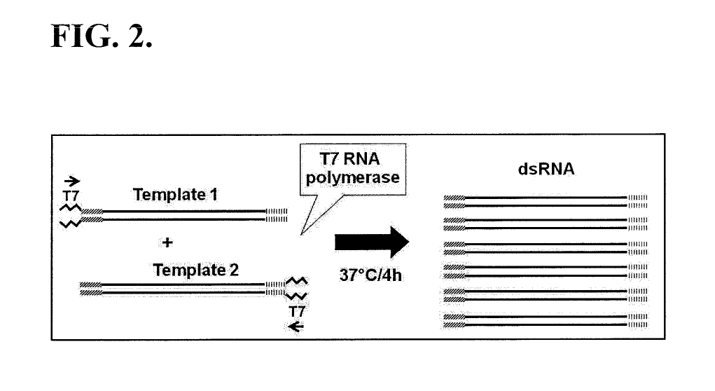

The strategies used to provide specific templates for Gho/Sec24B2, Sec24B1, and YFP dsRNA production are shown in FIG. 1 and FIG. 2. Template DNAs intended for use in Gho/Sec24B2 or Sec24B1dsRNA synthesis were prepared by PCR using the primer pairs in Table 1 and (as PCR template) first-strand cDNA prepared from total RNA isolated from WCR first-instar larvae. For each selected Gho/Sec24B2, Sec24B1, and YFP target gene region, PCR amplifications introduced a T7 promoter sequence at the 5′ ends of the amplified sense and antisense strands (the YFP segment was amplified from a DNA clone of the YFP coding region). The two PCR amplified fragments for each region of the target genes were then mixed in approximately equal amounts, and the mixture was used as transcription template for dsRNA production. See FIG. 1. The sequences of the dsRNA templates amplified with the particular primer pairs were: SEQ ID NO:3 (Gho/Sec24B2 reg1), SEQ ID NO:4 (Gho/Sec24B2 reg2), SEQ ID NO:5 (Gho/Sec24B2 ver1), SEQ ID NO:6 (Gho/Sec24B2 ver2), SEQ ID NO:109 (Sec24B2 reg3), GFP (SEQ ID:8), and YFP (SEQ ID NO:7). Double-stranded RNA for insect bioassay was synthesized and purified using an AMBION® MEGASCRIPT® RNAi kit following the manufacturer's instructions (INVITROGEN) or HiScribe® T7 In Vitro Transcription Kit following the manufacturer's instructions (New England Biolabs, Ipswich, Mass.). The concentrations of dsRNAs were measured using a NANODROP™ 8000 spectrophotometer (THERMO SCIENTIFIC, Wilmington, Del.).

Construction of Plant Transformation Vectors.

Entry vectors harboring a target gene construct for hairpin formation comprising a segment of Gho/Sec24B2 (SEQ ID NO:1) and/or Sec24B1 (SEQ ID NO:102) are assembled using a combination of chemically synthesized fragments (DNA2.0, Menlo Park, Calif.) and standard molecular cloning methods. Intramolecular hairpin formation by RNA primary transcripts is facilitated by arranging (within a single transcription unit) two copies of a segment of the Gho/Sec24B2 target gene sequence in opposite orientation to one another, the two segments being separated by a linker sequence (e.g., ST-LS1, Vancanneyt et al. (1990) Mol. Gen. Genet. 220(2):245-50) to form a loop structure. Thus, the primary mRNA transcript contains the two Gho/Sec24B2 gene segment sequences as large inverted repeats of one another, separated by the linker sequence. A copy of a promoter (e.g., maize ubiquitin 1, U.S. Pat. No. 5,510,474; 35S from Cauliflower Mosaic Virus (CaMV); promoters from rice actin genes; ubiquitin promoters; pEMU; MAS; maize H3 histone promoter; ALS promoter; phaseolin gene promoter; cab; rubisco; LAT52; Zm13; and/or apg) is used to drive production of the primary mRNA hairpin transcript, and a fragment comprising a 3′ untranslated region for example but not limited to a maize peroxidase 5 gene (ZmPer5 3′UTR v2; U.S. Pat. No. 6,699,984), AtUbi10, AtEf1, or StPinII is used to terminate transcription of the hairpin-RNA-expressing gene.

Entry vector pDAB114538 comprises a Gho/Sec24B2 hairpin v1-RNA construct (SEQ ID NO:18) that comprises a segment of Gho/Sec24B2 (SEQ ID NO:1). Entry vector pDAB114548 comprises a Gho/Sec24B2 hairpin v2-RNA construct (SEQ ID NO:19) that comprises a segment of Gho/Sec24B2 (SEQ ID NO:1) distinct from that found in pDAB114538. Entry vectors pDAB114538 and pDAB114548 described above are used in standard GATEWAY® recombination reactions with a typical binary destination vector (pDAB115765) to produce Gho/Sec24B2 hairpin RNA expression transformation vectors for Agrobacterium-mediated maize embryo transformations (pDAB114544 and pDAB114549, respectively).

A negative control binary vector which comprises a gene that expresses a YFP hairpin dsRNA, is constructed by means of standard GATEWAY® recombination reactions with a typical binary destination vector (pDAB109805) and entry vector pDAB101670. Entry Vector pDAB101670 comprises a YFP hairpin sequence (SEQ ID NO:20) under the expression control of a maize ubiquitin 1 promoter (as above) and a fragment comprising a 3′ untranslated region from a maize peroxidase 5 gene (as above).

A Binary destination vector comprises a herbicide tolerance gene (aryloxyalknoate dioxygenase; AAD-1 v3) (U.S. Pat. No. 7,838,733(B2), and Wright et al. (2010) Proc. Natl. Acad. Sci. U.S.A. 107:20240-20245) under the regulation of a plant operable promoter (e.g. sugarcane bacilliform badnavirus (ScBV) promoter (Schenk et al. (1999) Plant Molec. Biol. 39:1221-1230) or ZmUbi1 (U.S. Pat. No. 5,510,474)). 5′UTR and intron from these promoters, are positioned between the 3′ end of the promoter segment and the start codon of the AAD-1 coding region. A fragment comprising a 3′ untranslated region from a maize lipase gene (ZmLip 3′UTR; U.S. Pat. No. 7,179,902) is used to terminate transcription of the AAD-1 mRNA.

A further negative control binary vector, pDAB101556, which comprises a gene that expresses a YFP protein, is constructed by means of standard GATEWAY® recombination reactions with a typical binary destination vector (pDAB9989) and entry vector (pDAB100287). Binary destination vector pDAB9989 comprises a herbicide tolerance gene (aryloxyalknoate dioxygenase; AAD-1 v3) (as above) under the expression regulation of a maize ubiquitin 1 promoter (as above) and a fragment comprising a 3′ untranslated region from a maize lipase gene (ZmLip 3′UTR; as above). Entry Vector pDAB100287 comprises a YFP coding region (SEQ ID NO:32) under the expression control of a maize ubiquitin 1 promoter (as above) and a fragment comprising a 3′ untranslated region from a maize peroxidase 5 gene (as above).

### Example 5

### Screening of Candidate Target Genes in Diabrotica Larvae

Synthetic dsRNA designed to inhibit target gene sequences identified in EXAMPLE 2 caused mortality and growth inhibition when administered to WCR in diet-based assays.

Replicated bioassays demonstrated that ingestion of dsRNA preparations derived from Gho/Sec24B2 reg1, Gho/Sec24B2 reg2, Gho/Sec24B2 ver1, Gho/Sec24B2 ver2, Sec24B2 reg3 each resulted in mortality and/or growth inhibition of western corn rootworm larvae. Table 2 and Table 3 show the results of diet-based feeding bioassays of WCR larvae following 9-day exposure to these dsRNAs, as well as the results obtained with a negative control sample of dsRNA prepared from a yellow fluorescent protein (YFP) coding region (SEQ ID NO:8).

TABLE 2
Results of Gho/Sec24B2 dsRNA diet feeding assays obtained with western corn rootworm
larvae after 9 days of feeding. ANOVA analysis found significance differences in Mean %
Mortality and Mean % Growth Inhibition (GI). Means were separated using the Tukey-Kramer test.

| | Dose | | Mean (% Mortality) ± | Mean (GI) ± |
|---|---|---|---|---|
| Gene Name | (ng/cm²) | N | SEM* | SEM |
| Gho/Sec24B2 reg1 | 500 | 6 | 56.19 ± 12.82 (B) | 0.61 ± 0.23 (B) |
| Gho/Sec24B2 reg2 | 500 | 6 | 59.80 ± 8.10 (AB) | 0.69 ± 0.04 (AB) |
| Gho/Sec24B2 ver1 | 500 | 14 | 70.82 ± 4.00 (A) | 0.92 ± 0.03 (A) |
| Gho/Sec24B2 ver2 | 500 | 12 | 78.43 ± 4.93 (AB) | 0.94 ± 0.03 (A) |
| Sec24B2 reg3 | 500 | 4 | 1.47 ± 1.47 (C) | 0.66 ± 0.03 (AB) |
| TE** | 0 | 20 | 7.78 ± 2.19 (C) | −0.02 ± 0.03 (C) |
| WATER | 0 | 19 | 7.01 ± 1.81 (C) | 0.09 ± 0.04 (C) |
| YFP*** | 500 | 20 | 9.29 ± 1.63 (C) | 0.16 ± 0.05 (C) |

* SEM = Standard Error of the Mean. Letters in parentheses designate statistical levels. Levels not connected by same letter are significantly different (P < 0.05).
**TE = Tris HCl (10 mM) plus EDTA (1 mM) buffer, pH 8.
***YFP = Yellow Fluorescent Protein

TABLE 3
Summary of oral potency of Gho/Sec24B2
dsRNA onWCR larvae (ng/cm²).

| Gene Name | LC₅₀ | Range | GI₅₀ | Range |
|---|---|---|---|---|
| Gho/Sec24B2 ver1 | 45.48 | 35.53-58.56 | 8.41 | 5.60-12.61 |
| Gho/Sec24B2 ver2 | 39.34 | 30.93-50.31 | 10.65 | 8.06-14.07 |

It has previously been suggested that certain genes of Diabrotica spp. may be exploited for RNAi-mediated insect control. See U.S. Patent Publication No. 2007/0124836, which discloses 906 sequences, and U.S. Pat. No. 7,612,194, which discloses 9,112 sequences. However, it was determined that many genes suggested to have utility for RNAi-mediated insect control are not efficacious in controlling Diabrotica. It was also determined that sequences Gho/Sec24B2 reg1, Gho/Sec24B2 reg2, Gho/Sec24B2 ver1, Gho/Sec24B2 ver2, and Sec24B2 reg3 each provide surprising and unexpected superior control of Diabrotica, compared to other genes suggested to have utility for RNAi-mediated insect control.

For example, Annexin, Beta spectrin 2, and mtRP-L4 were each suggested in U.S. Pat. No. 7,612,194 to be efficacious in RNAi-mediated insect control. SEQ ID NO:23 is the DNA sequence of Annexin region 1 (Reg 1), and SEQ ID NO:24 is the DNA sequence of Annexin region 2 (Reg 2). SEQ ID NO:25 is the DNA sequence of Beta spectrin 2 region 1 (Reg 1), and SEQ ID NO:26 is the DNA sequence of Beta spectrin 2 region 2 (Reg2). SEQ ID NO:27 is the DNA sequence of mtRP-L4 region 1 (Reg 1), and SEQ ID NO:28 is the DNA sequence of mtRP-L4 region 2 (Reg 2). A YFP sequence (SEQ ID NO:8) was also used to produce dsRNA as a negative control.

Each of the aforementioned sequences was used to produce dsRNA by the methods of EXAMPLE 3. The strategy used to provide specific templates for dsRNA production is shown in FIG. 2. Template DNAs intended for use in dsRNA synthesis were prepared by PCR using the primer pairs in Table 4 and (as PCR template) first-strand cDNA prepared from total RNA isolated from WCR first-instar larvae. (YFP was amplified from a DNA clone.) For each selected target gene region, two separate PCR amplifications were performed. The first PCR amplification introduced a T7 promoter sequence at the 5′ end of the amplified sense strands. The second reaction incorporated the T7 promoter sequence at the 5′ ends of the antisense strands. The two PCR amplified fragments for each region of the target genes were then mixed in approximately equal amounts, and the mixture was used as transcription template for dsRNA production. See FIG. 2. Double-stranded RNA was synthesized and purified using an AMBION® MEGAscript® RNAi kit following the manufacturer's instructions (INVITROGEN). The concentrations of dsRNAs were measured using a NANODROP™ 8000 spectrophotometer (THERMO SCIENTIFIC, Wilmington, Del.). and the dsRNAs were each tested by the same diet-based bioassay methods described above. Table 4 lists the sequences of the primers used to produce the Annexin Reg1, Annexin Reg2, Beta spectrin 2 Reg1, Beta spectrin 2 Reg2, mtRP-L4 Reg1, mtRP-L4 Reg2, and YFP dsRNA molecules. Table 5 presents the results of diet-based feeding bioassays of WCR larvae following 9-day exposure to these dsRNA molecules. Replicated bioassays demonstrated that ingestion of these dsRNAs resulted in no mortality or growth inhibition of western corn rootworm larvae above that seen with control samples of TE buffer, Water, or YFP protein.

TABLE 4
Primers and Primer Pairs used to amplify
portions of coding regions of genes.

| | Gene | | |
|---|---|---|---|
| | (Region) | Primer ID | Sequence |
| Pair 8 | YFP | YFP-F_T7 | TTAATACGACTCACT |
| | | | ATAGGGAGACACCAT |
| | | | GGGCTCCAGCGGCGC |
| | | | CC |
| | | | (SEQ ID NO: 31) |
| | YFP | YFP-R | AGATCTTGAAGGCGC |
| | | | TCTTCAGG |
| | | | (SEQ ID NO: 32) |
| Pair 9 | YFP | YFP-F | CACCATGGGCTCCAG |
| | | | CGGCGCCC |
| | | | (SEQ ID NO: 33) |
| | YFP | YFP-R_T7 | TTAATACGACTCACT |
| | | | ATAGGGAGAAGATCT |
| | | | TGAAGGCGCTCTTCA |
| | | | GG |
| | | | (SEQ ID NO: 34) |
| Pair 10 | Annexin | Ann-F1_T7 | TTAATACGACTCACT |
| | (Reg 1) | | ATAGGGAGAGCTCCA |
| | | | ACAGTGGTTCCTTAT |
| | | | C |
| | | | (SEQ ID NO: 35) |
| | Annexin | Ann-R1 | CTAATAATTCTTTTT |
| | (Reg 1) | | TAATGTTCCTGAGG |
| | | | (SEQ ID NO: 36) |
| Pair 11 | Annexin | Ann-F1 | GCTCCAACAGTGGTT |
| | (Reg 1) | | CCTTATC |
| | | | (SEQ ID NO: 37) |
| | Annexin | Ann-R1_T7 | TTAATACGACTCACT |
| | (Reg 1) | | ATAGGGAGACTAATA |
| | | | ATTCTTTTTTAATGT |
| | | | TCCTGAGG |
| | | | (SEQ ID NO: 38) |
| Pair 12 | Annexin | Ann-F2_T7 | TTAATACGACTCACT |
| | (Reg 2) | | ATAGGGAGATTGTTA |
| | | | CAAGCTGGAGAACTT |
| | | | CTC |
| | | | (SEQ ID NO: 39) |
| | Annexin | Ann-R2 | CTTAACCAACAACGG |
| | (Reg 2) | | CTAATAAGG |
| | | | (SEQ ID NO: 40) |
| Pair 13 | Annexin | Ann-F2 | TTGTTACAAGCTGGA |
| | (Reg 2) | | GAACTTCTC |
| | | | (SEQ ID NO: 41) |
| | Annexin | Ann-R2T7 | TTAATACGACTCACT |
| | (Reg 2) | | ATAGGGAGACTTAAC |
| | | | CAACAACGGCTAATA |
| | | | AGG |
| | | | (SEQ ID NO: 42) |
| Pair 14 | Beta-spect2 | Betasp2-F1_T7 | TTAATACGACTCACT |
| | (Reg 1) | | ATAGGGAGAAGATGT |
| | | | TGGCTGCATCTAGAG |
| | | | AA |
| | | | (SEQ ID NO: 43) |
| | Beta-spect2 | Betasp2-R1 | GTCCATTCGTCCATC |
| | (Reg 1) | | CACTGCA |
| | | | (SEQ ID NO: 44) |
| Pair 15 | Beta-spect2 | Betasp2-F1 | AGATGTTGGCTGCAT |
| | (Reg 1) | | CTAGAGAA |
| | | | (SEQ ID NO: 45) |
| | Beta-spect2 | Betasp2-R1_T7 | TTAATACGACTCACT |
| | (Reg 1) | | ATAGGGAGAGTCCAT |
| | | | TCGTCCATCCACTGC |
| | | | A |
| | | | (SEQ ID NO: 46) |
| Pair 16 | Beta-spect2 | Betasp2-F2_T7 | TTAATACGACTCACT |
| | (Reg 2) | | ATAGGGAGAGCAGAT |
| | | | GAACACCAGCGAGAA |
| | | | A |
| | | | (SEQ ID NO: 47) |
| | Beta-spect2 | Betasp2-R2 | CTGGGCAGCTTCTTG |
| | (Reg 2) | | TTTCCTC |
| | | | (SEQ ID NO: 48) |
| Pair 17 | Beta-spect2 | Betasp2-F2 | GCAGATGAACACCAG |
| | (Reg 2) | | CGAGAAA |
| | | | (SEQ ID NO: 49) |
| | Beta-spect2 | Betasp2-R2_T7 | TTAATACGACTCACT |
| | (Reg 2) | | ATAGGGAGACTGGGC |
| | | | AGCTTCTTGTTTCCT |
| | | | C |
| | | | (SEQ ID NO: 50) |
| Pair 18 | mtRP-L4 | L4-F1_T7 | TTAATACGACTCACT |
| | (Reg 1) | | ATAGGGAGAAGTGAA |
| | | | ATGTTAGCAAATATA |
| | | | ACATCC |
| | | | (SEQ ID NO: 51) |
| | mtRP-L4 | L4-R1 | ACCTCTCACTTCAAA |
| | (Reg 1) | | TCTTGACTTTG |
| | | | (SEQ ID NO: 52) |
| Pair 19 | mtRP-L4 | L4-F1 | AGTGAAATGTTAGCA |
| | (Reg 1) | | AATATAACATCC |
| | | | (SEQ ID NO: 53) |
| | mtRP-L4 | L4-R1_T7 | TTAATACGACTCACT |
| | (Reg 1) | | ATAGGGAGAACCTCT |
| | | | CACTTCAAATCTTGA |
| | | | CTTTG |
| | | | (SEQ ID NO: 54) |
| Pair 20 | mtRP-L4 | L4-F2_T7 | TTAATACGACTCACT |
| | (Reg 2) | | ATAGGGAGACAAAGT |
| | | | CAAGATTTGAAGTGA |
| | | | GAGGT |
| | | | (SEQ ID NO: 55) |
| | mtRP-L4 | L4-R2 | CTACAAATAAAACAA |
| | (Reg 2) | | GAAGGACCCC |
| | | | (SEQ ID NO: 56) |
| Pair 21 | mtRP-L4 | L4-F2 | CAAAGTCAAGATTTG |
| | (Reg 2) | | AAGTGAGAGGT |
| | | | (SEQ ID NO: 57) |
| | mtRP-L4 | L4-R2_T7 | TTAATACGACTCACT |
| | (Reg 2) | | ATAGGGAGACTACAA |
| | | | ATAAAACAAGAAGGA |
| | | | CCCC |
| | | | (SEQ ID NO: 58) |

TABLE 5
Results of diet feeding assays obtained with western
corn rootworm larvae after 9 days.

| | | Mean Live | | Mean |
|---|---|---|---|---|
| Gene | Dose | Larval | Mean % | Growth |
| Name | (ng/cm²) | Weight (mg) | Mortality | Inhibition |
| Annexin-Reg 1 | 1000 | 0.545 | 0 | −0.262 |
| Annexin-Reg 2 | 1000 | 0.565 | 0 | −0.301 |
| Beta spectrin2 Reg 1 | 1000 | 0.340 | 12 | −0.014 |
| Beta spectrin2 Reg 2 | 1000 | 0.465 | 18 | −0.367 |
| mtRP-L4 Reg 1 | 1000 | 0.305 | 4 | −0.168 |
| mtRP-L4 Reg 2 | 1000 | 0.305 | 7 | −0.180 |
| TE buffer* | 0 | 0.430 | 13 | 0.000 |
| Water | 0 | 0.535 | 12 | 0.000 |
| YFP** | 1000 | 0.480 | 9 | −0.386 |

*TE = Tris HCl (10 mM) plus EDTA (1 mM) buffer, pH 8.
**YFP = Yellow Fluorescent Protein

### Example 6

### Sample Preparation and Bioassays for Adult Assays

RNA interference (RNAi) in western corn rootworms was conducted by feeding dsRNA corresponding to the segments of Gho/Sec24B2 or Sec24B1 target gene sequence to adults. Test insects were 24 to 48 hour old adults. Insects were obtained from Crop Characteristics, Inc. (Farmington, Minn.). Adults were reared at 23±1° C., relative humidity of >75%, and Light:Dark periods of 8 hr:16 hr for all bioassays. The insect rearing diet was adapted from Branson and Jackson (1988) J. Kansas Entomol. Soc. 61:353-5. Dry ingredients were added (48 gm/100 mL) to a solution comprising double distilled water with 2.9% agar and 7 mL of glycerol. In addition, 0.5 mL of a mixture comprising 47% propionic acid and 6% phosphoric acid solutions was added per 100 mL of diet to inhibit microbial growth. For all adult dsRNA feeding assays, the diet was modified to provide a consistency necessary to cut diet plugs. Dry ingredients were added at 60 gm/100 mL and agar was increased to 3.6%. The agar was dissolved in boiling water and the dry ingredients, glycerol, and propionic acid/phosphoric acid solution were added, mixed thoroughly, and poured to a depth of approximately 2 mm. Solidified diet plugs (approximately 4 mm in diameter by 2 mm height; 25.12 mm³) were cut from the diet with a No. 1 cork borer and were treated with 3 μl of dsRNA or water.

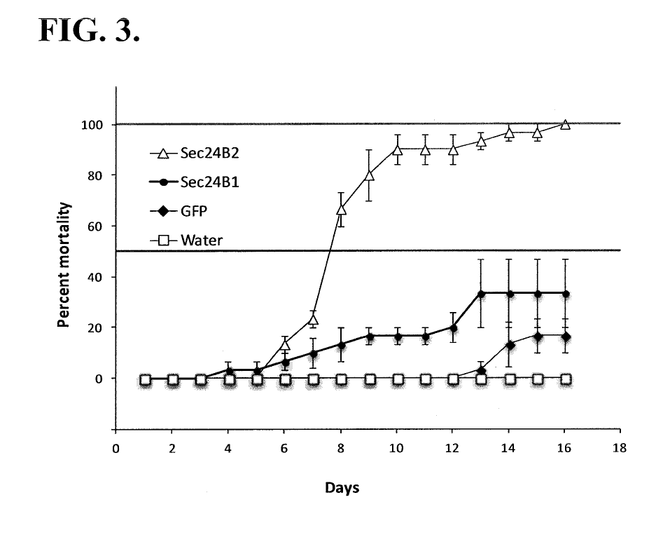

Adults were fed on artificial diet surface plugs treated with Gho/Sec24B2 reg1 (SEQ ID NO:3) or Sec24B1 reg1 (SEQ ID NO:104) gene-specific dsRNA (500 ng/diet plug; approximately 20 ng/mm³). Control treatments consisted of adults exposed to diet treated with the same concentration of GFP (green fluorescent protein) dsRNA (SEQ ID NO:9) or the same volume of water. GFP dsRNA was produced as described above using opposing primers having a T7 promoter sequence at their 5′ ends (SEQ ID NOs:29 and 30). Fresh artificial diet treated with dsRNA was provided every other day throughout the experiment. Three replications (Rep1, Rep2, and Rep3), each comprising ten adults, were run on separate days. FIG. 3 graphically represents the data presented in Table 6 showing the percent mortality of adult Diabrotica virgifera virgifera after exposure to 500 ng/diet plug of Gho/Sec24 reg1 dsRNA, Sec24B1 reg1 dsRNA, the same amount of GFP dsRNA, or the same volume of water. One μg total RNA was used for first strand cDNA synthesis. Primer efficiency tests were performed for Gho/Sec24B2 reg1 (SEQ ID NOs:10 and 11) and actin primer pairs (SEQ ID NOs:82 and 83) to determine the suitability for RT-qPCR analysis. RT-qPCR was performed using SYBR™ green master mix (APPLIED BIOSYSTEMS, Grand Island, N.Y.) with APPLIED BIOSYSTEMS 7500 fast real-time PCR system. The WCR actin gene was used as a reference gene to calculate relative transcript abundance. Freshly treated artificial was provided on day 1 and 3.

LC₅₀ determination.

Adult beetles are exposed to 0, 0.1, 1, 10, 100, or 1000 ng/diet plug concentrations of Gho/Sec24B2 reg1 (SEQ ID NO:3), Sec24B1 reg1 (SEQ ID NO:104), or GFP (SEQ ID NO:9) to determine the LC₅₀ value. Water alone establishes the control mortality. Fresh artificial diet (as described above) is treated with dsRNA, and provided every other day up to day 10. After day 10, adults are maintained on untreated artificial diet, with fresh diet provided every other day. Mortality is recorded daily for 15 days. The LC₅₀ is calculated using Polo Plus software (LeOra Software, Berkeley, Calif.). The LC₅₀ calculation shows that 0, 0.1, 1, 10, 100, and/or 1000 ng/diet is an effective concentration of dsRNA.

Exposure Time.

Adults were exposed to 50 ng/diet plug Gho/Sec24B2 reg1 (SEQ ID NO:3), Sec24B1 reg1 (SEQ ID NO:104), or GFP (SEQ ID NO:9) dsRNA, or an equal volume of water for 3, 6, or 48 hours, and then moved to untreated artificial diet to determine the minimum exposure time to achieve significant mortality. Mortality was recorded daily for 15 days. The mortality measurements show that 3, 6, and/or 48 hours is an effective exposure time for the dsRNAs.

TABLE 6
Percent mortality of adult Diabrotica virgifera virgifera after exposure to
500 ng/diet plug of Gho/Sec24B2 reg1 dsRNA, Sec24B1 reg1 dsRNA, the same amount of GFP
dsRNA, or same volume of water.

| | % Mortality Mean ± SEM* | | | | | |
|---|---|---|---|---|---|---|
| Treatment | Day 1 | Day 2 | Day 3 | Day 4 | Day 5 | Day 6 |
| Sec24B1 Reg 1 | 0.0 ± 0 | 0.0 ± 0 | 0.0 ± 0 | 3.33 ± 3.33 | 3.33 ± 3.33 | 6.67 ± 3.33 |
| Sec24B2 Reg 1 | 0.0 ± 0 | 0.0 ± 0 | 0.0 ± 0 | 0.0 ± 0 | 0.0 ± 0 | 13.33 ± 3.33 |
| Water | 0.0 ± 0 | 0.0 ± 0 | 0.0 ± 0 | 0.0 ± 0 | 0.0 ± 0 | 0.0 ± 0 |
| GFP** | 0.0 ± 0 | 0.0 ± 0 | 0.0 ± 0 | 0.0 ± 0 | 0.0 ± 0 | 0.0 ± 0 |

| | Day 7 | Day 8 | Day 9 | Day 10 | Day 11 |
|---|---|---|---|---|---|
| Sec24B1 Reg 1 | 10.00 ± 5.77 | 13.33 ± 6.67 | 16.67 ± 3.33 | 16.67 ± 3.33 | 16.67 ± 3.33 |
| Sec24B2 Reg 1 | 23.33 ± 3.33 | 66.67 ± 6.67 | 80.00 ± 10.0 | 90.00 ± 5.77 | 90.00 ± 5.77 |
| Water | 0.0 ± 0 | 0.0 ± 0 | 0.0 ± 0 | 0.0 ± 0 | 0.0 ± 0 |
| GFP** | 0.0 ± 0 | 0.0 ± 0 | 0.0 ± 0 | 0.0 ± 0 | 0.0 ± 0 |
| | Day 12 | Day 13 | Day 14 | Day 15 | Day 16 |
| Sec24B1 Reg 1 | 20.00 ± 5.77 | 33.33 ± 13.33 | 33.33 ± 13.33 | 33.33 ± 13.33 | 33.33 ± 13.33 |
| Sec24B2 Reg 1 | 90.00 ± 5.77 | 93.33 ± 3.33 | 96.67 ± 3.33 | 96.67 ± 3.33 | 100.00 ± 0 |
| Water | 0.0 ± 0 | 0.0 ± 0 | 0.0 ± 0 | 0.0 ± 0 | 0.0 ± 0 |
| GFP** | 0.0 ± 0 | 3.33 ± 3.33 | 13.33 ± 8.82 | 16.67 ± 6.67 | 16.67 ± 6.67 |

*± SEM = Standard Error of the Mean
**GFP = Green Fluorescent Protein

### Example 7

### Production of Transgenic Maize Tissues Comprising Insecticidal dsRNAs

Agrobacterium-Mediated Transformation.

Transgenic maize cells, tissues, and plants that produce one or more insecticidal dsRNA molecules (for example, at least one dsRNA molecule including a dsRNA molecule targeting a gene comprising Gho/Sec24B2 (e.g., SEQ ID NO:1)), through expression of a chimeric gene stably-integrated into the plant genome are produced following Agrobacterium-mediated transformation. Maize transformation methods employing superbinary or binary transformation vectors are known in the art, as described, for example, in U.S. Pat. No. 8,304,604, which is herein incorporated by reference in its entirety. Transformed tissues are selected by their ability to grow on Haloxyfop-containing medium and are screened for dsRNA production, as appropriate. Portions of such transformed tissue cultures are presented to neonate corn rootworm larvae for bioassay, essentially as described in EXAMPLE 1.

Agrobacterium Culture Initiation.

Glycerol stocks of Agrobacterium strain DAt13192 cells (WO 2012/016222A2) harboring a binary transformation vector pDAB114515, pDAB115770, pDAB110853 or pDAB110556 described above (EXAMPLE 4) are streaked on AB minimal medium plates (Watson et al. (1975) J. Bacteriol. 123:255-264) containing appropriate antibiotics and are grown at 20° C. for 3 days. The cultures are then streaked onto YEP plates (gm/L: yeast extract, 10; Peptone, 10; NaCl, 5) containing the same antibiotics and were incubated at 20° C. for 1 day.

Agrobacterium Culture.

On the day of an experiment, a stock solution of Inoculation Medium and acetosyringone is prepared in a volume appropriate to the number of constructs in the experiment and pipetted into a sterile, disposable, 250 mL flask. Inoculation Medium (Frame et al. (2011) Genetic Transformation Using Maize Immature Zygotic Embryos. IN Plant Embryo Culture Methods and Protocols: Methods in Molecular Biology. T. A. Thorpe and E. C. Yeung, (Eds), Springer Science and Business Media, LLC. pp 327-341) contained: 2.2 gm/L MS salts; 1×ISU Modified MS Vitamins (Frame et al., ibid.) 68.4 gm/L sucrose; 36 gm/L glucose; 115 mg/L L-proline; and 100 mg/L myo-inositol; at pH 5.4). Acetosyringone is added to the flask containing Inoculation Medium to a final concentration of 200 μM from a 1 M stock solution in 100% dimethyl sulfoxide and the solution is thoroughly mixed.

For each construct, 1 or 2 inoculating loops-full of Agrobacterium from the YEP plate are suspended in 15 mL of the Inoculation Medium/acetosyringone stock solution in a sterile, disposable, 50 mL centrifuge tube, and the optical density of the solution at 550 nm (OD₅₅₀) is measured in a spectrophotometer. The suspension is then diluted to OD₅₅₀ of 0.3 to 0.4 using additional Inoculation Medium/acetosyringone mixture. The tube of Agrobacterium suspension is then placed horizontally on a platform shaker set at about 75 rpm at room temperature and shaken for 1 to 4 hours while embryo dissection is performed.

Ear Sterilization and Embryo Isolation.

Maize immature embryos are obtained from plants of Zea mays inbred line B104 (Hallauer et al. (1997) Crop Science 37:1405-1406) grown in the greenhouse and self- or sib-pollinated to produce ears. The ears are harvested approximately 10 to 12 days post-pollination. On the experimental day, de-husked ears are surface-sterilized by immersion in a 20% solution of commercial bleach (ULTRA CLOROX® Germicidal Bleach, 6.15% sodium hypochlorite; with two drops of TWEEN 20) and shaken for 20 to 30 min, followed by three rinses in sterile deionized water in a laminar flow hood. Immature zygotic embryos (1.8 to 2.2 mm long) are aseptically dissected from each ear and randomly distributed into microcentrifuge tubes containing 2.0 mL of a suspension of appropriate Agrobacterium cells in liquid Inoculation Medium with 200 μM acetosyringone, into which 2 μL of 10% BREAK-THRU® S233 surfactant (EVONIK INDUSTRIES; Essen, Germany) had been added. For a given set of experiments, embryos from pooled ears are used for each transformation.

Agrobacterium Co-Cultivation.

Following isolation, the embryos are placed on a rocker platform for 5 minutes. The contents of the tube are then poured onto a plate of Co-cultivation Medium, which contains 4.33 gm/L MS salts; 1×ISU Modified MS Vitamins; 30 gm/L sucrose; 700 mg/L L-proline; 3.3 mg/L Dicamba in KOH (3,6-dichloro-o-anisic acid or 3,6-dichloro-2-methoxybenzoic acid); 100 mg/L myo-inositol; 100 mg/L Casein Enzymatic Hydrolysate; 15 mg/L AgNO₃; 200 μM acetosyringone in DMSO; and 3 gm/L GELZAN™, at pH 5.8. The liquid Agrobacterium suspension is removed with a sterile, disposable, transfer pipette. The embryos are then oriented with the scutellum facing up using sterile forceps with the aid of a microscope. The plate is closed, sealed with 3M™ MICROPORE™ medical tape, and placed in an incubator at 25° C. with continuous light at approximately 60 μmol m⁻²s⁻¹ of Photosynthetically Active Radiation (PAR).

Callus Selection and Regeneration of Transgenic Events.

Following the Co-Cultivation period, embryos are transferred to Resting Medium, which is composed of 4.33 gm/L MS salts; 1×ISU Modified MS Vitamins; 30 gm/L sucrose; 700 mg/L L-proline; 3.3 mg/L Dicamba in KOH; 100 mg/L myo-inositol; 100 mg/L Casein Enzymatic Hydrolysate; 15 mg/L AgNO₃; 0.5 gm/L MES (2-(N-morpholino)ethanesulfonic acid monohydrate; PHYTOTECHNOLOGIES LABR.; Lenexa, Kans.); 250 mg/L Carbenicillin; and 2.3 gm/L GELZAN™; at pH 5.8. No more than 36 embryos are moved to each plate. The plates are placed in a clear plastic box and incubated at 27° C. with continuous light at approximately 50 μmol m⁻²s⁻¹ PAR for 7 to 10 days. Callused embryos are then transferred (<18/plate) onto Selection Medium I, which is comprised of Resting Medium (above) with 100 nM R-Haloxyfop acid (0.0362 mg/L; for selection of calli harboring the AAD-1 gene). The plates are returned to clear boxes and incubated at 27° C. with continuous light at approximately 50 μmol m⁻²s⁻¹ PAR for 7 days. Callused embryos are then transferred (<12/plate) to Selection Medium II, which is comprised of Resting Medium (above) with 500 nM R-Haloxyfop acid (0.181 mg/L). The plates are returned to clear boxes and incubated at 27° C. with continuous light at approximately 50 μmol m⁻²s⁻¹ PAR for 14 days. This selection step allows transgenic callus to further proliferate and differentiate.

Proliferating, embryogenic calli are transferred (<9/plate) to Pre-Regeneration medium. Pre-Regeneration Medium contains 4.33 gm/L MS salts; 1×ISU Modified MS Vitamins; 45 gm/L sucrose; 350 mg/L L-proline; 100 mg/L myo-inositol; 50 mg/L Casein Enzymatic Hydrolysate; 1.0 mg/L AgNO₃; 0.25 gm/L MES; 0.5 mg/L naphthaleneacetic acid in NaOH; 2.5 mg/L abscisic acid in ethanol; 1 mg/L 6-benzylaminopurine; 250 mg/L Carbenicillin; 2.5 gm/L GELZAN™; and 0.181 mg/L Haloxyfop acid; at pH 5.8. The plates are stored in clear boxes and incubated at 27° C. with continuous light at approximately 50 μmol m⁻²s⁻¹ PAR for 7 days. Regenerating calli are then transferred (<6/plate) to Regeneration Medium in PHYTATRAYS™ (SIGMA-ALDRICH) and incubated at 28° C. with 16 hours light/8 hours dark per day (at approximately 160 μmol m⁻²s⁻¹ PAR) for 14 days or until shoots and roots develop. Regeneration Medium contains 4.33 gm/L MS salts; 1×ISU Modified MS Vitamins; 60 gm/L sucrose; 100 mg/L myo-inositol; 125 mg/L Carbenicillin; 3 gm/L GELLAN™ gum; and 0.181 mg/L R-Haloxyfop acid; at pH 5.8. Small shoots with primary roots are then isolated and transferred to Elongation Medium without selection. Elongation Medium contains 4.33 gm/L MS salts; 1×ISU Modified MS Vitamins; 30 gm/L sucrose; and 3.5 gm/L GELRITE™: at pH 5.8.

Transformed plant shoots selected by their ability to grow on medium containing Haloxyfop are transplanted from PHYTATRAYS™ to small pots filled with growing medium (PROMIX BX; PREMIER TECH HORTICULTURE), covered with cups or HUMI-DOMES (ARCO PLASTICS), and then hardened-off in a CONVIRON growth chamber (27° C. day/24° C. night, 16-hour photoperiod, 50-70% RH, 200 μmol m⁻²s⁻¹ PAR). In some instances, putative transgenic plantlets are analyzed for transgene relative copy number by quantitative real-time PCR assays using primers designed to detect the AAD1 herbicide tolerance gene integrated into the maize genome. Further, RNA qPCR assays are used to detect the presence of the linker sequence in expressed dsRNAs of putative transformants. Selected transformed plantlets are then moved into a greenhouse for further growth and testing.

Transfer and Establishment of T₀ Plants in the Greenhouse for Bioassay and Seed Production.

When plants reach the V3-V4 stage, they are transplanted into IE CUSTOM BLEND (PROFILE/METRO MIX 160) soil mixture and grown to flowering in the greenhouse (Light Exposure Type: Photo or Assimilation; High Light Limit: 1200 PAR; 16-hour day length; 27° C. day/24° C. night).

Plants to be used for insect bioassays are transplanted from small pots to TINUS™ 350-4 ROOTRAINERS® (SPENCER-LEMAIRE INDUSTRIES, Acheson, Alberta, Canada;) (one plant per event per ROOTRAINER®). Approximately four days after transplanting to ROOTRAINERS®, plants are infested for bioassay.

Plants of the T₁ generation are obtained by pollinating the silks of T₀ transgenic plants with pollen collected from plants of non-transgenic elite inbred line B104 or other appropriate pollen donors, and planting the resultant seeds. Reciprocal crosses are performed when possible.

### Example 8

### Molecular Analyses of Transgenic Maize Tissues

Molecular analyses (e.g. RNA qPCR) of maize tissues are performed on samples from leaves and roots that are collected from greenhouse grown plants on the same days that root feeding damage is assessed.

Results of RNA qPCR assays for the Per5 3′UTR are used to validate expression of hairpin transgenes. (A low level of Per5 3′UTR detection is expected in non-transformed maize plants, since there is usually expression of the endogenous Per5 gene in maize tissues.) Results of RNA qPCR assay for intervening sequence between repeat sequences (which is integral to the formation of dsRNA hairpin molecules) in expressed RNAs are used to validate the presence of hairpin transcripts. Transgene RNA expression levels are measured relative to the RNA levels of an endogenous maize gene.

DNA qPCR analyses to detect a portion of the AAD1 coding region in gDNA are used to estimate transgene insertion copy number. Samples for these analyses are collected from plants grown in environmental chambers. Results are compared to DNA qPCR results of assays designed to detect a portion of a single-copy native gene, and simple events (having one or two copies of the transgenes) are advanced for further studies in the greenhouse.

Additionally, qPCR assays designed to detect a portion of the spectinomycin-resistance gene (SpecR; harbored on the binary vector plasmids outside of the T-DNA) are used to determine if the transgenic plants contained extraneous integrated plasmid backbone sequences.

Hairpin RNA Transcript Expression Level: Per 5 3′UTR qPCR.

Callus cell events or transgenic plants are analyzed by real time quantitative PCR (qPCR) of the Per 5 3′UTR sequence to determine the relative expression level of the full length hairpin transcript, as compared to the transcript level of an internal maize gene (SEQ ID NO:59; GENBANK Accession No. BT069734), which encodes a TIP41-like protein (i.e. a maize homolog of GENBANK Accession No. AT4G34270; having a tBLASTX score of 74% identity). RNA is isolated using an RNEASY™ 96 kit (QIAGEN, Valencia, Calif.). Following elution, the total RNA is subjected to a DNAse1 treatment according to the kit's suggested protocol. The RNA is then quantified on a NANODROP 8000 spectrophotometer (THERMO SCIENTIFIC) and concentration is normalized to 25 ng/μL. First strand cDNA is prepared using a HIGH CAPACITY cDNA SYNTHESIS KIT (INVITROGEN) in a 10 μL reaction volume with 5 μL denatured RNA, substantially according to the manufacturer's recommended protocol. The protocol is modified slightly to include the addition of 10 μL of 100 μM T20VN oligonucleotide (IDT) (SEQ ID NO:60; TTTTTTTTTTTTTTTTTTTTVN, where V is A, C, or G, and N is A, C, G, or T/U) into the 1 mL tube of random primer stock mix, in order to prepare a working stock of combined random primers and oligo dT.

Following cDNA synthesis, samples are diluted 1:3 with nuclease-free water, and stored at −20° C. until assayed.

Separate real-time PCR assays for the StPIN II 3′ UTR and TIP41-like transcript are performed on a LIGHTCYCLER™ 480 (ROCHE DIAGNOSTICS, Indianapolis, Ind.) in 10 μL reaction volumes. For the PIN II assay, reactions are run with Primers StPinIIF2 TAG (SEQ ID NO:61) and StPinIIR2 TAG (SEQ ID NO:62), and a StPinIIFAM2 TAG (SEQ ID NO:101). For the TIP41-like reference gene assay, primers TIPmxF (SEQ ID NO:63) and TIPmxR (SEQ ID NO:64), and Probe HXTIP (SEQ ID NO:65) labeled with HEX (hexachlorofluorescein) are used.

All assays included negative controls of no-template (mix only). For the standard curves, a blank (water in source well) is also included in the source plate to check for sample cross-contamination. Primer and probe sequences are set forth in Table 7. Reaction components recipes for detection of the various transcripts are disclosed in Table 8, and PCR reactions conditions are summarized in Table 9. The FAM (6-Carboxy Fluorescein Amidite) fluorescent moiety is excited at 465 nm and fluorescence is measured at 510 nm; the corresponding values for the HEX (hexachlorofluorescein) fluorescent moiety are 533 nm and 580 nm.

TABLE 7
Oligonucleotide sequences for molecular
analyses of transcript levels in transgenic
maize.

| Target | Oligonucleotide | Sequence |
|---|---|---|
| PIN II | StPinIIF2 TAG | GGGTGACGGGAGAGATT |
| | | (SEQ ID NO: 61) |
| PIN II | StPinIIR2 TAG | CATAACACACAACTTTGATGCC |
| | | (SEQ ID NO: 62) |
| PIN II | StPinIIFAM2 TAG | AAGTCTAGGTTGTTTAAAGGTTAC |
| | | CGAGC (SEQ ID NO: 101) |
| TIP41 | TIPmxF | TGAGGGTAATGCCAACTGGTT |
| | | (SEQ ID NO: 63) |
| TIP41 | TIPmxR | GCAATGTAACCGAGTGTCTCTCAA |
| | | (SEQ ID NO: 64) |
| TIP41 | HXTIP | TTTTTGGCTTAGAGTTGATGGTGT |
| | (HEX-Probe) | ACTGATGA (SEQ ID NO: 65) |

TABLE 8
PCR reaction recipes for transcript detection.

| | Per5 3′UTR1 X | | TIP-like Gene | |
|---|---|---|---|---|
| Component | Final Concentration | | | |
| Roche Buffer | 1 X | | 1X | |
| StPinIIF2 TAG | 0.4 | μM | 0 | |
| StPinIIR2 TAG | 0.4 | μM | 0 | |
| StPinIIFAM2 TAG | 0.2 | μM | 0 | |
| HEXtipZM F | 0 | | 0.4 | μM |
| HEXtipZM R | 0 | | 0.4 | μM |
| HEXtipZMP (HEX) | 0 | | 0.2 | μM |
| cDNA (2.0 μL) | NA | | NA | |
| Water | To 10 | μL | To 10 | μL |

TABLE 9
Thermocycler conditions for RNA qPCR.
Per5 3′UTR and TIP41-like Gene Detection

| Process | Temp. | Time | | No. Cycles |
|---|---|---|---|---|
| Target Activation | 95° C. | 10 | min | 1 |
| Denature | 95° C. | 10 | sec | 40 |
| Extend | 60° C. | 40 | sec | |
| Acquire FAM or HEX | 72° C. | 1 | sec | |
| Cool | 40° C. | 10 | sec | 1 |

Data is analyzed using LIGHTCYCLER™ Software v1.5 by relative quantification using a second derivative max algorithm for calculation of Cq values according to the supplier's recommendations. For expression analyses, expression values are calculated using the ΔΔCt method (i.e., 2-(Cq TARGET-Cq REF)), which relies on the comparison of differences of Cq values between two targets, with the base value of 2 being selected under the assumption that, for optimized PCR reactions, the product doubles every cycle.

Hairpin Transcript Size and Integrity: Northern Blot Assay.

In some instances, additional molecular characterization of the transgenic plants is obtained by the use of Northern Blot (RNA blot) analysis to determine the molecular size of the Gho/Sec24B2 hairpin RNA in transgenic plants expressing a Gho/Sec24B2 hairpin dsRNA.

All materials and equipment are treated with RNaseZAP (AMBION/INVITROGEN) before use. Tissue samples (100 mg to 500 mg) are collected in 2 mL SAFELOCK EPPENDORF tubes, disrupted with a KLECKO™ tissue pulverizer (GARCIA MANUFACTURING, Visalia, Calif.) with three tungsten beads in 1 mL TRIZOL (INVITROGEN) for 5 min, then incubated at room temperature (RT) for 10 min. Optionally, the samples are centrifuged for 10 min at 4° C. at 11,000 rpm, and the supernatant is transferred into a fresh 2 mL SAFELOCK EPPENDORF tube. After 200 μL chloroform are added to the homogenate, the tube is mixed by inversion for 2 to 5 min, incubated at RT for 10 minutes, and centrifuged at 12,000×g for 15 min at 4° C. The top phase is transferred into a sterile 1.5 mL EPPENDORF tube, 600 μL 100% isopropanol is added, incubated at RT for 10 min to 2 hr, and then centrifuged at 12,000×g for 10 min at 4° C. to 25° C. The supernatant is discarded, and the RNA pellet is washed twice with 1 mL 70% ethanol, with centrifugation at 7,500×g for 10 min at 4° C. to 25° C. between washes. The ethanol is discarded, and the pellet is briefly air-dried for 3 to 5 min, before resuspending in 50 μL nuclease-free water.

Total RNA is quantified using the NANODROP8000® (THERMO-FISHER) and samples are normalized to 5 μg/10 μL. 10 μL glyoxal (AMBION/INVITROGEN) is then added to each sample. Five to 14 ng of DIG RNA standard marker mix (ROCHE APPLIED SCIENCE, Indianapolis, Ind.) is dispensed and added to an equal volume of glyoxal. Samples and marker RNAs are denatured at 50° C. for 45 min and stored on ice until loading on a 1.25% SEAKEM GOLD agarose (LONZA, Allendale, N.J.) gel in NORTHERNMAX 10× glyoxal running buffer (AMBION/INVITROGEN). RNAs are separated by electrophoresis at 65 volts/30 mA for 2 hr and 15 min.

Following electrophoresis, the gel is rinsed in 2×SSC for 5 min, and imaged on a GEL DOC station (BIORAD, Hercules, Calif.). Then, the RNA is passively transferred to a nylon membrane (MILLIPORE) overnight at RT, using 10×SSC as the transfer buffer (20×SSC consists of 3 M sodium chloride and 300 mM trisodium citrate, pH 7.0). Following the transfer, the membrane is rinsed in 2×SSC for 5 minutes, the RNA is UV-crosslinked to the membrane (AGILENT/STRATAGENE), and the membrane is allowed to dry at room temperature for up to 2 days.

The membrane is pre-hybridized in ULTRAHYB™ buffer (AMBION/INVITROGEN) for 1 to 2 hr. The probe consists of a PCR amplified product containing the sequence of interest, (for example, the antisense sequence portion of SEQ ID NO:18 or SEQ ID NO:19, as appropriate) labeled with digoxigenin by means of a ROCHE APPLIED SCIENCE DIG procedure. Hybridization in recommended buffer is overnight at a temperature of 60° C. in hybridization tubes. Following hybridization, the blot is subjected to DIG washes, wrapped, exposed to film for 1 to 30 minutes, then the film is developed, all by methods recommended by the supplier of the DIG kit.

Transgene Copy Number Determination.

Maize leaf pieces approximately equivalent to 2 leaf punches were collected in 96-well collection plates (QIAGEN™). Tissue disruption was performed with a KLECKO™ tissue pulverizer (GARCIA MANUFACTURING, Visalia, Calif.) in BIOSPRINT96™ AP1 lysis buffer (supplied with a BIOSPRINT96™ PLANT KIT; QIAGEN™) with one stainless steel bead. Following tissue maceration, genomic DNA (gDNA) was isolated in high-throughput format using a BIOSPRINT96™ PLANT KIT and a BIOSPRINT96™ extraction robot. Genomic DNA was diluted 2:3 DNA:water prior to setting up the qPCR reaction.

qPCR Analysis.

Transgene detection by hydrolysis probe assay was performed by real-time PCR using a LIGHTCYCLER®480 system. Oligonucleotides to be used in hydrolysis probe assays to detect the linker sequence (e.g. ST-LS1, SEQ ID NO:21), or to detect a portion of the SpecR gene (i.e., the spectinomycin resistance gene borne on the binary vector plasmids; SEQ ID NO:66; SPC1 oligonucleotides in Table 10), were designed using LIGHTCYCLER® PROBE DESIGN SOFTWARE 2.0. Further, oligonucleotides to be used in hydrolysis probe assays to detect a segment of the AAD-1 herbicide tolerance gene (SEQ ID NO:67; GAAD1 oligonucleotides in Table 10) were designed using PRIMER EXPRESS software (APPLIED BIOSYSTEMS). Table 10 shows the sequences of the primers and probes. Assays were multiplexed with reagents for an endogenous maize chromosomal gene (Invertase (SEQ ID NO:68; GENBANK Accession No: U16123; referred to herein as IVR1), which served as an internal reference sequence to ensure gDNA was present in each assay. For amplification, LIGHTCYCLER® 480 PROBES MASTER mix (ROCHE APPLIED SCIENCE) was prepared at 1× final concentration in a 10 μL volume multiplex reaction containing 0.4 μM of each primer and 0.2 μM of each probe (Table 11). A two-step amplification reaction was performed as outlined in Table 12. Fluorophore activation and emission for the FAM- and HEX-labeled probes were as described above; CY5 conjugates were excited maximally at 650 nm and fluoresce maximally at 670 nm.

Cp scores (the point at which the fluorescence signal crosses the background threshold) were determined from the real time PCR data using the fit points algorithm (LIGHTCYCLER® SOFTWARE release 1.5) and the Relative Quant module (based on the ΔΔCt method). Data were handled as described previously above (RNA qPCR).

TABLE 10
Sequences of primers and probes (with
fluorescent conjugate) used for gene copy
number determinations and binary
vector plasmid backbone detection.

| Name | Sequence |
|---|---|
| GAAD1-F | TGTTCGGTTCCCTCTACCAA |
| | (SEQ ID NO: 69) |
| GAAD1-R | CAACATCCATCACCTTGACTGA |
| | (SEQ ID NO: 70) |
| GAAD1-P (FAM) | CACAGAACCGTCGCTTCAGCAACA |
| | (SEQ ID NO: 71) |
| IVR1-F | TGGCGGACGACGACTTGT |
| | (SEQ ID NO: 72) |
| IVR1-R | AAAGTTTGGAGGCTGCCGT |
| | (SEQ ID NO: 73) |
| IVR1-P (HEX) | CGAGCAGACCGCCGTGTACTTCTAC |
| | C (SEQ ID NO: 74) |
| SPC1A | CTTAGCTGGATAACGCCAC |
| | (SEQ ID NO: 75) |
| SPC1S | GACCGTAAGGCTTGATGAA |
| | (SEQ ID NO: 76) |
| TQSPEC (CY5*) | CGAGATTCTCCGCGCTGTAGA |
| | (SEQ ID NO: 77) |
| ST-LS1-F | GTATGTTTCTGCTTCTACCTTTGAT |
| | (SEQ ID NO: 78) |
| ST-LSI-R | CCATGTTTTGGTCATATATTAGAAA |
| | AGTT (SEQ ID NO: 79) |
| ST-LS1-P (FAM) | AGTAATATAGTATTTCAAGTATTTT |
| | TTTCAAAAT (SEQ ID NO: 80) |

CY5 = Cyanine-5

TABLE 11
Reaction components for gene copy number
analyses and plasmid backbone detection.

| | Amt. | | | Final |
|---|---|---|---|---|
| Component | (μL) | Stock | | Concentration |
| 2× Buffer | 5.0 | 2× | | 1× |
| Appropriate Forward Primer | 0.4 | 10 | μM | 0.4 |
| Appropriate Reverse Primer | 0.4 | 10 | μM | 0.4 |
| Appropriate Probe | 0.4 | 5 | μM | 0.2 |
| IVR1-Forward Primer | 0.4 | 10 | μM | 0.4 |
| IVR1-Reverse Primer | 0.4 | 10 | μM | 0.4 |
| IVR1-Probe | 0.4 | 5 | μM | 0.2 |

| | | | |
|---|---|---|---|
| H₂O | 0.6 | NA* | NA |
| gDNA | 2.0 | ND** | ND |
| Total | 10.0 | | |

*NA = Not Applicable
**ND = Not Determined

TABLE 12
Thermocycler conditions for DNA qPCR.
Genomic copy number analyses

| Process | Temp. | Time | | No. Cycles |
|---|---|---|---|---|
| Target Activation | 95° C. | 10 | min | 1 |
| Denature | 95° C. | 10 | sec | 40 |
| Extend & Acquire | 60° C. | 40 | sec | |
| FAM, HEX, or CY5 | | | | |
| Cool | 40° C. | 10 | sec | 1 |

### Example 9

### Bioassay of Transgenic Maize

Insect Bioassays.

Bioactivity of dsRNA of the subject invention produced in plant cells is demonstrated by bioassay methods. See, e.g., Baum et al. (2007) Nat. Biotechnol. 25(11):1322-1326. One is able to demonstrate efficacy, for example, by feeding various plant tissues or tissue pieces derived from a plant producing an insecticidal dsRNA to target insects in a controlled feeding environment. Alternatively, extracts are prepared from various plant tissues derived from a plant producing the insecticidal dsRNA, and the extracted nucleic acids are dispensed on top of artificial diets for bioassays as previously described herein. The results of such feeding assays are compared to similarly conducted bioassays that employ appropriate control tissues from host plants that do not produce an insecticidal dsRNA, or to other control samples. Growth and survival of target insects on the test diet is reduced compared to that of the control group.

Insect Bioassays with Transgenic Maize Events.

Two western corn rootworm larvae (1 to 3 days old) hatched from washed eggs are selected and placed into each well of the bioassay tray. The wells are then covered with a “PULL N' PEEL” tab cover (BIO-CV-16, BIO-SERV) and placed in a 28° C. incubator with an 18 hr/6 hr light/dark cycle. Nine days after the initial infestation, the larvae are assessed for mortality, which is calculated as the percentage of dead insects out of the total number of insects in each treatment. Significant mortality is observed. The insect samples are frozen at −20° C. for two days, then the insect larvae from each treatment are pooled and weighed. The percent of growth inhibition is calculated as the mean weight of the experimental treatments divided by the mean of the average weight of two control well treatments. The data are expressed as a Percent Growth Inhibition (of the Negative Controls). Mean weights that exceed the control mean weight are normalized to zero. Significant growth inhibition is observed.

Insect Bioassays in the Greenhouse.

Western corn rootworm (WCR, Diabrotica virgifera virgifera LeConte) eggs were received in soil from CROP CHARACTERISTICS (Farmington, Minn.). WCR eggs were incubated at 28° C. for 10 to 11 days. Eggs were washed from the soil, placed into a 0.15% agar solution, and the concentration was adjusted to approximately 75 to 100 eggs per 0.25 mL aliquot. A hatch plate was set up in a Petri dish with an aliquot of egg suspension to monitor hatch rates.

The soil around the maize plants growing in ROOTRANERS® was infested with 150 to 200 WCR eggs. The insects were allowed to feed for 2 weeks, after which time a “Root Rating” was given to each plant. A Node-Injury Scale was utilized for grading, essentially according to Oleson et al. (2005) J. Econ. Entomol. 98:1-8. Plants which passed this bioassay, showing reduced injury, were transplanted to 5-gallon pots for seed production. Transplants were treated with insecticide to prevent further rootworm damage and insect release in the greenhouses. Plants were hand pollinated for seed production. Seeds produced by these plants were saved for evaluation at the T₁ and subsequent generations of plants.

Greenhouse bioassays included two kinds of negative control plants. Transgenic negative control plants were generated by transformation with vectors harboring genes designed to produce a yellow fluorescent protein (YFP) or a YFP hairpin dsRNA (See EXAMPLE 4). Non-transformed negative control plants were grown from seeds of parental corn varieties, 7sh382 or B104. Bioassays were conducted on two separate dates, with negative controls included in each set of plant materials.

Table 13 shows the combined results of molecular analyses and bioassays for Gho/Sec24B2-hairpin plants. Examination of the bioassay results summarized in Table 13 revealed the surprising and unexpected observation that the majority of the transgenic maize plants harboring constructs that express an Gho/Sec24B2 hairpin dsRNA comprising segments of SEQ ID NO:1 (e.g., SEQ ID NO:18 and SEQ ID NO:19), were protected against root damage incurred by feeding of western corn rootworm larvae. Twenty-two of the 37 graded events had a root rating of 0.5 or lower. Table 14 shows the combined results of molecular analyses and bioassays for negative control plants. Most of the plants had no protection against WCR larvae feeding.

TABLE 13
Greenhouse bioassay and molecular analyese results of
Gho/Sec24B2-hairpin-expressing maize plants.

| | | Leaf Tissue | | Root Tissue | | |
|---|---|---|---|---|---|---|
| | | PIN II | Loop | PIN II | Loop | Root |
| Sample ID | Batch | RTL* | RTL | RTL* | RTL | Rating |
| Gho/Sec24B2 v1 | | | | | | |
| Events | | | | | | |
| 114544[1]- | 2 | 0.89 | 0.209 | *** | *** | 0.5 |
| 005.001 | | | | | | |
| 114544[1]- | 2 | 0.32 | 0.188 | *** | *** | 0.25 |
| 007.001 | | | | | | |
| 114544[1]- | 3 | 0.29 | 0.004 | 0.77 | 0.007 | 0.5 |
| 011.001 | | | | | | |
| 114544[1]- | 3 | 0.39 | 0.100 | 0.67 | 0.250 | 1 |
| 016.001 | | | | | | |
| 114544[1]- | 3 | 0.23 | 1.729 | 0.36 | 1.986 | 0.5 |
| 017.001 | | | | | | |
| 114544[1]- | 3 | 0.18 | 0.070 | 0.54 | 0.064 | 1 |
| 019.001 | | | | | | |
| 114544[1]- | 3 | 0.24 | 0.095 | 0.36 | 0.076 | 1 |
| 024.001 | | | | | | |
| 114544[1]- | 3 | 0.15 | 0.049 | 0.51 | 0.125 | 0.5 |
| 025.001 | | | | | | |
| 114544[1]- | 3 | 9.45 | 0.369 | 0.87 | 0.140 | 0.01 |
| 026.001 | | | | | | |
| Gho/Sec24B2 v2 | | | | | | |
| Events | | | | | | |
| 114549[1]- | 1 | 0.84 | 0.230 | *** | *** | 0.01 |
| 001.001 | | | | | | |
| 114549[1]- | 2 | 2.81 | 0.633 | *** | *** | 0.1 |
| 007.001 | | | | | | |
| 114549[1]- | 2 | 2.08 | 0.547 | *** | *** | 0.75 |
| 009.001 | | | | | | |
| 114549[1]- | 2 | 1.48 | 0.409 | *** | *** | 0.01 |
| 011.001 | | | | | | |
| 114549[1]- | 2 | 1.26 | 0.261 | *** | *** | 0.25 |
| 013.001 | | | | | | |
| 114549[1]- | 2 | 1.47 | 0.351 | *** | *** | 0.5 |
| 014.001 | | | | | | |
| 114549[1]- | 2 | 1.36 | 0.379 | *** | *** | 0.25 |
| 015.001 | | | | | | |
| 114549[1]- | 2 | 13.09 | 0.138 | *** | *** | 0.5 |
| 016.001 | | | | | | |
| 114549[1]- | 2 | 1.80 | 0.395 | *** | *** | 0.75 |
| 019.001 | | | | | | |
| 114549[1]- | 2 | 1.82 | 0.235 | *** | *** | 1 |
| 020.001 | | | | | | |
| 114549[1]- | 2 | 1.27 | 0.232 | *** | *** | 1 |
| 021.001 | | | | | | |
| 114549[1]- | 2 | 2.16 | 0.349 | *** | *** | 1 |
| 023.001 | | | | | | |
| 114549[1]- | 2 | 0.16 | 0.031 | *** | *** | 1 |
| 025.001 | | | | | | |
| 114549[1]- | 2 | 1.92 | 0.392 | *** | *** | 0.5 |
| 026.001 | | | | | | |
| 114549[1]- | 2 | 2.06 | 0.768 | *** | *** | 0.02 |
| 029.001 | | | | | | |

*RTL = Relative Transcript Level as measured against TIP4-like gene transcript levels.
**NG = Not Graded due to small plant size.
***ND = Not Done.

TABLE 14
Greenhouse bioassay and molecular analyses results of negative control
plants comprising transgenic and non-transformed maize plants.

| | | Leaf Tissue | | Root Tissue | | |
|---|---|---|---|---|---|---|
| | | PIN II | Loop | PIN II | Loop | Root |
| Sample ID | Batch | RTL* | RTL | RTL* | RTL | Rating |
| YFP protein | | | | | | |
| Events | | | | | | |
| 101556[708]- | 3 | 0.03 | 0.002 | 0.03 | 0.008 | 1 |
| 11157.001 | | | | | | |
| 101556[708]- | 3 | 0.01 | 0.000 | 0.00 | 0.000 | 1 |
| 11158.001 | | | | | | |
| 101556[708]- | 3 | 0.01 | 0.002 | 0.01 | 0.000 | 1 |
| 11159.001 | | | | | | |
| 101556[708]- | 2 | 0.72 | 0.069 | *** | *** | 1 |
| 11165.001 | | | | | | |
| 101556[708]- | 2 | 0.82 | 0.067 | *** | *** | 1 |
| 11171.001 | | | | | | |
| 101556[708]- | 2 | 1.16 | 0.106 | *** | *** | 1 |
| 11172.001 | | | | | | |
| 101556[708]- | 2 | 0.01 | 0.003 | *** | *** | 1 |
| 11173.001 | | | | | | |
| 101556[708]- | 2 | 0.00 | 0.001 | *** | *** | 0.75 |
| 11174.001 | | | | | | |
| YFP hairpin | | | | | | |
| Events | | | | | | |
| 110853[11]- | 2 | 0.02 | 0.006 | *** | *** | 1 |
| 390.001 | | | | | | |
| 110853[11]- | 2 | 0.02 | 0.005 | *** | *** | 0.75 |
| 391.001 | | | | | | |
| 110853[11]- | 2 | 0.03 | 0.003 | *** | *** | 1 |
| 393.001 | | | | | | |
| 110853[11]- | 2 | 0.16 | 0.031 | *** | *** | 1 |
| 394.001 | | | | | | |
| 110853[11]- | 2 | 0.20 | 0.042 | *** | *** | 0.75 |
| 395.001 | | | | | | |
| 110853[11]- | 3 | 0.01 | 0.009 | 0.01 | 0.002 | 1 |
| 396.001 | | | | | | |
| 110853[11]- | 3 | 0.01 | 0.000 | 0.01 | 0.000 | 0.02 |
| 397.001 | | | | | | |
| 110853[11]- | 3 | 0.01 | 0.001 | 0.01 | 0.002 | 1 |
| 398.001 | | | | | | |
| 110853[11]- | 3 | 0.01 | 0.001 | 0.00 | 0.001 | 1 |
| 401.001 | | | | | | |
| Non-transformed | | | | | | |
| Plants | | | | | | |
| 7sh382 | 3 | 0.01 | 0.000 | 0.01 | 0.005 | 1 |
| 7sh382 | 3 | 0.01 | 0.003 | 0.00 | 0.000 | 1 |
| 7sh382 | 3 | 0.01 | 0.000 | 0.00 | 0.000 | 1 |
| 7sh382 | 2 | 0.01 | 0.004 | *** | *** | 0.25 |
| 7sh382 | 2 | 0.48 | 0.058 | *** | *** | 0.25 |
| 7sh382 | 2 | 0.34 | 0.090 | *** | *** | 1 |
| 7sh382 | 2 | 0.01 | 0.000 | *** | *** | 0.5 |
| 7sh382 | 2 | 0.01 | 0.002 | *** | *** | 0.75 |
| B104 | 3 | 0.01 | 0.000 | 0.00 | 0.000 | 0.75 |
| B104 | 3 | 0.01 | 0.003 | 0.00 | 0.000 | 1 |
| B104 | 3 | 0.07 | 0.017 | 0.00 | 0.000 | ** |
| B104 | 2 | 0.00 | 0.000 | *** | *** | 1 |
| B104 | 2 | 0.01 | 0.003 | *** | *** | 0.5 |
| B104 | 2 | 0.03 | 0.004 | *** | *** | *** |
| B104 | 2 | 0.01 | 0.000 | *** | *** | 1 |
| B104 | 2 | 0.10 | 0.003 | *** | *** | 1 |

*RTL = Relative Transcript Level as measured against TIP4-like gene transcript levels.
**NG = Not Graded due to small plant size.
***ND = Not Done.

### Example 10

### Transgenic Zea mays Comprising Coleopteran Pest Sequences

10-20 transgenic T₀ Zea mays plants are generated as described in EXAMPLE 6. A further 10-20 T₁ Zea mays independent lines expressing hairpin dsRNA for an RNAi construct are obtained for corn rootworm challenge. Hairpin dsRNA may be derived as set forth in SEQ ID NO:18, SEQ ID NO:19, or otherwise further comprising SEQ ID NO:1, SEQ ID NO:102, or SEQ ID NO:107. Additional hairpin dsRNAs are derived, for example, from coleopteran pest sequences such as, for example, Caf1-180 (U.S. Patent Application Publication No. 2012/0174258), VatpaseC (U.S. Patent Application Publication No. 2012/0174259), Rho1 (U.S. Patent Application Publication No. 2012/0174260), VatpaseH (U.S. Patent Application Publication No. 2012/0198586), PPI-87B (U.S. Patent Application Publication No. 2013/0091600), RPA70 (U.S. Patent Application Publication No. 2013/0091601), or RPS6 (U.S. Patent Application Publication No. 2013/0097730). These are confirmed through RT-PCR or other molecular analysis methods.

Total RNA preparations from selected independent T₁ lines are optionally used for RT-PCR with primers designed to bind in the linker of the hairpin expression cassette in each of the RNAi constructs. In addition, specific primers for each target gene in an RNAi construct are optionally used to amplify and confirm the production of the pre-processed mRNA required for siRNA production in planta. The amplification of the desired bands for each target gene confirms the expression of the hairpin RNA in each transgenic Zea mays plant. Processing of the dsRNA hairpin of the target genes into siRNA is subsequently optionally confirmed in independent transgenic lines using RNA blot hybridizations.

Moreover, RNAi molecules having mismatch sequences with more than 80% sequence identity to target genes affect corn rootworms in a way similar to that seen with RNAi molecules having 100% sequence identity to the target genes. The pairing of mismatch sequence with native sequences to form a hairpin dsRNA in the same RNAi construct delivers plant-processed siRNAs capable of affecting the growth, development and viability of feeding coleopteran pests.

In planta delivery of dsRNA, siRNA or miRNA corresponding to target genes and the subsequent uptake by coleopteran pests through feeding results in down-regulation of the target genes in the coleopteran pest through RNA-mediated gene silencing. When the function of a target gene is important at one or more stages of development, the growth and/or development of the coleopteran pest is affected, and in the case of at least one of WCR, NCR, SCR, MCR, D. balteata LeConte, D. u. tenella, and D. u. undecimpunctata Mannerheim, leads to failure to successfully infest, feed, and/or develop, or leads to death of the coleopteran pest. The choice of target genes and the successful application of RNAi is then used to control coleopteran pests.

Phenotypic Comparison of Transgenic RNAi Lines and Nontransformed Zea mays.

Target coleopteran pest genes or sequences selected for creating hairpin dsRNA have no similarity to any known plant gene sequence. Hence, it is not expected that the production or the activation of (systemic) RNAi by constructs targeting these coleopteran pest genes or sequences will have any deleterious effect on transgenic plants. However, development and morphological characteristics of transgenic lines are compared with non-transformed plants, as well as those of transgenic lines transformed with an “empty” vector having no hairpin-expressing gene. Plant root, shoot, foliage and reproduction characteristics are compared. There is no observable difference in root length and growth patterns of transgenic and non-transformed plants. Plant shoot characteristics, such as height, leaf numbers and sizes, time of flowering, floral size and appearance are similar. In general, there are no observable morphological differences between transgenic lines and those without expression of target iRNA molecules when cultured in vitro and in soil in the glasshouse.

### Example 11

### Transgenic Zea mays Comprising a Coleopteran Pest Sequence and Additional RNAi Constructs

A transgenic Zea mays plant comprising a heterologous coding sequence in its genome that is transcribed into an iRNA molecule that targets an organism other than a coleopteran pest is secondarily transformed via Agrobacterium or WHISKERS™ methodologies (see Petolino and Arnold (2009) Methods Mol. Biol. 526:59-67) to produce one or more insecticidal dsRNA molecules (for example, at least one dsRNA molecule including a dsRNA molecule targeting a gene comprising SEQ ID NO:1, SEQ ID NO:102, and/or SEQ ID NO:107). Plant transformation plasmid vectors prepared essentially as described in EXAMPLE 4 are delivered via Agrobacterium or WHISKERS™-mediated transformation methods into maize suspension cells or immature maize embryos obtained from a transgenic Hi II or B104 Zea mays plant comprising a heterologous coding sequence in its genome that is transcribed into an iRNA molecule that targets an organism other than a coleopteran pest. Doubly-transformed plants are obtained that produce iRNA molecules and insecticidal proteins for control of coleopteran pests.

### Example 12

### Transgenic Zea mays Comprising an RNAi Construct and Additional Coleopteran Pest Control Sequences

A transgenic Zea mays plant comprising a heterologous coding sequence in its genome that is transcribed into an iRNA molecule that targets a coleopteran pest organism (for example, at least one dsRNA molecule including a dsRNA molecule targeting a gene comprising SEQ ID NO:1, SEQ ID NO:102, or SEQ ID NO:107) is secondarily transformed via Agrobacterium or WHISKERS™ methodologies (see Petolino and Arnold (2009) Methods Mol. Biol. 526:59-67) to produce one or more insecticidal protein molecules, for example, Cry3, Cry34 and Cry35 insecticidal proteins. Plant transformation plasmid vectors prepared essentially as described in EXAMPLE 4 are delivered via Agrobacterium or WHISKERS™-mediated transformation methods into maize suspension cells or immature maize embryos obtained from a transgenic B104 Zea mays plant comprising a heterologous coding sequence in its genome that is transcribed into an iRNA molecule that targets a coleopteran pest organism. Doubly-transformed plants are obtained that produce iRNA molecules and insecticidal proteins for control of coleopteran pests.

### Example 13

### Screening of Candidate Target Genes in Neotropical Brown Stink Bug (Euschistus heros)

Neotropical Brown Stink Bug (BSB; Euschistus heros) Colony.

BSB were reared in a 27° C. incubator, at 65% relative humidity, with 16:8 hour light: dark cycle. One gram of eggs collected over 2-3 days were seeded in 5 L containers with filter paper discs at the bottom, and the containers were covered with #18 mesh for ventilation. Each rearing container yielded approximately 300-400 adult BSB. At all stages, the insects were fed fresh green beans three times per week, a sachet of seed mixture that contained sunflower seeds, soybeans, and peanuts (3:1:1 by weight ratio) was replaced once a week. Water was supplemented in vials with cotton plugs as wicks. After the initial two weeks, insects were transferred onto new container once a week.

BSB Artificial Diet.

Neotropical Brown Stink Bugs (BSB; Euschistus heros) were reared on BSB artificial diet prepared as follows. Lyophilized green beans were blended to a fine powder in a MAGIC BULLET® blender, while raw (organic) peanuts were blended in a separate MAGIC BULLET® blender. Blended dry ingredients were combined (weight percentages: green beans, 35%; peanuts, 35%; sucrose, 5%; Vitamin complex (e.g., Vanderzant Vitamin Mixture for insects, SIGMA-ALDRICH, Catalog No. V1007), 0.9%); in a large MAGIC BULLET® blender, which was capped and shaken well to mix the ingredients. The mixed dry ingredients were then added to a mixing bowl. In a separate container, water and benomyl anti-fungal agent (50 ppm; 25 μL of a 20,000 ppm solution/50 mL diet solution) were mixed well, and then added to the dry ingredient mixture. All ingredients were mixed by hand until the solution was fully blended. The diet was shaped into desired sizes, wrapped loosely in aluminum foil, heated for 4 hours at 60° C., and then cooled and stored at 4° C. The artificial diet was used within two weeks of preparation

BSB Transcriptome Assembly.

Six stages of BSB development were selected for mRNA library preparation. Total RNA was extracted from insects frozen at −70° C., and homogenized in 10 volumes of Lysis/Binding buffer in Lysing MATRIX A 2 mL tubes (MP BIOMEDICALS, Santa Ana, Calif.) on a FastPrep®-24 Instrument (MP BIOMEDICALS). Total mRNA was extracted using a mirVana™ miRNA Isolation Kit (AMBION; INVITROGEN) according to the manufacturer's protocol. RNA sequencing using an Illumina® HiSeg™ system (San Diego, Calif.) provided candidate target gene sequences for use in RNAi insect control technology. HiSeg™ generated a total of about 378 million reads for the six samples. The reads were assembled individually for each sample using TRINITY™ assembler software (Grabherr et al. (2011) Nature Biotech. 29:644-652). The assembled transcripts were combined to generate a pooled transcriptome. This BSB pooled transcriptome contained 378,457 sequences.

BSB_Gho/Sec24B2 Ortholog Identification.

A tBLASTn search of the BSB pooled transcriptome was performed using as query sequence a Drosophila Sec24CD ortholog (H. sapiens) protein (i.e., sten or gho) Sec24CD-PB; GENBANK Accession No. NP_001259917. BSB_Gho (SEQ ID NO:78 and SEQ ID NO:79) were identified as a Euschistus heros candidate target gene products.

Template Preparation and dsRNA Synthesis

cDNA was prepared from total BSB RNA extracted from a single young adult insect (about 90 mg) using TRIzol® Reagent (LIFE TECHNOLOGIES). The insect was homogenized at room temperature in a 1.5 mL microcentrifuge tube with 200 μL of TRIzol® using a pellet pestle (FISHERBRAND Catalog No. 12-141-363) and Pestle Motor Mixer (COLE-PARMER, Vernon Hills, Ill.). Following homogenization, an additional 800 μL of TRIzol® was added, the homogenate was vortexed, and then incubated at room temperature for five minutes. Cell debris was removed by centrifugation and the supernatant was transferred to a new tube. Following manufacturer-recommended TRIzol® extraction protocol for 1 mL of TRIzol®, the RNA pellet was dried at room temperature and resuspended in 200 μL of Tris Buffer from a GFX PCR DNA AND GEL EXTRACTION KIT (Illustra™; GE HEALTHCARE LIFE SCIENCES) using Elution Buffer Type 4 (i.e. 10 mM Tris-HCl pH8.0). RNA concentration was determined using a NANODROP™ 8000 spectrophotometer (THERMO SCIENTIFIC, Wilmington, Del.).

200 μL of chloroform were added and the mixture was vortexed for 15 seconds. After allowing the extraction to sit at room temperature for 2 to 3 min, the phases were separated by centrifugation at 12,000×g at 4° C. for 15 minutes. The upper aqueous phase was carefully transferred into another nuclease-free 1.5 mL microcentrifuge tube, and the RNA was precipitated with 500 μL of room temperature isopropanol. After ten-minute incubation at room temperature, the mixture was centrifuged for 10 minutes as above. The RNA pellet was rinsed with 1 mL of room-temperature 75% ethanol and centrifuged for an additional 10 minutes as above. The RNA pellet was dried at room temperature and resuspended in 200 μL of Tris Buffer from a GFX PCR DNA AND GEL EXTRACTION KIT (Illustra™; GE HEALTHCARE LIFE SCIENCES) using Elution Buffer Type 4 (i.e. 10 mM Tris-HCl pH8.0). RNA concentration was determined using a NANODROP™ 8000 spectrophotometer (THERMO SCIENTIFIC, Wilmington, Del.).

cDNA was reverse-transcribed from 5 μg of BSB total RNA template and oligo dT primer using a SUPERSCRIPT III FIRST-STRAND SYNTHESIS SYSTEM™ for RT-PCR (INVITROGEN), following the supplier's recommended protocol. The final volume of the transcription reaction was brought to 100 μL with nuclease-free water.

cDNA Amplification.

Primers BSB_Gho-1-For (SEQ ID NO:89) and BSB_Gho-1-Rev (SEQ ID NO:90) to amplify BSB_Gho-1 template BSB_Gho-2-For (SEQ ID NO:91) and BSB_Gho-2-Rev (SEQ ID NO:92) to amplify BSB_Gho-2 template, and BSB_Gho-3-For (SEQ ID NO:93) and BSB_Gho-3-Rev (SEQ ID NO:94) to amplify BSB_Gho-3 template, were used in touch-down PCR (annealing temperature lowered from 60° C. to 50° C. in a 1° C./cycle decrease) with 1 μL of cDNA (above) as the template. Fragments comprising 397 bp segment of Gho: BSB_Gho region 1, also referred to as BSB_Gho-1 (SEQ ID NO:86), 494 bp segments of Gho: BSB_Gho region 2, also referred to as BSB_Gho-2 (SEQ ID NO:87), and 485 bp BSB_Gho region 3 also referred to as BSB_Gho-3 (SEQ ID NO:88) respectively, were generated during 35 cycles of PCR. The above procedure was also used to amplify a 301 bp negative control template YFPv2 (SEQ ID NO:95) using YFPv2-F (SEQ ID NO:96) and YFPv2-R (SEQ ID NO:97) primers. The BSB_Gho and YFPv2 primers contained a T7 phage promoter sequence (SEQ ID NO:7) at their 5′ ends, and thus enabled the use of YFPv2 (SEQ ID NO:95), BSB_Gho-1 (SEQ ID NO:86), BSB_Gho-2 (SEQ ID NO:87), and BSB_Gho-3 (SEQ ID NO:88) DNA fragments for dsRNA transcription.

dsRNA Synthesis.

dsRNA was synthesized using 2 μL PCR product (above) as the template with a MEGAscript™ T7 RNAi kit (AMBION) used according to the manufacturer's instructions. See FIG. 1. dsRNA was quantified on a NANODROP™ 8000 spectrophotometer, and diluted to 500 ng/μL in nuclease-free 0.1×TE buffer (1 mM Tris HCL, 0.1 mM EDTA, pH 7.4).

Injection of dsRNA into BSB Hemocoel.

BSB were reared on a green bean and seed diet, as the colony, in a 27° C. incubator at 65% relative humidity and 16:8 hour light: dark photoperiod. Second instar nymphs (each weighing 1 to 1.5 mg) were gently handled with a small brush to prevent injury, and were placed in a Petri dish on ice to chill and immobilize the insects. Each insect was injected with 55.2 nL 500 ng/μL dsRNA solution (i.e., 27.6 ng dsRNA; dosage of 18.4 to 27.6 μg/g body weight). Injections were performed using a NANOJECT™ II injector (DRUMMOND SCIENTIFIC, Broomhall, Pa.), equipped with an injection needle pulled from a Drummond 3.5 inch #3-000-203-G/X glass capillary. The needle tip was broken, and the capillary was backfilled with light mineral oil and then filled with 2 to 3 μL of dsRNA. dsRNA was injected into the abdomen of the nymphs (10 insects injected per dsRNA per trial), and the trials were repeated on three different days. Injected insects (5 per well) were transferred into 32-well trays (Bio-RT-32 Rearing Tray; BIO-SERV, Frenchtown, N.J.) containing a pellet of artificial BSB diet, and covered with Pull-N-Peel™ tabs (BIO-CV-4; BIO-SERV). Moisture was supplied by means of 1.25 mL water in a 1.5 mL microcentrifuge tube with a cotton wick. The trays were incubated at 26.5° C., 60% humidity, and 16:8 hour light: dark photoperiod. Viability counts and weights were taken on day 7 after the injections.

BSB_Gho is a Lethal dsRNA Target.

As summarized in Table 15, 2^{nd} instar BSB nymphs were injected into the hemocoel with 27.6 ng of BSB_Gho-1 (SEQ ID NO:86), BSB_Gho-2 (SEQ ID NO:87), or BSB_Gho-3 (SEQ ID NO:88) dsRNA, which produced high mortality within seven days. The mortality determined for BSB_Gho-1, BSB_Gho-2, and BSB_Gho-3 dsRNA was significantly different from that seen with the same amount of injected YFPv2 dsRNA (negative control), with p=0.03135, p=0.003023, and p=0.005459, respectively (Student's t-test).

TABLE 15
Results of BSB_Gho-1, BSB_Gho-2 or BSB_Gho-3 dsRNA
injection into the hemocoel of ^{2nd} instar Neotropical Brown
Stink Bug nymphs seven days after injection.

| | | % Mortality +/− | |
|---|---|---|---|
| Treatment* | N Trials | SEM** | p value t-test |
| BSB_Gho-1 dsRNA | 3 | 66.7 ± 8.82 | 3.14E−02*** |
| BSB_Gho-2 dsRNA | 3 | 96.7 ± 3.33 | 3.02E−03*** |
| BSB_Gho-3 dsRNA | 3 | 90.0 ± 5.77 | 5.46E−03*** |
| Not injected | 3 | 6.7 ± 3.33 | 3.50E−01 |
| YFPv2 dsRNA | 3 | 19.3 ± 11.6 | |

*Ten insects injected per trial for each dsRNA.
**Standard error of the mean
***Significantly different from the YFPv2 dsRNA control using a Student's t-test.

### Example 14

### Transgenic Zea mays Comprising Hemipteran Pest Sequences

Ten to 20 transgenic T₀ Zea mays plants harboring expression vectors for nucleic acids comprising SEQ ID NO: 84, SEQ ID NO:85, SEQ ID NO:86, SEQ ID NO:87 and/or SEQ ID NO:88 are generated as described in EXAMPLE 4. A further 10-20 T₁ Zea mays independent lines expressing hairpin dsRNA for an RNAi construct are obtained for BSB challenge. Hairpin dsRNA are derived comprising SEQ ID NO:84 or SEQ ID NO:85, or segments thereof (e.g., SEQ ID NO:86, SEQ ID NO:87, and SEQ ID NO:88). These are confirmed through RT-PCR or other molecular analysis methods. Total RNA preparations from selected independent T₁ lines are optionally used for RT-PCR with primers designed to bind in the linker of the hairpin expression cassette in each of the RNAi constructs. In addition, specific primers for each target gene in an RNAi construct are optionally used to amplify and confirm the production of the pre-processed mRNA required for siRNA production in planta. The amplification of the desired bands for each target gene confirms the expression of the hairpin RNA in each transgenic Zea mays plant. Processing of the dsRNA hairpin of the target genes into siRNA is subsequently optionally confirmed in independent transgenic lines using RNA blot hybridizations.

Moreover, RNAi molecules having mismatch sequences with more than 80% sequence identity to target genes affect hemipterans in a way similar to that seen with RNAi molecules having 100% sequence identity to the target genes. The pairing of mismatch sequence with native sequences to form a hairpin dsRNA in the same RNAi construct delivers plant-processed siRNAs capable of affecting the growth, development, and viability of feeding hemipteran pests.

In planta delivery of dsRNA, siRNA, shRNA, hpRNA, or miRNA corresponding to target genes and the subsequent uptake by hemipteran pests through feeding results in down-regulation of the target genes in the hemipteran pest through RNA-mediated gene silencing. When the function of a target gene is important at one or more stages of development, the growth, development, and/or survival of the hemipteran pest is affected, and in the case of at least one of Euschistus heros, Piezodorus guildinii, Halyomorpha halys, Nezara viridula, Acrosternum hilare, and Euschistus servus leads to failure to successfully infest, feed, develop, and/or leads to death of the hemipteran pest. The choice of target genes and the successful application of RNAi is then used to control hemipteran pests.

Phenotypic Comparison of Transgenic RNAi Lines and Non-Transformed Zea mays.

Target hemipteran pest genes or sequences selected for creating hairpin dsRNA have no similarity to any known plant gene sequence. Hence it is not expected that the production or the activation of (systemic) RNAi by constructs targeting these hemipteran pest genes or sequences will have any deleterious effect on transgenic plants. However, development and morphological characteristics of transgenic lines are compared with non-transformed plants, as well as those of transgenic lines transformed with an “empty” vector having no hairpin-expressing gene. Plant root, shoot, foliage and reproduction characteristics are compared. There is no observable difference in root length and growth patterns of transgenic and non-transformed plants. Plant shoot characteristics such as height, leaf numbers and sizes, time of flowering, floral size and appearance are similar. In general, there are no observable morphological differences between transgenic lines and those without expression of target iRNA molecules when cultured in vitro and in soil in the glasshouse.

### Example 15

### Transgenic Glycine max Comprising Hemipteran Pest Sequences

Ten to 20 transgenic T₀ Glycine max plants harboring expression vectors for nucleic acids comprising SEQ ID NO:84 or SEQ ID NO:85, or segments thereof (e.g., SEQ ID NO:86, SEQ ID NO:87, and SEQ ID NO:88) are generated as is known in the art, including for example by Agrobacterium-mediated transformation, as follows. Mature soybean (Glycine max) seeds are sterilized overnight with chlorine gas for sixteen hours. Following sterilization with chlorine gas, the seeds are placed in an open container in a LAMINAR™ flow hood to dispel the chlorine gas. Next, the sterilized seeds are imbibed with sterile H₂O for sixteen hours in the dark using a black box at 24° C.

Preparation of Split-Seed Soybeans.

The split soybean seed comprising a portion of an embryonic axis protocol requires preparation of soybean seed material which is cut longitudinally, using a #10 blade affixed to a scalpel, along the hilum of the seed to separate and remove the seed coat, and to split the seed into two cotyledon sections. Careful attention is made to partially remove the embryonic axis, wherein about ½-⅓ of the embryo axis remains attached to the nodal end of the cotyledon.

Inoculation.

The split soybean seeds comprising a partial portion of the embryonic axis are then immersed for about 30 minutes in a solution of Agrobacterium tumefaciens (e.g., strain EHA 101 or EHA 105) containing binary plasmid comprising SEQ ID NO: 89 and/or SEQ ID NO:91. The A. tumefaciens solution is diluted to a final concentration of λ=0.6 OD₆₅₀ before immersing the cotyledons comprising the embryo axis.

Co-Cultivation.

Following inoculation, the split soybean seed is allowed to co-cultivate with the Agrobacterium tumefaciens strain for 5 days on co-cultivation medium (Agrobacterium Protocols, vol. 2, 2^{nd} Ed., Wang, K. (Ed.) Humana Press, New Jersey, 2006) in a Petri dish covered with a piece of filter paper.

Shoot Induction.

After 5 days of co-cultivation, the split soybean seeds are washed in liquid Shoot Induction (SI) media consisting of B5 salts, B5 vitamins, 28 mg/L Ferrous, 38 mg/L Na₂EDTA, 30 g/L sucrose, 0.6 g/L MES, 1.11 mg/L BAP, 100 mg/L TIMENTIN™, 200 mg/L cefotaxime, and 50 mg/L vancomycin (pH 5.7). The split soybean seeds are then cultured on Shoot Induction I (SI I) medium consisting of B5 salts, B5 vitamins, 7 g/L Noble agar, 28 mg/L Ferrous, 38 mg/L Na₂EDTA, 30 g/L sucrose, 0.6 g/L MES, 1.11 mg/L BAP, 50 mg/L TIMENTIN™, 200 mg/L cefotaxime, 50 mg/L vancomycin (pH 5.7), with the flat side of the cotyledon facing up and the nodal end of the cotyledon imbedded into the medium. After 2 weeks of culture, the explants from the transformed split soybean seed are transferred to the Shoot Induction II (SI II) medium containing SI I medium supplemented with 6 mg/L glufosinate (LIBERTY®).

Shoot Elongation.

After 2 weeks of culture on SI II medium, the cotyledons are removed from the explants and a flush shoot pad containing the embryonic axis are excised by making a cut at the base of the cotyledon. The isolated shoot pad from the cotyledon is transferred to Shoot Elongation (SE) medium. The SE medium consists of MS salts, 28 mg/L Ferrous, 38 mg/L Na₂EDTA, 30 g/L sucrose and 0.6 g/L MES, 50 mg/L asparagine, 100 mg/L L-pyroglutamic acid, 0.1 mg/L IAA, 0.5 mg/L GA3, 1 mg/L zeatin riboside, 50 mg/L TIMENTIN™, 200 mg/L cefotaxime, 50 mg/L vancomycin, 6 mg/L glufosinate, 7 g/L Noble agar, (pH 5.7). The cultures are transferred to fresh SE medium every 2 weeks. The cultures are grown in a CONVIRON™ growth chamber at 24° C. with an 18 h photoperiod at a light intensity of 80-90 μmol/m² sec.

Rooting.

Elongated shoots which developed from the cotyledon shoot pad are isolated by cutting the elongated shoot at the base of the cotyledon shoot pad, and dipping the elongated shoot in 1 mg/L IBA (Indole 3-butyric acid) for 1-3 minutes to promote rooting. Next, the elongated shoots are transferred to rooting medium (MS salts, B5 vitamins, 28 mg/L Ferrous, 38 mg/L Na₂EDTA, 20 g/L sucrose and 0.59 g/L MES, 50 mg/L asparagine, 100 mg/L L-pyroglutamic acid 7 g/L Noble agar, pH 5.6) in phyta trays.

Cultivation.

Following culture in a CONVIRON™ growth chamber at 24° C., 18 h photoperiod, for 1-2 weeks, the shoots which have developed roots are transferred to a soil mix in a covered sundae cup and placed in a CONVIRON™ growth chamber (models CMP4030 and CMP3244, Controlled Environments Limited, Winnipeg, Manitoba, Canada) under long day conditions (16 hours light/8 hours dark) at a light intensity of 120-150 μmol/m² sec under constant temperature (22° C.) and humidity (40-50%) for acclimatization of plantlets. The rooted plantlets are acclimated in sundae cups for several weeks before they are transferred to the greenhouse for further acclimatization and establishment of robust transgenic soybean plants.

A further 10-20 T₁ Glycine max independent lines expressing hairpin dsRNA for an RNAi construct are obtained for BSB challenge. Hairpin dsRNA may be derived comprising SEQ ID NO:84 or SEQ ID NO:85, or segments thereof (e.g., SEQ ID NO:86, SEQ ID NO:87, and SEQ ID NO:88). These are confirmed through RT-PCR or other molecular analysis methods as known in the art. Total RNA preparations from selected independent T₁ lines are optionally used for RT-PCR with primers designed to bind in the linker of the hairpin expression cassette in each of the RNAi constructs. In addition, specific primers for each target gene in an RNAi construct are optionally used to amplify and confirm the production of the pre-processed mRNA required for siRNA production in planta. The amplification of the desired bands for each target gene confirms the expression of the hairpin RNA in each transgenic Glycine max plant. Processing of the dsRNA hairpin of the target genes into siRNA is subsequently optionally confirmed in independent transgenic lines using RNA blot hybridizations.

RNAi molecules having mismatch sequences with more than 80% sequence identity to target genes affect BSB in a way similar to that seen with RNAi molecules having 100% sequence identity to the target genes. The pairing of mismatch sequence with native sequences to form a hairpin dsRNA in the same RNAi construct delivers plant-processed siRNAs capable of affecting the growth, development, and viability of feeding hemipteran pests.

In planta delivery of dsRNA, siRNA, shRNA, or miRNA corresponding to target genes and the subsequent uptake by hemipteran pests through feeding results in down-regulation of the target genes in the hemipteran pest through RNA-mediated gene silencing. When the function of a target gene is important at one or more stages of development, the growth, development, and/or survival of the hemipteran pest is affected, and in the case of at least one of Euschistus heros, Piezodorus guildinii, Halyomorpha halys, Nezara viridula, Chinavia hilare, Euschistus servus, Dichelops melacanthus, Dichelops furcatus, Edessa meditabunda, Thyanta perditor, Chinavia marginatum, Horcias nobilellus, Taedia stigmosa, Dysdercus peruvianus, Neomegalotomus parvus, Leptoglossus zonatus, Niesthrea sidae, and Lygus lineolaris leads to failure to successfully infest, feed, and/or develop, or leads to death of the hemipteran pest. The choice of target genes and the successful application of RNAi is then used to control hemipteran pests.

Phenotypic Comparison of Transgenic RNAi Lines and Non-Transformed Glycine max.

Target hemipteran pest genes or sequences selected for creating hairpin dsRNA have no similarity to any known plant gene sequence. Hence it is not expected that the production or the activation of (systemic) RNAi by constructs targeting these hemipteran pest genes or sequences will have any deleterious effect on transgenic plants. However, development and morphological characteristics of transgenic lines are compared with non-transformed plants, as well as those of transgenic lines transformed with an “empty” vector having no hairpin-expressing gene. Plant root, shoot, foliage and reproduction characteristics are compared. There is no observable difference in root length and growth patterns of transgenic and non-transformed plants. Plant shoot characteristics such as height, leaf numbers and sizes, time of flowering, floral size and appearance are similar. In general, there are no observable morphological differences between transgenic lines and those without expression of target iRNA molecules when cultured in vitro and in soil in the glasshouse.

### Example 16

### E. heros Bioassays on Artificial Diet

In Gho/Sec24B2 dsRNA feeding assays on artificial diet, 32-well trays are set up with an ˜18 mg pellet of artificial diet and water, as for injection experiments (See EXAMPLE 13). dsRNA at a concentration of 200 ng/μL is added to the food pellet and water sample; 100 μL to each of two wells. Five 2^{nd} instar E. heros nymphs are introduced into each well. Water samples and dsRNA that targets YFP transcript are used as negative controls. The experiments are repeated on three different days. Surviving insects are weighed, and the mortality rates are determined after 8 days of treatment. Significant mortality and/or growth inhibition is observed in the wells provided with BSB_Gho dsRNA, compared to the control wells.

### Example 17

### Transgenic Arabidopsis thaliana Comprising Hemipteran Pest Sequences

Arabidopsis transformation vectors containing a target gene construct for hairpin formation comprising segments of Gho/Sec24B2 (e.g., SEQ ID NO:84 and/or SEQ ID NO:85) are generated using standard molecular methods similar to EXAMPLE 4. Arabidopsis transformation is performed using standard Agrobacterium-based procedure. T₁ seeds are selected with glufosinate tolerance selectable marker. Transgenic T₁ Arabidopsis plants are generated and homozygous simple-copy T₂ transgenic plants are generated for insect studies. Bioassays are performed on growing Arabidopsis plants with inflorescences. Five to ten insects are placed on each plant and monitored for survival within 14 days.

Construction of Arabidopsis Transformation Vectors.

Entry clones based on entry vector pDAB3916 harboring a target gene construct for hairpin formation comprising a segment of Gho/Sec24B2 (e.g., SEQ ID NO:84 and/or SEQ ID NO:85) are assembled using a combination of chemically synthesized fragments (DNA2.0, Menlo Park, Calif.) and standard molecular cloning methods. Intramolecular hairpin formation by RNA primary transcripts is facilitated by arranging (within a single transcription unit) two copies of a target gene segment in opposite orientations, the two segments being separated by a linker sequence (e.g. ST-LS1 intron; SEQ ID NO:21) (Vancanneyt et al. (1990) Mol. Gen. Genet. 220(2):245-50). Thus, the primary mRNA transcript contains the two Gho/Sec24B2 gene segment sequences as large inverted repeats of one another, separated by the linker sequence. A copy of a promoter (e.g., Arabidopsis thaliana ubiquitin 10 promoter (Callis et al. (1990) J. Biological Chem. 265:12486-12493)) is used to drive production of the primary mRNA hairpin transcript, and a fragment comprising a 3′ untranslated region from Open Reading Frame 23 of Agrobacterium tumefaciens (AtuORF23 3′ UTR v1; U.S. Pat. No. 5,428,147) is used to terminate transcription of the hairpin-RNA-expressing gene.

The hairpin clones within entry vectors are used in standard GATEWAY® recombination reactions with a binary destination vector (pDAB101836) to produce hairpin RNA expression transformation vectors for Agrobacterium-mediated Arabidopsis transformation.

Binary destination vector pDAB101836 comprises an herbicide tolerance gene, DSM-2v2 (U.S. Patent App. No. 2011/0107455), under the regulation of a Cassava vein mosaic virus promoter (CsVMV Promoter v2, U.S. Pat. No. 7,601,885; Verdaguer et al. (1996) Plant Mol. Biol. 31:1129-39). A fragment comprising a 3′ untranslated region from Open Reading Frame 1 of Agrobacterium tumefaciens (AtuORF1 3′ UTR v6; Huang et al. (1990) J. Bacteriol. 172:1814-22) is used to terminate transcription of the DSM2v2 mRNA.

A negative control binary construct, pDAB114507, which comprises a gene that expresses a YFP hairpin RNA, is constructed by means of standard GATEWAY® recombination reactions with a typical binary destination vector (pDAB101836) and entry vector pDAB3916. Entry construct pDAB112644 comprises a YFP hairpin sequence (hpYFP v2-1, SEQ ID NO:100) under the expression control of an Arabidopsis Ubiquitin 10 promoter (as above) and a fragment comprising an ORF23 3′ untranslated region from Agrobacterium tumefaciens (as above).

Production of Transgenic Arabidopsis Comprising Insecticidal Hairpin RNAs: Agrobacterium-Mediated Transformation.

Binary plasmids containing hairpin sequences are electroporated into Agrobacterium strain GV3101 (pMP90RK). The recombinant Agrobacterium clones are confirmed by restriction analysis of plasmids preparations of the recombinant Agrobacterium colonies. A Qiagen Plasmid Max Kit (Qiagen, Cat#12162) is used to extract plasmids from Agrobacterium cultures following the manufacture recommended protocol.

Arabidopsis Transformation and T₁ Selection.

Twelve to fifteen Arabidopsis plants (c.v. Columbia) are grown in 4″ pots in the green house with light intensity of 250 μmol/m², 25° C., and 18:6 hours of light: dark conditions. Primary flower stems are trimmed one week before transformation. Agrobacterium inoculums are prepared by incubating 10 μL recombinant Agrobacterium glycerol stock in 100 mL LB broth (Sigma L3022)+100 mg/L Spectinomycin+50 mg/L Kanamycin at 28° C. and shaking at 225 rpm for 72 hours. Agrobacterium cells are harvested and suspended into 5% sucrose+0.04% Silwet-L77 (Lehle Seeds Cat #VIS-02)+10 μg/L benzamino purine (BA) solution to OD₆₀₀ 0.8˜1.0 before floral dipping. The above-ground parts of the plant are dipped into the Agrobacterium solution for 5-10 minutes, with gentle agitation. The plants are then transferred to the greenhouse for normal growth with regular watering and fertilizing until seed set.

### Example 18

### Growth and Bioassays of Transgenic Arabidopsis

Selection of T₁ Arabidopsis Transformed with Hairpin RNAi Constructs.

Up to 200 mg of T₁ seeds from each transformation are stratified in 0.1% agarose solution. The seeds are planted in germination trays (10.5″×21″×1″; T.O. Plastics Inc., Clearwater, Minn.) with #5 sunshine media. Transformants are selected for tolerance to Ignite® (glufosinate) at 280 g/ha at 6 and 9 days post planting. Selected events are transplanted into 4″ diameter pots. Insertion copy analysis is performed within a week of transplanting via hydrolysis quantitative Real-Time PCR (qPCR) using Roche LightCycler480™. The PCR primers and hydrolysis probes are designed against DSM2v2 selectable marker using LightCycler™ Probe Design Software 2.0 (Roche). Plants are maintained at 24° C., with a 16:8 hour light: dark photoperiod under fluorescent and incandescent lights at intensity of 100-150 mE/m²s.

E. heros Plant Feeding Bioassay.

At least four low copy (1-2 insertions), four medium copy (2-3 insertions), and four high copy (>4 insertions) events are selected for each construct. Plants are grown to a reproductive stage (plants containing flowers and siliques). The surface of soil is covered with ˜50 mL volume of white sand for easy insect identification. Five to ten 2^{nd} instar E. heros nymphs are introduced onto each plant. The plants are covered with plastic tubes that are 3″ in diameter, 16″ tall, and with wall thickness of 0.03″ (Item No. 484485, Visipack Fenton Mo.); the tubes are covered with nylon mesh to isolate the insects. The plants are kept under normal temperature, light, and watering conditions in a conviron. In 14 days, the insects are collected and weighed; percent mortality as well as growth inhibition (1−weight treatment/weight control) are calculated. YFP hairpin-expressing plants are used as controls. Significant mortality and/or growth inhibition is observed in nymphs feeding on transgenic Gho/Sec24B2 dsRNA plants, compared to that of nymphs on control plants.

T₂ Arabidopsis Seed Generation and T₂ Bioassays.

T₂ seed is produced from selected low copy (1-2 insertions) events for each construct. Plants (homozygous and/or heterozygous) are subjected to E. heros feeding bioassay, as described above. T₃ seed is harvested from homozygotes and stored for future analysis.

### Example 19

### Transformation of Additional Crop Species

Cotton is transformed with Gho/Sec24B2 and/or Sec24B1 (with or without a chloroplast transit peptide) to provide control of coleopteran and/or hemipteran insects by utilizing a method known to those of skill in the art, for example, substantially the same techniques previously described in EXAMPLE 14 of U.S. Pat. No. 7,838,733, or Example 12 of PCT International Patent Publication No. WO 2007/053482.

### Example 20

### Gho/Sec24B2 and/or Sec24B1 dsRNA in Insect Management

Gho/Sec24B2 and/or Sec24B1 dsRNA transgenes are combined with other dsRNA molecules in transgenic plants to provide redundant RNAi targeting and synergistic RNAi effects. Transgenic plants including, for example and without limitation, corn, soybean, and cotton expressing dsRNA that targets Gho/Sec24B2 and/or Sec24B1 are useful for preventing feeding damage by coleopteran and hemipteran insects. Gho/Sec24B2 and/or Sec24B1 dsRNA transgenes are also combined in plants with Bacillus thuringiensis insecticidal protein technology to represent new modes of action in Insect Resistance Management gene pyramids. When combined with other dsRNA molecules that target insect pests, and/or with Bacillus thuringiensis insecticidal proteins, in transgenic plants, a synergistic insecticidal effect is observed that also mitigates the development of resistant insect populations.

What may be claimed is:

1. An isolated nucleic acid comprising at least one polynucleotide operably linked to a heterologous promoter, wherein the polynucleotide is selected from the group consisting of:
SEQ ID NO:1; the complement of SEQ ID NO:1; a fragment of at least 15 contiguous nucleotides of SEQ ID NO:1; the complement of a fragment of at least 15 contiguous nucleotides of SEQ ID NO:1; a native coding sequence of a Diabrotica organism comprising SEQ ID NO:1; the complement of a native coding sequence of a Diabrotica organism comprising SEQ ID NO:1; a fragment of at least 15 contiguous nucleotides of a native coding sequence of a Diabrotica organism comprising SEQ ID NO:1; the complement of a fragment of at least 15 contiguous nucleotides of a native coding sequence of a Diabrotica organism comprising SEQ ID NO:1;
SEQ ID NO:102; the complement of SEQ ID NO:102; a fragment of at least 15 contiguous nucleotides of SEQ ID NO:102; the complement of a fragment of at least 15 contiguous nucleotides of SEQ ID NO:102; a native coding sequence of a Diabrotica organism comprising SEQ ID NO:102; the complement of a native coding sequence of a Diabrotica organism comprising SEQ ID NO:102; a fragment of at least 15 contiguous nucleotides of a native coding sequence of a Diabrotica organism comprising SEQ ID NO:102; the complement of a fragment of at least 15 contiguous nucleotides of a native coding sequence of a Diabrotica organism comprising SEQ ID NO:102;
SEQ ID NO:107; the complement of SEQ ID NO:107; a fragment of at least 15 contiguous nucleotides of SEQ ID NO:107; the complement of a fragment of at least 15 contiguous nucleotides of SEQ ID NO:107; a native coding sequence of a Diabrotica organism comprising SEQ ID NO:107; the complement of a native coding sequence of a Diabrotica organism comprising SEQ ID NO:107; a fragment of at least 15 contiguous nucleotides of a native coding sequence of a Diabrotica organism comprising SEQ ID NO:107; the complement of a fragment of at least 15 contiguous nucleotides of a native coding sequence of a Diabrotica organism comprising SEQ ID NO:107;
SEQ ID NO:84; the complement of SEQ ID NO:84; a fragment of at least 15 contiguous nucleotides of SEQ ID NO:84; the complement of a fragment of at least 15 contiguous nucleotides of SEQ ID NO:84; a native coding sequence of a Euschistus organism comprising SEQ ID NO:84; the complement of a native coding sequence of a Euschistus organism comprising SEQ ID NO:84; a fragment of at least 15 contiguous nucleotides of a native coding sequence of a Euschistus organism comprising SEQ ID NO:84; the complement of a fragment of at least 15 contiguous nucleotides of a native coding sequence of a Euschistus organism comprising SEQ ID NO:84;
SEQ ID NO:85; the complement of SEQ ID NO:85; a fragment of at least 15 contiguous nucleotides of SEQ ID NO:85; the complement of a fragment of at least 15 contiguous nucleotides of SEQ ID NO:85; a native coding sequence of a Euschistus organism comprising SEQ ID NO:85; the complement of a native coding sequence of a Euschistus organism comprising SEQ ID NO:85; a fragment of at least 15 contiguous nucleotides of a native coding sequence of a Euschistus organism comprising SEQ ID NO:85; and the complement of a fragment of at least 15 contiguous nucleotides of a native coding sequence of a Euschistus organism comprising SEQ ID NO:85.

2. The polynucleotide of claim 1, wherein the polynucleotide is selected from the group consisting of SEQ ID NO:1, SEQ ID NO:3, SEQ ID NO:4, SEQ ID NO:5, SEQ ID NO:6, SEQ ID NO:18, SEQ ID NO:19, SEQ ID NO:84, SEQ ID NO:85, SEQ ID NO:86, SEQ ID NO:87, SEQ ID NO:88, SEQ ID NO:102, SEQ ID NO:104, SEQ ID NO:107, SEQ ID NO:109, and the complements of any of the foregoing.

3. A plant transformation vector comprising the polynucleotide of claim 1.

4. The polynucleotide of claim 1, wherein the organism is selected from the group consisting of D. v. virgifera LeConte; D. barberi Smith and Lawrence; D. u. howardi; D. v. zeae; D. balteata LeConte; D. u. tenella; D. u. undecimpunctata Mannerheim; Euschistus heros (Fabr.) (Neotropical Brown Stink Bug), Nezara viridula (L.) (Southern Green Stink Bug), Piezodorus guildinii (Westwood) (Red-banded Stink Bug), Halyomorpha halys (Stål) (Brown Marmorated Stink Bug), Chinavia hilare (Say) (Green Stink Bug), Euschistus servus (Say) (Brown Stink Bug), Dichelops melacanthus (Dallas), Dichelops furcatus (F.), Edessa meditabunda (F.), Thyanta perditor (F.) (Neotropical Red Shouldered Stink Bug), Chinavia marginatum (Palisot de Beauvois), Horcias nobilellus (Berg) (Cotton Bug), Taedia stigmosa (Berg), Dysdercus peruvianus (Guérin-Méneville), Neomegalotomus parvus (Westwood), Leptoglossus zonatus (Dallas), Niesthrea sidae (F.), Lygus hesperus (Knight) (Western Tarnished Plant Bug), and Lygus lineolaris (Palisot de Beauvois).

5. A ribonucleic acid (RNA) molecule transcribed from the polynucleotide of claim 1.

6. A double-stranded ribonucleic acid molecule produced from the expression of the polynucleotide of claim 1.

7. The double-stranded ribonucleic acid molecule of claim 6, wherein contacting the polynucleotide sequence with a coleopteran or hemipteran pest inhibits the expression of an endogenous nucleotide sequence specifically complementary to the polynucleotide.

8. The double-stranded ribonucleic acid molecule of claim 7, wherein contacting said ribonucleotide molecule with a coleopteran or hemipteran pest kills or inhibits the growth, and/or feeding of the pest.

9. The double stranded RNA of claim 6, comprising a first, a second and a third RNA segment, wherein the first RNA segment comprises the polynucleotide, wherein the third RNA segment is linked to the first RNA segment by the second polynucleotide sequence, and wherein the third RNA segment is substantially the reverse complement of the first RNA segment, such that the first and the third RNA segments hybridize when transcribed into a ribonucleic acid to form the double-stranded RNA.

10. The RNA of claim 5, selected from the group consisting of a double-stranded ribonucleic acid molecule and a single-stranded ribonucleic acid molecule of between about 15 and about 30 nucleotides in length.

11. A plant transformation vector comprising the polynucleotide of claim 1, wherein the heterologous promoter is functional in a plant cell.

12. A cell transformed with the polynucleotide of claim 1.

13. The cell of claim 12, wherein the cell is a prokaryotic cell.

14. The cell of claim 12, wherein the cell is a eukaryotic cell.

15. The cell of claim 14, wherein the cell is a plant cell.

16. A plant transformed with the polynucleotide of claim 1.

17. A seed of the plant of claim 16, wherein the seed comprises the polynucleotide.

18. A commodity product produced from the plant of claim 16, wherein the commodity product comprises a detectable amount of the polynucleotide.

19. The plant of claim 16, wherein the at least one polynucleotide is expressed in the plant as a double-stranded ribonucleic acid molecule.

20. The cell of claim 15, wherein the cell is a maize, soybean, or cotton cell.

21. The plant of claim 16, wherein the plant is maize, soybean, or cotton.

22. The plant of claim 16, wherein the at least one polynucleotide is expressed in the plant as a ribonucleic acid molecule, and the ribonucleic acid molecule inhibits the expression of an endogenous polynucleotide that is specifically complementary to the at least one polynucleotide when a coleopteran or hemipteran pest ingests a part of the plant.

23. The polynucleotide of claim 1, further comprising at least one additional polynucleotide that encodes an RNA molecule that inhibits the expression of an endogenous pest gene.

24. A plant transformation vector comprising the polynucleotide of claim 23, wherein the additional polynucleotide(s) are each operably linked to a heterologous promoter functional in a plant cell.

25. A method for controlling an insect pest population, the method comprising providing an agent comprising a ribonucleic acid (RNA) molecule that functions upon contact with the insect pest to inhibit a biological function within the pest, wherein the RNA is specifically hybridizable with a polynucleotide selected from the group consisting of any of SEQ ID NOs:112-127; the complement of any of SEQ ID NOs:112-127; a fragment of at least 15 contiguous nucleotides of any of SEQ ID NOs:112-127; the complement of a fragment of at least 15 contiguous nucleotides of any of SEQ ID NOs:112-127; a transcript of any of SEQ ID NOs:1, 84, 85, 102, and 107; and the complement of a transcript of any of SEQ ID NOs:1, 84, 85, 102, and 107.

26. The method according to claim 25, wherein the agent is a double-stranded RNA molecule.

27. The method according to claim 25, wherein the insect pest is a coleopteran or hemipteran pest.

28. A method for controlling a coleopteran or a hemipteran pest population, the method comprising:
providing an agent comprising a first and a second polynucleotide sequence that functions upon contact with the coleopteran pest to inhibit a biological function within the coleopteran pest, wherein the first polynucleotide sequence comprises a region that exhibits from about 90% to about 100% sequence identity to from about 15 to about 30 contiguous nucleotides of a sequence selected from the group consisting of SEQ ID NOs:112-116 and 124-127, and wherein the first polynucleotide sequence is specifically hybridized to the second polynucleotide sequence.

29. A method for controlling a coleopteran or hemipteran pest population, the method comprising:
providing in a host plant of a coleopteran or hemipteran pest a transformed plant cell comprising the polynucleotide of claim 1, wherein the polynucleotide is expressed to produce a ribonucleic acid molecule that functions upon contact with a coleopteran or hemipteran pest belonging to the population to inhibit the expression of a target sequence within the coleopteran or hemipteran pest and results in decreased growth and/or survival of the coleopteran or hemipteran pest or pest population, relative to the same pest species on a plant of the same host plant species that does not comprise the polynucleotide.

30. The method according to claim 29, wherein the ribonucleic acid molecule is a double-stranded ribonucleic acid molecule.

31. The method according to claim 29, wherein the coleopteran or hemipteran pest population is reduced relative to a population of the same pest species infesting a host plant of the same host plant species lacking the transformed plant cell.

32. The method according to claim 29, wherein the ribonucleic acid molecule is a double-stranded ribonucleic acid molecule.

33. The method according to claim 30, wherein the coleopteran or hemipteran pest population is reduced relative to a coleopteran or hemipteran pest population infesting a host plant of the same species lacking the transformed plant cell.

34. A method of controlling an insect pest infestation in a plant, the method comprising providing in the diet of the insect pest a ribonucleic acid (RNA) that is specifically hybridizable with a polynucleotide selected from the group consisting of:
SEQ ID NOs:112-127;
the complement of any of SEQ ID NOs:112-127;
a fragment of at least 15 contiguous nucleotides of any of SEQ ID NOs:112-116 and 119-127;
the complement of a fragment of at least 15 contiguous nucleotides of any of SEQ ID NOs:112-116 and 119-127;
a transcript of any of SEQ ID NOs:1, 84, 85, 102, and 107;
the complement of a transcript of any of SEQ ID NOs:1, 84, 85, 102, and 107;
a fragment of at least 15 contiguous nucleotides of a transcript of any of SEQ ID NOs:1, 84, 85, 102, and 107; and
the complement of a fragment of at least 15 contiguous nucleotides of a transcript of any of SEQ ID NOs:1, 84, 85, 102, and 107.

35. The method according to claim 34, wherein the diet comprises a plant cell transformed to express the polynucleotide.

36. The method according to claim 34, wherein the specifically hybridizable RNA is comprised in a double-stranded RNA molecule.

37. A method for improving the yield of a corn crop, the method comprising:
introducing the nucleic acid of claim 1 into a corn plant to produce a transgenic corn plant; and
cultivating the corn plant to allow the expression of the at least one polynucleotide; wherein expression of the at least one polynucleotide inhibits the development or growth of a coleopteran and/or hemipteran pest and loss of yield due to infection by the coleopteran and/or hemipteran pest.

38. The method according to claim 37, wherein expression of the at least one polynucleotide produces an RNA molecule that suppresses at least a first target gene in a coleopteran and/or hemipteran pest that has contacted a portion of the corn plant.

39. A method for producing a transgenic plant cell, the method comprising:
transforming a plant cell with a vector comprising the nucleic acid of claim 1;
culturing the transformed plant cell under conditions sufficient to allow for development of a plant cell culture comprising a plurality of transformed plant cells;
selecting for transformed plant cells that have integrated the at least one polynucleotide into their genomes;
screening the transformed plant cells for expression of a ribonucleic acid (RNA) molecule encoded by the at least one polynucleotide; and
selecting a plant cell that expresses the RNA.

40. The method according to claim 39, wherein the RNA molecule is a double-stranded RNA molecule.

41. A method for producing a coleopteran and/or hemipteran pest-resistant transgenic plant, the method comprising:
providing the transgenic plant cell produced by the method of claim 39; and
regenerating a transgenic plant from the transgenic plant cell, wherein expression of the ribonucleic acid molecule encoded by the at least one polynucleotide is sufficient to modulate the expression of a target gene in a coleopteran and/or hemipteran pest that contacts the transformed plant.

42. A method for producing a transgenic plant cell, the method comprising:
transforming a plant cell with a vector comprising a means for protecting a plant from coleopteran pests;
culturing the transformed plant cell under conditions sufficient to allow for development of a plant cell culture comprising a plurality of transformed plant cells;
selecting for transformed plant cells that have integrated the means for providing coleopteran pest resistance to a plant into their genomes;
screening the transformed plant cells for expression of a means for inhibiting expression of an essential gene in a coleopteran pest; and
selecting a plant cell that expresses the means for inhibiting expression of an essential gene in a coleopteran pest.

43. A method for producing a coleopteran pest-resistant transgenic plant, the method comprising:
providing the transgenic plant cell produced by the method of claim 42; and
regenerating a transgenic plant from the transgenic plant cell, wherein expression of the means for inhibiting expression of an essential gene in a coleopteran pest is sufficient to modulate the expression of a target gene in a coleopteran pest that contacts the transformed plant.

44. A method for producing a transgenic plant cell, the method comprising:
transforming a plant cell with a vector comprising a means for providing hemipteran pest resistance to a plant;
culturing the transformed plant cell under conditions sufficient to allow for development of a plant cell culture comprising a plurality of transformed plant cells;
selecting for transformed plant cells that have integrated the means for providing hemipteran pest resistance to a plant into their genomes;
screening the transformed plant cells for expression of a means for inhibiting expression of an essential gene in a hemipteran pest; and
selecting a plant cell that expresses the means for inhibiting expression of an essential gene in a hemipteran pest.

45. A method for producing a hemipteran pest-resistant transgenic plant, the method comprising:
providing the transgenic plant cell produced by the method of claim 44; and
regenerating a transgenic plant from the transgenic plant cell, wherein expression of the means for inhibiting expression of an essential gene in a hemipteran pest is sufficient to modulate the expression of a target gene in a hemipteran pest that contacts the transformed plant.

46. The nucleic acid of claim 1, further comprising a polynucleotide encoding a polypeptide from Bacillus thuringiensis.

47. The nucleic acid of claim 46, wherein the polypeptide from B. thuringiensis is selected from a group comprising Cry3, Cry34, and Cry35.

48. The cell of claim 15, wherein the cell comprises a polynucleotide encoding a polypeptide from Bacillus thuringiensis.

49. The cell of claim 48, wherein the polypeptide from B. thuringiensis is selected from a group comprising Cry3, Cry34, and Cry35.

50. The plant of claim 16, wherein the plant comprises a polynucleotide encoding a polypeptide from Bacillus thuringiensis.

51. The plant of claim 50, wherein the polypeptide from B. thuringiensis is selected from a group comprising Cry3, Cry34, and Cry35.

52. The method according to claim 39, wherein the transformed plant cell comprises a nucleotide sequence encoding a polypeptide from Bacillus thuringiensis.

53. The method according to claim 52, wherein the polypeptide from B. thuringiensis is selected from a group comprising Cry3, Cry34, and Cry35.

54. A method for improving the yield of a plant crop, the method comprising:
introducing a nucleic acid of into a corn plant to produce a transgenic plant, wherein the nucleic acid comprises more than one of
a polynucleotide encoding at least one siRNA targeting a Gho/Sec24B2 gene and/or a Sec24B1 gene,
a polynucleotide encoding an insecticidal polypeptide from Bacillus thuringiensis, and
and
cultivating the plant to allow the expression of the at least one polynucleotide; wherein expression of the at least one polynucleotide inhibits coleopteran and/or hemipteran pest development or growth and loss of yield due to coleopteran and/or hemipteran pest infection.

55. The method according to claim 54, wherein the plant is maize, soybean or cotton.


SEQUENCE LISTING (127 sequences)

SEQ ID NO: 1 - DNA, 3664 bp, Diabrotica virgifera
gatgtcaactggacctccaacattatcaaatgtgcctccaacgttatctagtgggcctccaacaagtggttctcctcaaacaggccatttaggtgctccaccaaatcaatctcccttgtctggaggagttccacctcaaatgggacctaatcaacaattaggacagccaccatcggcagctggtccaccaagccaccttggacagacttctttgactaaccccccaccccatccaggtcaaccgaatctcccctggcgcccacctcaatctgtaggtcaacctggtggccctcctggatatcctccattgccaggacatcaaggacaacccacatcacagttcgacacacaaggtccaatgtcacaaaatggacctccaaacatgtatggaaatccaccaaatcaatttaataatcagatgggtcctccaaaagtgggacaatttcctcaacaacaaaggccaatgcaacctcccctacctggacagccgcctatgccgggacaaggtcctttaatcagtgctccaggtccatacggaccttcttcaggaccagcacaccaaatgccacctcatcaaggacaaccacctcatcaaggacaatcaccatatggacctggccaaataactagtcagttgcagcaaatgaatttatctggtccaaagccggcttatccagtaccaccaggcggtcccatgagaccgatgaacggagacagcggtccgcatatgcctccagcaatgaaccaaccgggatatatgaataatcaacagggcagagttcctcctggacctggttatccaccgatgccggggcaagcaccgatgcaaggacaaggacacatgcctggtcaagggcaatacccaggacctggtggggggtatccgcaaggcaactaccaacaagctgcgccggcgcaacacaagattgatcctgatcatgtgccgaatccaattcaagttatccgagatgatcagcaagacagggacagcgtttttgttactaatcaaaaaggacttgtaccgcctatggtaactaccaattttattgttcaagatcaaggaaattgcagtccacgattcatgagatctaccatatataatgttccaatttcacaggatttgttaaaacaatctgcacttccattcagtcttttaataagtccaatggccaggcaagtagagcaagaataccctccaccaatcgttaatttcggaagcctcggtcctgtcagatgcatccgttgcaaggcctacatgtgtccgttcatgcagttcgtcgattctggaaggaggttccagtgtctgttttgtaacgcaactactgatgttccaacagaatatttccagcatctagatcagaccggcctaagaatggaccgctttgaacgaccagaattgatccttggtacctacgaattcgtcgctacccccgattactgccgaaacaacgttctgcccaaaccgccagccgtcattttcgttatcgacgtttcatataacaacattaaatccggaatggtttccttgttgtgcaatcagatgaaagagatcattcaaaatcttccggtggaccaaggccacgaaaagagcaacatgaaagttggatttattacgtataatagttcggtgcatttttataatatcaagggaagtttgacagctccacaaatgttggtggtaggagatgtccaagaaatgttcatgcctttgttggatggtttcttatgtactccagaagaatcgggacccgtaatagatctactcatgcaacagattcccgcaatgtttgcagatactaaggaaaccgaagtcgttttgcttcccgcaattcaagctggattagaagccctaaaggcttccgaaagtacaggcaaacttctagtattccactccactttaccaatagcagaggctccaggtaaattgaagaaccgcgacgatagaaaagtcttaggaaccgataaagaaaaaactgtcttgacaccacaaacacaagcatacaaccaattgggccaggaatgcgtcagcaacggttgctccgttgatatgtatatcttcaataacgcttacatcgatatagcgactattggtcaagtgtctagattgacgggaggagaagtgtttaagtatacttatttccaggctgatattgatggagaacgtttcataacagacgttatcttaaatattagtcgaccaatagcgtttgatgctgtaatgagggttagaacgtcaacaggagtgaggcccactgacttttatggtcatttctacatgtcaaatactacggatatcgaactagcggcagtagattgcgataaagccatagcagtcgaaataaaacacgacgacaaactgaatgaagacacgggggtattcattcaaacggcgctgttatacacatcgtgctcaggacagcgacggttgcgaattatgaatctttcactgaagacttgctcacaaatggccgatctctttagaagttgtgatttagatactttaatcaattacatgagtaaacaggctacgtataaattattggacggcagccccagcgttgtaaaggagggacttgtccatagagccgctcagatcttagcaatatacaggaagcactgcgcaagtccaagtagcgcgggtcaactaattcttcccgaatgcatgaagctgctaccgatctacaccaattgtcttctcaagaacgacgctatctcaggaggttcggatatgaccatcgacgacaaatcgttcgtcatgcaggtggtcttgagcatggaccttaacttctcggtgtactatttctatcctaggttaattccactacacgatatcgatcccaaccaggatcctatcacagttccgaatcctatgaggtgtagttatgataaaatgaatgaacagggagtgtatatattagaaaacggaatccatatgttcttatggtttggtctcggcgtgaatcccaactttattcagcaactctttggtgcgccttcagcaatacaagttgatatcgataggagtagtttgccggaattagataacccattgtcggtagcagttaggacaataatagacgaaatcaggatacagaaacataggtgtatgaggttaaccctggttagacaaagagaaaaactggaaccagtcttcaagcatttcttagtagaggaccgcggcacagacggttcagccagctatgtcgacttcctatgtcatatgcacagagaaatcagaaacatcctcagctagcacagaaggtgatccaaaggcagacggaagataagatgatagaaaatcttgaaatttgtactctgatcctcgataacatatttcctcttgtataaagtattattaagatctatttttgtatagcgcatgcgtttgtaaagggtgccagacggtgttcttttggatttctagatattctattatattatgcattattttggggtctagcttgtcggtgcttttacatattaaagaaaatcagtttgtttccgtatgctcaggaaacaaacaacgcttttttttctattttattggttattacacgtcgacagaactatctgaaaggtcagatcgaaaactttcgttacgcgacgttgtcagattaatcgaagtttaaaggttttccggtttttatttgttacctgtttcaca

SEQ ID NO: 2 - protein, 1083 aa, Diabrotica virgifera
MSTGPPTLSNVPPTLSSGPPTSGSPQTGHLGAPPNQSPLSGGVPPQMGPNQQLGQPPSAAGPPSHLGQTSLTNPPPHPGQPNLPWRPPQSVGQPGGPPGYPPLPGHQGQPTSQFDTQGPMSQNGPPNMYGNPPNQFNNQMGPPKVGQFPQQQRPMQPPLPGQPPMPGQGPLISAPGPYGPSSGPAHQMPPHQGQPPHQGQSPYGPGQITSQLQQMNLSGPKPAYPVPPGGPMRPMNGDSGPHMPPAMNQPGYMNNQQGRVPPGPGYPPMPGQAPMQGQGHMPGQGQYPGPGGGYPQGNYQQAAPAQHKIDPDHVPNPIQVIRDDQQDRDSVFVTNQKGLVPPMVTTNFIVQDQGNCSPRFMRSTIYNVPISQDLLKQSALPFSLLISPMARQVEQEYPPPIVNFGSLGPVRCIRCKAYMCPFMQFVDSGRRFQCLFCNATTDVPTEYFQHLDQTGLRMDRFERPELILGTYEFVATPDYCRNNVLPKPPAVIFVIDVSYNNIKSGMVSLLCNQMKEIIQNLPVDQGHEKSNMKVGFITYNSSVHFYNIKGSLTAPQMLVVGDVQEMFMPLLDGFLCTPEESGPVIDLLMQQIPAMFADTKETEVVLLPAIQAGLEALKASESTGKLLVFHSTLPIAEAPGKLKNRDDRKVLGTDKEKTVLTPQTQAYNQLGQECVSNGCSVDMYIFNNAYIDIATIGQVSRLTGGEVFKYTYFQADIDGERFITDVILNISRPIAFDAVMRVRTSTGVRPTDFYGHFYMSNTTDIELAAVDCDKAIAVEIKHDDKLNEDTGVFIQTALLYTSCSGQRRLRIMNLSLKTCSQMADLFRSCDLDTLINYMSKQATYKLLDGSPSVVKEGLVHRAAQILAIYRKHCASPSSAGQLILPECMKLLPIYTNCLLKNDAISGGSDMTIDDKSFVMQVVLSMDLNFSVYYFYPRLIPLHDIDPNQDPITVPNPMRCSYDKMNEQGVYILENGIHMFLWFGLGVNPNFIQQLFGAPSAIQVDIDRSSLPELDNPLSVAVRTIIDEIRIQKHRCMRLTLVRQREKLEPVFKHFLVEDRGTDGSASYVDFLCHMHREIRNILS

SEQ ID NO: 3 - DNA, 320 bp, Diabrotica virgifera
tatatcttcaataacgcttacatcgatatagcgactattggtcaagtgtctagattgacgggaggagaagtgtttaagtatacttatttccaggctgatattgatggagaacgtttcataacagacgttatcttaaatattagtcgaccaatagcgtttgatgctgtaatgagggttagaacgtcaacaggagtgaggcccactgacttttatggtcatttctacatgtcaaatactacggatatcgaactagcggcagtagattgcgataaagccatagcagtcgaaataaaacacgacgacaaactgaatgaagacac

SEQ ID NO: 4 - DNA, 418 bp, Diabrotica virgifera
ctaaggaaaccgaagtcgttttgcttcccgcaattcaagctggattagaagccctaaaggcttccgaaagtacaggcaaacttctagtattccactccactttaccaatagcagaggctccaggtaaattgaagaaccgcgacgatagaaaagtcttaggaaccgataaagaaaaaactgtcttgacaccacaaacacaagcatacaaccaattgggccaggaatgcgtcagcaacggttgctccgttgatatgtatatcttcaataacgcttacatcgatatagcgactattggtcaagtgtctagattgacgggaggagaagtgtttaagtatacttatttccaggctgatattgatggagaacgtttcataacagacgttatcttaaatattagtcgaccaatagcgtttgatgc

SEQ ID NO: 5 - DNA, 287 bp, Diabrotica virgifera
tcgttttgcttcccgcaattcaagctggattagaagccctaaaggcttccgaaagtacaggcaaacttctagtattccactccactttaccaatagcagaggctccaggtaaattgaagaaccgcgacgatagaaaagtcttaggaaccgataaagaaaaaactgtcttgacaccacaaacacaagcatacaaccaattgggccaggaatgcgtcagcaacggttgctccgttgatatgtatatcttcaataacgcttacatcgatatagcgactattggtcaagtg

SEQ ID NO: 6 - DNA, 128 bp, Diabrotica virgifera
gtcgttttgcttcccgcaattcaagctggattagaagccctaaaggcttccgaaagtacaggcaaacttctagtattccactccactttaccaatagcagaggctccaggtaaattgaagaaccgcga

SEQ ID NO: 7 - DNA, 24 bp, Artificial Sequence
  T7 promoter oligonucleotide
ttaatacgactcactatagggaga

SEQ ID NO: 8 - DNA, 503 bp, Artificial Sequence
  YFP partial coding region
caccatgggctccagcggcgccctgctgttccacggcaagatcccctacgtggtggagatggagggcaatgtggatggccacaccttcagcatccgcggcaagggctacggcgatgccagcgtgggcaaggtggatgcccagttcatctgcaccaccggcgatgtgcccgtgccctggagcaccctggtgaccaccctgacctacggcgcccagtgcttcgccaagtacggccccgagctgaaggatttctacaagagctgcatgcccgatggctacgtgcaggagcgcaccatcaccttcgagggcgatggcaatttcaagacccgcgccgaggtgaccttcgagaatggcagcgtgtacaatcgcgtgaagctgaatggccagggcttcaagaaggatggccacgtgctgggcaagaatctggagttcaatttcaccccccactgcctgtacatctggggcgatcaggccaatcacggcctgaagagcgccttcaagatct

SEQ ID NO: 9 - DNA, 376 bp, Artificial Sequence
  GFP partial coding region
gggagtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaccca

SEQ ID NO: 10 - DNA, 45 bp, Artificial Sequence
  Primer sec24BT7_F
ttaatacgactcactatagggagatatatcttcaataacgcttac

SEQ ID NO: 11 - DNA, 42 bp, Artificial Sequence
  Primer sec24BT7_R
ttaatacgactcactatagggagagtgtcttcattcagtttg

SEQ ID NO: 12 - DNA, 48 bp, Artificial Sequence
  Primer gho-2F
ttaatacgactcactatagggagactaaggaaaccgaagtcgttttgc

SEQ ID NO: 13 - DNA, 46 bp, Artificial Sequence
  Primer gho-2R
ttaatacgactcactatagggagagcatcaaacgctattggtcgac

SEQ ID NO: 14 - DNA, 45 bp, Artificial Sequence
  Primer Gho_v1F
ttaatacgactcactatagggagatcgttttgcttcccgcaattc

SEQ ID NO: 15 - DNA, 49 bp, Artificial Sequence
  Primer Gho_v1R
ttaatacgactcactatagggagacacttgaccaatagtcgctatatcg

SEQ ID NO: 16 - DNA, 46 bp, Artificial Sequence
  Primer Gho_v2F
ttaatacgactcactatagggagagtcgttttgcttcccgcaattc

SEQ ID NO: 17 - DNA, 46 bp, Artificial Sequence
  Primer Gho_v2R
ttaatacgactcactatagggagatcgcggttcttcaatttacctg

SEQ ID NO: 18 - DNA, 839 bp, Artificial Sequence
  DNA encoding Sec24B2 v1 hpRNA
tcgttttgcttcccgcaattcaagctggattagaagccctaaaggcttccgaaagtacaggcaaacttctagtattccactccactttaccaatagcagaggctccaggtaaattgaagaaccgcgacgatagaaaagtcttaggaaccgataaagaaaaaactgtcttgacaccacaaacacaagcatacaaccaattgggccaggaatgcgtcagcaacggttgctccgttgatatgtatatcttcaataacgcttacatcgatatagcgactattggtcaagtggaatccttgcgtcatttggtgactagtaccggttgggaaaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggttgatccgcggttaagttgtgcgtgagtccattgcacttgaccaatagtcgctatatcgatgtaagcgttattgaagatatacatatcaacggagcaaccgttgctgacgcattcctggcccaattggttgtatgcttgtgtttgtggtgtcaagacagttttttctttatcggttcctaagacttttctatcgtcgcggttcttcaatttacctggagcctctgctattggtaaagtggagtggaatactagaagtttgcctgtactttcggaagcctttagggcttctaatccagcttgaattgcgggaagcaaaacga

SEQ ID NO: 19 - DNA, 521 bp, Artificial Sequence
  DNA encoding Sec24B2 v2 hpRNA
gtcgttttgcttcccgcaattcaagctggattagaagccctaaaggcttccgaaagtacaggcaaacttctagtattccactccactttaccaatagcagaggctccaggtaaattgaagaaccgcgagaatccttgcgtcatttggtgactagtaccggttgggaaaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggttgatccgcggttaagttgtgcgtgagtccattgtcgcggttcttcaatttacctggagcctctgctattggtaaagtggagtggaatactagaagtttgcctgtactttcggaagcctttagggcttctaatccagcttgaattgcgggaagcaaaacgac

SEQ ID NO: 20 - DNA, 471 bp, Artificial Sequence
  DNA encoding YFP v2 hpRNA
atgtcatctggagcacttctctttcatgggaagattccttacgttgtggagatggaagggaatgttgatggccacacctttagcatacgtgggaaaggctacggagatgcctcagtgggaaaggactagtaccggttgggaaaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggttgatccgcggttactttcccactgaggcatctccgtagcctttcccacgtatgctaaaggtgtggccatcaacattcccttccatctccacaacgtaaggaatcttcccatgaaagagaagtgctccagatgacat

SEQ ID NO: 21 - DNA, 225 bp, Solanum tuberosum
gactagtaccggttgggaaaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggttgatccgcggtta

SEQ ID NO: 22 - DNA, 705 bp, Artificial Sequence
  YFP gene
atgtcatctggagcacttctctttcatgggaagattccttacgttgtggagatggaagggaatgttgatggccacacctttagcatacgtgggaaaggctacggagatgcctcagtgggaaaggttgatgcacagttcatctgcacaactggtgatgttcctgtgccttggagcacacttgtcaccactctcacctatggagcacagtgctttgccaagtatggtccagagttgaaggacttctacaagtcctgtatgccagatggctatgtgcaagagcgcacaatcacctttgaaggagatggcaacttcaagactagggctgaagtcacctttgagaatgggtctgtctacaatagggtcaaactcaatggtcaaggcttcaagaaagatggtcatgtgttgggaaagaacttggagttcaacttcactccccactgcctctacatctggggtgaccaagccaaccacggtctcaagtcagccttcaagatctgtcatgagattactggcagcaaaggcgacttcatagtggctgaccacacccagatgaacactcccattggtggaggtccagttcatgttccagagtatcatcacatgtcttaccatgtgaaactttccaaagatgtgacagaccacagagacaacatgtccttgaaagaaactgtcagagctgttgactgtcgcaagacctacctttga

SEQ ID NO: 23 - DNA, 218 bp, Diabrotica virgifera
tagctctgatgacagagcccatcgagtttcaagccaaacagttgcataaagctatcagcggattgggaactgatgaaagtacaatmgtmgaaattttaagtgtmcacaacaacgatgagattataagaatttcccaggcctatgaaggattgtaccaacgmtcattggaatctgatatcaaaggagatacctcaggaacattaaaaaagaattattag

SEQ ID NO: 24 - DNA, 424 bp, Diabrotica virgifera
  misc_feature (393)..(395): n is a, c, g, or t
ttgttacaagctggagaacttctctttgctggaaccgaagagtcagtatttaatgctgtattctgtcaaagaaataaaccacaattgaatttgatattcgacaaatatgaagaaattgttgggcatcccattgaaaaagccattgaaaacgagttttcaggaaatgctaaacaagccatgttacaccttatccagagcgtaagagatcaagttgcatatttggtaaccaggctgcatgattcaatggcaggcgtcggtactgacgatagaactttaatcagaattgttgtttcgagatctgaaatcgatctagaggaaatcaaacaatgctatgaagaaatctacagtaaaaccttggctgataggatagcggatgacacatctggcgactannnaaaagccttattagccgttgttggttaag

SEQ ID NO: 25 - DNA, 397 bp, Diabrotica virgifera
agatgttggctgcatctagagaattacacaagttcttccatgattgcaaggatgtactgagcagaatagtggaaaaacaggtatccatgtctgatgaattgggaagggacgcaggagctgtcaatgcccttcaacgcaaacaccagaacttcctccaagacctacaaacactccaatcgaacgtccaacaaatccaagaagaatcagctaaacttcaagctagctatgccggtgatagagctaaagaaatcaccaacagggagcaggaagtggtagcagcctgggcagccttgcagatcgcttgcgatcagagacacggaaaattgagcgatactggtgatctattcaaattctttaacttggtacgaacgttgatgcagtggatggacgaatggac

SEQ ID NO: 26 - DNA, 490 bp, Diabrotica virgifera
gcagatgaacaccagcgagaaaccaagagatgttagtggtgttgaattgttgatgaacaaccatcagacactcaaggctgagatcgaagccagagaagacaactttacggcttgtatttctttaggaaaggaattgttgagccgtaatcactatgctagtgctgatattaaggataaattggtcgcgttgacgaatcaaaggaatgctgtactacagaggtgggaagaaagatgggagaacttgcaactcatcctcgaggtataccaattcgccagagatgcggccgtcgccgaagcatggttgatcgcacaagaaccttacttgatgagccaagaactaggacacaccattgacgacgttgaaaacttgataaagaaacacgaagcgttcgaaaaatcggcagcggcgcaagaagagagattcagtgctttggagagactgacgacgttcgaattgagagaaataaagaggaaacaagaagctgcccag

SEQ ID NO: 27 - DNA, 330 bp, Diabrotica virgifera
agtgaaatgttagcaaatataacatccaagtttcgtaattgtacttgctcagttagaaaatattctgtagtttcactatcttcaaccgaaaatagaataaatgtagaacctcgcgaacttgcctttcctccaaaatatcaagaacctcgacaagtttggttggagagtttagatacgatagacgacaaaaaattgggtattcttgagctgcatcctgatgtttttgctactaatccaagaatagatattatacatcaaaatgttagatggcaaagtttatatagatatgtaagctatgctcatacaaagtcaagatttgaagtgagaggt

SEQ ID NO: 28 - DNA, 320 bp, Diabrotica virgifera
caaagtcaagatttgaagtgagaggtggaggtcgaaaaccgtggccgcaaaagggattgggacgtgctcgacatggttcaattagaagtccactttggagaggtggaggagttgttcatggaccaaaatctccaacccctcatttttacatgattccattctacacccgtttgctgggtttgactagcgcactttcagtaaaatttgcccaagatgacttgcacgttgtggatagtctagatctgccaactgacgaacaaagttatatagaagagctggtcaaaagccgcttttgggggtccttcttgttttatttgtag

SEQ ID NO: 29 - DNA, 43 bp, Artificial Sequence
  GFP primer
ttaatacgactcactatagggaggtgatgctacatacggaaag

SEQ ID NO: 30 - DNA, 39 bp, Artificial Sequence
  GFP primer
ttaatacgactcactatagggttgtttgtctgccgtgat

SEQ ID NO: 31 - DNA, 47 bp, Artificial Sequence
  Primer YFP-F_T7
ttaatacgactcactatagggagacaccatgggctccagcggcgccc

SEQ ID NO: 32 - DNA, 23 bp, Artificial Sequence
  Primer YFP-R
agatcttgaaggcgctcttcagg

SEQ ID NO: 33 - DNA, 23 bp, Artificial Sequence
  Primer YFP-F
caccatgggctccagcggcgccc

SEQ ID NO: 34 - DNA, 47 bp, Artificial Sequence
  Primer YFP-R_T7
ttaatacgactcactatagggagaagatcttgaaggcgctcttcagg

SEQ ID NO: 35 - DNA, 46 bp, Artificial Sequence
  Primer Ann-F1_T7
ttaatacgactcactatagggagagctccaacagtggttccttatc

SEQ ID NO: 36 - DNA, 29 bp, Artificial Sequence
  Primer Ann-R1
ctaataattcttttttaatgttcctgagg

SEQ ID NO: 37 - DNA, 22 bp, Artificial Sequence
  Primer Ann-F1
gctccaacagtggttccttatc

SEQ ID NO: 38 - DNA, 53 bp, Artificial Sequence
  Primer Ann-R1_T7
ttaatacgactcactatagggagactaataattcttttttaatgttcctgagg

SEQ ID NO: 39 - DNA, 48 bp, Artificial Sequence
  Primer Ann-F2_T7
ttaatacgactcactatagggagattgttacaagctggagaacttctc

SEQ ID NO: 40 - DNA, 24 bp, Artificial Sequence
  Primer Ann-R2
cttaaccaacaacggctaataagg

SEQ ID NO: 41 - DNA, 24 bp, Artificial Sequence
  Primer Ann-F2
ttgttacaagctggagaacttctc

SEQ ID NO: 42 - DNA, 48 bp, Artificial Sequence
  Primer Ann-R2_T7
ttaatacgactcactatagggagacttaaccaacaacggctaataagg

SEQ ID NO: 43 - DNA, 47 bp, Artificial Sequence
  Primer Betasp2-F1_T7
ttaatacgactcactatagggagaagatgttggctgcatctagagaa

SEQ ID NO: 44 - DNA, 22 bp, Artificial Sequence
  Primer Betasp2-R1
gtccattcgtccatccactgca

SEQ ID NO: 45 - DNA, 23 bp, Artificial Sequence
  Primer Betasp2-F1
agatgttggctgcatctagagaa

SEQ ID NO: 46 - DNA, 46 bp, Artificial Sequence
  Primer Betasp2-R1_T7
ttaatacgactcactatagggagagtccattcgtccatccactgca

SEQ ID NO: 47 - DNA, 46 bp, Artificial Sequence
  Primer Betasp2-F2_T7
ttaatacgactcactatagggagagcagatgaacaccagcgagaaa

SEQ ID NO: 48 - DNA, 22 bp, Artificial Sequence
  Primer Betasp2-R2
ctgggcagcttcttgtttcctc

SEQ ID NO: 49 - DNA, 22 bp, Artificial Sequence
  Primer Betasp2-F2
gcagatgaacaccagcgagaaa

SEQ ID NO: 50 - DNA, 46 bp, Artificial Sequence
  Primer Betasp2-R2_T7
ttaatacgactcactatagggagactgggcagcttcttgtttcctc

SEQ ID NO: 51 - DNA, 51 bp, Artificial Sequence
  Primer L4-F1_T7
ttaatacgactcactatagggagaagtgaaatgttagcaaatataacatcc

SEQ ID NO: 52 - DNA, 26 bp, Artificial Sequence
  Primer L4-R1
acctctcacttcaaatcttgactttg

SEQ ID NO: 53 - DNA, 27 bp, Artificial Sequence
  Primer L4-F1
agtgaaatgttagcaaatataacatcc

SEQ ID NO: 54 - DNA, 50 bp, Artificial Sequence
  Primer L4-R1_T7
ttaatacgactcactatagggagaacctctcacttcaaatcttgactttg

SEQ ID NO: 55 - DNA, 50 bp, Artificial Sequence
  Primer L4-F2_T7
ttaatacgactcactatagggagacaaagtcaagatttgaagtgagaggt

SEQ ID NO: 56 - DNA, 25 bp, Artificial Sequence
  Primer L4-R2
ctacaaataaaacaagaaggacccc

SEQ ID NO: 57 - DNA, 26 bp, Artificial Sequence
  Primer L4-F2
caaagtcaagatttgaagtgagaggt

SEQ ID NO: 58 - DNA, 49 bp, Artificial Sequence
  Primer L4-R2_T7
ttaatacgactcactatagggagactacaaataaaacaagaaggacccc

SEQ ID NO: 59 - DNA, 1150 bp, Zea mays
caacggggcagcactgcactgcactgcaactgcgaatttccgtcagcttggagcggtccaagcgccctgcgaagcaaactacgccgatggcttcggcggcggcgtgggagggtccgacggccgcggagctgaagacagcgggggcggaggtgattcccggcggcgtgcgagtgaaggggtgggtcatccagtcccacaaaggccctatcctcaacgccgcctctctgcaacgctttgaagatgaacttcaaacaacacatttacctgagatggtttttggagagagtttcttgtcacttcaacatacacaaactggcatcaaatttcattttaatgcgcttgatgcactcaaggcatggaagaaagaggcactgccacctgttgaggttcctgctgcagcaaaatggaagttcagaagtaagccttctgaccaggttatacttgactacgactatacatttacgacaccatattgtgggagtgatgctgtggttgtgaactctggcactccacaaacaagtttagatggatgcggcactttgtgttgggaggatactaatgatcggattgacattgttgccctttcagcaaaagaacccattcttttctacgacgaggttatcttgtatgaagatgagttagctgacaatggtatctcatttcttactgtgcgagtgagggtaatgccaactggttggtttctgcttttgcgtttttggcttagagttgatggtgtactgatgaggttgagagacactcggttacattgcctgtttggaaacggcgacggagccaagccagtggtacttcgtgagtgctgctggagggaagcaacatttgctactttgtctgcgaaaggatatccttcggactctgcagcgtacgcggacccgaaccttattgcccataagcttcctattgtgacgcagaagacccaaaagctgaaaaatcctacctgactgacacaaaggcgccctaccgcgtgtacatcatgactgtcctgtcctatcgttgccttttgtgtttgccacatgttgtggatgtacgtttctatgacgaaacaccatagtccatttcgcctgggccgaacagagatagctgattgtcatgtcacgtttgaattagaccattccttagccctttttccccc

SEQ ID NO: 60 - DNA, 22 bp, Artificial Sequence
  T20VN primer
ttttttttttttttttttttvn

SEQ ID NO: 61 - DNA, 17 bp, Artificial Sequence
  Primer StPinIIF2 TAG
gggtgacgggagagatt

SEQ ID NO: 62 - DNA, 22 bp, Artificial Sequence
  Primer StPinIIR2 TAG
cataacacacaactttgatgcc

SEQ ID NO: 63 - DNA, 21 bp, Artificial Sequence
  Primer TIPmxF
tgagggtaatgccaactggtt

SEQ ID NO: 64 - DNA, 24 bp, Artificial Sequence
  Primer TIPmxR
gcaatgtaaccgagtgtctctcaa

SEQ ID NO: 65 - DNA, 32 bp, Artificial Sequence
  HXTIP Probe
tttttggcttagagttgatggtgtactgatga

SEQ ID NO: 66 - DNA, 151 bp, Escherichia coli
gaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaag

SEQ ID NO: 67 - DNA, 69 bp, Artificial Sequence
  AAD1 coding region
tgttcggttccctctaccaagcacagaaccgtcgcttcagcaacacctcagtcaaggtgatggatgttg

SEQ ID NO: 68 - DNA, 4233 bp, Zea mays
agcctggtgtttccggaggagacagacatgatccctgccgttgctgatccgacgacgctggacggcgggggcgcgcgcaggccgttgctcccggagacggaccctcgggggcgtgctgccgccggcgccgagcagaagcggccgccggctacgccgaccgttctcaccgccgtcgtctccgccgtgctcctgctcgtcctcgtggcggtcacagtcctcgcgtcgcagcacgtcgacgggcaggctgggggcgttcccgcgggcgaagatgccgtcgtcgtcgaggtggccgcctcccgtggcgtggctgagggcgtgtcggagaagtccacggccccgctcctcggctccggcgcgctccaggacttctcctggaccaacgcgatgctggcgtggcagcgcacggcgttccacttccagccccccaagaactggatgaacggttagttggacccgtcgccatcggtgacgacgcgcggatcgtttttttcttttttcctctcgttctggctctaacttggttccgcgtttctgtcacggacgcctcgtgcacatggcgatacccgatccgccggccgcgtatatctatctacctcgaccggcttctccagatccgaacggtaagttgttggctccgatacgatcgatcacatgtgagctcggcatgctgcttttctgcgcgtgcatgcggctcctagcattccacgtccacgggtcgtgacatcaatgcacgatataatcgtatcggtacagagatattgtcccatcagctgctagctttcgcgtattgatgtcgtgacattttgcacgcaggtccgctgtatcacaagggctggtaccacctcttctaccagtggaacccggactccgcggtatggggcaacatcacctggggccacgccgtctcgcgcgacctcctccactggctgcacctaccgctggccatggtgcccgatcacccgtacgacgccaacggcgtctggtccgggtcggcgacgcgcctgcccgacggccggatcgtcatgctctacacgggctccacggcggagtcgtcggcgcaggtgcagaacctcgcggagccggccgacgcgtccgacccgctgctgcgggagtgggtcaagtcggacgccaacccggtgctggtgccgccgccgggcatcgggccgacggacttccgcgacccgacgacggcgtgtcggacgccggccggcaacgacacggcgtggcgggtcgccatcgggtccaaggaccgggaccacgcggggctggcgctggtgtaccggacggaggacttcgtgcggtacgacccggcgccggcgctgatgcacgccgtgccgggcaccggcatgtgggagtgcgtggacttctacccggtggccgcgggatcaggcgccgcggcgggcagcggggacgggctggagacgtccgcggcgccgggacccggggtgaagcacgtgctcaaggctagcctcgacgacgacaagcacgactactacgcgatcggcacctacgacccggcgacggacacctggacccccgacagcgcggaggacgacgtcgggatcggcctccggtacgactatggcaagtactacgcgtcgaagaccttctacgaccccgtccttcgccggcgggtgctctgggggtgggtcggcgagaccgacagcgagcgcgcggacatcctcaagggctgggcatccgtgcaggtacgtctcagggtttgaggctagcatggcttcaatcttgctggcatcgaatcattaatgggcagatattataacttgataatctgggttggttgtgtgtggtggggatggtgacacacgcgcggtaataatgtagctaagctggttaaggatgagtaatggggttgcgtataaacgacagctctgctaccattacttctgacacccgattgaaggagacaacagtaggggtagccggtagggttcgtcgacttgccttttcttttttcctttgttttgttgtggatcgtccaacacaaggaaaataggatcatccaacaaacatggaagtaatcccgtaaaacatttctcaaggaaccatctagctagacgagcgtggcatgatccatgcatgcacaaacactagataggtctctgcagctgtgatgttcctttacatataccaccgtccaaactgaatccggtctgaaaattgttcaagcagagaggccccgatcctcacacctgtacacgtccctgtacgcgccgtcgtggtctcccgtgatcctgccccgtcccctccacgcggccacgcctgctgcagcgctctgtacaagcgtgcaccacgtgagaatttccgtctactcgagcctagtagttagacgggaaaacgagaggaagcgcacggtccaagcacaacactttgcgcgggcccgtgacttgtctccggttggctgagggcgcgcgacagagatgtatggcgccgcggcgtgtcttgtgtcttgtcttgcctatacaccgtagtcagagactgtgtcaaagccgtccaacgacaatgagctaggaaacgggttggagagctgggttcttgccttgcctcctgtgatgtctttgccttgcatagggggcgcagtatgtagctttgcgttttacttcacgccaaaggatactgctgatcgtgaattattattattatatatatatcgaatatcgatttcgtcgctctcgtggggttttattttccagactcaaacttttcaaaaggcctgtgttttagttcttttcttccaattgagtaggcaaggcgtgtgagtgtgaccaacgcatgcatggatatcgtggtagactggtagagctgtcgttaccagcgcgatgcttgtatatgtttgcagtattttcaaatgaatgtctcagctagcgtacagttgaccaagtcgacgtggagggcgcacaacagacctctgacattattcactttttttttaccatgccgtgcacgtgcagtcaatccccaggacggtcctcctggacacgaagacgggcagcaacctgctccagtggccggtggtggaggtggagaacctccggatgagcggcaagagcttcgacggcgtcgcgctggaccgcggatccgtcgtgcccctcgacgtcggcaaggcgacgcaggtgacgccgcacgcagcctgctgcagcgaacgaactcgcgcgttgccggcccgcggccagctgacttagtttctctggctgatcgaccgtgtgcctgcgtgcgtgcagttggacatcgaggctgtgttcgaggtggacgcgtcggacgcggcgggcgtcacggaggccgacgtgacgttcaactgcagcaccagcgcaggcgcggcgggccggggcctgctcggcccgttcggccttctcgtgctggcggacgacgacttgtccgagcagaccgccgtgtacttctacctgctcaagggcacggacggcagcctccaaactttcttctgccaagacgagctcaggtatgtatgttatgacttatgaccatgcatgcatgcgcatttcttagctaggctgtgaagcttcttgttgagttgtttcacagatgcttaccgtctgctttgtttcgtatttcgactaggcatccaaggcgaacgatctggttaagagagtatacgggagcttggtccctgtgctagatggggagaatctctcggtcagaatactggtaagtttttacagcgccagccatgcatgtgttggccagccagctgctggtactttggacactcgttcttctcgcactgctcattattgcttctgatctggatgcactacaaattgaaggttgaccactccatcgtggagagctttgctcaaggcgggaggacgtgcatcacgtcgcgagtgtaccccacacgagccatctacgactccgcccgcgtcttcctcttcaacaacgccacacatgctcacgtcaaagcaaaatccgtcaagatctggcagctcaactccgcctacatccggccatatccggcaacgacgacttctctatgactaaattaagtgacggacagataggcgatattgcatacttgcatcatgaactcatttgtacaacagtgattgtttaatttatttgctgccttccttatccttcttgtgaaactatatggtacacacatgtatcattaggtctagtagtgttgttgcaaagacacttagacaccagaggttccaggagtatcagagataaggtataagagggagcagggagcag

SEQ ID NO: 69 - DNA, 20 bp, Artificial Sequence
  Primer GAAD1-F
tgttcggttccctctaccaa

SEQ ID NO: 70 - DNA, 22 bp, Artificial Sequence
  Primer GAAD1-R
caacatccatcaccttgactga

SEQ ID NO: 71 - DNA, 24 bp, Artificial Sequence
  Primer GAAD1-P (FAM)
cacagaaccgtcgcttcagcaaca

SEQ ID NO: 72 - DNA, 18 bp, Artificial Sequence
  Primer IVR1-F
tggcggacgacgacttgt

SEQ ID NO: 73 - DNA, 19 bp, Artificial Sequence
  Primer IVR1-R
aaagtttggaggctgccgt

SEQ ID NO: 74 - DNA, 26 bp, Artificial Sequence
  IVR1-P (HEX)
cgagcagaccgccgtgtacttctacc

SEQ ID NO: 75 - DNA, 19 bp, Artificial Sequence
  Primer SPC1A
cttagctggataacgccac

SEQ ID NO: 76 - DNA, 19 bp, Artificial Sequence
  Primer SPC1S
gaccgtaaggcttgatgaa

SEQ ID NO: 77 - DNA, 21 bp, Artificial Sequence
  TQSPEC (CY5) Probe
cgagattctccgcgctgtaga

SEQ ID NO: 78 - DNA, 25 bp, Artificial Sequence
  Primer ST-LS1-F
gtatgtttctgcttctacctttgat

SEQ ID NO: 79 - DNA, 29 bp, Artificial Sequence
  Primer ST-LS1-R
ccatgttttggtcatatattagaaaagtt

SEQ ID NO: 80 - DNA, 34 bp, Artificial Sequence
  Probe ST-LS1-P (FAM)
agtaatatagtatttcaagtatttttttcaaaat

SEQ ID NO: 81 - DNA, 633 bp, Diabrotica virgifera
ccagagctgtattcccttcaattgttggacgtccaagacatcagggtgtgatggtaggaatgggccaaaaagattcctatgttggcgatgaagctcaaagcaaaagaggtatccttacattaaagtaccccatcgagcatggaatagtcacaaactgggatgatatggagaaaatttggcatcatacattctacaatgaactcagagtagccccagaagaacaccccgttctgttgacggaagctcctctcaaccccaaggccaacagggaaaagatgacacaaataatgtttgaaactttcaacaccccagccatgtatgttgccatccaggctgtactctccttgtatgcatctggtcgtactactggtatcgtattggattctggtgatggtgtatcccacactgtcccaatctatgaaggttatgcacttccccatgcaatccttcgtttggacttggctggcagagatttaactgattacctcatgaaaatcttgactgaacgtggctactctttcaccaccacagcagaaagagaaattgttagggatattaaagaaaaactctgctatgtagctttggacttcgaacaagaaatggcaacggctgctagttccagttcccttgaaa

SEQ ID NO: 82 - DNA, 20 bp, Artificial Sequence
  Actin primer
tccaggctgtactctccttg

SEQ ID NO: 83 - DNA, 20 bp, Artificial Sequence
  Actin primer
caagtccaaacgaaggattg

SEQ ID NO: 84 - DNA, 4273 bp, Euschistus heros
acgtaacctcactttcttgacagcttccgccagactgtttttcatttaggctagtttgccttcgcagtcttgttatattgataaaaactttcgttaagcttagttaaaattaaagatacaacaatctcgtaagtatttacaactcgggcgaagtaaaaatgttactgtttcgctgtttggtttcatgtgtgctataaccaaagatttatcttaaggggaaaaacggtgctatttcatgcgtctcgaagcttaaactaatttaaacaagtagttttaatttaaggaacagttgagttttatatattatcttttaaatggtaccgttaatgcttacacggagcgcatcgtagtaacttgggaaaggggagtgacatataagtgtaaccgtccatatatcagacttctatttgtaatttaattaatcatttgaaagtttttaagctgattcatgttttcaaattaactaaggagccctcaactaccttttgtaattttgaataatgaacggccaatcttgcacttattctgactctggaaatggtacaccaacaccttcatccacaagctatccagctagtttatcatcacaatcttcccgtgatacatccccctcccgccttcatcctaaccttaatcatataaattctgaaaaatcaattaattcatctggtaactatatgaattataaaatacacgatacgtatacaaatgccaattctgtttatgggcaaatatattcagactcaactacacctactaacagggcaacagttcccccgtacatcagtgacactaataacgacattaatcaatctcaaagactggggcaaccgcagctccgaccttcaacaacatcatcacaaataataactagtttagggtcttcggtttctaaacctgtctatagttcatcacatttaaatcaaatatcgaatgatcagaaacagtatgttaatcaatatagcacacaaaagttagatagcgttatgcagcctaaaacatcagagagtaacatcattaaaaatcatgaaactatgcctacatctaatttagcaatatctgattattatcagggatatactcaaacgatgaataatccctacaggcaagaaaatgtattgcctaaccagacaatgaagcccgaacaacagtaccatgctcaaacccaagggtatcaagttcaaaaacccttgatgtctccaacatcaaatccatacatgaattcagtgcctcaagataaccaaaactacccccaatcaccaggtgatgtccccaggtctactttccagcagggttattatcagcatcaacctcaacctcaacctcaaccacaaccaccttcagtaatgagtggaagaccgcagatgaatttgcctttgactcagtctagatcacttgatgaacctatttcttcagggcctccaagaacaaacgtcttgggaatcattccttatgccactgaacctgctacttcgcaagtttcgaggcctaaattacccgatggtggagggtattatcagcccatgcaaccacaacagcaaccaccgcagatgcagcagccacagatgcagcaaccgcagatgcagcagcaacagccaccacgagtggcaccaagacccccagcgcctaaacctaaaggctaccctccaccaccatatcaacaatatccatcttattcccatcctcaaaacaatgctggtttacctccttacagtcaaacaatgggtggttattacccgagcggagatgaacttgctaatcagatgtcacagcttagcgtttctcaacttggttttaataaattatggggaagggatacagtggacttgatgaagagtcgtgatgttttgccccctactcgggtcgaagctcctccagttcgtctttctcaggagtactatgattcgactaaagttagccctgagatatttagatgtacgctaactaaaatacccgagaccaaatctcttcttgataaatctaggcttccccttggcgtcttgatccacccattcaaggacctaaatcaattgtcggtgatccagtgcacagtaatagtacgatgtagagcgtgtaggacttatataaatccttttgtattctttgtcgactcgaagcattggaaatgcaatctctgctttagggtgaatgatttgccagaagaatttcaatatgacccattaacaaagacttatggagaccctactagacgaccagaaataaaatctgctactatagaattcatagctccatcggaatatatggtgaggccgccgcaaccggctgcttacgtgtttgtattagacgtgtcaagactagcggtcgagagtggttacttgcgtatcttctgtgactgcctcctttcccagctggaggcgttgccaggcgattcgaggacagctgtggcttttatcacctacgactctgctgtccactattatagccttgctgatacccaggctcagccacatcagatggtcgtagtggacattgatgatatgttcgtaccatgccctgaaaacctgctggtgaacctgagtgagtgcctggggctagtacgggaccttctgcgggaactgcctaataagtatagagattcctatgacacaggcactgccgtcggtcctgctttacaagcagcttacaaattattggccgcaactggtggaagagtgactttggtaacgagctgcttggcgaacagcggaccaggaaaactgccatctcgagaggacccgaaccagaggagcggggaagggttgaaccagtcacatctcaacccagtcactgacttctacaagaaattggccctcgattgctcaggccaacagattgctgtcgatcttttcgtacttaacagtcaatttgttgaccttgcttctctgagtggtgtttcgaggttttccggtgggtgtatccatcatttccctctgttctctgtgaagaaccctcatcatgttgaatcattccagcgtagtctacagaggtatctgtgtcgtaagattggttttgaatctgtcatgaggttgcgctgcaccagggggttatctattcatacattccatggaaacttctttgttcgttcaacggacctcctctctctacccaatgtaaacccagatgctggtttcggaatgcaggtgtctattgacgagaacctgactgatatacagaccgtatgtttccaagcagcacttctgtatacttcgagtaaaggagaaagaagaatccgtgttcacactttgtgccttccaatagcttctaacctttcagacgttctgcatggagcagaccagcaatgtatcgtaggtcttctggctaagatggctgttgataggtgtcatcagtcgtcgctgagtgatgcaagggaggcttttgtgaacgtagttgctgatatgttatcagcgttccggatcacccagtctggcgtatcacctacctcactagtcgctcccattagtctctcccttcttccactctatgtactcgctttgctcaaatatattgctttccgtgtcggccagagcacaaggctggacgatcgagtcttcgctatgtgccaaatgaagtctctacctctctctcagttaatacaggccatttaccctgatctctatccaatagccaatatcaacgaattgccacttgttactattggagaagaccaagtagtccaaccaccattacttcacctctcagctgaaagaatagactcgacgggggtctacttgatggatgatggaacaacaataattatctacgtcggccacaacattaatccatcaattgctgtttccttcttcggggtaccttcattttcagctataaattctaatatgtttgaactacctgaactgaatacgccggagtctaaaaaactgagaggtttcattagctatttacagaatgagaagcccgtagctccgactgtactcatcattagggatgacagccagcagagacatttatttgtcgagaagctcatagaagacaaaactgaatccggtcattcttactacgaatttttgcagagagtgaaggtactcgttaagtaacaaacagctgagatattctcactctataccaatctaccaaagactatgtcgtgtgttgatggggcatggcaacacatcttatgtccattatagatttctaacttttttatattttctgcttcttattcgtcgtaatgagaagttttaattgatgtttcatcaactacaaaacttttatcctgtataacacatcattttatatagtattatatatataa

SEQ ID NO: 85 - DNA, 4809 bp, Euschistus heros
atggaataaaatttttatttacagaaaataatcatcaacattatctacaaatttattttctataatttatatataataacacattaccaaacaaaaataacatatcgtagttataacaattgtttatatataaatacatacacatgtcacaccatacaccgcataaccttcgaactcggctacacaagatcttaaggagcgcacaacataaatacaacataaagcaaagtatcaatgtaaataagggaaacttaggtacaagtgtctgttcatggggaacatatatatctatatatgatataacaattattagtgttaaaaataatatttaattaaaataatatttactggcaacatataataaaaatatttgattacataaattacctagataaagcaacagcttgatataatcctcgttaaacatatactgcacgcagttggttcttttataatgtactgtaggaaattttgatacataaaaaaaaaaaaaaaataatggaaagaagaagaaaagtgcactggtggcaagtttaatttgacaagttggaagtatacgtatcatacgccattttttatctttagatagtaagtactcagatgcactatcaataacttttgctaatatttttaaaatttttattttttaagtccaattcacgtagatatatttatgtacagtttaataaatttcctccctctgtaaaaaataaaataaaacaaaatataaccaatgatataaacaaattttgataattaaatttaaaacaataatattaatcacatcccacattttaaaggaagtagaaagaaaacaatacattatttatgatacaatcccgttataatatacatcatcaaacaaacagttgtaagcttacccgttaaatgagaaactgttacttaataataatgaattataacaatttcatcagctataaaaatatcaaatcgaaatttcatacaattgaaggataatgataaattttacaggttcgataggaaatgtcaagccaacaattggcagtcgtaatctgcataatagtctgctgtggaggtcgctaactaagcatattacgaatttctttgtgaagatgacatagaaaatctacataagatgaagatccatccaaacctctgtcctcgaccaagaagtgcttcatcaccatttccattttgtcccgttgtctcactattgtcagcctcattgtcctatgattgctgtcagcaattgatgagattgcattcctgactctttctgaaatcgggttttcaaggggtggtaatctatgtctatcggtatcgacctgagctgcacttggaactccaaatactgacatcacccaatctgaaggagtagctagacccagccagatgaacatgtaaataccgtttactagtaaatatactccactatccaccattttttcagatgaacatcttatgcacggtggtggtacagaatcctctagctctaatagagaatataaccgtgggtagaagtatacaagagaagaaggaacatccatcgtcagaactgcagccatcacaaaccatttgtcgtcaactgtcatgtctttgcctccagagatagcatcacttttcaagaggcagttgacatacagaggtaacaacttcatgcactcaggaaggatcagctgtccagcagaagtaggagaagcacaattcttacgatagcacgccagaatctgagctgacctgtttattaatgattctttaacagcttttgccgatgcatctaaaagcttgaacacactctgtttggaaaagaagttgatgatagtgtcgagttcacaggttctatagaggtcggacatctgtgagcaagccttcaataccaggttgagaactctgatcctccgctgtcctgacagcgaagtatacaacaatgcgacttggatatatacaccttcttcttcagaaagtttgtcatcatgcttaatctcgacagctattcccttgtctggatctatagaggcaagttcaacatctgtggtattcgacatgtagaaatgtccatagaaatcagtcggtcgaatacccgttgatgtcctaactctcataatagcatcaaaagcgcaaagcctcctgatatttttctcaacatcagctacaagcctctctccatctagttcagcctggaagtatgtatacttgtaaatttctccaccagtgagccttgaaacttgaccgatagttgccaggtcaatataggaattgttagtaataaataaatcaacgctcactccagcaccaacacagtcctgtcccaaggtgttgtaaacagtgttctgtggcaataaaattgtcttttctttatcagtccccaataacgacctgtcatccctatttttcaactttccaggagcttctgcgataggaagagacgagtggaacacgagcagtttaccagcgcacccagacgctttaagagcttcaaggccggcctgtatagcaggagccagtattgtttctgtctcacgggtgtcagcaaacatcatcggtatattcgtcattagtgcgtctattaaaccttcagactcttcaggatcgaccaggaaaccgtccaatagaggcatgaacatttcttgagtatcaccgactactaacatctggggttgtcctaggttaggtctaatattgtagaaatggacagcactgttataagttataaatccaactttcatagtagacttctccattcccctttctttaggaagattgcgaagaatatttttcatttgatgacataacagtgaaacgagtccagatttaacattattgtaagacacatcaataacgaatataagtgcaggtggattagggaattgattgtctttacaatattctcttgttgctataatatcataggtccctaacacaagttcagctctttcaaaacgatcaactcgttgaccagtatggtctaaatgctggaagtattcagctggtacatcagtagttgctttgcatagaagacagtggaagcgcctaccaccatcaatgaactgcatgttcgggcacatataagccttgcaacgaatacatcttactggaccgagctcgccaaaagaaaccaacggaggaggatgttctttatctgcgacttccgccataggactcaacaccaaaccaaaaggtacagacgcctgtttcatcaaatcagaagttataggaacgttgtacatcgttgacctcataaaccttggactggcattgccctgatcttgaacgacgaattccgtagtaacaagtggagggacttggcctttctggtgtgtataaaacacgcctgatcttgtcttctggtcatcttccattacctgcattggactaggcatctggtctgggtcaagcctgcgaggttgctgttgaggatactgcggctgcccaactccaccaggataaccaggttgaggctgcggtgggaaaccaggttgagggggatagccctgctgcggcgatggaaggtatgcagaagtttgtcctcctgattcaggcattggaggatatctggattgaggaggcctgccaggaccacttgtatcaggaaggccattcatcgcctgactgggtgggccaccattcacggctgggtatcgagatggttgtccaggaggagcataccccatagatggcggaccttgcaaaccacctccaggatagtccccttggtgttgctgattcattggtggcatcggctgcccatttatgttcatgctggacatctgccctgccagctggttcacctgaggcattggtggcctgccgatctgctgagaacctggaggatacatggatgggtgtggtggaccaccaggaccgggagagctgacaggaccaggcatcgaaggggctcctacagcaggtggtgctcctgggtgcgaaggtgctgagttatatcctagaggcacattactagatggtggttgaaagctgttaggcataggagcaccgcgctgttgaggaggcattggaccaccatggtgttggtgtggcaaagaaccaggatgctgttgaggtggcatttgaccactctgttgaggtggcacagaaccaactggtttttgaggttgcattggactaagctgttgttgaggtggcatgggaccaccaggctgttgaggaggcatagaactattctgctgctgaaggggctttggaccaccatagtgttgagacatcattggaccaccctgctgttgaggaggggctggaccgccatgctgtggaggaaccattggaccactgtgttgctgagggggcaccggaccgccctgtagtggaggaggtggcatcatgttggcaggggaagtcccagctggtcggtaaggttgagaaaatgctgatggtgatgccatatttgttttagaaggaatacctggataactttgctgtggtggaaaagcattaggttgaagagggcttgcagctggtggcggaggcgaatttggaacaccataaccagtatgaggtccataaccacctggttgtgatacatactgaggattcatcttgtaagtcttgccttcacttatatggaatctaaaacttaataatcttcataattttaacaaaacaaaaaaaaacacgaaactaaataatataagctactaatatcagctgcagtagcaccactccactacccctgccacgtaaggcagaactgcacaggcgcagtaagattacacgtcaagaaatcttcagcgctaccccttgtggtggtctacaatacaactaggttatcctaatcaaaatcagtgctactctagtgaaaactaatttcag

SEQ ID NO: 86 - DNA, 397 bp, Euschistus heros
gattcgactaaagttagccctgagatatttagatgtacgctaactaaaatacccgagaccaaatctcttcttgataaatctaggcttccccttggcgtcttgatccacccattcaaggacctaaatcaattgtcggtgatccagtgcacagtaatagtacgatgtagagcgtgtaggacttatataaatccttttgtattctttgtcgactcgaagcattggaaatgcaatctctgctttagggtgaatgatttgccagaagaatttcaatatgacccattaacaaagacttatggagaccctactagacgaccagaaataaaatctgctactatagaattcatagctccatcggaatatatggtgaggccgccgcaaccggctgcttacgtgtttg

SEQ ID NO: 87 - DNA, 494 bp, Euschistus heros
cttttcaagaggcagttgacatacagaggtaacaacttcatgcactcaggaaggatcagctgtccagcagaagtaggagaagcacaattcttacgatagcacgccagaatctgagctgacctgtttattaatgattctttaacagcttttgccgatgcatctaaaagcttgaacacactctgtttggaaaagaagttgatgatagtgtcgagttcacaggttctatagaggtcggacatctgtgagcaagccttcaataccaggttgagaactctgatcctccgctgtcctgacagcgaagtatacaacaatgcgacttggatatatacaccttcttcttcagaaagtttgtcatcatgcttaatctcgacagctattcccttgtctggatctatagaggcaagttcaacatctgtggtattcgacatgtagaaatgtccatagaaatcagtcggtcgaatacccgttgatgtcctaactctcataatagcatc

SEQ ID NO: 88 - DNA, 485 bp, Euschistus heros
ggactggcattgccctgatcttgaacgacgaattccgtagtaacaagtggagggacttggcctttctggtgtgtataaaacacgcctgatcttgtcttctggtcatcttccattacctgcattggactaggcatctggtctgggtcaagcctgcgaggttgctgttgaggatactgcggctgcccaactccaccaggataaccaggttgaggctgcggtgggaaaccaggttgagggggatagccctgctgcggcgatggaaggtatgcagaagtttgtcctcctgattcaggcattggaggatatctggattgaggaggcctgccaggaccacttgtatcaggaaggccattcatcgcctgactgggtgggccaccattcacggctgggtatcgagatggttgtccaggaggagcataccccatagatggcggaccttgcaaaccacctccaggatagtccccttggtgttgctgattcattgg

SEQ ID NO: 89 - DNA, 48 bp, Artificial Sequence
  Primer BSB_Gho-1-For
ttaatacgactcactatagggagagattcgactaaagttagccctgag

SEQ ID NO: 90 - DNA, 46 bp, Artificial Sequence
  Primer BSB_Gho-1-Rev
ttaatacgactcactatagggagacaaacacgtaagcagccggttg

SEQ ID NO: 91 - DNA, 48 bp, Artificial Sequence
  Primer BSB_Gho-2-For
ttaatacgactcactatagggagacttttcaagaggcagttgacatac

SEQ ID NO: 92 - DNA, 50 bp, Artificial Sequence
  Primer BSB_Gho-2-Rev
ttaatacgactcactatagggagagatgctattatgagagttaggacatc

SEQ ID NO: 93 - DNA, 44 bp, Artificial Sequence
  Primer BSB_Gho-3-For
ttaatacgactcactatagggagaggactggcattgccctgatc

SEQ ID NO: 94 - DNA, 46 bp, Artificial Sequence
  Primer BSB_Gho-3-Rev
ttaatacgactcactatagggagaccaatgaatcagcaacaccaag

SEQ ID NO: 95 - DNA, 301 bp, Artificial Sequence
  YFPv2 gene
catctggagcacttctctttcatgggaagattccttacgttgtggagatggaagggaatgttgatggccacacctttagcatacgtgggaaaggctacggagatgcctcagtgggaaaggttgatgcacagttcatctgcacaactggtgatgttcctgtgccttggagcacacttgtcaccactctcacctatggagcacagtgctttgccaagtatggtccagagttgaaggacttctacaagtcctgtatgccagatggctatgtgcaagagcgcacaatcacctttgaaggagatgg

SEQ ID NO: 96 - DNA, 47 bp, Artificial Sequence
  Primer YFPv2-F
ttaatacgactcactatagggagagcatctggagcacttctctttca

SEQ ID NO: 97 - DNA, 46 bp, Artificial Sequence
  Primer YFPv2-R
ttaatacgactcactatagggagaccatctccttcaaaggtgattg

SEQ ID NO: 98 - protein, 1184 aa, Euschistus heros
MNGQSCTYSDSGNGTPTPSSTSYPASLSSQSSRDTSPSRLHPNLNHINSEKSINSSGNYMNYKIHDTYTNANSVYGQIYSDSTTPTNRATVPPYISDTNNDINQSQRLGQPQLRPSTTSSQIITSLGSSVSKPVYSSSHLNQISNDQKQYVNQYSTQKLDSVMQPKTSESNIIKNHETMPTSNLAISDYYQGYTQTMNNPYRQENVLPNQTMKPEQQYHAQTQGYQVQKPLMSPTSNPYMNSVPQDNQNYPQSPGDVPRSTFQQGYYQHQPQPQPQPQPPSVMSGRPQMNLPLTQSRSLDEPISSGPPRTNVLGIIPYATEPATSQVSRPKLPDGGGYYQPMQPQQQPPQMQQPQMQQPQMQQQQPPRVAPRPPAPKPKGYPPPPYQQYPSYSHPQNNAGLPPYSQTMGGYYPSGDELANQMSQLSVSQLGFNKLWGRDTVDLMKSRDVLPPTRVEAPPVRLSQEYYDSTKVSPEIFRCTLTKIPETKSLLDKSRLPLGVLIHPFKDLNQLSVIQCTVIVRCRACRTYINPFVFFVDSKHWKCNLCFRVNDLPEEFQYDPLTKTYGDPTRRPEIKSATIEFIAPSEYMVRPPQPAAYVFVLDVSRLAVESGYLRIFCDCLLSQLEALPGDSRTAVAFITYDSAVHYYSLADTQAQPHQMVVVDIDDMFVPCPENLLVNLSECLGLVRDLLRELPNKYRDSYDTGTAVGPALQAAYKLLAATGGRVTLVTSCLANSGPGKLPSREDPNQRSGEGLNQSHLNPVTDFYKKLALDCSGQQIAVDLFVLNSQFVDLASLSGVSRFSGGCIHHFPLFSVKNPHHVESFQRSLQRYLCRKIGFESVMRLRCTRGLSIHTFHGNFFVRSTDLLSLPNVNPDAGFGMQVSIDENLTDIQTVCFQAALLYTSSKGERRIRVHTLCLPIASNLSDVLHGADQQCIVGLLAKMAVDRCHQSSLSDAREAFVNVVADMLSAFRITQSGVSPTSLVAPISLSLLPLYVLALLKYIAFRVGQSTRLDDRVFAMCQMKSLPLSQLIQAIYPDLYPIANINELPLVTIGEDQVVQPPLLHLSAERIDSTGVYLMDDGTTIIIYVGHNINPSIAVSFFGVPSFSAINSNMFELPELNTPESKKLRGFISYLQNEKPVAPTVLIIRDDSQQRHLFVEKLIEDKTESGHSYYEFLQRVKVLVK

SEQ ID NO: 99 - protein, 1157 aa, Euschistus heros
MNPQYVSQPGGYGPHTGYGVPNSPPPPAASPLQPNAFPPQQSYPGIPSKTNMASPSAFSQPYRPAGTSPANMMPPPPLQGGPVPPQQHSGPMVPPQHGGPAPPQQQGGPMMSQHYGGPKPLQQQNSSMPPQQPGGPMPPQQQLSPMQPQKPVGSVPPQQSGQMPPQQHPGSLPHQHHGGPMPPQQRGAPMPNSFQPPSSNVPLGYNSAPSHPGAPPAVGAPSMPGPVSSPGPGGPPHPSMYPPGSQQIGRPPMPQVNQLAGQMSSMNINGQPMPPMNQQHQGDYPGGGLQGPPSMGYAPPGQPSRYPAVNGGPPSQAMNGLPDTSGPGRPPQSRYPPMPESGGQTSAYLPSPQQGYPPQPGFPPQPQPGYPGGVGQPQYPQQQPRRLDPDQMPSPMQVMEDDQKTRSGVFYTHQKGQVPPLVTTEFVVQDQGNASPRFMRSTMYNVPITSDLMKQASVPFGLVLSPMAEVADKEHPPPLVSFGELGPVRCIRCKAYMCPNMQFIDGGRRFHCLLCKATTDVPAEYFQHLDHTGQRVDRFERAELVLGTYDIIATREYCKDNQFPNPPALIFVIDVSYNNVKSGLVSLLCHQMKNILRNLPKERGMEKSTMKVGFITYNSAVHFYNIRPNLGQPQMLVVGDTQEMFMPLLDGFLVDPEESEGLIDALMTNIPMMFADTRETETILAPAIQAGLEALKASGCAGKLLVFHSSLPIAEAPGKLKNRDDRSLLGTDKEKTILLPQNTVYNTLGQDCVGAGVSVDLFITNNSYIDLATIGQVSRLTGGEIYKYTYFQAELDGERLVADVEKNIRRLCAFDAIMRVRTSTGIRPTDFYGHFYMSNTTDVELASIDPDKGIAVEIKHDDKLSEEEGVYIQVALLYTSLSGQRRIRVLNLVLKACSQMSDLYRTCELDTIINFFSKQSVFKLLDASAKAVKESLINRSAQILACYRKNCASPTSAGQLILPECMKLLPLYVNCLLKSDAISGGKDMTVDDKWFVMAAVLTMDVPSSLVYFYPRLYSLLELEDSVPPPCIRCSSEKMVDSGVYLLVNGIYMFIWLGLATPSDWVMSVFGVPSAAQVDTDRHRLPPLENPISERVRNAISSIADSNHRTMRLTIVRQRDKMEMVMKHFLVEDRGLDGSSSYVDFLCHLHKEIRNMLS

SEQ ID NO: 100 - DNA, 410 bp, Artificial Sequence
  DNA encoding YFP v2-1 hpRNA
atgtcatctggagcacttctctttcatgggaagattccttacgttgtggagatggaagggaatgttgatggccacacctttagcatacgtgggaaaggctacggagatgcctcagtgggaaagtccggcaacatgtttgacgtttgtttgacgttgtaagtctgatttttgactcttcttttttctccgtcacaatttctacttccaactaaaatgctaagaacatggttataactttttttttataacttaatatgtgatttggacccagcagatagagctcattactttcccactgaggcatctccgtagcctttcccacgtatgctaaaggtgtggccatcaacattcccttccatctccacaacgtaaggaatcttcccatgaaagagaagtgctccagatgacat

SEQ ID NO: 101 - DNA, 29 bp, Artificial Sequence
  Primer StPinIIFAM2 TAG
aagtctaggttgtttaaaggttaccgagc

SEQ ID NO: 102 - DNA, 4297 bp, Diabrotica virgifera
tctactccctgaaattcaagaatacgggccctggaataatagatataacgttaatatcatctgtgacatatccacatacttgtggaatagaagtatttctgcaataaaagcagaagcagaactccgaagagttggcaacattgtgccagccacgtaagattgacaatgacgtttgtgaaaatgattatttctgtccaaaaagattattcagaaaaaatgtacagtgcactaatttttaactgatatttttaataggaaattatttatttaatacataatttcaatgtcatcatggctgacagaaacgttaatggaatttcaccgaaccctgaaaccctaaaacacaatgctatatacgaggaaaaactacatcaacaatttaatggggtccattcatcacaatcatcaaggagttcatcacctggtacacgcctcggatatgtacccccttctcagctgcctccaagtaggcctatccctcaatctcaacttcctccttcccgatctgcgccgggaaatataactcaacaattcggggcattaaaccttaaccaaaatgctcccagacatagtccacaattcggagctcctgcaactcaacccactagttccagcccctacacaattcctccttttagtcaagtcagtaaggaaagtataaatagtcaatcatctgctatcttaccgccaacttcaaatacttcgagtacagtaacttcgtcgcaaatgtctacacctcttcaacaaggaccattcagtgctcaacctacaagtggttttcagaaacctgatccatttcaagcaattaaaccagcacaaaccaataatactcagccgacttctaatgtaaataatcaaccatcgcaaaatccaatgcaatttaatcagaactctcctaatgtcaggcttcaacctaaccaagtaccagtgcaaaataatatgggcgttccaactaattcaaacatgcctaggataagcccggttccacctcaacagaactttcaacctagtcctaatagatcagcttttggtccaataccaccgcctggaatacagaatccgatagttagtcaaattagtccaaacaggacaggtttagttcagggaccaccgttacaaacacaatacagagctcctaatcaaattcctgggccaccgccacaagctggtgtacttcaagcaaaccagcaaaggtcataccaagcatccccaattcaacaaaataataaccaaagatttaacaatgctattgctacccaaaatatcaataatggtccaactatgaacgcaaattttcctccacaagctgcaccttctaactacccacaaatgaatagtgcaccaccgccccaaacaaacgtggcaccgaaaacgaatgtacattcaaacaggtatcctacgatgcagtcaaacagctaccaacaacccgccccatctcaatatcagcaacagccaccttctggccagtatcagtatcaacaaccaatgcaacaaccagtacaacaaccaatgaattcgtatccaagtcaaaataatcagcagtctccttaccaaggagtagtaaatactggctttaataaattatggggtatggaacagtttgaccttcttcaaactccaaatatattgcaaccatcgaaagtcgaagctcctcaaattcgtttgggccaagacttgttggatcaagccaattgcagcccagacgtgtttcgttgcactatgacgaaaattccagaaaataattctcttttacagaagtcgagattgcctttaggggtgttaattcatccgtttagggatctttctcatttacctgtaattcagtgcagtgtaatagttaggtgtagagcgtgtcgcacctatataaatccctttgtcctttttgttgataataaacgctggaagtgcaatttgtgctatagaatcaacgagttacccgaagaatttcagtacgatccgatgacgaaaacgtacggagacccttctagaagaccagagattaaatccagcactttggaatacattgcacctgctgaatatatgttgaggccaccccagcctgcagtatacctttatttactggacgtatctcgattggcaatggaaagtggttatttgaatattgtatgtagtattttattggaagaattgaagaatttgcctggagatgcaagaacgcaaattggatttattgcttataactctgctctacatttttattctttgccagagggtatcacccaaccacacgagatgacaattctcgacatagacgatatattcctccctacacccgataatttattagtcaatttaaaggatagaatggacttaatagcagaccttttgaggctcttaccgaacagatttgccaacacatttgacaccaactctgctcttggtgctgcattgcaagttgcattcaagatgatgggtgcaacaggtggtagagttactgtattccaagcatcactgccaaacatcggacctggagcgcttatctcaagagaagatccatccaatagagcatcagccgaagttgcgcatctaaaccctgctaacgatttctataaacgcttggcgttggagtgcagcggtcagcagattgcagtcgatctgttcgtagtaaactctcagtatgtagatatagctactatttcaggaattagcagattcagcgggggttgtatgcatcacttccctttactcaaacctacaaagccagtagtctgtgatcgttttgctagatcttttaggaggtatatcaccaggaaaattggttttgaggccgtgatgagattgaggtgtacaagaggactttctattcataccttccacggtaatttcttcgttcgatcgacagatttactatctttgcctaacattaatcccgatgcagggtttggcatgcaagttgctatcgaagagagtttatccgatgttcagactgtatgtttccaggcagcattactatacacgtcgagcaaaggcgaaagaagaataagagttcatacgatgtgcttgccggtggctacgactatacaagacgtcatccactctgccgaccagcaatgcatcataggcttattgtcaaaaatggctgttgatagatcgatgcaatctagtctttcagatgcccgcgaggcgtttatcaacgtagcaatagatattctatcgagttttaaaatgagtctgaacatgggtagtcccgtaacgggtctgttagtgccgaattgtatgcgaatattgcctttgtatatatcagctcttcttaaacatttagcgtttagaacaggtagttctactaggttagatgacagagtaatgaaaatgatagagatgaaaacgaaaccattgtacatgctcatacaggatatataccccgatctgttccccatccataatttagaacaccaagaagtgatcatgaattctgaagaggaaccagtttctatgccacctaggttacaactcaccgccagatgtctggagaataaaggtgcgtttttgctggatacgggcgagcatatgatcatcctagtttgtccaaatgtgccacaagaatttttaaccgaagctctgggagtttcccaatatagcgccattccggatgatatgtatgaaatacccgtgttagataatcttagaaatcaaagacttcatcaatttattacatatttaaatgaggaaaagccgtatccggccacgttacaagtgattagagacaatagtacgaatagagttgtatttttcgagagattaatagaggaccgagtcgaagatgcactttcttatcacgaatttttgcaacatttaaaaactcaagtgaagtaaggttaagtgtacatttattatttttatctttttatttaaattgtgcagatttattgcttgtgcaaagaccactccgaaattatttccgtataaaataactaggtattttacagatccaggaacgtccaattatatgtttgtaacttcagagtatggtcaaaccacagccatataatacccaagactgcgcgctgtaatataaaaccgtgcagtccttacatcactttttaatgagcggggtttatcgaccacgtgacaatcccactagggattgtttagtagttagaaagagatgcaaggactgctcgcaatctgctttctctgtcgcattggggaaatggttttaaattacagcgtgtagtctaagtattatatgtctatgggtgaaacaatgtatccagtgacatgttccatttcaacttaaacttaacgactatattaaatttacagtcaagatgcagtg

SEQ ID NO: 103 - protein, 1180 aa, Diabrotica virgifera
MADRNVNGISPNPETLKHNAIYEEKLHQQFNGVHSSQSSRSSSPGTRLGYVPPSQLPPSRPIPQSQLPPSRSAPGNITQQFGALNLNQNAPRHSPQFGAPATQPTSSSPYTIPPFSQVSKESINSQSSAILPPTSNTSSTVTSSQMSTPLQQGPFSAQPTSGFQKPDPFQAIKPAQTNNTQPTSNVNNQPSQNPMQFNQNSPNVRLQPNQVPVQNNMGVPTNSNMPRISPVPPQQNFQPSPNRSAFGPIPPPGIQNPIVSQISPNRTGLVQGPPLQTQYRAPNQIPGPPPQAGVLQANQQRSYQASPIQQNNNQRFNNAIATQNINNGPTMNANFPPQAAPSNYPQMNSAPPPQTNVAPKTNVHSNRYPTMQSNSYQQPAPSQYQQQPPSGQYQYQQPMQQPVQQPMNSYPSQNNQQSPYQGVVNTGFNKLWGMEQFDLLQTPNILQPSKVEAPQIRLGQDLLDQANCSPDVFRCTMTKIPENNSLLQKSRLPLGVLIHPFRDLSHLPVIQCSVIVRCRACRTYINPFVLFVDNKRWKCNLCYRINELPEEFQYDPMTKTYGDPSRRPEIKSSTLEYIAPAEYMLRPPQPAVYLYLLDVSRLAMESGYLNIVCSILLEELKNLPGDARTQIGFIAYNSALHFYSLPEGITQPHEMTILDIDDIFLPTPDNLLVNLKDRMDLIADLLRLLPNRFANTFDTNSALGAALQVAFKMMGATGGRVTVFQASLPNIGPGALISREDPSNRASAEVAHLNPANDFYKRLALECSGQQIAVDLFVVNSQYVDIATISGISRFSGGCMHHFPLLKPTKPVVCDRFARSFRRYITRKIGFEAVMRLRCTRGLSIHTFHGNFFVRSTDLLSLPNINPDAGFGMQVAIEESLSDVQTVCFQAALLYTSSKGERRIRVHTMCLPVATTIQDVIHSADQQCIIGLLSKMAVDRSMQSSLSDAREAFINVAIDILSSFKMSLNMGSPVTGLLVPNCMRILPLYISALLKHLAFRTGSSTRLDDRVMKMIEMKTKPLYMLIQDIYPDLFPIHNLEHQEVIMNSEEEPVSMPPRLQLTARCLENKGAFLLDTGEHMIILVCPNVPQEFLTEALGVSQYSAIPDDMYEIPVLDNLRNQRLHQFITYLNEEKPYPATLQVIRDNSTNRVVFFERLIEDRVEDALSYHEFLQHLKTQVK

SEQ ID NO: 104 - DNA, 205 bp, Diabrotica virgifera
ctcagtatgtagatatagctactatttcaggaattagcagattcagcgggggttgtatgcatcacttccctttactcaaacctacaaagccagtagtctgtgatcgttttgctagatcttttaggaggtatatcaccaggaaaattggttttgaggccgtgatgagattgaggtgtacaagaggactttctattcataccttcca

SEQ ID NO: 105 - DNA, 43 bp, Artificial Sequence
  Primer Sec24B1_F
ttaatacgactcactatagggagactcagtatgtagatatagc

SEQ ID NO: 106 - DNA, 42 bp, Artificial Sequence
  Primer Sec24B1_R
ttaatacgactcactatagggagatggaaggtatgaatagaa

SEQ ID NO: 107 - DNA, 4488 bp, Diabrotica virgifera
gacacttgtctaagttccgaacttggtataattttcaggttatggtcattcaatgccaaaaaaaatatgatcacgtgtcacttatctgtcaacagtacgaatatttatttaacaatcatttatgatgaagaaataaaaaataaataattatttttgataaacttgcttctagaagatgattaaaatgctggaataatagatataacgttaatatcatctgtgacatatccacatacttgtggaatagaagtatttctgcaataaaagcagaagcagaactccgaagagttggcaacattgtgccagccacgtaagattgacaatgacgtttgtgaaaatgattatttctgtccaaaaagattattcagaaaaaatgtacagtgcactaatttttaactgatatttttaataggaaattatttatttaatacataatttcaatgtcatcatggctgacagaaacgttaatggaatttcaccgaaccctgaaaccctaaaacacaatgctatatacgaggaaaaactacatcaacaatttaatggggtccattcatcacaatcatcaaggagttcatcacctggtacacgcctcggatatgtacccccttctcagctgcctccaagtaggcctatccctcaatctcaacttcctccttcccgatctgcgccgggaaatataactcaacaattcggggcattaaaccttaaccaaaatgctcccagacatagtccacaattcggagctcctgcaactcaacccactagttccagcccctacacaattcctccttttagtcaagtcagtaaggaaagtataaatagtcaatcatctgctatcttaccgccaacttcaaatacttcgagtacagtaacttcgtcgcaaatgtctacacctcttcaacaaggaccattcagtgctcaacctacaagtggttttcagaaacctgatccatttcaagcaattaaaccagcacaaaccaataatactcagccgacttctaatgtaaataatcaaccatcgcaaaatccaatgcaatttaatcagaactctcctaatgtcaggcttcaacctaaccaagtaccagtgcaaaataatatgggcgttccaactaattcaaacatgcctaggataagcccggttccacctcaacagaactttcaacctagtcctaatagatcagcttttggtccaataccaccgcctggaatacagaatccgatagttagtcaaattagtccaaacaggacaggtttagttcagggaccaccgttacaaacacaatacagagctcctaatcaaattcctgggccaccgccacaagctggtgtacttcaagcaaaccagcaaaggtcataccaagcatccccaattcaacaaaataataaccaaagatttaacaatgctattgctacccaaaatatcaataatggtccaactatgaacgcaaattttcctccacaagctgcaccttctaactacccacaaatgaatagtgcaccaccgccccaaacaaacgtggcaccgaaaacgaatgtacattcaaacaggtatcctacgatgcagtcaaacagctaccaacaacccgccccatctcaatatcagcaacagccaccttctggccagtatcagtatcaacaaccaatgcaacaaccagtacaacaaccaatgaattcgtatccaagtcaaaataatcagcagtctccttaccaaggagtagtaaatactggctttaataaattatggggtatggaacagtttgaccttcttcaaactccaaatatattgcaaccatcgaaagtcgaagctcctcaaattcgtttgggccaagacttgttggatcaagccaattgcagcccagacgtgtttcgttgcactatgacgaaaattccagaaaataattctcttttacagaagtcgagattgcctttaggggtgttaattcatccgtttagggatctttctcatttacctgtaattcagtgcagtgtaatagttaggtgtagagcgtgtcgcacctatataaatccctttgtcctttttgttgataataaacgctggaagtgcaatttgtgctatagaatcaacgagttacccgaagaatttcagtacgatccgatgacgaaaacgtacggagacccttctagaagaccagagattaaatccagcactttggaatacattgcacctgctgaatatatgttgaggccaccccagcctgcagtatacctttatttactggacgtatctcgattggcaatggaaagtggttatttgaatattgtatgtagtattttattggaagaattgaagaatttgcctggagatgcaagaacgcaaattggatttattgcttataactctgctctacatttttattctttgccagagggtatcacccaaccacacgagatgacaattctcgacatagacgatatattcctccctacacccgataatttattagtcaatttaaaggatagaatggacttaatagcagaccttttgaggctcttaccgaacagatttgccaacacatttgacaccaactctgctcttggtgctgcattgcaagttgcattcaagatgatgggtgcaacaggtggtagagttactgtattccaagcatcactgccaaacatcggacctggagcgcttatctcaagagaagatccatccaatagagcatcagccgaagttgcgcatctaaaccctgctaacgatttctataaacgcttggcgttggagtgcagcggtcagcagattgcagtcgatctgttcgtagtaaactctcagtatgtagatatagctactatttcaggaattagcagattcagcgggggttgtatgcatcacttccctttactcaaacctacaaagccagtagtctgtgatcgttttgctagatcttttaggaggtatatcaccaggaaaattggttttgaggccgtgatgagattgaggtgtacaagaggactttctattcataccttccacggtaatttcttcgttcgatcgacagatttactatctttgcctaacattaatcccgatgcagggtttggcatgcaagttgctatcgaagagagtttatccgatgttcagactgtatgtttccaggcagcattactatacacgtcgagcaaaggcgaaagaagaataagagttcatacgatgtgcttgccggtggctacgactatacaagacgtcatccactctgccgaccagcaatgcatcataggcttattgtcaaaaatggctgttgatagatcgatgcaatctagtctttcagatgcccgcgaggcgtttatcaacgtagcaatagatattctatcgagttttaaaatgagtctgaacatgggtagtcccgtaacgggtctgttagtgccgaattgtatgcgaatattgcctttgtatatatcagctcttcttaaacatttagcgtttagaacaggtagttctactaggttagatgacagagtaatgaaaatgatagagatgaaaacgaaaccattgtacatgctcatacaggatatataccccgatctgttccccatccataatttagaacaccaagaagtgatcatgaattctgaagaggaaccagtttctatgccacctaggttacaactcaccgccagatgtctggagaataaaggtgcgtttttgctggatacgggcgagcatatgatcatcctagtttgtccaaatgtgccacaagaatttttaaccgaagctctgggagtttcccaatatagcgccattccggatgatatgtatgaaatacccgtgttagataatcttagaaatcaaagacttcatcaatttattacatatttaaatgaggaaaagccgtatccggccacgttacaagtgattagagacaatagtacgaatagagttgtatttttcgagagattaatagaggaccgagtcgaagatgcactttcttatcacgaatttttgcaacatttaaaaactcaagtgaagtaaggttaagtgtacatttattatttttatctttttatttaaattgtgcagatttattgcttgtgcaaagaccactccgaaattatttccgtataaaataactaggtattttacagatccaggaacgtccaattatatgtttgtaacttcagagtatggtcaaaccacagccatataatacccaagactgcgcgctgtaatataaaaccgtgcagtccttacatcactttttaatgagcggggtttatcgaccacgtgacaatcccactagggattgtttagtagttagaaagagatgcaaggactgctcgcaatctgctttctctgtcgcattggggaaatggttttaaattacagcgtgtagtctaagtattatatgtctatgggtgaaacaatgtatccagtgacatgttccatttcaacttaaacttaacgactatattaaatttacagtcaagatgcagtggaggtggacagaccaagacacgttaaatgctact

SEQ ID NO: 108 - protein, 1180 aa, Diabrotica virgifera; identical to SEQ ID NO: 103

SEQ ID NO: 109 - DNA, 444 bp, Diabrotica virgifera
gcttataactctgctctacatttttattctttgccagagggtatcacccaaccacacgagatgacaattctcgacatagacgatatattcctccctacacccgataatttattagtcaatttaaaggatagaatggacttaatagcagaccttttgaggctcttaccgaacagatttgccaacacatttgacaccaactctgctcttggtgctgcattgcaagttgcattcaagatgatgggtgcaacaggtggtagagttactgtattccaagcatcactgccaaacatcggacctggagcgcttatctcaagagaagatccatccaatagagcatcagccgaagttgcgcatctaaaccctgctaacgatttctataaacgcttggcgttggagtgcagcggtcagcagattgcagtcgatctgttcgtagtaaactctcag

SEQ ID NO: 110 - DNA, 53 bp, Artificial Sequence
  Primer Sec24B2_Reg3_F
ttaatacgactcactatagggagagcttataactctgctctacatttttattc

SEQ ID NO: 111 - DNA, 49 bp, Artificial Sequence
  Primer Sec24B2_Reg3_R
ttaatacgactcactatagggagactgagagtttactacgaacagatcg

SEQ ID NO: 112 - RNA, 3664 nt, Artificial Sequence
  Antisense Sec24B2 polynucleotide
cuacaguugaccuggagguuguaauaguuuacacggagguugcaauagaucacccggagguuguucaccaagaggaguuuguccgguaaauccacgaggugguuuaguuagagggaacagaccuccucaagguggaguuuacccuggauuaguuguuaauccugucggugguagccgucgaccaggugguucgguggaaccugucugaagaaacugauuggggggugggguagguccaguuggcuuagaggggaccgcggguggaguuagacauccaguuggaccaccgggaggaccuauaggagguaacgguccuguaguuccuguuggguguagugucaagcuguguguuccagguuacaguguuuuaccuggagguuuguacauaccuuuaggugguuuaguuaaauuauuagucuacccaggagguuuucacccuguuaaaggaguuguuguuuccgguuacguuggaggggauggaccugucggcggauacggcccuguuccaggaaauuagucacgagguccagguaugccuggaagaaguccuggucgugugguuuacgguggaguaguuccuguugguggaguaguuccuguuagugguauaccuggaccgguuuauugaucagucaacgucguuuacuuaaauagaccagguuucggccgaauaggucauggugguccgccaggguacucuggcuacuugccucugucgccaggcguauacggaggucguuacuugguuggcccuauauacuuauuaguugucccgucucaaggaggaccuggaccaauagguggcuacggccccguucguggcuacguuccuguuccuguguacggaccaguucccguuauggguccuggaccaccccccauaggcguuccguugaugguuguucgacgcggccgcguuguguucuaacuaggacuaguacacggcuuagguuaaguucaauaggcucuacuagucguucugucccugucgcaaaaacaaugauuaguuuuuccugaacauggcggauaccauugaugguuaaaauaacaaguucuaguuccuuuaacgucaggugcuaaguacucuagaugguauauauuacaagguuaaaguguccuaaacaauuuuguuagacgugaagguaagucagaaaauuauucagguuaccgguccguucaucucguucuuaugggaggugguuagcaauuaaagccuucggagccaggacagucuacguaggcaacguuccggauguacacaggcaaguacgucaagcagcuaagaccuuccuccaaggucacagacaaaacauugcguugaugacuacaagguugucuuauaaaggucguagaucuagucuggccggauucuuaccuggcgaaacuugcuggucuuaacuaggaaccauggaugcuuaagcagcgaugggggcuaaugacggcuuuguugcaagacggguuuggcggucggcaguaaaagcaauagcugcaaaguauauuguuguaauuuaggccuuaccaaaggaacaacacguuagucuacuuucucuaguaaguuuuagaaggccaccugguuccggugcuuuucucguuguacuuucaaccuaaauaaugcauauuaucaagccacguaaaaauauuauaguucccuucaaacugucgagguguuuacaaccaccauccucuacagguucuuuacaaguacggaaacaaccuaccaaagaauacaugaggucuucuuagcccugggcauuaucuagaugaguacguugucuaagggcguuacaaacgucuaugauuccuuuggcuucagcaaaacgaagggcguuaaguucgaccuaaucuucgggauuuccgaaggcuuucauguccguuugaagaucauaaggugaggugaaaugguuaucgucuccgagguccauuuaacuucuuggcgcugcuaucuuuucagaauccuuggcuauuucuuuuuugacagaacugugguguuuguguucguauguugguuaacccgguccuuacgcagucguugccaacgaggcaacuauacauauagaaguuauugcgaauguagcuauaucgcugauaaccaguucacagaucuaacugcccuccucuucacaaauucauaugaauaaagguccgacuauaacuaccucuugcaaaguauugucugcaauagaauuuauaaucagcugguuaucgcaaacuacgacauuacucccaaucuugcaguuguccucacuccgggugacugaaaauaccaguaaagauguacaguuuaugaugccuauagcuugaucgccgucaucuaacgcuauuucgguaucgucagcuuuauuuugugcugcuguuugacuuacuucugugcccccauaaguaaguuugccgcgacaauauguguagcacgaguccugucgcugccaacgcuuaauacuuagaaagugacuucugaacgaguguuuaccggcuagagaaaucuucaacacuaaaucuaugaaauuaguuaauguacucauuuguccgaugcauauuuaauaaccugccgucggggucgcaacauuuccucccugaacagguaucucggcgagucuagaaucguuauauguccuucgugacgcguucagguucaucgcgcccaguugauuaagaagggcuuacguacuucgacgauggcuagaugugguuaacagaagaguucuugcugcgauagaguccuccaagccuauacugguagcugcuguuuagcaagcaguacguccaccagaacucguaccuggaauugaagagccacaugauaaagauaggauccaauuaaggugaugugcuauagcuaggguugguccuaggauagugucaaggcuuaggauacuccacaucaauacuauuuuacuuacuugucccucacauauauaaucuuuugccuuagguauacaagaauaccaaaccagagccgcacuuaggguugaaauaagucguugagaaaccacgcggaagucguuauguucaacuauagcuauccucaucaaacggccuuaaucuauuggguaacagccaucgucaauccuguuauuaucugcuuuaguccuaugucuuuguauccacauacuccaauugggaccaaucuguuucucuuuuugaccuuggucagaaguucguaaagaaucaucuccuggcgccgugucugccaagucggucgauacagcugaaggauacaguauacgugucucuuuagucuuuguaggagucgaucgugucuuccacuagguuuccgucugccuucuauucuacuaucuuuuagaacuuuaaacaugagacuaggagcuauuguauaaaggagaacauauuucauaauaauucuagauaaaaacauaucgcguacgcaaacauuucccacggucugccacaagaaaaccuaaagaucuauaagauaauauaauacguaauaaaaccccagaucgaacagccacgaaaauguauaauuucuuuuagucaaacaaaggcauacgaguccuuuguuuguugcgaaaaaaaagauaaaauaaccaauaaugugcagcugucuugauagacuuuccagucuagcuuuugaaagcaaugcgcugcaacagucuaauuagcuucaaauuuccaaaaggccaaaaauaaacaauggacaaagugu

SEQ ID NO: 113 - RNA, 320 nt, Artificial Sequence
  Antisense Sec24B2 reg1 polynucleotide
auauagaaguuauugcgaauguagcuauaucgcugauaaccaguucacagaucuaacugcccuccucuucacaaauucauaugaauaaagguccgacuauaacuaccucuugcaaaguauugucugcaauagaauuuauaaucagcugguuaucgcaaacuacgacauuacucccaaucuugcaguuguccucacuccgggugacugaaaauaccaguaaagauguacaguuuaugaugccuauagcuugaucgccgucaucuaacgcuauuucgguaucgucagcuuuauuuugugcugcuguuugacuuacuucugug

SEQ ID NO: 114 - RNA, 418 nt, Artificial Sequence
  Antisense Sec24B2 reg2 polynucleotide
gauuccuuuggcuucagcaaaacgaagggcguuaaguucgaccuaaucuucgggauuuccgaaggcuuucauguccguuugaagaucauaaggugaggugaaaugguuaucgucuccgagguccauuuaacuucuuggcgcugcuaucuuuucagaauccuuggcuauuucuuuuuugacagaacugugguguuuguguucguauguugguuaacccgguccuuacgcagucguugccaacgaggcaacuauacauauagaaguuauugcgaauguagcuauaucgcugauaaccaguucacagaucuaacugcccuccucuucacaaauucauaugaauaaagguccgacuauaacuaccucuugcaaaguauugucugcaauagaauuuauaaucagcugguuaucgcaaacuacg

SEQ ID NO: 115 - RNA, 287 nt, Artificial Sequence
  Antisense Sec24B2 ver1 polynucleotide
agcaaaacgaagggcguuaaguucgaccuaaucuucgggauuuccgaaggcuuucauguccguuugaagaucauaaggugaggugaaaugguuaucgucuccgagguccauuuaacuucuuggcgcugcuaucuuuucagaauccuuggcuauuucuuuuuugacagaacugugguguuuguguucguauguugguuaacccgguccuuacgcagucguugccaacgaggcaacuauacauauagaaguuauugcgaauguagcuauaucgcugauaaccaguucac

SEQ ID NO: 116 - RNA, 128 nt, Artificial Sequence
  Antisense Sec24B2 ver2 polynucleotide
cagcaaaacgaagggcguuaaguucgaccuaaucuucgggauuuccgaaggcuuucauguccguuugaagaucauaaggugaggugaaaugguuaucgucuccgagguccauuuaacuucuuggcgcu

SEQ ID NO: 117 - RNA, 839 nt, Artificial Sequence
  Sec24B2 v1 hpRNA
ucguuuugcuucccgcaauucaagcuggauuagaagcccuaaaggcuuccgaaaguacaggcaaacuucuaguauuccacuccacuuuaccaauagcagaggcuccagguaaauugaagaaccgcgacgauagaaaagucuuaggaaccgauaaagaaaaaacugucuugacaccacaaacacaagcauacaaccaauugggccaggaaugcgucagcaacgguugcuccguugauauguauaucuucaauaacgcuuacaucgauauagcgacuauuggucaaguggaauccuugcgucauuuggugacuaguaccgguugggaaagguauguuucugcuucuaccuuugauauauauauaauaauuaucacuaauuaguaguaauauaguauuucaaguauuuuuuucaaaauaaaagaauguaguauauagcuauugcuuuucuguaguuuauaaguguguauauuuuaauuuauaacuuuucuaauauaugaccaaaacauggugaugugcagguugauccgcgguuaaguugugcgugaguccauugcacuugaccaauagucgcuauaucgauguaagcguuauugaagauauacauaucaacggagcaaccguugcugacgcauuccuggcccaauugguuguaugcuuguguuuguggugucaagacaguuuuuucuuuaucgguuccuaagacuuuucuaucgucgcgguucuucaauuuaccuggagccucugcuauugguaaaguggaguggaauacuagaaguuugccuguacuuucggaagccuuuagggcuucuaauccagcuugaauugcgggaagcaaaacga

SEQ ID NO: 118 - RNA, 521 nt, Artificial Sequence
  Sec24B2 v2 hpRNA
gucguuuugcuucccgcaauucaagcuggauuagaagcccuaaaggcuuccgaaaguacaggcaaacuucuaguauuccacuccacuuuaccaauagcagaggcuccagguaaauugaagaaccgcgagaauccuugcgucauuuggugacuaguaccgguugggaaagguauguuucugcuucuaccuuugauauauauauaauaauuaucacuaauuaguaguaauauaguauuucaaguauuuuuuucaaaauaaaagaauguaguauauagcuauugcuuuucuguaguuuauaaguguguauauuuuaauuuauaacuuuucuaauauaugaccaaaacauggugaugugcagguugauccgcgguuaaguugugcgugaguccauugucgcgguucuucaauuuaccuggagccucugcuauugguaaaguggaguggaauacuagaaguuugccuguacuuucggaagccuuuagggcuucuaauccagcuugaauugcgggaagcaaaacgac

SEQ ID NO: 119 - RNA, 4273 nt, Artificial Sequence
  antisense BSB_Gho polynucleotide
acguaaccucacuuucuugacagcuuccgccagacuguuuuucauuuaggcuaguuugccuucgcagucuuguuauauugauaaaaacuuucguuaagcuuaguuaaaauuaaagauacaacaaucucguaaguauuuacaacucgggcgaaguaaaaauguuacuguuucgcuguuugguuucaugugugcuauaaccaaagauuuaucuuaaggggaaaaacggugcuauuucaugcgucucgaagcuuaaacuaauuuaaacaaguaguuuuaauuuaaggaacaguugaguuuuauauauuaucuuuuaaaugguaccguuaaugcuuacacggagcgcaucguaguaacuugggaaaggggagugacauauaaguguaaccguccauauaucagacuucuauuuguaauuuaauuaaucauuugaaaguuuuuaagcugauucauguuuucaaauuaacuaaggagcccucaacuaccuuuuguaauuuugaauaaugaacggccaaucuugcacuuauucugacucuggaaaugguacaccaacaccuucauccacaagcuauccagcuaguuuaucaucacaaucuucccgugauacaucccccucccgccuucauccuaaccuuaaucauauaaauucugaaaaaucaauuaauucaucugguaacuauaugaauuauaaaauacacgauacguauacaaaugccaauucuguuuaugggcaaauauauucagacucaacuacaccuacuaacagggcaacaguucccccguacaucagugacacuaauaacgacauuaaucaaucucaaagacuggggcaaccgcagcuccgaccuucaacaacaucaucacaaauaauaacuaguuuagggucuucgguuucuaaaccugucuauaguucaucacauuuaaaucaaauaucgaaugaucagaaacaguauguuaaucaauauagcacacaaaaguuagauagcguuaugcagccuaaaacaucagagaguaacaucauuaaaaaucaugaaacuaugccuacaucuaauuuagcaauaucugauuauuaucagggauauacucaaacgaugaauaaucccuacaggcaagaaaauguauugccuaaccagacaaugaagcccgaacaacaguaccaugcucaaacccaaggguaucaaguucaaaaacccuugaugucuccaacaucaaauccauacaugaauucagugccucaagauaaccaaaacuacccccaaucaccaggugauguccccaggucuacuuuccagcaggguuauuaucagcaucaaccucaaccucaaccucaaccacaaccaccuucaguaaugaguggaagaccgcagaugaauuugccuuugacucagucuagaucacuugaugaaccuauuucuucagggccuccaagaacaaacgucuugggaaucauuccuuaugccacugaaccugcuacuucgcaaguuucgaggccuaaauuacccgaugguggaggguauuaucagcccaugcaaccacaacagcaaccaccgcagaugcagcagccacagaugcagcaaccgcagaugcagcagcaacagccaccacgaguggcaccaagacccccagcgccuaaaccuaaaggcuacccuccaccaccauaucaacaauauccaucuuauucccauccucaaaacaaugcugguuuaccuccuuacagucaaacaaugggugguuauuacccgagcggagaugaacuugcuaaucagaugucacagcuuagcguuucucaacuugguuuuaauaaauuauggggaagggauacaguggacuugaugaagagucgugauguuuugcccccuacucgggucgaagcuccuccaguucgucuuucucaggaguacuaugauucgacuaaaguuagcccugagauauuuagauguacgcuaacuaaaauacccgagaccaaaucucuucuugauaaaucuaggcuuccccuuggcgucuugauccacccauucaaggaccuaaaucaauugucggugauccagugcacaguaauaguacgauguagagcguguaggacuuauauaaauccuuuuguauucuuugucgacucgaagcauuggaaaugcaaucucugcuuuagggugaaugauuugccagaagaauuucaauaugacccauuaacaaagacuuauggagacccuacuagacgaccagaaauaaaaucugcuacuauagaauucauagcuccaucggaauauauggugaggccgccgcaaccggcugcuuacguguuuguauuagacgugucaagacuagcggucgagagugguuacuugcguaucuucugugacugccuccuuucccagcuggaggcguugccaggcgauucgaggacagcuguggcuuuuaucaccuacgacucugcuguccacuauuauagccuugcugauacccaggcucagccacaucagauggucguaguggacauugaugauauguucguaccaugcccugaaaaccugcuggugaaccugagugagugccuggggcuaguacgggaccuucugcgggaacugccuaauaaguauagagauuccuaugacacaggcacugccgucgguccugcuuuacaagcagcuuacaaauuauuggccgcaacugguggaagagugacuuugguaacgagcugcuuggcgaacagcggaccaggaaaacugccaucucgagaggacccgaaccagaggagcggggaaggguugaaccagucacaucucaacccagucacugacuucuacaagaaauuggcccucgauugcucaggccaacagauugcugucgaucuuuucguacuuaacagucaauuuguugaccuugcuucucugagugguguuucgagguuuuccgguggguguauccaucauuucccucuguucucugugaagaacccucaucauguugaaucauuccagcguagucuacagagguaucugugucguaagauugguuuugaaucugucaugagguugcgcugcaccaggggguuaucuauucauacauuccauggaaacuucuuuguucguucaacggaccuccucucucuacccaauguaaacccagaugcugguuucggaaugcaggugucuauugacgagaaccugacugauauacagaccguauguuuccaagcagcacuucuguauacuucgaguaaaggagaaagaagaauccguguucacacuuugugccuuccaauagcuucuaaccuuucagacguucugcauggagcagaccagcaauguaucguaggucuucuggcuaagauggcuguugauaggugucaucagucgucgcugagugaugcaagggaggcuuuugugaacguaguugcugauauguuaucagcguuccggaucacccagucuggcguaucaccuaccucacuagucgcucccauuagucucucccuucuuccacucuauguacucgcuuugcucaaauauauugcuuuccgugucggccagagcacaaggcuggacgaucgagucuucgcuaugugccaaaugaagucucuaccucucucucaguuaauacaggccauuuacccugaucucuauccaauagccaauaucaacgaauugccacuuguuacuauuggagaagaccaaguaguccaaccaccauuacuucaccucucagcugaaagaauagacucgacgggggucuacuugauggaugauggaacaacaauaauuaucuacgucggccacaacauuaauccaucaauugcuguuuccuucuucgggguaccuucauuuucagcuauaaauucuaauauguuugaacuaccugaacugaauacgccggagucuaaaaaacugagagguuucauuagcuauuuacagaaugagaagcccguagcuccgacuguacucaucauuagggaugacagccagcagagacauuuauuugucgagaagcucauagaagacaaaacugaauccggucauucuuacuacgaauuuuugcagagagugaagguacucguuaaguaacaaacagcugagauauucucacucuauaccaaucuaccaaagacuaugucguguguugauggggcauggcaacacaucuuauguccauuauagauuucuaacuuuuuuauauuuucugcuucuuauucgucguaaugagaaguuuuaauugauguuucaucaacuacaaaacuuuuauccuguauaacacaucauuuuauauaguauuauauauauaa

SEQ ID NO: 120 - RNA, 4809 nt, Artificial Sequence
  Antisense BSB_Gho polynucleotide
uaccuuauuuuaaaaauaaaugucuuuuauuaguaguuguaauagauguuuaaauaaaagauauuaaauauauauuauuguguaaugguuuguuuuuauuguauagcaucaauauuguuaacaaauauauauuuauguauguguacagugugguauguggcguauuggaagcuugagccgauguguucuagaauuccucgcguguuguauuuauguuguauuucguuucauaguuacauuuauucccuuugaauccauguucacagacaaguaccccuuguauauauagauauauacuauauuguuaauaaucacaauuuuuauuauaaauuaauuuuauuauaaaugaccguuguauauuauuuuuauaaacuaauguauuuaauggaucuauuucguugucgaacuauauuaggagcaauuuguauaugacgugcgucaaccaagaaaauauuacaugacauccuuuaaaacuauguauuuuuuuuuuuuuuuuauuaccuuucuucuucuuuucacgugaccaccguucaaauuaaacuguucaaccuucauaugcauaguaugcgguaaaaaauagaaaucuaucauucaugagucuacgugauaguuauugaaaacgauuauaaaaauuuuaaaaauaaaaaauucagguuaagugcaucuauauaaauacaugucaaauuauuuaaaggagggagacauuuuuuauuuuauuuuguuuuauauugguuacuauauuuguuuaaaacuauuaauuuaaauuuuguuauuauaauuaguguaggguguaaaauuuccuucaucuuucuuuuguuauguaauaaauacuauguuagggcaauauuauauguaguaguuuguuugucaacauucgaaugggcaauuuacucuuugacaaugaauuauuauuacuuaauauuguuaaaguagucgauauuuuuauaguuuagcuuuaaaguauguuaacuuccuauuacuauuuaaaauguccaagcuauccuuuacaguucgguuguuaaccgucagcauuagacguauuaucagacgacaccuccagcgauugauucguauaaugcuuaaagaaacacuucuacuguaucuuuuagauguauucuacuucuagguagguuuggagacaggagcugguucuucacgaaguagugguaaagguaaaacagggcaacagagugauaacagucggaguaacaggauacuaacgacagucguuaacuacucuaacguaaggacugagaaagacuuuagcccaaaaguuccccaccauuagauacagauagccauagcuggacucgacgugaaccuugagguuuaugacuguaguggguuagacuuccucaucgaucugggucggucuacuuguacauuuauggcaaaugaucauuuauaugaggugauaggugguaaaaaagucuacuuguagaauacgugccaccaccaugucuuaggagaucgagauuaucucuuauauuggcacccaucuucauauguucucuucuuccuuguagguagcagucuugacgucgguaguguuugguaaacagcaguugacaguacagaaacggaggucucuaucguagugaaaaguucuccgucaacuguaugucuccauuguugaaguacgugaguccuuccuagucgacaggucgucuucauccucuucguguuaagaaugcuaucgugcggucuuagacucgacuggacaaauaauuacuaagaaauugucgaaaacggcuacguagauuuucgaacuugugugagacaaaccuuuucuucaacuacuaucacagcucaaguguccaagauaucuccagccuguagacacucguucggaaguuaugguccaacucuugagacuaggaggcgacaggacugucgcuucauauguuguuacgcugaaccuauauauguggaagaagaagucuuucaaacaguaguacgaauuagagcugucgauaagggaacagaccuagauaucuccguucaaguuguagacaccauaagcuguacaucuuuacagguaucuuuagucagccagcuuaugggcaacuacaggauugagaguauuaucguaguuuucgcguuucggaggacuauaaaaagaguuguagucgauguucggagagagguagaucaagucggaccuucauacauaugaacauuuaaagagguggucacucggaacuuugaacuggcuaucaacgguccaguuauauccuuaacaaucauuauuuauuuaguugcgagugaggucgugguugugucaggacaggguuccacaacauuugucacaagacaccguuauuuuaacagaaaagaaauagucagggguuauugcuggacaguagggauaaaaaguugaaagguccucgaagacgcuauccuucucugcucaccuugugcucgucaaauggucgcgugggucugcgaaauucucgaaguuccggccggacauaucguccucggucauaacaaagacagagugcccacagucguuuguaguagccauauaagcaguaaucacgcagauaauuuggaagucugagaaguccuagcugguccuuuggcagguuaucuccguacuuguaaagaacucauaguggcugaugauuguagaccccaacaggauccaauccagauuauaacaucuuuaccugucgugacaauauucaauauuuagguugaaaguaucaucugaagagguaaggggaaagaaauccuucuaacgcuucuuauaaaaaguaaacuacuguauugucacuuugcucaggucuaaauuguaauaacauucuguguaguuauugcuuauauucacguccaccuaaucccuuaacuaacagaaauguuauaagagaacaacgauauuauaguauccagggauuguguucaagucgagaaaguuuugcuaguugagcaacuggucauaccagauuuacgaccuucauaagucgaccauguagucaucaacgaaacguaucuucugucaccuucgcggauggugguaguuacuugacguacaagcccguguauauucggaacguugcuuauguagaaugaccuggcucgagcgguuuucuuugguugccuccuccuacaagaaauagacgcugaaggcgguauccugaguugugguuugguuuuccaugucugcggacaaaguaguuuagucuucaauauccuugcaacauguagcaacuggaguauuuggaaccugaccguaacgggacuagaacuugcugcuuaaggcaucauuguucaccucccugaaccggaaagaccacacauauuuugugcggacuagaacagaagaccaguagaagguaauggacguaaccugauccguagaccagacccaguucggacgcuccaacgacaacuccuaugacgccgacggguugaggugguccuauugguccaacuccgacgccacccuuugguccaacucccccuaucgggacgacgccgcuaccuuccauacgucuucaaacaggaggacuaaguccguaaccuccuauagaccuaacuccuccggacgguccuggugaacauaguccuuccgguaaguagcggacugacccacccggugguaagugccgacccauagcucuaccaacagguccuccucguaugggguaucuaccgccuggaacguuugguggagguccuaucaggggaaccacaacgacuaaguaaccaccguagccgacggguaaauacaaguacgaccuguagacgggacggucgaccaaguggacuccguaaccaccggacggcuagacgacucuuggaccuccuauguaccuacccacaccaccuggugguccuggcccucucgacuguccugguccguagcuuccccgaggaugucguccaccacgaggacccacgcuuccacgacucaauauaggaucuccguguaaugaucuaccaccaacuuucgacaauccguauccucguggcgcgacaacuccuccguaaccuggugguaccacaaccacaccguuucuugguccuacgacaacuccaccguaaacuggugagacaacuccaccgugucuugguugaccaaaaacuccaacguaaccugauucgacaacaacuccaccguacccuggugguccgacaacuccuccguaucuugauaagacgacgacuuccccgaaaccuggugguaucacaacucuguaguaaccuggugggacgacaacuccuccccgaccuggcgguacgacaccuccuugguaaccuggugacacaacgacucccccguggccuggcgggacaucaccuccuccaccguaguacaaccguccccuucagggucgaccagccauuccaacucuuuuacgacuaccacuacgguauaaacaaaaucuuccuuauggaccuauugaaacgacaccaccuuuucguaauccaacuucucccgaacgucgaccaccgccuccgcuuaaaccuugugguauuggucauacuccagguauugguggaccaacacuauguaugacuccuaaguagaacauucagaacggaagugaauauaccuuagauuuugaauuauuagaaguauuaaaauuguuuuguuuuuuuuugugcuuugauuuauuauauucgaugauuauagucgacgucaucguggugaggugauggggacggugcauuccgucuugacguguccgcgucauucuaaugugcaguucuuuagaagucgcgauggggaacaccaccagauguuauguugauccaauaggauuaguuuuagucacgaugagaucacuuuugauuaaaguc

SEQ ID NO: 121 - RNA, 397 nt, Artificial Sequence
  Antisense BSB_Gho-1 polynucleotide
cuaagcugauuucaaucgggacucuauaaaucuacaugcgauugauuuuaugggcucugguuuagagaagaacuauuuagauccgaaggggaaccgcagaacuagguggguaaguuccuggauuuaguuaacagccacuaggucacgugucauuaucaugcuacaucucgcacauccugaauauauuuaggaaaacauaagaaacagcugagcuucguaaccuuuacguuagagacgaaaucccacuuacuaaacggucuucuuaaaguuauacuggguaauuguuucugaauaccucugggaugaucugcuggucuuuauuuuagacgaugauaucuuaaguaucgagguagccuuauauaccacuccggcggcguuggccgacgaaugcacaaac

SEQ ID NO: 122 - RNA, 494 nt, Artificial Sequence
  Antisense BSB_Gho-2 polynucleotide
gaaaaguucuccgucaacuguaugucuccauuguugaaguacgugaguccuuccuagucgacaggucgucuucauccucuucguguuaagaaugcuaucgugcggucuuagacucgacuggacaaauaauuacuaagaaauugucgaaaacggcuacguagauuuucgaacuugugugagacaaaccuuuucuucaacuacuaucacagcucaaguguccaagauaucuccagccuguagacacucguucggaaguuaugguccaacucuugagacuaggaggcgacaggacugucgcuucauauguuguuacgcugaaccuauauauguggaagaagaagucuuucaaacaguaguacgaauuagagcugucgauaagggaacagaccuagauaucuccguucaaguuguagacaccauaagcuguacaucuuuacagguaucuuuagucagccagcuuaugggcaacuacaggauugagaguauuaucguag

SEQ ID NO: 123 - RNA, 485 nt, Artificial Sequence
  Antisense BSB_Gho-3 polynucleotide
ccugaccguaacgggacuagaacuugcugcuuaaggcaucauuguucaccucccugaaccggaaagaccacacauauuuugugcggacuagaacagaagaccaguagaagguaauggacguaaccugauccguagaccagacccaguucggacgcuccaacgacaacuccuaugacgccgacggguugaggugguccuauugguccaacuccgacgccacccuuugguccaacucccccuaucgggacgacgccgcuaccuuccauacgucuucaaacaggaggacuaaguccguaaccuccuauagaccuaacuccuccggacgguccuggugaacauaguccuuccgguaaguagcggacugacccacccggugguaagugccgacccauagcucuaccaacagguccuccucguaugggguaucuaccgccuggaacguuugguggagguccuaucaggggaaccacaacgacuaaguaacc

SEQ ID NO: 124 - RNA, 4297 nt, Artificial Sequence
  Antisense Sec24B1 polynucleotide
agaugagggacuuuaaguucuuaugcccgggaccuuauuaucuauauugcaauuauaguagacacuguauagguguaugaacaccuuaucuucauaaagacguuauuuucgucuucgucuugaggcuucucaaccguuguaacacggucggugcauucuaacuguuacugcaaacacuuuuacuaauaaagacagguuuuucuaauaagucuuuuuuacaugucacgugauuaaaaauugacuauaaaaauuauccuuuaauaaauaaauuauguauuaaaguuacaguaguaccgacugucuuugcaauuaccuuaaaguggcuugggacuuugggauuuuguguuacgauauaugcuccuuuuugauguaguuguuaaauuaccccagguaaguaguguuaguaguuccucaaguaguggaccaugugcggagccuauacaugggggaagagucgacggagguucauccggauagggaguuagaguugaaggaggaagggcuagacgcggcccuuuauauugaguuguuaagccccguaauuuggaauugguuuuacgagggucuguaucagguguuaagccucgaggacguugaguugggugaucaaggucggggauguguuaaggaggaaaaucaguucagucauuccuuucauauuuaucaguuaguagacgauagaauggcgguugaaguuuaugaagcucaugucauugaagcagcguuuacagauguggagaaguuguuccugguaagucacgaguuggauguucaccaaaagucuuuggacuagguaaaguucguuaauuuggucguguuugguuauuaugagucggcugaagauuacauuuauuaguugguagcguuuuagguuacguuaaauuagucuugagaggauuacaguccgaaguuggauugguucauggucacguuuuauuauacccgcaagguugauuaaguuuguacggauccuauucgggccaagguggaguugucuugaaaguuggaucaggauuaucuagucgaaaaccagguuaugguggcggaccuuaugucuuaggcuaucaaucaguuuaaucagguuuguccuguccaaaucaagucccugguggcaauguuuguguuaugucucgaggauuaguuuaaggacccgguggcgguguucgaccacaugaaguucguuuggucguuuccaguaugguucguagggguuaaguuguuuuauuauugguuucuaaauuguuacgauaacgauggguuuuauaguuauuaccagguugauacuugcguuuaaaaggagguguucgacguggaagauugauggguguuuacuuaucacgugguggcgggguuuguuugcaccguggcuuuugcuuacauguaaguuuguccauaggaugcuacgucaguuugucgaugguuguugggcgggguagaguuauagucguugucgguggaagaccggucauagucauaguuguugguuacguuguuggucauguuguugguuacuuaagcauagguucaguuuuauuagucgucagaggaaugguuccucaucauuuaugaccgaaauuauuuaauaccccauaccuugucaaacuggaagaaguuugagguuuauauaacguugguagcuuucagcuucgaggaguuuaagcaaacccgguucugaacaaccuaguucgguuaacgucgggucugcacaaagcaacgugauacugcuuuuaaggucuuuuauuaagagaaaaugucuucagcucuaacggaaauccccacaauuaaguaggcaaaucccuagaaagaguaaauggacauuaagucacgucacauuaucaauccacaucucgcacagcguggauauauuuagggaaacaggaaaaacaacuauuauuugcgaccuucacguuaaacacgauaucuuaguugcucaaugggcuucuuaaagucaugcuaggcuacugcuuuugcaugccucugggaagaucuucuggucucuaauuuaggucgugaaaccuuauguaacguggacgacuuauauacaacuccgguggggucggacgucauauggaaauaaaugaccugcauagagcuaaccguuaccuuucaccaauaaacuuauaacauacaucauaaaauaaccuucuuaacuucuuaaacggaccucuacguucuugcguuuaaccuaaauaacgaauauugagacgagauguaaaaauaagaaacggucucccauaguggguuggugugcucuacuguuaagagcuguaucugcuauauaaggagggaugugggcuauuaaauaaucaguuaaauuuccuaucuuaccugaauuaucgucuggaaaacuccgagaauggcuugucuaaacgguuguguaaacugugguugagacgagaaccacgacguaacguucaacguaaguucuacuacccacguuguccaccaucucaaugacauaagguucguagugacgguuuguagccuggaccucgcgaauagaguucucuucuagguagguuaucucguagucggcuucaacgcguagauuugggacgauugcuaaagauauuugcgaaccgcaaccucacgucgccagucgucuaacgucagcuagacaagcaucauuugagagucauacaucuauaucgaugauaaaguccuuaaucgucuaagucgcccccaacauacguagugaagggaaaugaguuuggauguuucggucaucagacacuagcaaaacgaucuagaaaauccuccauauagugguccuuuuaaccaaaacuccggcacuacucuaacuccacauguucuccugaaagauaaguauggaaggugccauuaaagaagcaagcuagcugucuaaaugauagaaacggauuguaauuagggcuacgucccaaaccguacguucaacgauagcuucucucaaauaggcuacaagucugacauacaaagguccgucguaaugauaugugcagcucguuuccgcuuucuucuuauucucaaguaugcuacacgaacggccaccgaugcugauauguucugcaguaggugagacggcuggucguuacguaguauccgaauaacaguuuuuaccgacaacuaucuagcuacguuagaucagaaagucuacgggcgcuccgcaaauaguugcaucguuaucuauaagauagcucaaaauuuuacucagacuuguacccaucagggcauugcccagacaaucacggcuuaacauacgcuuauaacggaaacauauauagucgagaagaauuuguaaaucgcaaaucuuguccaucaagaugauccaaucuacugucucauuacuuuuacuaucucuacuuuugcuuugguaacauguacgaguauguccuauauauggggcuagacaagggguagguauuaaaucuugugguucuucacuaguacuuaagacuucuccuuggucaaagauacgguggauccaauguugaguggcggucuacagaccucuuauuuccacgcaaaaacgaccuaugcccgcucguauacuaguaggaucaaacagguuuacacgguguucuuaaaaauuggcuucgagacccucaaaggguuauaucgcgguaaggccuacuauacauacuuuaugggcacaaucuauuagaaucuuuaguuucugaaguaguuaaauaauguauaaauuuacuccuuuucggcauaggccggugcaauguucacuaaucucuguuaucaugcuuaucucaacauaaaaagcucucuaauuaucuccuggcucagcuucuacgugaaagaauagugcuuaaaaacguuguaaauuuuugaguucacuucauuccaauucacauguaaauaauaaaaauagaaaaauaaauuuaacacgucuaaauaacgaacacguuucuggugaggcuuuaauaaaggcauauuuuauugauccauaaaaugucuagguccuugcagguuaauauacaaacauugaagucucauaccaguuuggugucgguauauuauggguucugacgcgcgacauuauauuuuggcacgucaggaauguagugaaaaauuacucgccccaaauagcuggugcacuguuagggugaucccuaacaaaucaucaaucuuucucuacguuccugacgagcguuagacgaaagagacagcguaaccccuuuaccaaaauuuaaugucgcacaucagauucauaauauacagauacccacuuuguuacauaggucacuguacaagguaaaguugaauuugaauugcugauauaauuuaaaugucaguucuacgucac

SEQ ID NO: 125 - RNA, 205 nt, Artificial Sequence
  Antisense Sec24B1 reg1 polynucleotide
gagucauacaucuauaucgaugauaaaguccuuaaucgucuaagucgcccccaacauacguagugaagggaaaugaguuuggauguuucggucaucagacacuagcaaaacgaucuagaaaauccuccauauagugguccuuuuaaccaaaacuccggcacuacucuaacuccacauguucuccugaaagauaaguauggaaggu

SEQ ID NO: 126 - RNA, 4488 nt, Artificial Sequence
  Antisense Sec24B2 polynucleotide
cugugaacagauucaaggcuugaaccauauuaaaaguccaauaccaguaaguuacgguuuuuuuuauacuagugcacagugaauagacaguugucaugcuuauaaauaaauuguuaguaaauacuacuucuuuauuuuuuauuuauuaauaaaaacuauuugaacgaagaucuucuacuaauuuuacgaccuuauuaucuauauugcaauuauaguagacacuguauagguguaugaacaccuuaucuucauaaagacguuauuuucgucuucgucuugaggcuucucaaccguuguaacacggucggugcauucuaacuguuacugcaaacacuuuuacuaauaaagacagguuuuucuaauaagucuuuuuuacaugucacgugauuaaaaauugacuauaaaaauuauccuuuaauaaauaaauuauguauuaaaguuacaguaguaccgacugucuuugcaauuaccuuaaaguggcuugggacuuugggauuuuguguuacgauauaugcuccuuuuugauguaguuguuaaauuaccccagguaaguaguguuaguaguuccucaaguaguggaccaugugcggagccuauacaugggggaagagucgacggagguucauccggauagggaguuagaguugaaggaggaagggcuagacgcggcccuuuauauugaguuguuaagccccguaauuuggaauugguuuuacgagggucuguaucagguguuaagccucgaggacguugaguugggugaucaaggucggggauguguuaaggaggaaaaucaguucagucauuccuuucauauuuaucaguuaguagacgauagaauggcgguugaaguuuaugaagcucaugucauugaagcagcguuuacagauguggagaaguuguuccugguaagucacgaguuggauguucaccaaaagucuuuggacuagguaaaguucguuaauuuggucguguuugguuauuaugagucggcugaagauuacauuuauuaguugguagcguuuuagguuacguuaaauuagucuugagaggauuacaguccgaaguuggauugguucauggucacguuuuauuauacccgcaagguugauuaaguuuguacggauccuauucgggccaagguggaguugucuugaaaguuggaucaggauuaucuagucgaaaaccagguuaugguggcggaccuuaugucuuaggcuaucaaucaguuuaaucagguuuguccuguccaaaucaagucccugguggcaauguuuguguuaugucucgaggauuaguuuaaggacccgguggcgguguucgaccacaugaaguucguuuggucguuuccaguaugguucguagggguuaaguuguuuuauuauugguuucuaaauuguuacgauaacgauggguuuuauaguuauuaccagguugauacuugcguuuaaaaggagguguucgacguggaagauugauggguguuuacuuaucacgugguggcgggguuuguuugcaccguggcuuuugcuuacauguaaguuuguccauaggaugcuacgucaguuugucgaugguuguugggcgggguagaguuauagucguugucgguggaagaccggucauagucauaguuguugguuacguuguuggucauguuguugguuacuuaagcauagguucaguuuuauuagucgucagaggaaugguuccucaucauuuaugaccgaaauuauuuaauaccccauaccuugucaaacuggaagaaguuugagguuuauauaacguugguagcuuucagcuucgaggaguuuaagcaaacccgguucugaacaaccuaguucgguuaacgucgggucugcacaaagcaacgugauacugcuuuuaaggucuuuuauuaagagaaaaugucuucagcucuaacggaaauccccacaauuaaguaggcaaaucccuagaaagaguaaauggacauuaagucacgucacauuaucaauccacaucucgcacagcguggauauauuuagggaaacaggaaaaacaacuauuauuugcgaccuucacguuaaacacgauaucuuaguugcucaaugggcuucuuaaagucaugcuaggcuacugcuuuugcaugccucugggaagaucuucuggucucuaauuuaggucgugaaaccuuauguaacguggacgacuuauauacaacuccgguggggucggacgucauauggaaauaaaugaccugcauagagcuaaccguuaccuuucaccaauaaacuuauaacauacaucauaaaauaaccuucuuaacuucuuaaacggaccucuacguucuugcguuuaaccuaaauaacgaauauugagacgagauguaaaaauaagaaacggucucccauaguggguuggugugcucuacuguuaagagcuguaucugcuauauaaggagggaugugggcuauuaaauaaucaguuaaauuuccuaucuuaccugaauuaucgucuggaaaacuccgagaauggcuugucuaaacgguuguguaaacugugguugagacgagaaccacgacguaacguucaacguaaguucuacuacccacguuguccaccaucucaaugacauaagguucguagugacgguuuguagccuggaccucgcgaauagaguucucuucuagguagguuaucucguagucggcuucaacgcguagauuugggacgauugcuaaagauauuugcgaaccgcaaccucacgucgccagucgucuaacgucagcuagacaagcaucauuugagagucauacaucuauaucgaugauaaaguccuuaaucgucuaagucgcccccaacauacguagugaagggaaaugaguuuggauguuucggucaucagacacuagcaaaacgaucuagaaaauccuccauauagugguccuuuuaaccaaaacuccggcacuacucuaacuccacauguucuccugaaagauaaguauggaaggugccauuaaagaagcaagcuagcugucuaaaugauagaaacggauuguaauuagggcuacgucccaaaccguacguucaacgauagcuucucucaaauaggcuacaagucugacauacaaagguccgucguaaugauaugugcagcucguuuccgcuuucuucuuauucucaaguaugcuacacgaacggccaccgaugcugauauguucugcaguaggugagacggcuggucguuacguaguauccgaauaacaguuuuuaccgacaacuaucuagcuacguuagaucagaaagucuacgggcgcuccgcaaauaguugcaucguuaucuauaagauagcucaaaauuuuacucagacuuguacccaucagggcauugcccagacaaucacggcuuaacauacgcuuauaacggaaacauauauagucgagaagaauuuguaaaucgcaaaucuuguccaucaagaugauccaaucuacugucucauuacuuuuacuaucucuacuuuugcuuugguaacauguacgaguauguccuauauauggggcuagacaagggguagguauuaaaucuugugguucuucacuaguacuuaagacuucuccuuggucaaagauacgguggauccaauguugaguggcggucuacagaccucuuauuuccacgcaaaaacgaccuaugcccgcucguauacuaguaggaucaaacagguuuacacgguguucuuaaaaauuggcuucgagacccucaaaggguuauaucgcgguaaggccuacuauacauacuuuaugggcacaaucuauuagaaucuuuaguuucugaaguaguuaaauaauguauaaauuuacuccuuuucggcauaggccggugcaauguucacuaaucucuguuaucaugcuuaucucaacauaaaaagcucucuaauuaucuccuggcucagcuucuacgugaaagaauagugcuuaaaaacguuguaaauuuuugaguucacuucauuccaauucacauguaaauaauaaaaauagaaaaauaaauuuaacacgucuaaauaacgaacacguuucuggugaggcuuuaauaaaggcauauuuuauugauccauaaaaugucuagguccuugcagguuaauauacaaacauugaagucucauaccaguuuggugucgguauauuauggguucugacgcgcgacauuauauuuuggcacgucaggaauguagugaaaaauuacucgccccaaauagcuggugcacuguuagggugaucccuaacaaaucaucaaucuuucucuacguuccugacgagcguuagacgaaagagacagcguaaccccuuuaccaaaauuuaaugucgcacaucagauucauaauauacagauacccacuuuguuacauaggucacuguacaagguaaaguugaauuugaauugcugauauaauuuaaaugucaguucuacgucaccuccaccugucugguucugugcaauuuacgauga

SEQ ID NO: 127 - RNA, 444 nt, Artificial Sequence
  Antisense Sec24B2 reg3 polynucleotide
cgaauauugagacgagauguaaaaauaagaaacggucucccauaguggguuggugugcucuacuguuaagagcuguaucugcuauauaaggagggaugugggcuauuaaauaaucaguuaaauuuccuaucuuaccugaauuaucgucuggaaaacuccgagaauggcuugucuaaacgguuguguaaacugugguugagacgagaaccacgacguaacguucaacguaaguucuacuacccacguuguccaccaucucaaugacauaagguucguagugacgguuuguagccuggaccucgcgaauagaguucucuucuagguagguuaucucguagucggcuucaacgcguagauuugggacgauugcuaaagauauuugcgaaccgcaaccucacgucgccagucgucuaacgucagcuagacaagcaucauuugagaguc